(19) United States
(12) Patent Application Publication
(10) Pub. No.: US 2010/0009950 A1
(43) Pub. Date: Jan. 14, 2010

(54) SUBSTITUTED ETHANOLAMINES

(75) Inventors: Thomas G. Gant, Carlsbad, CA (US); Sepehr Sarshar, Cardiff by the Sea, CA (US)

Correspondence Address: GLOBAL PATENT GROUP - APX, 10411 Clayton Road, Suite 304, ST. LOUIS, MO, 63131, US

(73) Assignee: AUSPEX PHARMACEUTICALS, INC., Vista, CA (US)

(21) Appl. No.: 12/494,002

(22) Filed: Jun. 29, 2009

Related U.S. Application Data
(60) Provisional application No. 61/076,903, filed on Jun. 30, 2008.

(51) Int. Cl. A61K 31/135; C07C 217/44; A61P 11/00; A61P 11/06; A61P 11/08; A61K 31/567

(52) U.S. Cl. 514/171; 564/346; 514/651

(57) ABSTRACT

The present invention relates to new substituted ethanolamine adrenergic receptor modulators, pharmaceutical compositions thereof, and methods of use thereof.

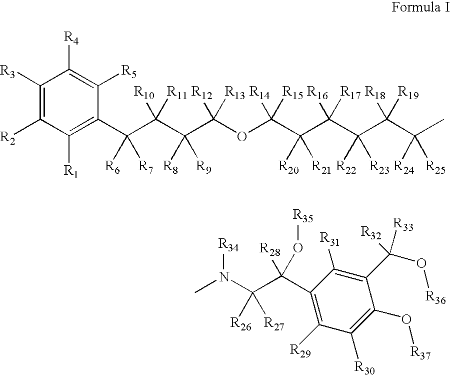

This application claims the benefit of priority of U.S. provisional application No. 61/076,903, filed Jun. 30, 2008, the disclosure of which is hereby incorporated by reference as if written herein in its entirety.

### FIELD

Disclosed herein are new substituted ethanolamine compounds, pharmaceutical compositions made thereof, and methods to modulate adrenergic receptor activity with such pharmaceuticals for the treatment of disorders, such as asthma, chronic obstructive pulmonary disease (COPD), respiratory syncytial virus (RSV), pseudomonas aeruginosa, pneumoconiosis, exercise-induced bronchospasm, chronic bronchitis, any disorder ameliorated by administering a bronchodilator and/or any disorder ameliorated by the modulation of adrenergic receptors.

### BACKGROUND

Salmeterol (Servent Diskus®, a component of Advair Diskus®), 2-(hydroxymethyl)-4-[1-hydroxy-2-[6-(4-phenylbutoxy)hexylamino]ethyl]-phenol, is a long-acting adrenergic receptor agonist. Salmeterol is administered as the xinafoate salt of the racemic mixture of the two optical isomers, (R)- and (S)-salmeterol, by a metered-dose inhaler. The agonistic activity of (R)-salmeterol is 40 times greater than that of (S)-salmeterol (Johnson M, Med Res Rev 1995, 15, 225-57). Salmeterol is commonly prescribed to treat asthma (Wolfe et al., Annals of Allergy, Asthma, & Immunology 2000, 84(3), 334-340; Moore et al., Chest 1998, 113, 1095-1108; Adkins et al., Drugs 1997, 54, 331-354). In addition to asthma, salmeterol can effectively treat the following: COPD (Stockley et al., Respir Res 2006, 7, 147; Boyd et al., Eur Respir J. 1997, 10(4), 815-21), RSV (Singam et al., Virology J 2006, 3, 32), pseudomonas aerginosa (Dowling et al., Am J of Respir and Critical Care Med 1997, 155(1), 327-36), pneumoconiosis (Igarashi et al., Japanese Journal of Occupational Medicine and Traumatology 2006, 54(4), 156-59), exercise-induced bronchospasm (Weiler et al., Ann Allergy Asthma Immunol 2005, 94(1), 65-72), and chronic bronchitis (Bennett et al., Pulm Pharmacol Ther 2006, 19(2), 96-100).

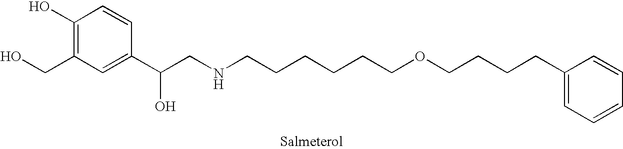

Salmeterol is a long-acting beta₂-adrenergic agonist. Salmeterol is selective for beta₂-adrenoceptors compared with isoproterenol, which has approximately equal agonist activity on beta₁- and beta₂-adrenoceptors. Salmeterol is at least 50 times more selective for beta₂-adrenoceptors than albuterol. The pharmacologic effects of beta₂-adrenoceptor agonist drugs, including salmeterol, are at least in part attributable to stimulation of intracellular adenyl cyclase, the enzyme that catalyzes the conversion of adenosine triphosphate (ATP) to cyclic-3′,5′-adenosine monophosphate (cyclic AMP). The resulting increased level of cyclic AMP (cAMP) inhibits the release of cytokines, hydrolytic enzymes and other proinflammatory mediators, and causes the relaxation of bronchial smooth muscles (Sekut et al., Clin Exp Immunol 1995, 99, 461-466). Salmeterol is a potent and long-lasting inhibitor of the release of mast cell mediators such as histamine, leukotrienes, and prostaglandin D₂ from human lung tissue. Salmeterol inhibits histamine-induced plasma protein extravasation and inhibits platelet-activating factor-induced eosinophil accumulation in the lungs of guinea pigs when administered by the inhaled route. In humans, single doses of salmeterol administered via inhalation aerosol attenuate allergen-induced bronchial hyper-responsiveness.

### Deuterium Kinetic Isotope Effect

In order to eliminate foreign substances such as therapeutic agents, organisms express various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C-¹H bond. If a C-¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C-¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects.

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O . Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

Salmeterol is a substituted ethanolamine-based adrenergic receptor modulator. The carbon-hydrogen bonds of salmeterol contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Kinetic Isotope Effect (KIE) that could affect the pharmacokinetic, pharmacologic and/or toxicologic profiles of such adrenergic receptor modulators in comparison with compounds having naturally occurring levels of deuterium.

Based on discoveries made in our laboratory, as well as considering the KIE literature, salmeterol is likely metabolized in humans at the methylene carbons located between the phenyl group and the secondary amine group. The current approach has the potential to prevent or impede oxidation at these sites. Other sites on the molecule may also undergo transformations leading to metabolites with as-yet-unknown pharmacology/toxicology. Limiting the production of such metabolites has the potential to decrease the danger of the administration of salmeterol and may even allow increased dosage and concomitant increased efficacy. All of these transformations, among other potential transformations, can and do occur through polymorphically-expressed enzymes. Such polymorphisms may account for the wide variance seen in interpatient phramacodynamic responses. In addition, salmeterol has well documented adverse side effects. As the salmeterol concentration in blood plasma increases, there is a likewise increase in the severity of the side effects. Further, it is quite typical for diseases ameliorated by the present invention, such as asthma, to produce symptoms that are best medicated around the clock for extended periods of time. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of salmeterol and attenuate interpatient variability.

Novel compounds and pharmaceutical compositions, certain of which have been found to modulate adrenergic receptors have been discovered, together with methods of synthesizing and using the compounds, including methods for the treatment of adrenergic receptor-mediated disorders in a subject by administering the compounds.

In certain embodiments of the present invention, compounds have structural Formula I:

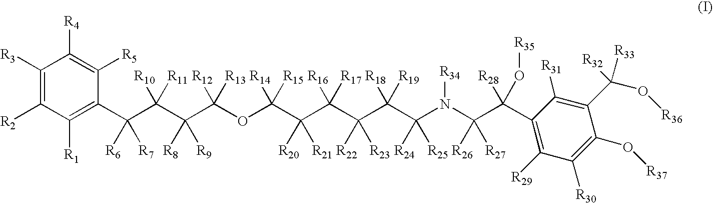

or a salt, solvate, or prodrug thereof, wherein:
R₁-R₃₇ are independently selected from the group consisting of hydrogen and deuterium; and
at least one of R₁-R₃₇ is deuterium.

Certain compounds disclosed herein may possess useful adrenergic receptor modulating activity, and may be used in the treatment or prophylaxis of a disorder in which adrenergic receptors play an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for modulating adrenergic receptors. Other embodiments provide methods for treating an adrenergic receptor-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disorder ameliorated by the modulation of adrenergic receptors.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions. In another aspect are processes for preparing a compound as disclosed herein or other pharmaceutically acceptable derivative thereof such as a salt, solvate, or prodrug, as an adrenergic receptor modulator.

In certain embodiments, if R₃₂, R₃₃, and R₂₈ are deuterium then at least one of R₁-R₂₇, R₂₉-R₃₁, or R₃₄-R₃₇ is deuterium.

In other embodiments, at least at least one of R₁-R₃₇ independently has deuterium enrichment of no less than about 10%, 50%, 90%, or 98%.

In other embodiments, a pharmaceutical composition comprises a compound disclosed herein together with a pharmaceutically acceptable carrier.

In certain embodiments of the present invention a method of treating a subject suffering from an adrenergic receptor-mediated disorder comprises the administration of a therapeutically effective amount of a compound as disclosed herein.

In other embodiments said adrenergic receptor-mediated disorder is selected from the group consisting of asthma, chronic obstructive pulmonary disease, respiratory syncytial virus, pseudomonas aeruginosa, pneumoconiosis, exercise-induced bronchospasm, chronic bronchitis, and disorders associated with bronchoconstriction.

In yet other embodiments, said method further comprises the administration of an additional therapeautic agent.

In further embodiments said therapeutic agent is selected from the group consisting of: adrenergics, anti-cholinergics, mast cell stabilizers, xanthines, leukotriene antagonists, glucocorticoids, decongestants, anti-tussives, mucolytics, anti-histamines, sepsis treatments, antibacterial agents, antifungal agents, anticoagulants, thrombolytics, non-steroidal anti-inflammatory agents, antiplatelet agents, norepinephrine reuptake inhibitors (NRIs), dopamine reuptake inhibitors (DARIs), serotonin-norepinephrine reuptake inhibitors (SNRIs), sedatives, norepinephrine-dopamine reuptake inhibitor (NDRIs), serotonin-norepinephrine-dopamine-reuptake-inhibitors (SNDRIs), monoamine oxidase inhibitors, hypothalamic phospholipids, ECE inhibitors, opioids, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIIa Inhibitors and Factor Xa Inhibitors, renin inhibitors, NEP inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-muscarinic agents, beta-muscarinic agents, antiarrhythmic agents, diuretics, thrombolytic agents, anti-diabetic agents, mineralocorticoid receptor antagonists, growth hormone secretagogues, aP2 inhibitors, phosphodiesterase inhibitors, protein tyrosine kinase inhibitors, antiinflammatories, antiproliferatives, chemotherapeutic agents, immunosuppressants, anticancer agents and cytotoxic agents, antimetabolites, antibiotics, farnesyl-protein transferase inhibitors, hormonal agents, microtubule-disruptor agents, microtubule-stablizing agents, plant-derived products, epipodophyllotoxins, taxanes, topoisomerase inhibitors, prenyl-protein transferase inhibitors, cyclosporins, cytotoxic drugs, TNF-alpha inhibitors, anti-TNF antibodies and soluble TNF receptors, cyclooxygenase-2 (COX-2) inhibitors, and miscellaneous agents.

In other embodiments said therapeutic agent is selected from the group consisting of: adrenergics, anti-cholinergics, mast cell stabilizers, xanthines, leukotriene antagonists, glucocorticoids, decongestants, anti-tussives, mucolytics, and anti-histamines.

In yet further embodiments said glucocorticoid is fluticasone.

In other embodiments said method further results in at least one effect selected from the group consisting of:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In certain embodiments said method further results in at least two effects selected from the group consisting of:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In certain embodiments said method effects a decreased metabolism by at least one polymorphically-expressed cytochrome P450 isoform in said subject per dosage unit thereof as compared to the non-isotopically enriched compound.

In other embodiments said cytochrome P450 isoform is selected from the group consisting of CYP2C8, CYP2C9, CYP2C19, and CYP2D6.

In yet further embodiments said compound is characterized by decreased inhibition of at least one cytochrome P450 or monoamine oxidase isoform in said subject per dosage unit thereof as compared to the non-isotopically enriched compound.

In certain embodiments said cytochrome P450 or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAOA, and MAOB.

In certain embodiments, said method reduces a deleterious change in a diagnostic hepatobiliary function endpoint, as compared to the corresponding non-isotopically enriched compound.

In yet other embodiments, said diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

In another embodiment a compound disclosed herein can be used as a medicament.

In a further embodiment a compound disclosed herein can be used in the manufacture of a medicament for the prevention or treatment of a disorder ameliorated by the modulation of adrenergic receptors.

In certain embodiments, disclosed herein is a deuterium-enriched compound of formula I or a pharmaceutically acceptable salt thereof:

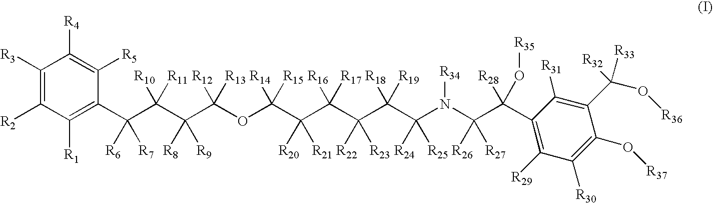

wherein R₁-R₃₇ are independently selected from H and D; and the abundance of deuterium in R₁-R₃₇ is at least 3%, provided that if R₅-R₆ and R₁₀ are D, then at least one other R is a D.

In certain embodiments, disclosed herein is an isolated deuterium-enriched compound of formula I or a pharmaceutically acceptable salt thereof:

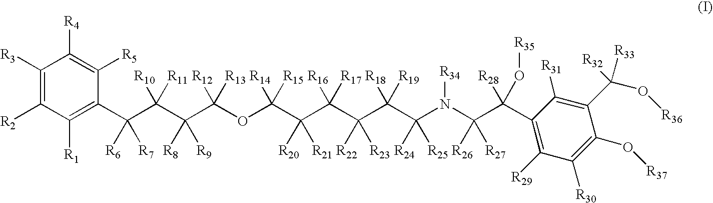

wherein R₁-R₃₇ are independently selected from H and D; and the abundance of deuterium in R₁-R₃₇ is at least 3%, provided that if R₅-R₆ and R₁₀ are D, then at least one other R is a D.

In certain embodiments, disclosed herein is a mixture of deuterium-enriched compounds of formula I or a pharmaceutically acceptable salt thereof:

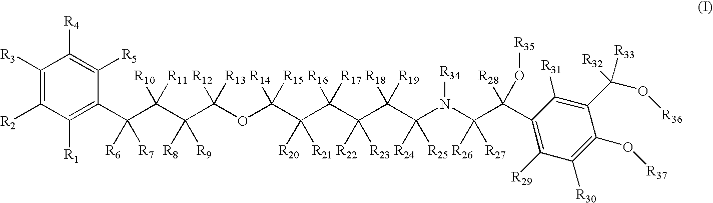

wherein R₁-R₃₇ are independently selected from H and D; and the abundance of deuterium in R₁-R₃₇ is at least 3%, provided that if R₅-R₆ and R₁₀ are D, then at least one other R is a D.

In further embodiments, the abundance of deuterium in R₁-R₃₇ is selected from at least 3%, at least 5%, at least 11%, at least 16%, at least 22%, at least 27%, at least 32%, at least 38%, at least 43%, at least 49%, at least 54%, at least 59%, at least 65%, at least 70%, at least 76%, at least 81%, at least 86%, at least 92%, at least 97%, and 100%.

In yet further embodiments, the abundance of deuterium in R₃₄-R₃₇ is selected from at least 25%, at least 50%, at least 75%, and 100%.

In yet further embodiments, the abundance of deuterium in R₃₂-R₃₃, is selected from at least 50% and 100%.

In yet further embodiments, the abundance of deuterium in R₂₉-R₃₁ is selected from at least 33%, at least 67%, and 100%.

In yet further embodiments, the abundance of deuterium in R₂₆-R₂₈ is selected from at least 33%, at least 67%, and 100%.

In yet further embodiments, the abundance of deuterium in R₁₄-R₂₅ is selected from at least 8%, at least 17%, at least 25%, at least 33%, at least 42%, at least 50%, at least 58%, at least 67%, at least 75%, at least 83%, at least 92%, and 100%.

In yet further embodiments, the abundance of deuterium in R6-R₁₃ is selected from at least 13%, at least 25%, at least 38%, at least 50%, at least 63%, at least 75%, at least 88%, and 100%.

In yet further embodiments, the abundance of deuterium in R₁-R₅ is selected from at least 20%, at least 40%, at least 60%, at least 80%, and 100%.

In yet further embodiments, the compound, isolated compound, or mixture of compounds has a structural formula selected from the group consisting of:

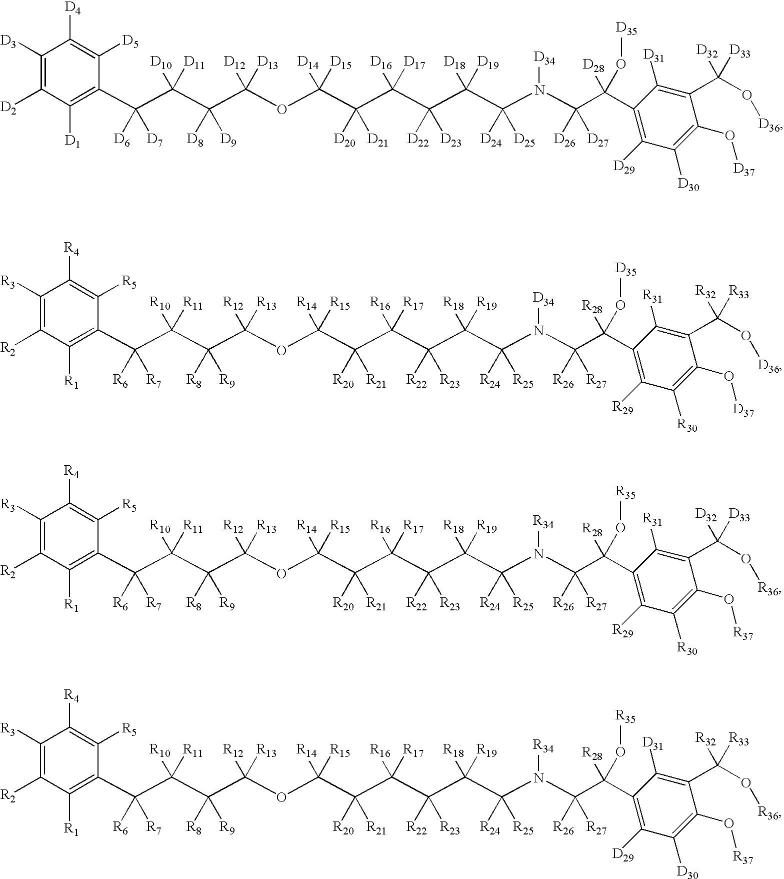

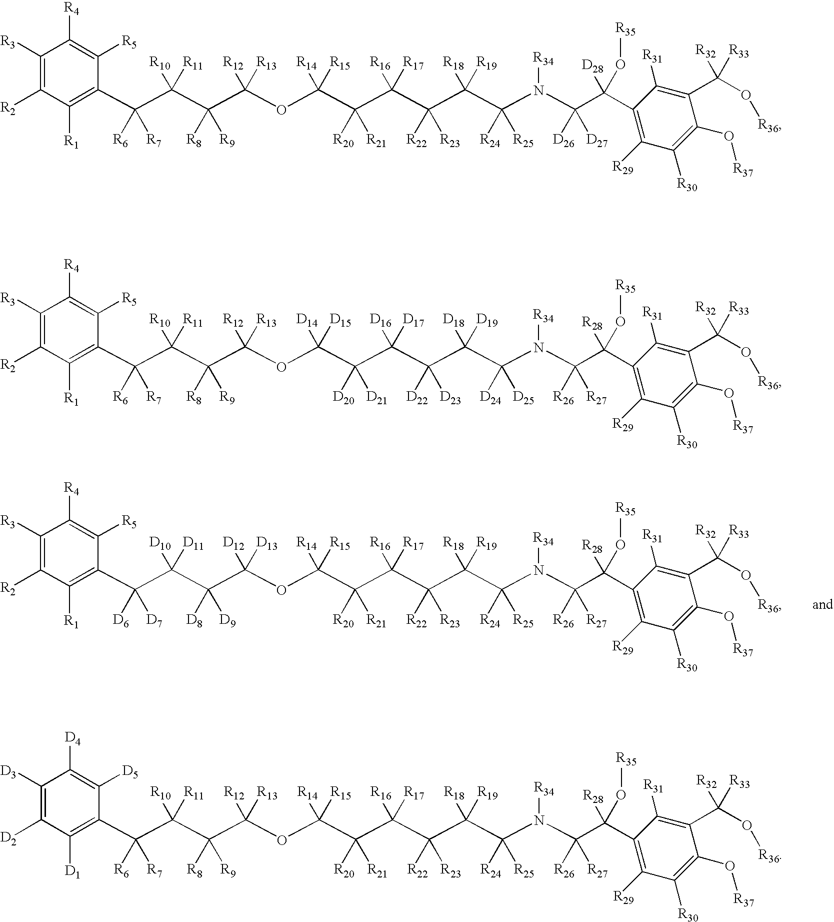

In yet further embodiments, the compound, isolated compound, or mixture of compounds has a structural formula selected from the group consisting of:

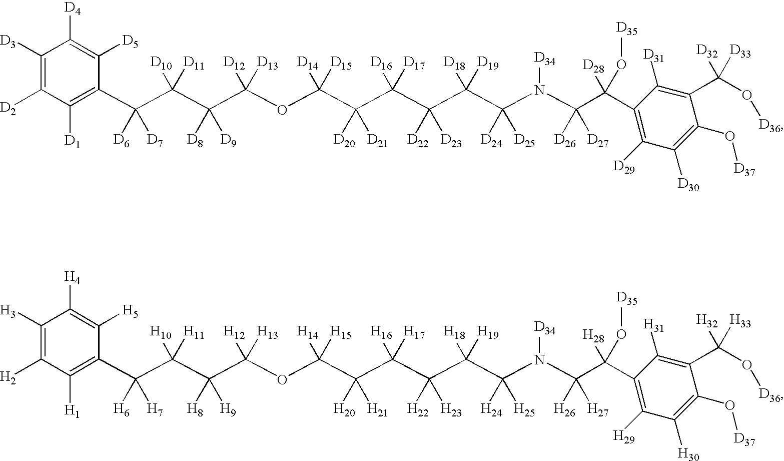

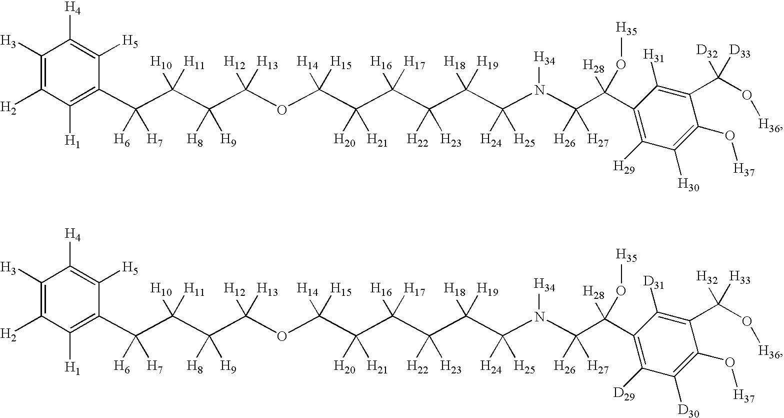

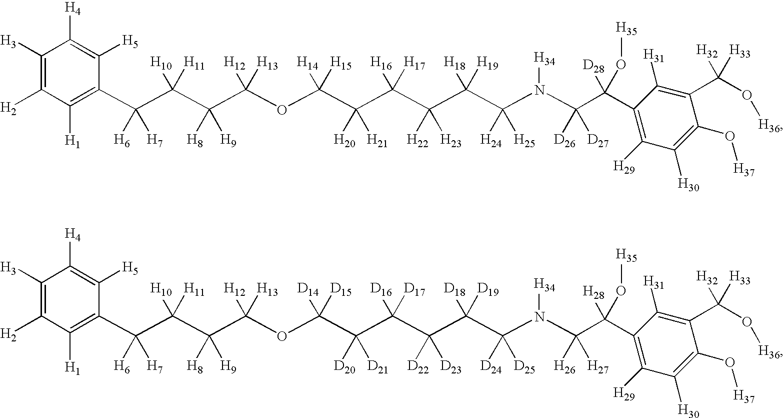

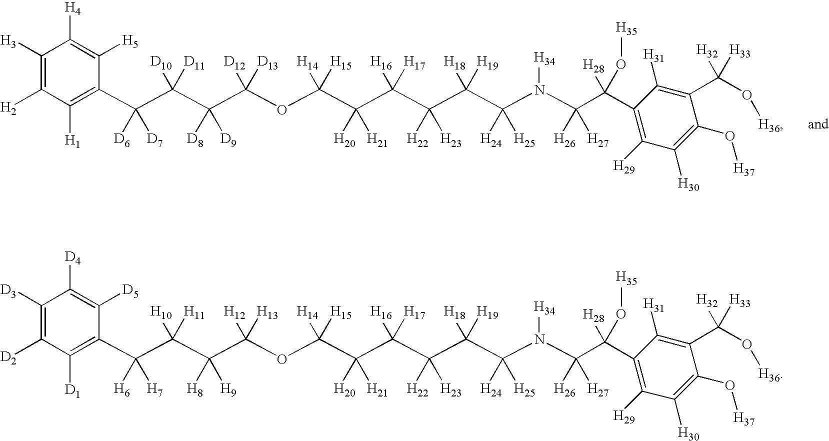

In certain embodiments, disclosed herein is a pharmaceutical composition, comprising: a pharmaceutically acceptable carrier and a therapeutically effective amount of a compound of Formula I or a pharmaceutically acceptable salt form thereof.

In certain embodiments, disclosed herein is a method for treating asthma comprising: administering, to a patient in need thereof, a therapeutically effective amount of a compound of Formula I or a pharmaceutically acceptable salt form thereof.

All publications and references cited herein are expressly incorporated herein by reference in their entirety. However, with respect to any similar or identical terms found in both the incorporated publications or references and those expressly put forth or defined in this document, then those terms definitions or meanings expressly put forth in this document shall control in all respects.

As used herein, the terms below have the meanings indicated.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

In representing a range of positions on a structure, the notation notation “from n₁ . . . to n₂” or “n₁-n₂” is used, where n₁ and n₂ represent numbers. Then unless otherwise specified, this notation is intended to include not only the numbers represented by x and xx themselves, but all the numbered positions that are bounded by n₁ and n₂. For example, “from R₁ . . . to R₄” or “R₁-R₄” would, unless otherwise specified, be equivalent to R₁, R₂, R₃, and R₄.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₃₇ or the symbol “D,” when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as D-isomers and L-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a linkage between two atoms, or two moieties when the atoms joined by the bond are considered to be part of larger substructure. A bond may be ionic, metallic, or covalent. If covalent, the bond can be either result from the sharing of one pair of electrons, a single bond; a sharing of 2 pairs of electrons, a double bond; a sharing of 3 pairs of electrons, or a triple bond; or sharing of more than 3 pairs of electrons. A dashed line between two atoms in a drawing of a molecule indicates that an additional bond may be present or absent at that position.

The term “disorder” as used herein is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “adrenergic receptors” refers to a family of receptors which are linked to Gₛ proteins, which in turn, are linked to adenylate cyclase. Therefore, when these receptors are agonized, the intracellular concentration of the secondary messenger, cAMP, increases. The higher concentration of cAMP then triggers a cascade of downstream events. Antagonizing adrenergic receptors decreases sympathetic neuronal activity.

The term “adrenergic receptor modulator”, “modulation of adrenergic receptors”, or “modulating adrenergic receptors” are meant to be interchangeable, and refer to the ability of a compound disclosed herein to alter the function of an adrenergic receptor. An adrenergic receptor modulator may activate the activity of an adrenergic receptor, may activate or inhibit the activity of an adrenergic receptor depending on the concentration of the compound exposed to the adrenergic receptor, or may inhibit the activity of an adrenergic receptor. Such activation or inhibition may be contingent on the occurrence of a specific event, such as activation of a signal transduction pathway, and/or may be manifest only in particular cell types. The term “adrenergic receptor modulator” also refers to altering the function of an adrenergic receptor by increasing or decreasing the probability that a complex forms between an adrenergic receptor and a natural binding partner. An adrenergic receptor modulator may increase the probability that such a complex forms between the adrenergic receptor and the natural binding partner, may increase or decrease the probability that a complex forms between the adrenergic receptor and the natural binding partner depending on the concentration of the compound exposed to the adrenergic receptor, and or may decrease the probability that a complex forms between the adrenergic receptor and the natural binding partner.

The terms “adrenergic receptor-mediated disorder” refers to a disorder that is characterized by abnormal adrenergic receptor activity or normal adrenergic receptor receptor activity that, when that activity is modified, leads to the amelioration of other abnormal biological processes. Adrenergic receptor-mediated disorders may be completely or partially mediated by modulation of adrenergic receptors. In particular, an adrenergic receptor-mediated disorder is one in which modulation of adrenergic receptors' activity results in some effect on the underlying disorder, e.g., administering an adrenergic receptor modulator results in some improvement in at least some of the patients being treated.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al., in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev.1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The term “chlorinating reagent” refers to a reactive chemical reagent used in chlorination reactions, whereby chlorine is transferred to a substrate. Examples of chlorinating agents include, but are not limited to, thionyl chloride, chlorine gas, carbon tetrachloride, hydrochloric acid, cyanuric chloride, hexachloro-2-propanone, N-chlorosuccinimide, phosphorus oxychloride, phosphorus pentachloride, phosphorus trichloride, phosphorus (V) oxychloride, and sulfuryl chloride.

The term “reducing reagent” refers to any reagent that will decrease the oxidation state of an atom in the starting material by either adding a hydrogen to this atom, or adding an electron to this atom, or by removing an oxygen from this atom and as such would be obvious to one of ordinary skill and knowledge in the art. The definition of “reducing reagent” includes but is not limited to: borane-dimethyl sulfide complex, 9-borabicyclo[3.3.1.]nonane (9-BBN), catechol borane, lithium borohydride, lithium borodeuteride, sodium borohydride, sodium borodeuteride, sodium borohydride-methanol complex, potassium borohydride, sodium hydroxyborohydride, lithium triethylborohydride, lithium n-butylborohydride, sodium cyanoborohydride, sodium cyanoborodeuteride, calcium (II) borohydride, lithium aluminum hydride, lithium aluminum deuteride, diisobutylaluminum hydride, n-butyl-diisobutylaluminum hydride, sodium bis-methoxyethoxy, aluminum hydride, triethoxysilane, diethoxymethylsilane, lithium hydride, lithium, sodium, hydrogen Ni/B, and the like. Certain acidic and Lewis acidic reagents enhance the activity of reducing reagents. Examples of such acidic reagents include: acetic acid, methanesulfonic acid, hydrochloric acid, and the like. Examples of such Lewis acidic reagents include: trimethoxyborane, triethoxyborane, aluminum trichloride, lithium chloride, vanadium trichloride, dicyclopentadienyl titanium dichloride, cesium fluoride, potassium fluoride, zinc (II) chloride, zinc (II) bromide, zinc (II) iodide, and the like.

The term “oxidizing reagent” refers to any reagent that will increase the oxidation state of an atom, such as for example, hydrogen, carbon, nitrogen, sulfur, phosphorus and the like in the starting material by either adding an oxygen to this atom or removing an electron from this atom and as such would be obvious to one of ordinary skill and knowledge in the art. The definition of “oxidant” includes but is not limited to: osmium tetroxide, ruthenium tetroxide, ruthenium trichloride, potassium permanganate, meta-chloroperbenzoic acid, hydrogen peroxide, dimethyl dioxirane, 3-chlorobenzoic acid, and the like.

The definition of “hydroxyl protecting group” includes but is not limited to:
a) methyl, tert-butyl, allyl, propargyl, p-chlorophenyl, p-methoxyphenyl, p-nitrophenyl, 2,4-dinitrophenyl, 2,3,5,6-tetrafluoro-4-(trifluoromethyl)phenyl, methoxymethyl, methylthiomethyl, (phenyldimethylsilyl)methoxymethyl, benzyloxymethyl, p-methoxy-benzyloxymethyl, p-nitrobenzyloxymethyl, o-nitrobenzyloxymethyl, (4-methoxyphenoxy)methyl, guaiacolmethyl, tert-butoxymethyl, 4-pentenyloxymethyl, tert-butyldimethylsiloxymethyl, thexyldimethylsiloxymethyl, tert-butyldiphenylsiloxymethyl, 2-methoxyethoxymethyl, 2,2,2-trichloroethoxymethyl, bis(2-chloroethoxy)methyl, 2-(trimethylsilyl)ethoxymethyl, menthoxymethyl, 1-ethoxyethyl, 1-(2-chloroethoxy)ethyl, 1-[2-(trimethylsilyl)ethoxy]ethyl, 1-methyl-1-ethoxyethyl, 1-methyl-1-benzyloxyethyl, 1-methyl-1-benzyloxy-2-fluoroethyl, 1-methyl-1-phenoxyethyl, 2,2,2-trichloroethyl, 1-dianisyl-2,2,2-trichloroethyl, 1,1,1,3,3,3-hexafluoro-2-phenylisopropyl, 2-trimethylsilylethyl, 2-(benzylthio)ethyl, 2-(phenylselenyl)ethyl, tetrahydropyranyl, 3-bromotetrahydropyranyl, tetrahydrothiopyranyl, 1-methoxycyclohexyl, 4-methoxytetrahydropyranyl, 4-methoxytetrahydrothiopyranyl, 4-methoxytetrahydropyranyl S,S-dioxide, 1-[(2-chloro-4-methyl)phenyl]-4-methoxypiperidin-4-yl, 1-(2-fluorophenyl)-4-methoxypiperidin-4-yl, 1,4-dioxan-2-yl, tetrahydrofuranyl, tetrahydrothiofuranyl and the like;
b) benzyl, 2-nitrobenzyl, 2-trifluoromethylbenzyl, 4-methoxybenzyl, 4-nitrobenzyl, 4-chlorobenzyl, 4-bromobenzyl, 4-cyanobenzyl, 4-phenylbenzyl, 4-acylaminobenzyl, 4-azidobenzyl, 4-(methylsulfinyl)benzyl, 2,4-dimethoxybenzyl, 4-azido-3-chlorobenzyl, 3,4-dimethoxybenzyl, 2,6-dichlorobenzyl, 2,6-difluorobenzyl, 1-pyrenylmethyl, diphenylmethyl, 4,4′-dinitrobenzhydryl, 5-benzosuberyl, triphenylmethyl(trityl), α-naphthyldiphenylmethyl, (4-methoxyphenyl)-diphenyl-methyl, di-(p-methoxyphenyl)-phenylmethyl, tri-(p-methoxyphenyl)methyl, 4-(4′-bromophenacyloxy)-phenyldiphenylmethyl, 4,4′,4″-tris(4,5-dichlorophthalimidophenyl)methyl, 4,4′,4″-tris(levulinoyloxyphenyl)methyl, 4,4′-dimethoxy-3″-[N-(imidazolylmethyl)]trityl, 4,4′-dimethoxy-3″-[N-(imidazolylethyl)carbamoyl]trityl, 1,1-bis(4-methoxyphenyl)-1′-pyrenylmethyl, 4-(17-tetrabenzo[a,c,g,i]fluorenylmethyl)-4,4′-dimethoxytrityl, 9-anthryl, 9-(9-phenyl)xanthenyl, 9-(9-phenyl-10-oxo)anthryl and the like;
c) trimethylsilyl, triethylsilyl, triisopropylsilyl, dimethylisopropylsilyl, diethylisopropylsilyl, dimethylhexylsilyl, tert-butyldimethylsilyl, tert-butyldiphenylsilyl, tribenzylsilyl, tri-p-xylylsilyl, triphenylsilyl, diphenylmethylsilyl, di-tert-butylmethylsilyl, tris(trimethylsilyl)silyl, (2-hydroxystyryl)dimethylsilyl, (2-hydroxystyryl)diisopropylsilyl, tert-butylmethoxyphenylsilyl, tert-butoxydiphenylsilyl and the like;
d) —C(O)R₈₀, where R₈₀ is selected from the group consisting of alkyl, substituted alkyl, aryl and more specifically R₈₀=hydrogen, methyl, ethyl, tert-butyl, adamantyl, crotyl, chloromethyl, dichloromethyl, trichloromethyl, trifluoromethyl, methoxymethyl, triphenylmethoxymethyl, phenoxymethyl, 4-chlorophenoxymethyl, phenylmethyl, diphenylmethyl, 4-methoxycrotyl, 3-phenylpropyl, 4-pentenyl, 4-oxopentyl, 4,4-(ethylenedithio)pentyl, 5-[3-bis(4-methoxyphenyl)hydroxymethylphenoxy]-4-oxopentyl, phenyl, 4-methylphenyl, 4-nitrophenyl, 4-fluorophenyl, 4-chlorophenyl, 4-methoxyphenyl, 4-phenylphenyl, 2,4,6-trimethylphenyl, α-naphthyl, benzoyl and the like;
e) —C(O)OR₈₀, where R₈₀ is selected from the group consisting of alkyl, substituted alkyl, aryl and more specifically R₈₀=methyl, methoxymethyl, 9-fluorenylmethyl, ethyl, 2,2,2-trichloromethyl, 1,1-dimethyl-2,2,2-trichloroethyl, 2-(trimethylsilyl)ethyl, 2-(phenylsulfonyl)ethyl, isobutyl, tert-butyl, vinyl, allyl, 4-nitrophenyl, benzyl, 2-nitrobenzyl, 4-nitrobenzyl, 4-methoxybenzyl, 2,4-dimethoxybenzyl, 3,4-dimethoxybenzyl, 2-(methylthiomethoxy)ethyl, 2-dansenylethyl, 2-(4-nitrophenyl)ethyl, 2-(2,4-dinitrophenyl)ethyl, 2-cyano-1-phenylethyl, thiobenzyl, 4-ethoxy-1-naphthyl and the like. Other examples of hydroxyl protecting groups are given in Greene and Wutts, above.

The definition of “amino protecting group” includes but is not limited to:
2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 1-methyl-1-(triphenylphosphonio)ethyl, 1,1-dimethyl-2-cyanoethyl, 2-dansylethyl, 2-(4-nitrophenyl)ethyl, 4-phenylacetoxybenzyl, 4-azidobenzyl, 4-azidomethoxybenzyl, m-chloro-p-acyloxybenzyl, p-(dihydroxyboryl)benzyl, 5-benzisoxazolylmethyl, 2-(trifluoromethyl)-6-chromonytmethyl, m-nitrophenyl, 3.5-dimethoxybenzyl, 1-methyl-1-(3,5-dimethoxyphenyl)ethyl, o-nitrobenzyl, α-methylnitropiperonyl, 3,4-dimethoxy-6-nitrobenzyl, N-benzenesulfenyl, N-o-nitrobenzenesulfenyl, N-2,4-dinitrobenzenesulfenyl, N-pentachlorobenzenesulfenyl. N-2-nitro-4-methoxybenzenesulfenyl, N-triphenylmethylsulfenyl, N-1-(2,2,2-trifluoro-1,1-diphenyl)ethylsulfenyl, N-3-nitro-2-pyridinesulfenyl, N-p-toluenesulfonyl, N-benzenesulfonyl, N-2,3,6-trimethyl-4-methoxybenzenesulfonyl, N-2,4,6-trimethoxybenzene-sulfonyl, N-2,6-dimethyl-4-methoxybenzenesulfonyl, N-pentamethylbenzenesulfonyl, N-2,3,5,6-tetramethyl-4-methoxybenzenesulfonyl and the like;
—C(O)OR₈₀, where R₈₀ is selected from the group consisting of alkyl, substituted alkyl, aryl and more specifically R₈₀=methyl, ethyl, 9-fluorenylmethyl, 9-(2-sulfo)fluorenylmethyl. 9-(2,7-dibromo)fluorenylmethyl, 17-tetrabenzo[a,c,g,i]fluorenylmethyl. 2-chloro-3-indenylmethyl, benz[f]inden-3-ylmethyl, 2,7-di-t-butyl-[9-(10,10-dioxo-10,10,10,10-tetrahydrothloxanthyl)]methyl, 1,1-dioxobenzo[b]thiophene-2-ylmethyl, 2,2,2-trichloroethyl, 2-trimethylsilylethyl, 2-phenylethyl, 1-(1-adamantyl)-1-methylethyl, 2-chloroethyl, 1.1-dimethyl-2-haloethyl, 1,1-dimethyl-2,2-dibromoethyl, 1,1-dimethyl-2,2,2-trichloroethyl, 1-methyl-1-(4-biphenylyl)ethyl, 1-(3,5-di-tert-butylphenyl)-1-methylethyl, 2-(2′-pyridyl)ethyl, 2-(4′-pyridyl)ethyl, 2,2-bis(4′-nitrophenyl)ethyl, N-(2-pivaloylamino)-1,1-dimethylethyl, 2-[(2-nitrophenyl)dithio]-1-phenylethyl, tert-butyl, 1-adamantyl, 2-adamantyl, Vinyl, allyl, 1-Isopropylallyl, cinnamyl. 4-nitrocinnamyl, 3-(3-pyridyl)prop-2-enyl, 8-quinolyl, N-Hydroxypiperidinyl, alkyldithio, benzyl, p-methoxybenzyl, p-nitrobenzyl, p-bromobenzyl. p-chlorobenzyl, 2,4-dichlorobenzyl, 4-methylsulfinylbenzyl, 9-anthrylmethyl, diphenylmethyl, tert-amyl, S-benzyl thiocarbamate, butynyl, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N′-dimethylcarboxamido)benzyl, 1,1-dimethyl-3-(N,N′-dimethylcarboxamido)propyl, 1,1-dimethylpropynyl, di(2-pyridyl)methyl, 2-furanylmethyl, 2-Iodoethyl, isobornyl, isobutyl, isonicotinyl, p-(p′-methoxyphenylazo)benzyl, 1-methylcyclobutyl, 1-methylcyclohexyl, 1-methyl-1-cyclopropylmethyl, 1-methyl-1-(p-phenylazophenyl)ethyl, 1-methyl-1-phenylethyl, 1-methyl-1-4′-pyridylethyl, phenyl, p-(phenylazo)benzyl, 2,4,6-trimethylphenyl, 4-(trimethylammonium)benzyl, 2,4,6-trimethylbenzyl and the like. Other examples of amino protecting groups are given in Greene and Wutts, above.

The term “alkylating reagent” as used herein refers to a substituent capable of attaching an alkyl group onto a nucleophilic or an electrophilic site.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed. ;(Wiley-VCH and VHCA, Zurich, 2002) and Berge et al., J. Pharm. Sci. 1977, 66, 1-19.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, a-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration. The most suitable route for administration depends on a variety of factors, including interpatient variation or disorder type, and therefore the invention is not limited to just one form of administration. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating an adrenergic receptor-mediated disorder comprising administering to a subject having or suspected to have such a disorder, a therapeutically effective amount of a compound as disclosed herein or a pharmaceutically acceptable salt, solvate, or prodrug thereof.

Adrenergic receptor-mediated disorders, include, but are not limited to, asthma, chronic obstructive pulmonary disease (COPD), respiratory syncytial virus (RSV), pseudomonas aeruginosa, pneumoconiosis, exercise-induced bronchospasm, chronic bronchitis, any disorder ameliorated by administering a bronchodilator and/or any disorder ameliorated by the modulation of adrenergic receptors.

In certain embodiments, a method of treating an adrenergic receptor-mediated disorder comprises administering to the subject a therapeutically effective amount of a compound of as disclosed herein, or a pharmaceutically acceptable salt, solvate, or prodrug thereof, so as to affect: (1) decreased inter-individual variation in plasma levels of the compound or a metabolite thereof, (2) increased average plasma levels of the compound or decreased average plasma levels of at least one metabolite of the compound per dosage unit; (3) decreased inhibition of, and/or metabolism by at least one cytochrome P₄₅₀ or monoamine oxidase isoform in the subject; (4) decreased metabolism via at least one polymorphically-expressed cytochrome P₄₅₀ isoform in the subject; (5) at least one statistically-significantly improved disorder-control and/or disorder-eradication endpoint; (6) an improved clinical effect during the treatment of the disorder; (7) prevention of recurrence, or delay of decline or appearance, of abnormal alimentary or hepatic parameters as the primary clinical benefit; or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described by Li et al. Rapid Communications in Mass Spectrometry 2005, 19, 1943-1950, Zhang et al., Journal of Chromatography B 1999, 729, 225-230, Manchee et al., Drug Metab Dispos 1993, 21, 1022, De Boer et al., Recent Advances in Doping Analysis (4), Proceedings of Manfred Donike workshop, Cologne workshop on Dope Analysis, 14^{th} Cologne, Mar. 17-22, 1996 (1997), and Colthup et al., J of Pharmaceutical Sciences 1993, 82(3), 323-5.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP1A1, CYP1B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P₄₅₀ isoform is measured by the method of Ko et al. (British Journal of Clinical Pharmacology, 2000, 49, 343-351). The inhibition of the MAO_{A} isoform is measured by the method of Weyler et al. (J. Biol Chem. 1985, 260, 13199-13207). The inhibition of the MAO_{B} isoform is measured by the method of Uebelhack et al. (Pharmacopsychiatry, 1998, 31, 187-192).

Examples of polymorphically-expressed cytochrome P₄₅₀ isoforms in a mammalian subject include, but are not limited to, CYP2C8, CYP2C9, CYP2C19, and CYP2D6.

The metabolic activities of liver microsomes, cytochrome P₄₅₀ isoforms, and monoamine oxidase isoforms are measured by the methods described herein.

Examples of improved disorder-control and/or disorder-eradication endpoints, or improved clinical effects include, but are not limited to, statistically-significant improvement in the number and severity of asthma attacks; statistically-significant improvement in bronchoconstriction, dyspnea, wheezing, chronic bronchitis, bronchiolitis, lung inflammation, fibrosis, formation of nodular legions in the lung, Unified Parkinson's Disease Rating Scale, Hoehn and Yahr scale, Schwab and England Activities of Daily Living Scale, Beck Depression Inventory, Beck Anxiety Inventory, Beck Hopelessness Scale, executive functions, proprioception, hyposmia, anosmia, weight loss, episodic memory, semantic memory, implicit memory, and diuresis; statistically-significant decrease in the occurrence of tremors, muscular hypertonicity, akinesia, bradykinesia, postural instability, gait and posture disturbances, aboulia, dementia, short term memory loss, somnolence, insomnia, disturbingly vivid dreams, REM Sleep Disorder, dizziness, fainting, pain, altered sexual function, long term memory loss, inability to perform activities of daily learning, oral and dental disease, pressure ulcers, malnutrition, infections, and swallowing difficulties; reduction in need for hemodialysis, and/or diminution of toxicity including but not limited to, hepatotoxicity or other toxicity, or a decrease in aberrant liver enzyme levels as measured by standard laboratory protocols, as compared to the corresponding non-isotopically enriched compound when given under the same dosing protocol including the same number of doses per day and the same quantity of drug per dose.

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment of adrenergic receptor-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds disclosed herein can be combined with one or more adrenergics known in the art, including, but not limited to, salbutamol, levosalbutamol, fenoterol, terbutaline, bambuterol, clenbuterol, formoterol, salmeterol, epinephrine, isoproterenol, and orciprenaline.

In certain embodiments, the compounds disclosed herein can be combined with one or more anti-cholinergics known in the art, including, but not limited to, ipratropium, and tiotropium.

In certain embodiments, the compounds disclosed herein can be combined with one or more mast cell stabilizers known in the art, including, but not limited to, cromoglicate, and nedocromil.

In certain embodiments, the compounds disclosed herein can be combined with one or more xanthines known in the art, including, but not limited to, aminophylline, theobromine, and theophylline.

In certain embodiments, the compounds disclosed herein can be combined with one or more leukotriene antagonists known in the art, including, but not limited to, montelukast, pranlukast and zafirlukast.

In certain embodiments, the compounds disclosed herein can be combined with one or more glucocorticoids treatments known in the art, including, but not limited to, beclometasone, budesonide, ciclesonide, fluticasone and mometasone.

In certain embodiments, the compounds disclosed herein can be combined with one or more decongestants known in the art, including, but not limited to, phenylpropanolamine hydrochloride, pseudoephedrine, phenylephrine, ephedrine, tuaminoheptane, xylometazoline, tetryzoline, naphazoline, cyclopentamine, tramazoline, metizoline, fenoxazoline, tymazoline, and oxymetazoline.

In certain embodiments, the compounds disclosed herein can be combined with one or more anti-tussives known in the art, including, but not limited to, dextromethorphan, ethylmorphine, hydrocodone, codeine, normetahdone, noscapine, pholcodine, thebacon, dimemorfan, and actyldihydrocodeine, benzonatate, benproperine, clobutinol, isoaminile, pentoxyverine, oxolamine, oxeladin, clofedanol, pipazetate, bibenzonium bromide, butamirate, fedrilate, zipeprol, dibunate, droxypropine, prenoxdiazine, dropropizine, cloperastine, meprotixol, piperidione, tipepidine, morclofone, nepinalone, levodropropizine, and dimethoxanate.

In certain embodiments, the compounds disclosed herein can be combined with one or more mucolytics known in the art, including, but not limited to, acetylcysteine, bromhexine, carbocisteine, eprazinone, mesna, ambroxol, sobrerol, domiodol, letosteine, stepronin, tiopronin, dornase alfa, neltenezine and erdosteine.

In certain embodiments, the compounds disclosed herein can be combined with one or more expectorant treatments known in the art, including, but not limited to, tyloxapol, potassium iodide, guaifenesin, ipecacuanha, althea root, senega, antimony pentasulfide, creosote, guaiacolsulfonate, and levoverbenone.

In certain embodiments, the compounds disclosed herein can be combined with one or more anti-histamines known in the art, including, but not limited to, bromazine, carbinoxamine, clemastine, chlorphenoxamine,diphenylpyraline, diphenhydramine, doxylamine, brompheniramine, chlorphenamine, dexbrompheniramine, dexchlorpheniramine, dimetindene, pheniramine, talastine, chloropyramine, histapyrrodine, mepyramine, methapyrilene, tripelennamine (Pyribenzamine), alimemazine, hydroxyethylpromethazine, isothipendyl, mequitazine, methdilazine, oxomemazine, promethazine, buclizine, cetirizine, chlorcyclizine, cinnarizine, cyclizine, hydroxyzine, levocetirizine, meclizine, niaprazine, oxatomide, antazoline, azatadine, bamipine, cyproheptadine, deptropine, dimebon, ebastine, epinastine, ketotifen, mebhydrolin, mizolastine, phenindamine, pimethixene, pyrrobutamine, rupatadine, triprolidine, acrivastine, astemizole, azelastine, desloratadine, fexofenadine, loratadine, terfenadine, antazoline, azelastine, emedastine, epinastine, ketotifen, olopatadine, cromylin sodium and theophylline.

The compounds disclosed herein can also be administered in combination with other classes of compounds, including, but not limited to, sepsis treatments, such as drotrecogin-α; antibacterial agents, such as ampicillin; antifungal agents such as terbinafine; anticoagulants, such as bivalirudin; thrombolytics, such as streptokinase; non-steroidal anti-inflammatory agents, such as aspirin; antiplatelet agents, such as clopidogrel; norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating adrenergic receptor-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of adrenergic receptor-mediated disorders.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in U.S. Pat. No. 4,992,474, Zhang et al., Chinese Chemical Letters 2006, 17(2), 163-164, Gisch et al., Journal of Medicinal Chemistry 2007, 50(7), 1658-1667, Jia et al., Synlett Letter 2007, 5, 806-808, and de Napoli et al., Phytochemistry 1990, 29(3), 701-703, and references cited therein and routine modifications thereof. Compounds as disclosed herein can also be prepared as shown in any of the following schemes and routine modifications thereof.

The following schemes can be used to practice the present invention. Any position shown as hydrogen may optionally be replaced with deuterium.

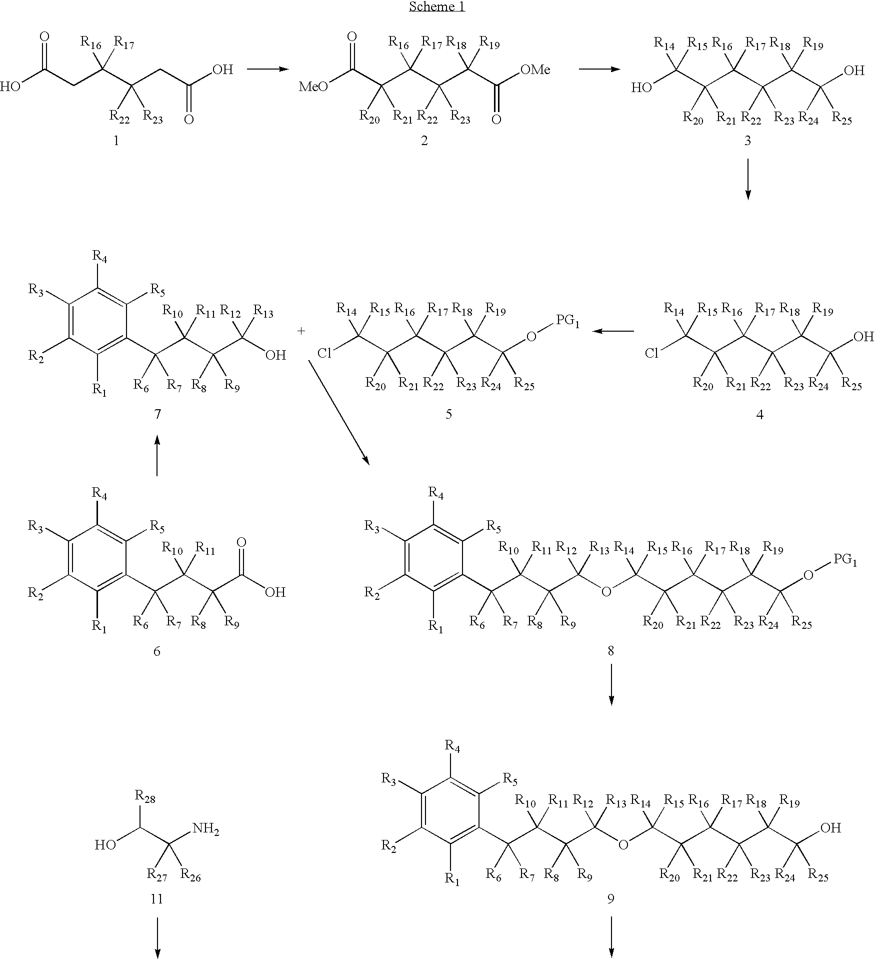

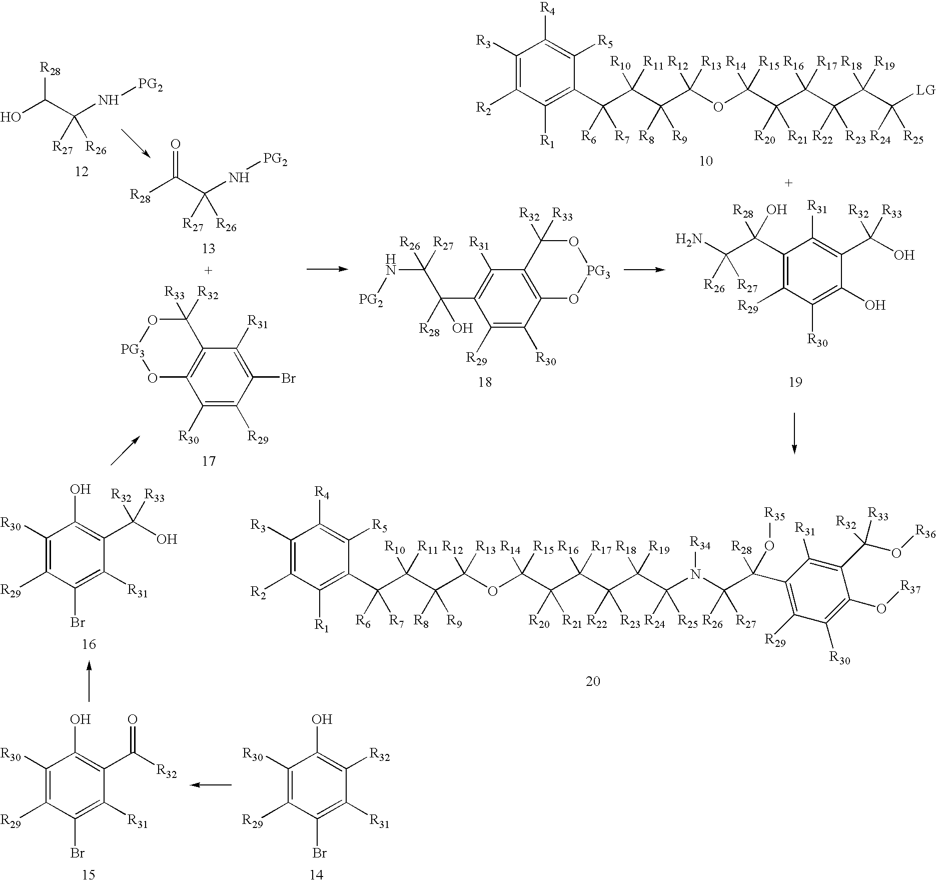

Compound 1 is reacted with an alcohol, such as methanol, in the presence of an appropriate acid, such as hydrochloric acid, to give compound 2, which is reacted with an appropriate reducing agent, such as lithium aluminum hydride, in an appropriate solvent, such as tetrahydrofuran, to give compound 3. Compound 3 is reacted with an appropriate chlorinating agent, such as hydrochloric acid, in an appropriate solvent, such as a combination of toluene and water, to generate compound 4. Compound 4 is protected with a hydroxyl protecting group (PG₁) by reacting with an appropriate hydroxyl protecting reagent, such as tetrahydropyran, to give compound 5. Compound 6 is reacted with an appropriate reducing agent, such as lithium aluminum hydride, in an appropriate solvent, such as tetrahydrofuran, to generate compound 7. Compound 5 is reacted with compound 7 in an appropriate solvent, such as tetrahydrofuran, in the presence of an appropriate base, such as sodium hydride, and an approrpriate catalyst, such as potassium iodide, to give compound 8. Compound 8 is treated with an appropriate acid, such as acetic acid, in an appropriate solvent, such as methanol, to give compound 9. Compound 9 is reacted with an appropriate leaving group reagent, such as methanesulfonyl chloride, in an appropriate solvent such as dichloromethane, in the presence of an appropriate base, such as triethylamine, to give compound 1O. Compound 11 is protected with an amino protecting group (PG₂) by reacting with an appropriate reagent, such as di-tert-butyl dicarbonate, to yield compound 12. Compound 12 is reacted with an appropriate oxidizing agent, such as pyridinium chlorochromate, to give compound 13. Compound 14 is reacted with an appropriate formylating agent, such as chloroform, in the presence of an approrpriate base, such as triethylamine, and an appropriate hydroxide base, such as sodium hydroxide, to generate compound 15. Compound 15 is then treated with an appropriate reducing agent, such as lithium aluminum hydride, in an appropriate solvent, such as tetrahydrofuran, at an elevated temperature to give compound 16. Compound 16 is protected with a hydroxyl protecting group (PG₃) by reacting with an appropriate reagent, such as 2,2-dimethoxypropane, in the presence of an appropriate acid, such as p-toluenesulfonic acid monohydrate, to give compound 17. Compound 17 is reacted with an appropriate organolithium reagent, such as n-butyllithium, in an appropriate solvent, such as tetrahydrofuran, and is then reacted with compound 13 to generate compound 18. Compound 18 is treated with an appropriate acid, such as hydrochloric acid, in an appropriate solvent, such as methanol, to give compound 19. Compound 19 is reacted with compound 10 in an appropriate solvent, such as N,N-dimethylformamide, in the presence of an appropriate base, such as triethylamine, and a catalyst, such as potassium iodide, at an elevated temperature to give compound 20 of Formula I.

Selective synthesis of (R)-Salmeterol can be accomplished by following the protocols and procedures as described in Buchanan, et al., Synlett 2005, 12, 1948-50 and Bream et al., J Chem Soc 2002, Perkin Times 1, 2237-242, which are hereby incorporated by reference in their entirety.

Selective synthesis of the (S)-Salmeterol can be accomplished by following the protocols and procedures as described in Procopiou et al., Tetrahedron Asymmetry 2001, 12, 2005-8, which is hereby incorporated by reference in its entirety.

Deuterium can be incorporated to different positions synthetically, according to the synthetic procedures as shown in Scheme 1, by using appropriate deuterated intermediates. For example, to introduce deuterium at one or more positions of R₁₆, R₁₇, R₂₂, and R₂₃, compound 1 with the corresponding deuterium substitutions can be used. To introduce deuterium at one or more positions of R₁, R₂, R₃, R₄, R₅, R₆, R₇, R₈, R₉, R₁₀and R₁₁, compound 6 with the corresponding deuterium substitutions can be used. To introduce deuterium at one or more positions of R₂₆, R₂₇ and R₂₈, compound 11 with the corresponding deuterium substitutions can be used. To introduce deuterium at one or more positions of R₂₉, R₃₀ R₃₁, and R₃₂, compound 14 with the corresponding deuterium substitutions can be used. To introduce deuterium at position R₁₂, R₁₃, R₁₄, R₁₅, R₂₄, R₂₅, and R₃₃, lithium aluminum deuteride can be used. To introduce deuterium at position R₁₈, R₁₉, R₂₀, and R₂₁, d₄-methanol can be used. These deuterated intermediates are either commercially available, or can be prepared by methods known to one of skill in the art or following procedures similar to those described in the Example section herein and routine modifications thereof.

Deuterium can also be incorporated to various positions having an exchangeable proton, such as the amine N—H group and the hydroxyl O—H groups, via proton-deuterium equilibrium exchange. For example, to introduce deuterium at R₃₄, R₃₅, R₃₆ and R₃₇ these protons may be replaced with deuterium selectively or non-selectively through a proton-deuterium exchange method known in the art.

The invention is further illustrated by the following examples. All IUPAC names were generated using CambridgeSoft's ChemDraw 10.0.

### EXAMPLE 1

### N-(2-Hydroxy-2-(4-hydroxy-3-(hydroxymethyl)phenyl)ethyl)-6-(4-phenylbutoxy)hexan-1-aminium 1-hydroxy-2-naphthoate

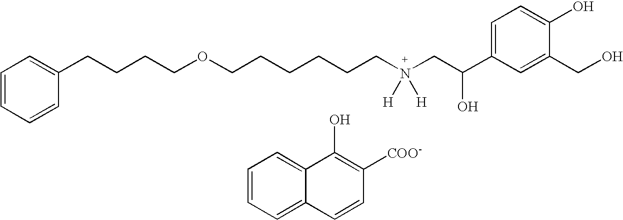

### Step 1

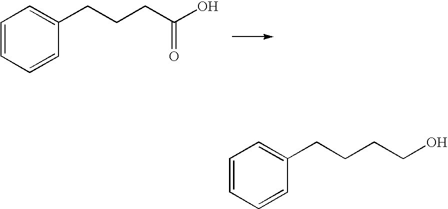

4-Phenylbutan-1-ol: At 0˜10° C., a solution of 4-phenylbutanoic acid (10 g; 60.4 mmol; 1.00 equiv) in tetrahydrofuran (50 mL) was added to a suspension of lithium aluminum hydride (4.6 g; 121 mmol; 2.01 equiv) in tetrahydrofuran (100 mL). The suspension was virgously stirred at ambient temperature for about 2 hours and then quenched by adding water. Standard extractive workup with ethyl acetate afforded the title product as a yellow liquid (8.4 g; 92% yield). ¹H NMR (300 MHz, CDCl₃) δ: 7.31 (t, J=7.5, 7.2 Hz, 2H), 7.15 (m, 3H), 3.68 (t, J=6.3, 6.3 Hz, 2H), 2.68 (t, J=7.2, 7.8 Hz, 2H), 1.69 (m, 4H).

### Step 2

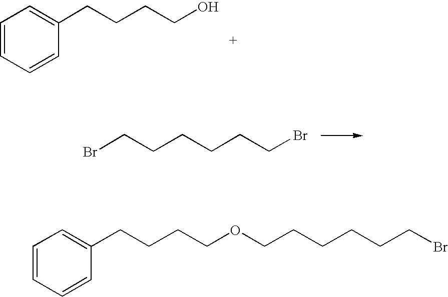

1-(4-(6-Bromohexyloxy)butyl)benzene: At 0˜10° C., a solution of 4-phenylbutan-1-ol (8.4 g; 53.2 mmol; 1.00 equiv) in tetrahydrofuran (150 mL) was added to a suspension of sodium hydride (2.5 g; 62.5 mmol; 1.17 equiv) in tetrahydrofuran (50 mL). The resulting mixture was stirred at ambient temperature for about 1 hour and then 1,6-dibromohexane (41.0 g; 166 mmol; 3.13 equiv) and tetra-N-butylammonium bromide (100 mg; 0.27 mmol; 0.01 equiv) were added. The mixture was maintained at ambient temperature for about 16 hours, and then water was added. Following standard extractive workup with ethyl acetate, the crude residue was purified by silica gel column chromatography with ethyl acetate/petroleum ether (1/8) to afford the title product as a yellow liquid (14.2 g; 84% yield). ¹H NMR (300 MHz, CDCl₃) δ: 7.30 (m, 2H), 7.15 (m, 3H), 3.43 (m, 6H), 2.66 (t, J=7.2, 7.5 Hz, 2H), 1.89 (m, 2H), 1.75˜1.58 (m, 6H), 1.56˜1.37 (m, 4H).

### Step 3

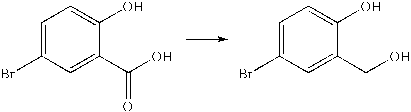

4-Bromo-2-(hydroxymethyl)phenol: A solution of 5-bromo-2-hydroxybenzoic acid (21.7 g; 100 mmol; 1.00 equiv) in tetrahydrofuran (200 mL) was added to a suspension of lithium aluminum hydride (5.7 g; 150 mmol; 1.50 equiv) in tetrahydrofuran (100 mL). The suspension was virgously stirred for about 16 hours at ambient temperature and then quenched by adding 3M hydrochloric acid (300 mL). Following standard extractive workup with ethyl acetate, the crude residue was re-crystallized from ethyl acetate/hexane (1/10) to give the title product as gray solid (12.4 g; 60% yield). ¹H NMR (400 MHz, CDCl₃) 6: 9.68 (s, 1H), 7.39 (s, 1H), 7.19 (dd, J=2.0, 2.0 Hz, 1H), 6.72 (d, J=8.4 Hz, 1H), 5.10 (s, 1H), 4.44 (s, 2H).

### Step 4

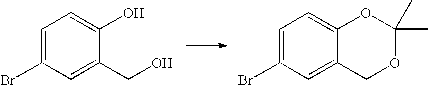

6-Bromo-2,2-dimethyl-4H-benzo[d][1,3]dioxine: Zinc chloride (8 g; 58.8 mmol; 2.01 equiv) was added to acetone (100 mL) and stirred at ambient temperature for about 30 minutes. A solution of 4-bromo-2-(hydroxymethyl)phenol (6 g; 29.3 mmol; 1. 00 equiv) in acetone (100 mL) was added, and the resulting solution was stirred at about 40° C. for about 16 hours. The pH of the solution was adjusted to 8 with a saturated sodium carbonate solution. Following standard extractive workup with ethyl acetate, the crude residue was purified by silica gel column chromotagraphy with ethyl acetate/petroleum ether (1/6) to give the title product as a colorless liquid (5.2 g; 72% yield). ¹H NMR (400 MHz, CDCl₃) δ: 7.30 (s, 1H), 7.28 (s, 1H), 6.75 (d, J=6.6 Hz, 1H), 4.80 (s, 2H), 1.45 (s, 6H).

### Step 5

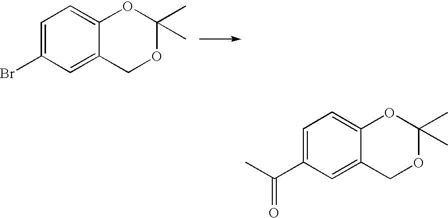

1-(2,2-Dimethyl-4H-benzo[d][1,3]dioxin-6-yl)ethanone: At about −78° C. and under an atmosphere of nitrogen, n-butyllithium in tetrahydrofuran (14.8 mL; 2.5M) was added to a solution of 6-bromo-2,2-dimethyl-4H-benzo[d][1,3]dioxine (8.2 g; 33.07 mmol; 1.00 equiv) in tetrahydrofuran (200 mL). The mixture was stirred at −40 to −60° C. for about 2 hours. At about −78° C., N-methoxy-N-methylacetamide (5.2 g; 49.5 mmol; 1.50 equiv) was added, the mixture was stirred at about −78° C. for about 2 hours, and then quenched by adding a solution of saturated ammonium chloride (50 mL). Following standard extractive workup with ethyl acetate, the crude residue was purified by silica gel column chromotagraphy with ethyl acetate/petroleum ether (1/10) to give the title product as a white solid (4.1 g; 60% yield). ¹H NMR (300 MHz, CDCl₃) δ: 7.82 (dd, J=2.1, 2.1 Hz, 1H), 7.68 (s, 1H), 6.88 (d, J=8.7 Hz, 1H), 4.91 (s, 2H), 2.57 (s, 3H), 1.59 (s, 6H).

### Step 6

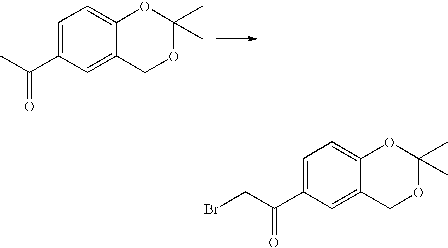

2-Bromo-1-(2,2-dimethyl-4H-benzo[d][1,3]dioxin-6-yl)ethanone: At about −78° C., sodium bis(trimethylsilyl)amide in tetrahydrofuran (1.0M) (2.9 mL) was added to a solution of 1-(2,2-dimethyl-4H-benzo[d][1,3]dioxin-6-yl)ethanone (500 mg; 2.43 mmol; 1.00 equiv) in tetrahydrofuran (20 mL). The solution was stirred at about −78° C. for about 1 hour and then chlorotrimethylsilane (290 mg, 2.64 mmol, 1.09 equiv, 99%) was added. The solution was stirred at about −78° C. for about 30 min, and then bromine (390 mg; 2.41 mmol; 0.99 equiv) was added. The solution was stirred at about −78° C. for about 1 hour, and then an aqueous solution of 5% sodium sulfite and 5% sodium bicarbonate (50 mL) was added. Following standard extractive workup with ethyl acetate, the crude residue was then purified by silica gel column chromotagraphy with ethyl acetate/petroleum ether (1/12) to give the title product as a pale yellow oil (400 mg; 57% yield). ¹H NMR (300 MHz, CDCl₃) δ: 7.84 (dd, J=2.1, 2.1 Hz, 1H), 7.72 (d, J=1.8 Hz, 1H), 6.91 (d, J=8.7 Hz, 1H), 4.92 (s, 2H), 4.40 (s, 2H), 1.60 (s, 6H).

### Step 7

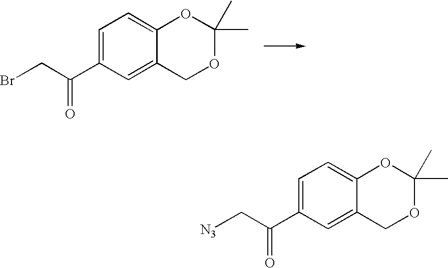

2-Azido-1-(2,2-dimethyl-4H-benzo[d][1,3]dioxin-6-yl)ethanone: Sodium azide (100 mg; 1.54 mmol; 1.15 equiv) was added to a solution of 2-bromo-1-(2,2-dimethyl-4H-benzo[d][1,3]dioxin-6-yl)ethanone (400 mg; 1.33 mmol; 1.00 equiv) in N,N-dimethylformamide (20 mL). The soultion was maintained at ambient temperature for about 2 hours, and then water (20 mL) was added. Standard extractive workup with ethyl acetate afforded the title product as a pale yellow solid (310 mg; 89% yield). ¹H NMR (400 MHz, CDCl₃) δ: 7.72 (dd, J=2.0, 2.0 Hz, 1H), 7.63 (t, J=0.8, 1.2 Hz, 1H), 6.88 (d, J=8.4 Hz, 1H), 4.89 (s, 2H), 4.49 (s, 2H), 1.57 (s, 6H).

### Step 8

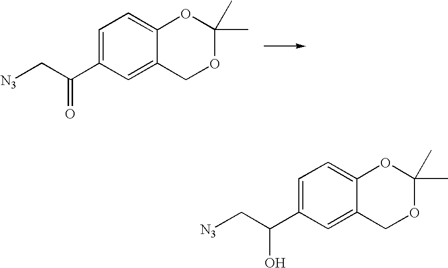

2-Azido-1-(2,2-dimethyl-4H-benzo[d][1,3]dioxin-6-yl)ethanol: At about 0° C., sodium borohdyride (111 mg, 2.92 mmol, 1.53 equiv) was added to a solution of 2-azido-1-(2,2-dimethyl-4H-benzo[d][1,3]dioxin-6-yl)ethanone (480 mg; 1.90 mmol; 1.00 equiv) in tetrahydrofuran/methanol (20/20 mL). The soultuion was maintained at ambient temperature for about 2 hours, and then saturated ammonium chloride (10 mL) was added. Following standard extractive workup with ethyl acetate, the crude residue was purified by silica gel column chromatography with ethyl acetate/petroleum ether (1/4) to give the title product as a white solid (410 mg; 86% yield). ¹H NMR (300 MHz, DMSO) δ: 7.16 (dd, J=1.8, 1.8 Hz, 1H), 7.09 (s, 1H), 6.75 (d, J=8.1 Hz, 1H), 5.72 (d, J=4.5 Hz, 1H), 4.80 (s, 2H), 4.69 (m, 1H), 3.29 (m, 2H), 1.48 (s, 6H).

### Step 9

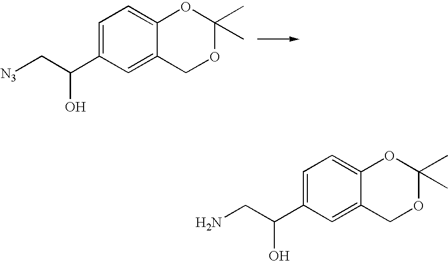

2-Amino-1-(2,2-dimethyl-4H-benzo[d][1,3]dioxin-6-yl)ethanol: Palladium on carbon (100 mg) was added to a solution of 2-azido-1-(2,2-dimethyl-4H-benzo[d][1,3]dioxin-6-yl)ethanol (400 mg, 1.45 mmol, 1.00 equiv, 90%) in ethanol (30 mL). Hydrogen gas was then introduced and the mixture was maintained at ambient temperature for about 2 hours. The catalyst was removed by filtration and the filtrate was concentrated in vacuo. The residue was washed with ether (20 mL) and then filtered to give the title product as a white solid (350 mg; 90% yield). ¹H NMR (400 MHz, DMSO) δ: 7.07 (d, J=8.4 Hz, 1H), 6.99 (s, 1H), 6.71 (d, J=8.0 Hz, 1H), 5.18 (br, 1H), 4.79 (s, 2H), 4.33 (dd, J=4.8, 4.8 Hz, 1H), 2.58 (m, 2H), 1.95 (br, 2H), 1.44 (s, 6H).

### Step 10

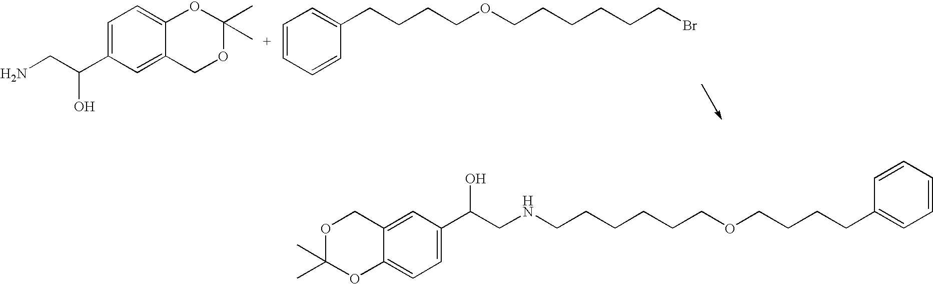

1-(2,2-Dimethyl-4H-benzo[d][1,3]dioxin-6-yl)-2-(6-(4 phenylbutoxy)hexylamino)ethanol: 1-(4-(6-bromohexyloxy)butyl)benzene (122 mg; 0.38 mmol; 0.69 equiv) and N-ethyl-N-isopropylpropan-2-amine (83 mg; 0.64 mmol; 1.16 equiv) was added to a solution of 2-amino-1-(2,2-dimethyl-4H-benzo[d][1,3]dioxin-6-yl)ethanol (130 mg; 0.55 mmol; 1.00 equiv) in N,N-dimethylformamide (3 mL). The resulting solution was stirred at about 50° C. for about 16 hours. Following standard extractive workup with ethyl acetate, the residue was purified with preparative HPLC to give the title product as white solid (60 mg; 24% yield). ¹H NMR (300 MHz, CDCl₃) δ: 8.17 (s, 2H), 7.26 (m, 2H), 7.16 (m, 4H), 7.02 (s, 1H), 6.77 (d, J=8.4 Hz, 1H), 5.04 (t, J=6.6, 6.6 Hz, 1H), 4.80 (s, 2H), 3.37 (m, 4H), 3.01 (d, J=6.6 Hz, 2H), 2.89 (d, J=5.4 Hz, 2H), 2.62 (t, J=7.2, 7.5 Hz, 2H), 1.71˜1.56 (m, 8H), 1.52 (s, 6H), 1.33 (m, 4H).

### Step 11

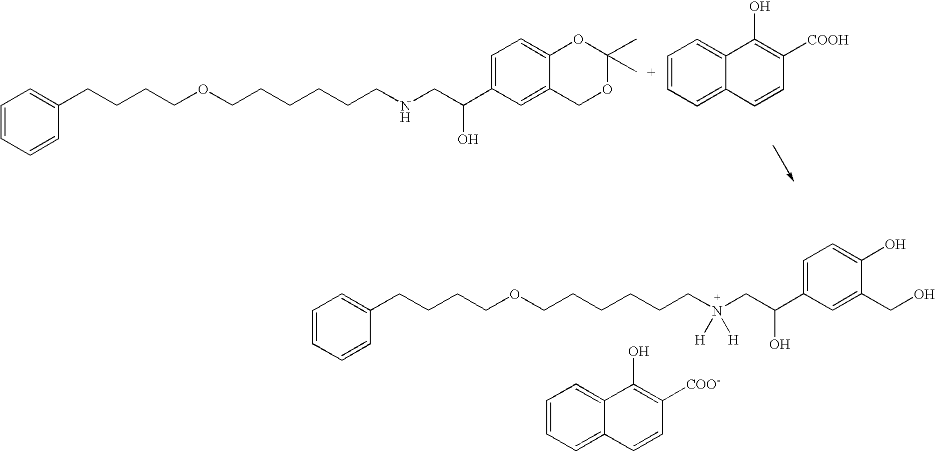

N-(2-Hydroxy-2-(4-hydroxy-3-(hydroxymethyl)phenyl)ethyl)-6-(4-phenylbutoxy)hexan-1-aminium 1-hydroxy-2-naphthoate: Acetic acid (5 mL) and water (1 mL) was added to 1-(2,2-dimethyl-4H-benzo[d][1,3]dioxin-6-yl)-2-(6-(4-phenylbutoxy)hexylamino)ethanol (60 mg; 0. 13 mmol; 1. 00 equiv). The solution was stirred at about 70° C. for about 3 hours, and then concentrated in vacuo. The resulting residue was dissolved with water (40 mL), washed with ethyl acetate (20 mL) and ether (20 mL), and then the pH of the aqueous layer was adjusted to 8 with a saturated sodium bicarbonate solution. Following standard extractive workup with ethyl acetate, the crude residue was dissolved in diethyl ether (10 mL). 1-hydroxy-2-naphthoic acid (50 mg; 0.27 mmol; 2.12 equiv) was added and the mixture was stirred at ambient temperature for about 16 hours. The solid was collected by filtration and washed with diethyl ether to give the title compound as white solid (30 mg; 39% yield). ¹H NMR (300 MHz, DMSO) δ: 9.43 (s, 1H), 8.49 (s, 2H), 8.20 (d, J=7.8 Hz, 1H), 7.70 (m, 2H), 7.44 (t, J=7.2, 7.8 Hz, 1H), 7.38˜7.25 (m, 4H), 7.17 (m, 3H), 7.07 (d, J=8.1 Hz, 1H), 6.96 (s, d, J=8.4 Hz, 1H), 6.77 (d, J=8.1 Hz, 1H), 6.00 (s, 1H), 5.03 (t, J=4.8, 4.8 Hz, 1H), 4.78 (d, J=9.0 Hz, 1H), 4.49 (d, J=4.5 Hz, 2H), 3.34 (m, 4H), 3.07˜2.90 (m, 4H), 2.58 (t, J=6.9, 7.8 Hz, 2H), 1.63˜1.50 (m, 8H), 1.33 (m, 4H). LC-MS: m/z=416 (MH)⁺.

### EXAMPLE 2

### N-(2-Hydroxy-2-(4-hydroxy-3-(hydroxymethyl)phenyl)ethyl)-6-(4-d₃-phenyl-d₈-butoxy)hexan-1-aminium 1-hydroxy-2-naphthoate

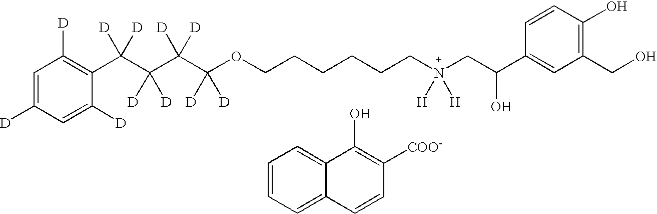

### Step 1

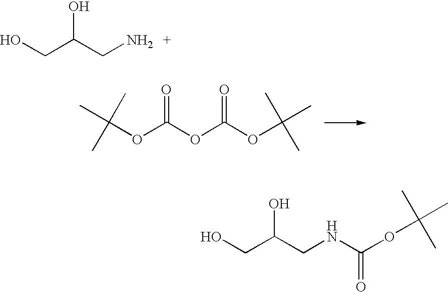

tert-Butyl 2,3-dihydroxypropylcarbamate: di-tert-Butyl dicarbonate (43.6 g; 200 mmol; 1.00 equiv) was added in several batches to a solution of 3-aminopropane-1,2-diol (18.2 g; 200 mmol; 1.00 equiv) in acetonitrile (200 mL). The mixture was stirred at about 40° C. for about 16 hours. The resulting mixture was concentrated in vacuo to give the title product as colorless oil (37 g; 97% yield). LC-MS: m/z=192 (MH)⁺.

### Step 2

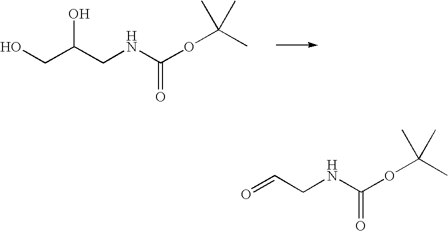

tert-Butyl 2-oxoethylcarbamate: Sodium periodate (41.52 g; 194 mmol; 1.00 equiv) was added in several batches to tert-butyl 2,3-dihydroxypropylcarbamate (37 g; 194 mmol; 1.00 equiv) dissolved in water (300 mL). The resulting solution was stirred at ambient temperature for about 2 hours. The solids were were removed by filtration. Standard extractive workup with dichloromethane, gave the title product as a white solid (17 g; 56% yield). 1H NMR (300 MHz, CDCl3) δ 9.65 (s, 1H), 5.26 (s, 1H), 4.07 (d, J=4.5 Hz, 2H), 1.46 (s, 9H).

### Step 3

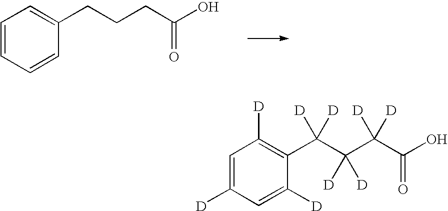

4-d₃-Phenyl-d₈-butanoic acid: Into a high pressure vessel (150 mL), was added tert-butyl 2-oxoethylcarbamate (10 g; 61 mmol) and a freshly prepared d₁-sodium hydroxide/deuterium oxide (10 g) solution. The mixture was stirred at ambient temperature for about 2 hours, 10% palladium on carbon (2 g) was added, and hydrogen gas was introduced. The vessel was sealed and heated at about 150° C. for about 48 hours with vigorous stirring. The solution was cooled to ambient temperature, filtered to remove solids, and the pH of the filtrate was adjusted to 2. Standard extractive workup with ethyl acetate gave a white solid. The procedure of was then repeated with this white solid, to yield the title product (9.8 g; 95% yield). H NMR (para-hydroxyl anisole was used as internal standard) (300 MHz, CDCl3) δ 12.00 (s, 1H), 8.88 (s, 1H), 7.18 (s, 2H), 6.75˜6.65 (m, 4H), 3.65 (s, 3H), 2.65 (s, 0.08H), 2.34 (s, 0.10H), 1.93 (0.10H).

### Step 4

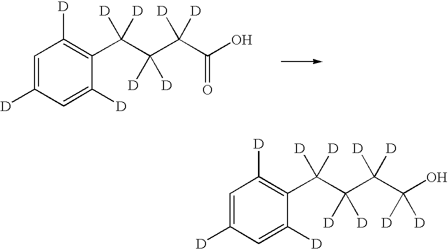

4-d₃-Phenyl-d₈-butan-1-ol: At about 0° C., 4-d₃-phenyl-d₈-butanoic acid (3 g; 18 mmol; 1 equiv) in tetrahydrofuran (20 mL) was added dropwise to the suspension of lithium aluminum deuteride (1.1 g; 26 mmol; 1.5 equiv) in tetrahydrofuran (30 mL). The mixture was stirred at about 0° C. for about 2 hours, and then water (20 mL) was carefully added. The solids were removed by filtration. Standard extractive workup with ethyl acetate gave the title product as a white solid (2.6 g; 93% yield). H NMR (300 MHz, CDCl₃) δ 7.22 (s, 2H), 3.65 (s, 0.04H), 2.65 (s, 0.08H), 2.32 (m, 0.10H), 1.71 (s, 1H), 1.59 (s, 0.10H).

### Step 5

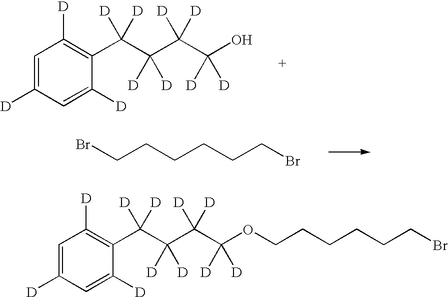

1-(4-(6-Bromohexyloxy)-d₈-butyl)-d₃-benzene: Under an atmosphere of nitrogen, 1,6-dibromohexane (3.66 g; 15.00 mmol; 3.00 equiv), sodium hydride (240 mg; 6.00 mmol; 1.20 equiv), tetra-n-butylammonium bromide (48 mg; 0.15 mmol; 0.03 equiv) were added to 4-d₃-phenyl-d₈-butan-1-ol (805 mg; 5.00 mmol; 1.00 equiv) dissolved in tetrahydrofuran (20 mL). The resulting solution was heated at reflux for about 16 hours, and then quenched by adding ammonium chloride. Following standard extractive workup with ethyl acetate, the crude residue was purified by silica gel column chromotagraphy with ethyl acetate/petroleum ether (1/50) to afford the title product as a colorless oil (1.1 g; 68% yield). LC-MS: m/z=324 (MH)⁺.

### Step 6

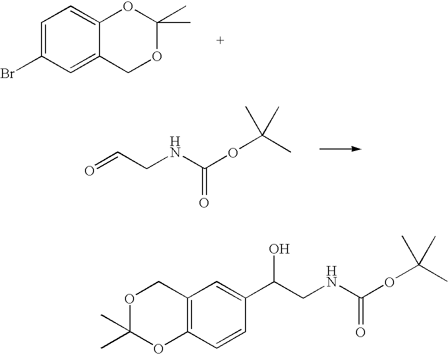

tert-Butyl-(2,2-dimethyl-4H-benzo[d][1,3]dioxin-6-yl)-2-hydroxyethylcarbamate: Under atmosphere of nitrogen, a solution of 6-bromo-2,2-dimethyl-4H-benzo[d][1,3]dioxine (4.86 g; 20.00 mmol; 2.00 equiv) was dissolved in tetrahydrofuran (50 mL). At about −78° C., 2.5M n-butyllithium (8 mL; 2.00 equiv) was added dropwise with stirring. The resulting solution was stirred at about −78° C. for about 1 hour, and then tert-butyl 2-oxoethylcarbamate (1.59 g; 10.00 mmol; 1.00 equiv) was added. The reaction mixture was stirred at about −78° C. for about 10 minutes, and then 5% acetic acid was added. Following standard extractive workup with ethyl acetate, the crude residue was purified by silica gel column chromatography with ethyl acetate/petroleum ether (1/4-1/2) to give the title product as a white powder (1.4 g; 43% yield). LC-MS: m/z=324 (MH)+.

### Step 7

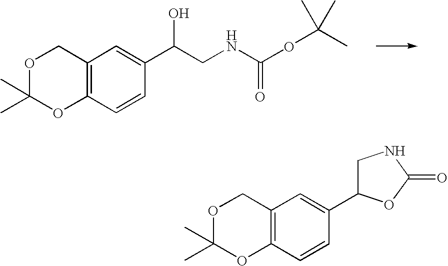

5-(2,2-Dimethyl-4H-benzo[d][1,3]dioxin-6-yl)oxazolidin-2-one: tert-Butyl 2-(2,2-dimethyl-4H-benzo[d][1,3]dioxin-6-yl)-2-hydroxyethylcarbamate (646 mg; 2.00 mmol; 1.00 equiv.) was dissolved in N,N-dimethylformamide (10 mL), and then sodium hydride (96 mg; 2.40 mmol; 1.20 equiv) was added. The resulting solution was stirred at about 30° C. for about 2 hours and concentrated in vacuo. The residue was diluted with ethyl acetate (20 mL), washed with water (10 mL), and dried over magnesium sulfate. The crude product was purified by re-crystallization from ethyl acetate to give the title product as a white powder (280 mg; 56% yield). 1H NMR (300 MHz, CDCl3) δ 7.15 (dd, J=8.4, 2.1 Hz, 1H), 7.04 (s, 1H), 6.85 (d, J=8.4 Hz, 1H), 5.54 (t, J=8.1 Hz, 1H), 4.85 (s, 2H), 3.93 (t, J=8.4 Hz, 1H), 3.54 (t, J=8.4 Hz, 1H), 1.55 (s, 6H). LC-MS: m/z=250 (MH)+.

### Step 8

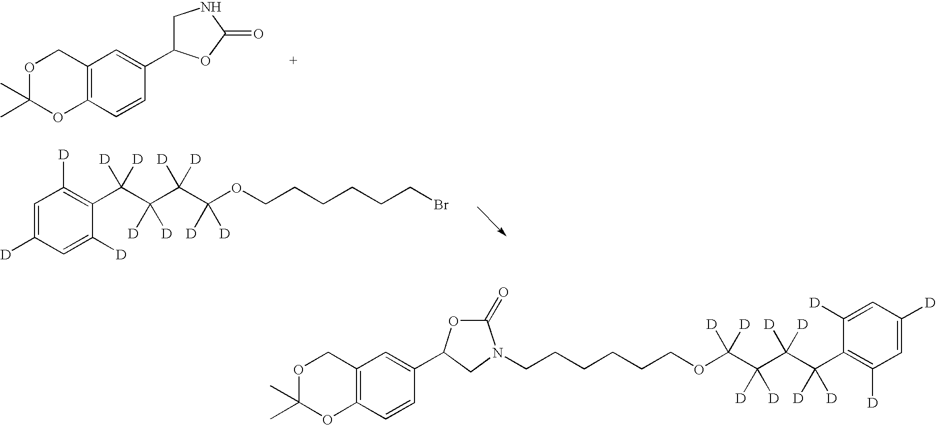

5-(2,2-Dimethyl-4H-benzo[d][1,3]dioxin-6-yl)-3-(6-(4-d₃-phenyl-d₈-butoxy)hexyl)oxazolidin-2-one: Under an atmosphere of nitrogen, 5-(2,2-dimethyl-4H-benzo[d][1,3]dioxin-6-yl)oxazolidin-2-one (150 mg; 0.60 mmol; 1.00 equiv) was dissolved in N,N-dimethylformamide (10 mL). Sodium hydride (29 mg; 0.72 mmol; 1.20 equiv) was added, and the resulting solution was stirred at about 30° C. for about 1 hour. 1-(4-(6-Bromohexyloxy)-d₈-butyl)-d₃-benzene (389 mg; 1.20 mmol; 2.00 equiv) was added, and the reaction mixture was stirred at about 30° C. for about 16 hours. Following standard extractive workup with ethyl acetate, the crude residue was purified by silica gel column chromotagraphy with ethyl acetate/petroleum ether (1/5-1/2) to give the title product as a colorless oil (270 mg; 91% yield). LC-MS: m/z=493 (MH)+.

### Step 9

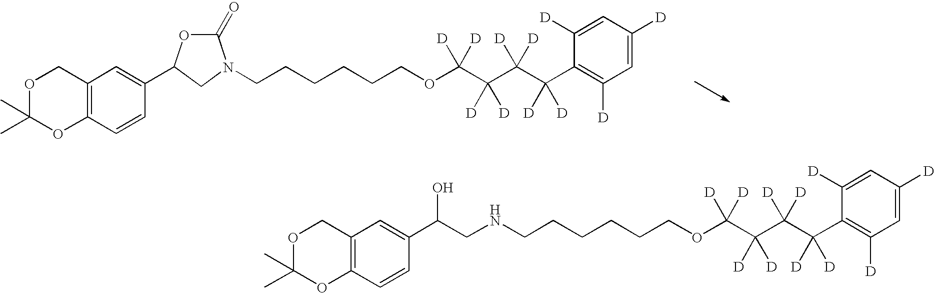

1-(2,2-Dimethyl-4H-benzo[d][1,3]dioxin-6-yl)-2-(6-(4-d₃-phenyl-d₈-butoxy)hexylamino)ethanol: Under an atmosphere of nitrogen, potassium trimethylsilanolate (211 mg, 1.65 mmol, 3.00 equiv) was added to a solution of 5-(2,2-dimethyl-4H-benzo[d][1,3]dioxin-6-yl)-3-(6-(4-d₃-phenyl-d₈-butoxy)hexyl)oxazolidin-2-one (270 mg, 0.55 mmol, 1.00 equiv) in tetrahydrofuran (10 mL). The resulting solution was has heated at reflux for about 16 hours, and then water (10 mL) was added. Standard extractive workup with ethyl acetate gave the title product as a colorless oil (150 mg; yield 59%). LC-MS: m/z=467 (MH)+.

### Step 10

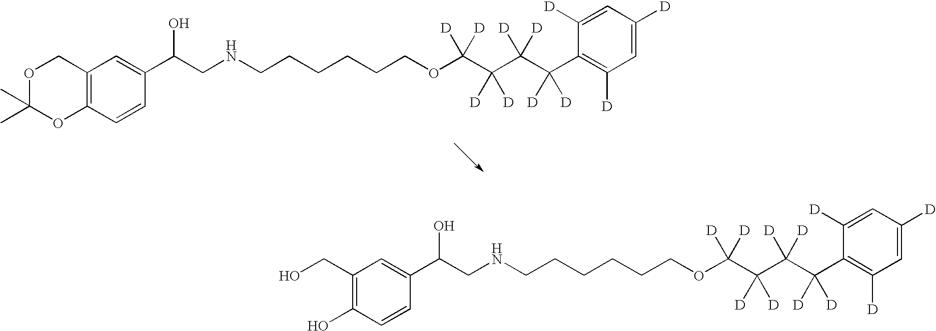

4-( 1-Hydroxy-2-(6-(4-d₃-phenyl-d₈-butoxy)hexylamino)ethyl)-2-(hydroxymethyl)phenol: 1-(2,2-Dimethyl-4H-benzo[d][1,3]dioxin-6-yl)-2-(6-(4-d₃-phenyl-d₈-butoxy)hexylamino)ethanol (150 mg; 0.32 mmol; 1.00 equiv) was dissolved in acetic acid (5 mL) and water (1 mL). The resulting solution was stirred at about 80° C. for about 2 hours, and then concentrated in vacuo. The resulting residue was diluted with water (5 mL), and the pH of the solution was adjusted to 8 with sodium bicarbonate. Standard extractive workup with ethyl acetate afforded the title product as colorless oil (120 mg; 87% yield). LC-MS: m/z=427 (MH)+.

### Step 11

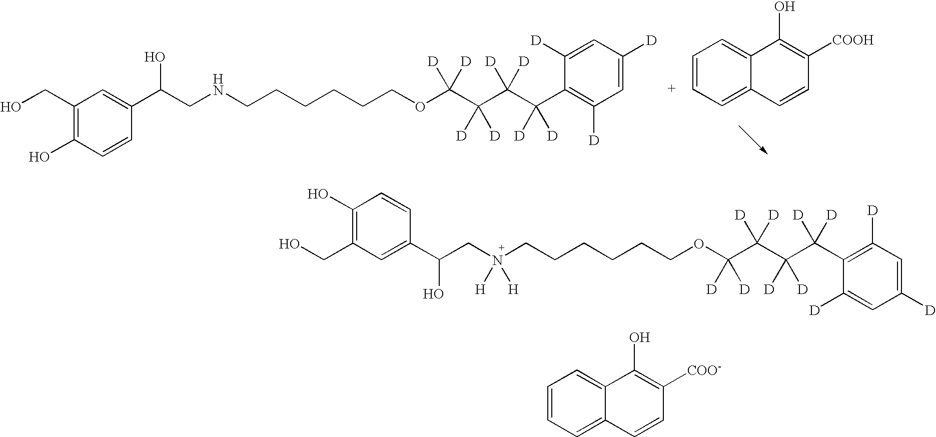

N-(2-hydroxy-2-(4-hydroxy-3-(hydroxymethyl)phenyl)ethyl)-6-(4-d₃-phenyl-d₈-butoxy)hexan-1-aminium 1-hydroxy-2-naphthoate: 4-(1-Hydroxy-2-(6-(4-d₃-phenyl-d₈-butoxy)hexylamino)ethyl)-2-(hydroxymethyl)phenol (120 mg; 0.28 mmol; 1.00 equiv) was dissolved in ethyl acetate (10 mL). A solution of 1-hydroxy-2-naphthoic acid (105 mg; 0.56 mmol; 2.00 equiv) in ether (2 mL) was then added dropwise. The mixture was stirred at ambient temperature for about 2 hours, and then the solids were collected by filtration. The title compound was isolated as a white solid (130 mg; 75% yield).¹H NMR (300 MHz, d₆-DMSO) δ 9.45 (s, 1H), 8.59 (s, br, 1H), 8.19 (d, J=7.8 Hz, 1H), 7.71 (t, J=8.7 Hz, 2H), 7.38-7.44 (m, 3H), 7.17 (s, 1H), 7.06 (d, J=7.5 Hz, 1H), 6.97 (d, J=8.7 Hz, 1H), 6.76 (d, J=8.1 Hz, 1H), 6.04 (s, br, 1H), 5.03 (s, br, 1H), 4.78 (s, br, 1H), 4.49 (s, 2H), 3.31 (s, 2H), 2.92-3.07 (m, 4H), 1.61 (m, 2H), 1.46 (m, 2H), 1.29 (m, 4H).

### EXAMPLE 3

### N-(2-Hydroxy-2-(4-hydroxy-3-(hydroxymethyl)phenyl)ethyl)-6-(4-d₃-phenyl-d₈-butoxy)-d₂-hexan-1-aminium 1-hydroxy-2-naphthoate

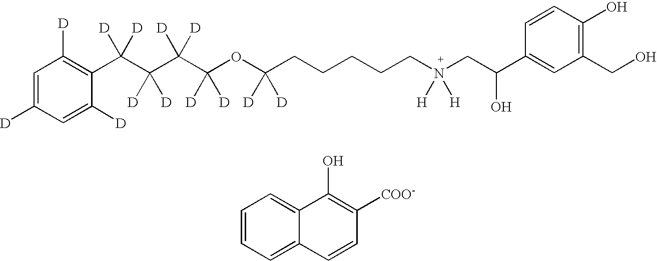

### Step 1

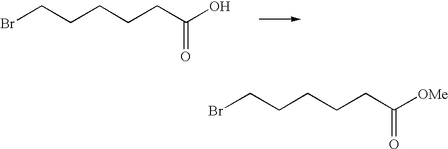

Methyl 6-bromohexanoate: At about 0° C., thionyl chloride (11.9 g; 100 mmol; 1.00 equiv) was added dropwise to a stirred solution of 6-bromohexanoic acid (19.5 g; 100 mmol; 1.00 equiv) in methanol (100 mL). The resulting solution was stirred at ambient temperature for about 1 hour and then concentrated in vacuo, to give the title product as a colorless oil (19 g; 91% yield). 1H NMR (300 MHz, CDCl3) δ 3.68 (s, 3H), 3.41 (t, J=6.6 Hz, 2H), 2.43 (t, J=6.6 Hz, 2H), 1.88 (m, 2H), 1.64 (m, 2H), 1.49 (m, 2H).

### Step 2

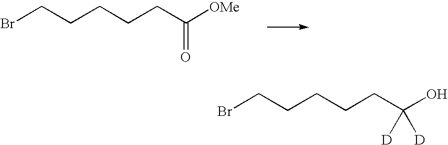

6-Bromohexan-d₂-1-ol: At about 0° C. and under an atmosphere of nitrogen, a solution of methyl 6-bromohexanoate (2.09 g; 10.00 mmol; 1.00 equiv) in tetrahydrofuran (10 mL) was added dropwise to a solution of lithium aluminum deuteride (630 mg; 15.00 mmol; 1.50 equiv) in tetrahydrofuran (20 mL). The resulting solution was stirred for about 2 hours at ambient temperature, and then 5% aqueous acetic acid was added. Following standard extractive workup with ethyl acetate, the crude residue was purified by silica gel column chromotagraphy with ethyl aceate/ petroleum ether (1/5-1/2) to give the title product as a colorless oil (550 mg; 30% yield). 1H NMR (300 MHz, CDCl3) δ 3.68 (t, J=2.7 Hz, 0.04H), 3.43 (t, J=6.6 Hz, 2H), 1.89 (m, 2H), 1.54 (m, 2H), 1.32-1.42 (m, 4H).

### Step 3

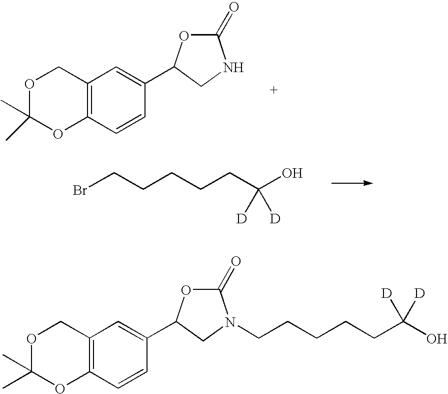

5-(2,2-Dimethyl-4H-benzo[d][1,3]dioxin-6-yl)-3-(6-d₂-hydroxyhexyl)oxazolidin-2-one: The procedure of Example 2, Step 8 was followed, but substituting 6-bromo-hexan-d₂-1-ol for 6-bromohexan-1-ol. The title product was isolated as a colorless oil (393 mg; 70% yield). LC-MS: m/z=352 (MH)+.

### Step 4

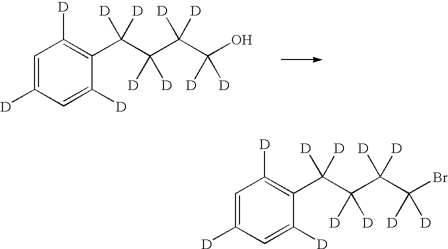

1-(4-Bromo-d₈-butyl)-d₃-benzene: 4-d₃-Phenyl-d₈-butan-1-ol (160 mg; 1 mmol; 1 equiv) and tetrabromomethane (400 mg; 1.2 mmol; 1.2 eq) were dissolved in dichloromethane (15 mL). Triphenylphosphine (315 mg; 1.2 mmol; 1.2 equiv) was added to the solution, the solution was stirred for about 16 hours at ambient temperature, and then concentrated in vacuo. The crude product was then purified by silica gel column chromotagraphy and directly eluted with petroleum ether to afford the title compound as a colorless oil (200 mg; 90% yield).

### Step 5

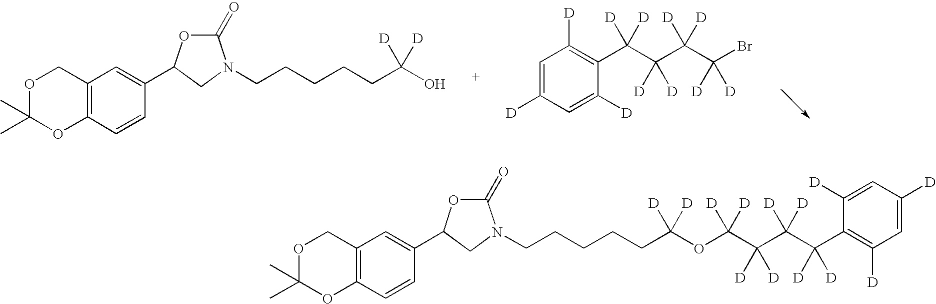

5-(2,2-Dimethyl-4H-benzo[d][1,3]dioxin-6-yl)-3-(6-(4-d₃-phenyl-d₈-butoxy)-d₂-hexyl)oxanzolidin-2-one: 1-(4-Bromo-d₈-butyl)-d₃-benzene (336 mg; 1.50 mmol; 1.50 equiv), sodium hydride (48 mg; 1.20 mmol; 1.20 equiv) and tetra-n-butylammonium bromide were added to a solution of 5-(2,2-dimethyl-4H-benzo[d][1,3]dioxin-6-yl)-3-(6-hydroxy-d₂-hexyl)oxazolidin-2-one (351 mg; 1.00 mmol; 1.00 equiv) in N,N-dimethylformamide (10 mL). The resulting solution was stirred at about 40° C. for about 16 hours. Following standard extractive workup with ethyl acetate, the crude residue was purified by silica gel column chromotagraphy with ethyl acetate/petroleum ether (1/1) to give the title product as a colorless oil (320 mg; 65% yield). LC-MS: m/z=495 (MH)+.

### Step 6

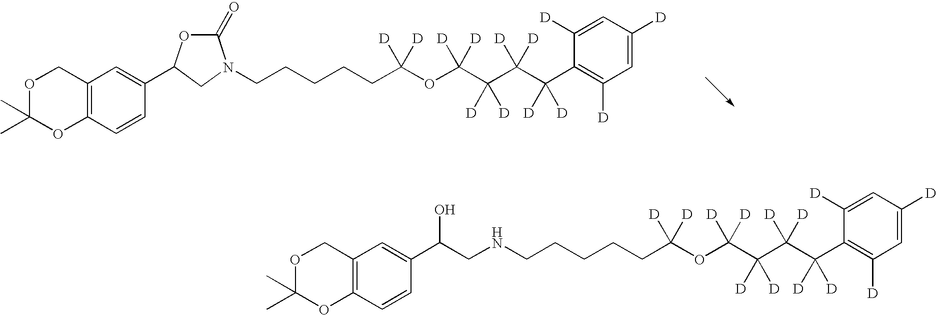

1-(2,2-Dimethyl-4H-benzo[d][1,3]dioxin-6-yl)-2-(6-(4-d₃-phenyl-d₈-butoxy)-d₂-hexylamino)ethanol: The procedure of Example 2, Step 9 was followed but substituting 5-(2,2-dimethyl-4H-benzo[d][1,3]dioxin-6-yl)-3-(6-(4-d₃-phenyl-d₈-butoxy)-d₂-hexyl)oxanzolidin-2-one for 5-(2,2-dimethyl-4H-benzo[d][1,3]dioxin-6-yl)-3-(6-(4-d₃-phenyl-d₈-butoxy)hexyl)oxanzolidin-2-one. The title product was isolated as a colorless oil (210 mg; 75% yield). LC-MS: m/z=469 (MH)+.

### Step 7

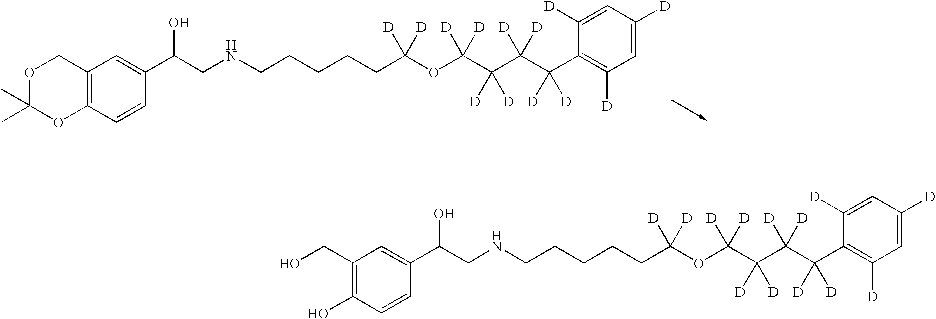

4-(1-Hydroxy-2-(6-(4-d₃-phenyl-d₈-butoxy)-d₂-hexylamino)ethyl)-2-(hydroxymethyl)phenol: The procedure of Example 2, Step 10 was followed, but substituting 1-(2,2-dimethyl-4H-benzo[d][1,3]dioxin-6-yl)-2-(6-(4-d₃-phenyl-d₈-butoxy)-d₂-hexylamino)ethanol for 1-(2,2-dimethyl-4H-benzo [d][1,3]dioxin-6-yl)-2-(6-(4-d₃-phenyl-d₈-butoxy)hexylamino)ethanol. The title product was isolated as a colorless oil (40 mg; 76% yield). LC-MS: m/z=429 (MH)+.

### Step 8

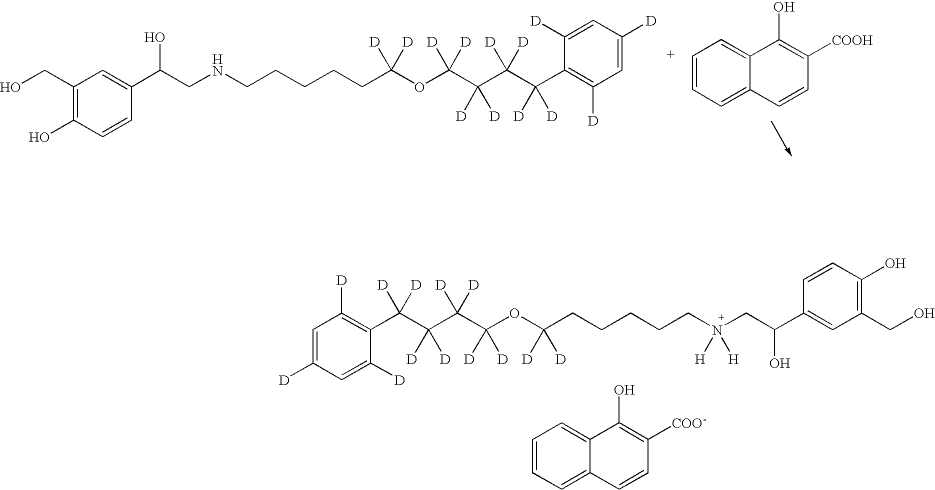

N-(2-Hydroxy-2-(4-hydroxy-3-(hydroxymethyl)phenyl)ethyl)-6-(4-d₃-phenyl-d₈-butoxy)-d₂-hexan-1-aminium 1-hydroxy-2-naphthoate: The procedure of Example 2, Step 11 was followed but substituting 4-(1-hydroxy-2-(6-(4-d₃-phenyl-d₈-butoxy)-d₂-hexylamino)ethyl)-2-(hydroxymethyl)phenol for 4-(1-hydroxy-2-(6-(4-d₃-phenyl-d₈-butoxy)hexylamino)ethyl)-2-(hydroxymethyl)phenol. The title product was isolated as a white solid (75 mg; 37% yield). ¹H NMR (300 MHz, DMSO-d₆) δ 9.47 (s, 1H), 8.62 (s, br, 2H), 8.20 (d, J=8.4 Hz, 1H), 7.72 (t, J=8.4 Hz, 2H), 7.37-7.48 (m, 3H), 7.18 (s, 2H), 7.07 (d, J=8.4 Hz, 1H), 7.00 (d, J=8.4 Hz, 1H), 6.77 (d, J=8.1 Hz, 1H), 6.05 (s, br, 1H), 5.04 (s, br, 1H), 4.80 (d, J=9.6 Hz, 1H), 4.50 (s, 2H), 2.93-3.04 (m, 4H), 1.62 (m, 2H), 1.46 (m, 2H), 1.30 (m, 4H).

### EXAMPLE 4

### N-(2-Hydroxy-2-(4-hydroxy-3-(hydroxymethyl)phenyl)ethyl)-6-(4-d₃-phenyl-d₈-butoxy)-d₄-hexan-1-aminium 1-hydroxy-2-naphthoate

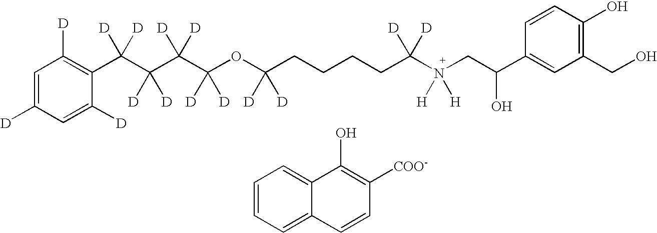

### Step 1

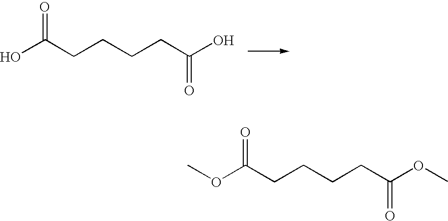

Dimethyl adipate: Thionyl chloride (1 mL) was added dropwise to a solution of adipic acid (30 g, 205.48 mmol, 1.00 equiv) in methanol (200 mL). The mixture was stirred for about 16 hours at ambient temperature. The reaction mixture was concentrated in vacuo and the resulting residue was then purified by silica gel column chromotagraphy with ethyl acetate/petroleum ether (1/15) to give the title product as light yellow liquid (21.1 g, 58% yield). ¹H NMR (300 MHz, CDCl₃) δ: 3.67 (s, 6H), 2.34 (m, 4H), 1.67 (m, 4H).

### Step 2

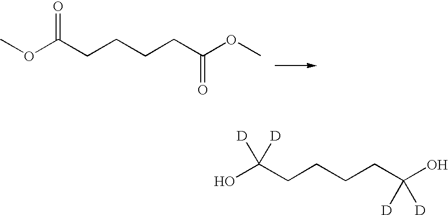

d₄-Hexane-1,6-diol: Under an atmosphere of nitrogen, a suspension of lithium aluminum deuteride (1.8 g; 42.86 mmol; 2.49 equiv) in tetrahydrofuran (20 mL) was added to a solution of dimethyl adipate (3 g; 17.2 mmol; 1.00 equiv) in tetrahydrofuran (30 mL). The suspension was vigorously stirred for about 2 hours at ambient temperature, and then decahydrated sodium sulphate (2g) was added. The solids were removed by filtration and washed with ethyl acetate. The filtrate and the washings were combined and concentrated in vacuo to give the title product as white solid (1.8 g; 86% yield). ¹H NMR (300 MHz, CDCl₃) δ: 1.58 (m, 4H), 1.41 (m, 4H)

### Step 3

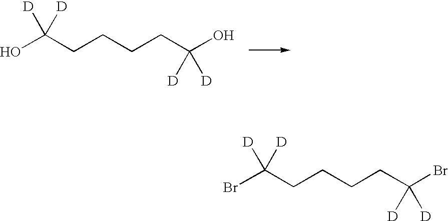

d₄-1,6-Dibromohexane: A solution of d₄-hexane-1,6-diol (400 mg; 3.11 mmol; 1.00 equiv) in tribromophosphine (900 mg; 3.33 mmol; 2.00 equiv) was stirred at about 100° C. for about 3 hours, and then water (20 mL) was added. Following standard extractive workup with ethyl acetate, the crude residue was purified by silica gel column chromotagraphy with petroleum ether to give the title product as colorless liquid (550 mg; 68% yield). ¹H NMR (300 MHz, CDCl₃) δ: 1.87 (m, 4H), 1.47 (m, 4H).

### Step 4

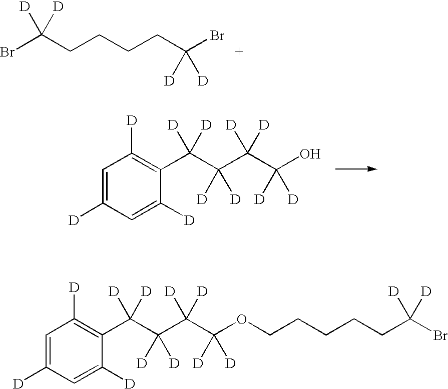

1-(4-(6-Bromo-d₄-hexyloxy)-d₈-butyl)-d₃-benzene: Into a solution of d₄-1,6-dibromohexane (800 mg; 3.16 mmol; 2.00 equiv), potassium hydroxide (334 mg; 5.96 mmol; 3.77 equiv) and tetrabutylammonium hydrogen sulfate (55 mg; 0.16 mmol; 0.10 equiv) in toluene (20 mL) was added a solution of d₁₁-4-phenylbutan-1-ol (260 mg; 1.58 mmol; 1.00 equiv) in toluene (10 mL). The mixture was stirred at ambient temperature for about 16 hours. Following standard extractive workup with ethyl acetate, the crude residue was purified by silica gel column chromotagraphy with petroleum ether to give the title product as colorless liquid (360 mg; 66% yield). ¹H NMR (300 MHz, CDCl₃) δ: 7.21 (s, 2H), 1.88 (t, J=6.6, 7.5 Hz, 2H), 1.59 (m, 2H), 1.45 (m, 4H).

### Step 5

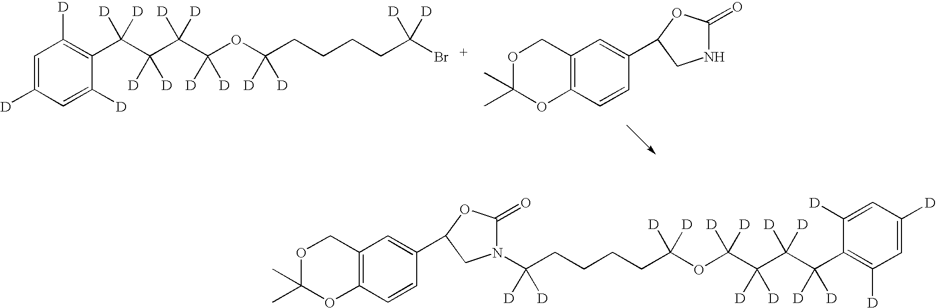

5-(2,2-Dimethyl-4H-benzo[d][1,3]dioxin-6-yl)-3-(6-(4-d₃-phenyl-d₈-butoxy)-d₄-hexyl)oxazolidin-2-one: At about 30° C., a solution of 5-(2,2-dimethyl-4H-benzo[d][1,3]dioxin-6-yl)oxazolidin-2-one (228 mg; 0.91 mmol; 1.00 equiv) in N,N-dimethylformamide (10 mL) was added to a suspension of sodium hydride (40 mg; 1.00 mmol; 1.10 equiv) in N,N-dimethylformamide (10 mL). The suspension was stirred at about 30° C. for about 1 hour. A solution of 1-(4-(6-bromo-d₄-hexyloxy)-d₈-butyl)-d₃-benzene (360 mg; 1.04 mmol; 1.15 equiv) in N,N-dimethylformamide (10 mL) was added and the resulting solution was stirred at about 30° C. for about 16 hours. Following standard extractive workup with ethyl acetate, the crude residue was purified by silica gel column chromotagraphy with ethyl acetate/petroleum ether (1/5˜1/3) to give the title product as colorless oil (340 mg; 75% yield). ¹H NMR (300 MHz, CDCl₃) δ: 7.21 (s, 2H), 7.14 (dd, J=1.8, 1.8 Hz, 1H), 7.02 (s, 1H), 6.86 (d, J=8.1 Hz, 1H), 5.41 (t, J=7.8, 8.1 Hz, 1H), 4.86 (s, 2H), 3.86 (t, J=8.7, 8.7 Hz, 1H), 3.41 (t, J=7.8, 8.4 Hz, 1H), 1.59 (m, 10H), 1.38 (m, 4H).

### Step 6

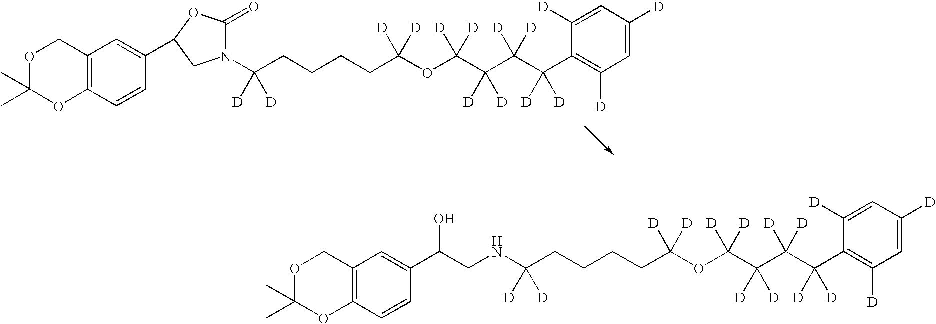

1-(2,2-Dimethyl-4H-benzo[d][1,3]dioxin-6-yl)-2-(6-(4-d₃-phenyl-d₈-butoxy)-d₄-hexylamino)ethanol: The procedure of Example 2, Step 9 was followed, but substituting 5-(2,2-dimethyl-4H-benzo[d][1,3]dioxin-6-yl)-3-(6-(4-d₃-phenyl-d₈-butoxy)-d₄-hexyl)oxazolidin-2-one for 5-(2,2-dimethyl-4H-benzo[d][1,3]dioxin-6-yl)-3-(6-(4-d₃-phenyl-d₈-butoxy)hexyl)oxazolidin-2-one. The title product was isolated as a pale red oil (300 mg; 90% yield). LC-MS: m/z =471 (MH)⁺.

### Step 7

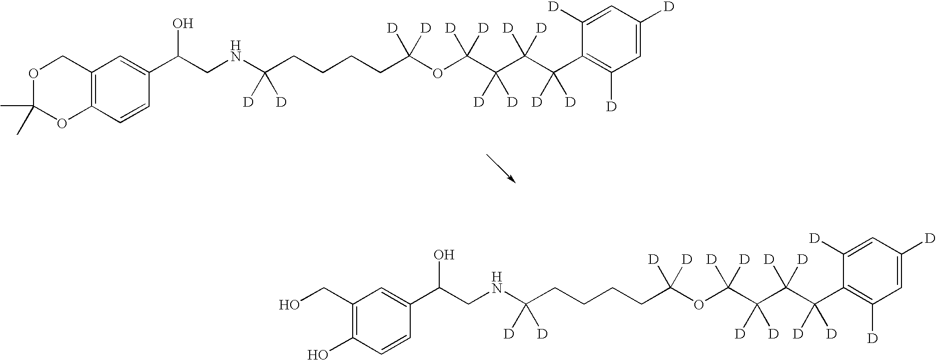

4-(1-Hydroxy-2-(6-(4-d₃-phenyl-d₈-butoxy)-d₄-hexylamino)ethyl)-2-(hydroxymethyl)phenol: The procedure of Example 2, Step 10 was followed, but substituting 1-(2,2-dimethyl-4H-benzo[d][1,3]dioxin-6-yl)-2-(6-(4-d₃-phenyl-d₈-butoxy)-d₄-hexylamino)ethanol for 1-(2,2-dimethyl-4H-benzo[d][1,3]dioxin-6-yl)-2-(6-(4-d₃-phenyl-d₈-butoxy)hexylamino)ethanol. The title product was isolated as a white solid.

### Step 8

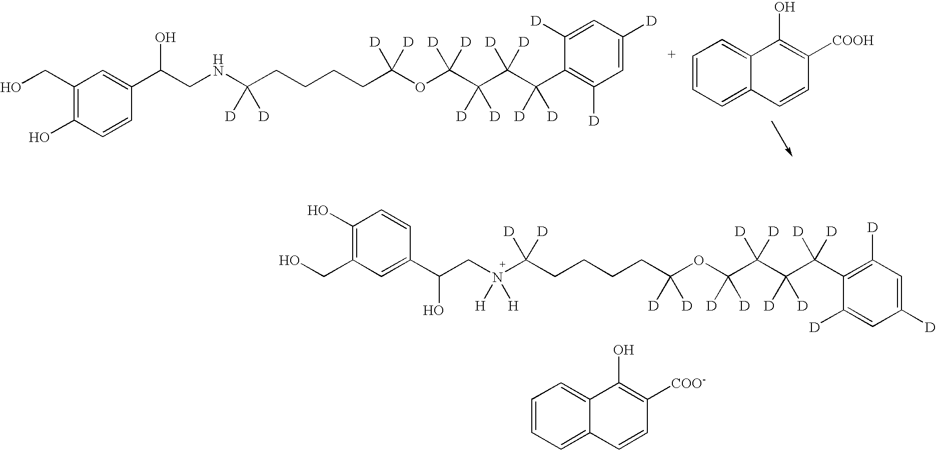

d₁₅-N-(2-hydroxy-2-(4-hydroxy-3-(hydroxymethyl)phenyl)ethyl)-6-(4-d₃-phenyl-d₈-butoxy)-d₄-hexan-1-aminium 1-hydroxy-2-naphthoate: The procedure of Example 2, Step 11 was followed, but substituting 4-(1-hydroxy-2-(6-(4-d₃-phenyl-d₈-butoxy)-d₄-hexylamino)ethyl)-2-(hydroxymethyl)phenol for 4-(1-hydroxy-2-(6-(4-d₃-phenyl-d₈-butoxy)hexylamino)ethyl)-2-(hydroxymethyl)phenol. The title product was isolated as a white solid (130 mg; 34% yield). ¹H NMR (300 MHz, DMSO) δ: 9.47 (s, 1H), 8.61 (s, 2H), 8.20 (d, J=8.1 Hz, 1H), 7.72 (t, J=8.4, 8.7 Hz, 2H), 7.45 (t, J=6.9, 6.9 Hz, 1H), 7.37 (m, 2H), 7.18 (s, 2H), 7.06 (d, J=7.8 Hz, 1H), 6.99 (d, J=8.4 Hz, 1H), 6.77 (d, J=8.1 Hz, 1H), 6.05 (s, 1H), 5.05 (s, 1H), 4.81 (d, J=9.0 Hz, 1H), 4.49 (s, 2H), 3.07˜2.94 (m, 4H), 1.61 (s, 2H), 1.46 (s, 2H), 1.30 (m, 4H). LC-MS: m/z=431 (MH)⁺.

### EXAMPLE 5

### N-(2-Hydroxy-2-(4-hydroxy-3-(hydroxymethyl)phenyl)ethyl)-6-(4-d₃-phenyl-d₈-butoxy)hexan-1-d₂-aminium 1-hydroxy-2-naphthoate

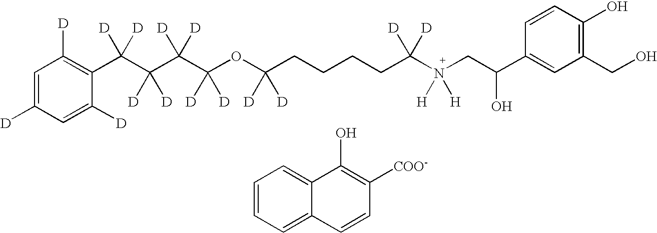

### Step 1

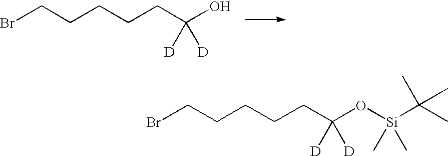

d₂-(6-Bromohexyloxy)(tert-butyl)dimethylsilane: A solution of tert-butylchlorodimethylsilane (609 mg, 4.04 mmol, 2.04 equiv) in dichloromethane (10 mL) was added to a solution of d₂-6-bromohexan-1-ol (370 mg; 1.98 mmol; 1.00 equiv) and 1H-imidazole (412 mg; 6.06 mmol; 3.06 equiv) in dichloromethane (20 mL). The resulting solution was stirred at about 30° C. for about 1 hour and then washed with water (3×20 mL). The organic layer was dried over anhydrous sodium sulfate and concentrated in vacuo. The resulting residue was purified by silica gel column chromotagraphy with petroleum ether to give the title product as pale yellow liquid (400 mg; 67% yield). ¹H NMR (300 MHz, CDCl₃) δ 3.43 (t, J=6.9 Hz, 2H), 1.89 (m, 2H), 1.53˜1.37 (m, 6H), 0.91 (s, 9H), 0.07 (s, 6H).

### Step 2

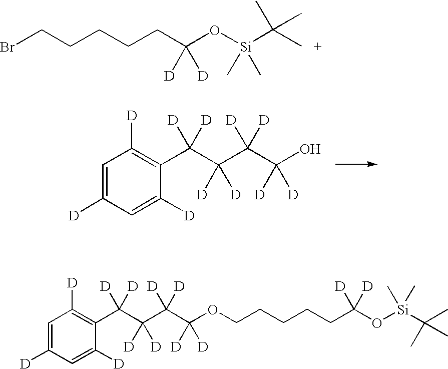

tert-Butyldimethyl(6-(4-d₃-phenyl-d₈-butoxy)-d₂-hexyloxy)silane: The procedure of Example 4, Step 4 was followed but substituting d₂-(6-bromohexyloxy)(tert-butyl)dimethylsilane for d₄-1,6-dibromohexane. The title product was isolated as light yellow oil (460 mg; 51% yield). ¹H NMR (300 MHz, CDCl₃) δ 7.20 (s, 2H), 3.40 (t, J=6.6 Hz, 2H), 1.61˜1.53 (m, 4H), 1.37˜1.35 (m, 4H), 0.91 (s, 9H), 0.07 (s, 6H); LC-MS: m/z=378 (MH)⁺.

### Step 3

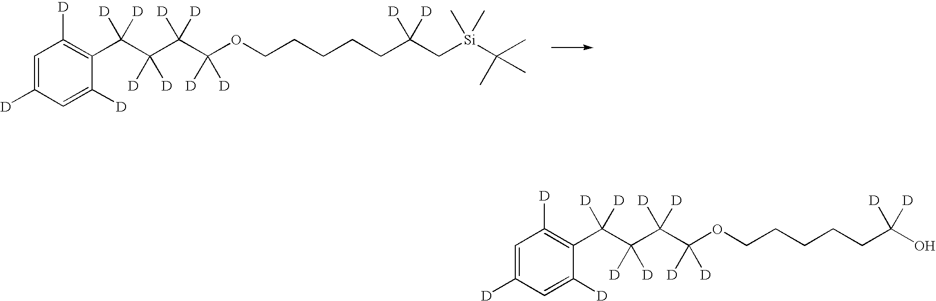

6-(4-d₃-Phenyl-d₈-butoxy)hexan-1-d₂-ol: Tetrabutylammonium fluoride (686 mg; 2.63 mmol; 1.88 equiv) was added to a solution of tert-butyldimethyl(6-(4-d₃-phenyl-d₈-butoxy)-d₂-hexyloxy)silane (660 mg; 1.40 mmol; 1.00 equiv) in tetrahydrofuran (50 mL). The mixture was stirred at about 30° C. for about 2 hours. Following standard extractive workup with ethyl acetate, the crude residue was purified by preparative thin layer chromotagraphy with petroleum ether/ethyl acetate (1/2) to give the title product as colorless liquid (330 mg; 88% yield). ¹H NMR (300 MHz, CDCl₃) δ 7.20 (s, 2H), 3.41 (t, J=6.6 Hz, 2H), 1.62˜1.58 (m, 4H), 1.53 (s, 1H), 1.41˜1.37 (m, 4H); LC-MS: m/z=264 (MH)⁺.

### Step 4

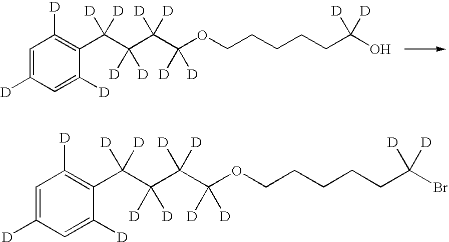

1-(4-(6-Bromo-d₂-hexyloxy)-d₈-butyl)-d₃-benzene: The procedure of Example 3, Step 4 was followed, but substituting 6-(4-d₃-phenyl-d₈-butoxy)hexan-1-d₂-ol for 4-d₃-phenyl-d₈-butan-1-ol. The title product was isolated as colorless liquid (370 mg; 90% yield). ¹H NMR (300 MHz, CDCl₃) δ 7.21 (s, 2H), 3.41 (t, J=6.6 Hz, 2H), 1.88 (t, J=6.6 Hz, 2H), 1.60 (m, 2H), 1.53˜1.35 (m, 4H).

### Step 5

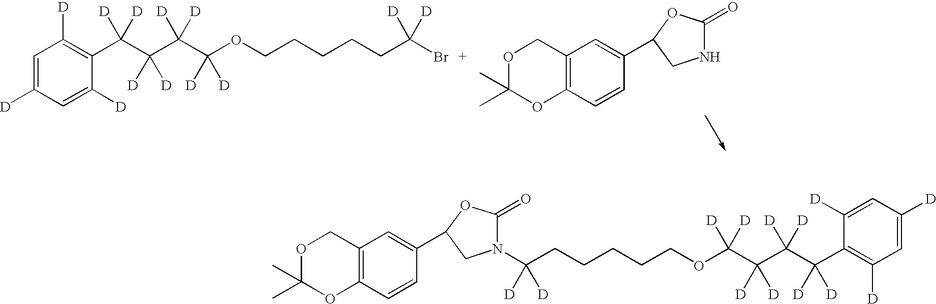

5-(2,2-Dimethyl-4H-benzo[d][1,3]dioxin-6-yl)-3-(6-(4-d₃-phenyl-d₈-butoxy)-d₂-hexyl)oxazolidin-2-one: The procedure of Example 2, Step 8 was followed but substituting 1-(4-(6-bromo-d₂-hexyloxy)-d₈-butyl)-d₃-benzene for 1-(4-(6-bromo hexyloxy)-d₈-butyl)-d₃-benzene. The title product was isolated as a pale yellow oil (390 mg; 96% yield). ¹H NMR (300 MHz, CDCl₃) δ 7.20 (s, 2H), 7.13 (d, J=8.7 Hz, 1H), 7.02 (s, 1H), 6.86 (d, J=8.4 Hz, 1H), 5.41 (t, J=8.1 Hz, 1H), 4.86 (s, 2H), 3.86 (t, J=8.4 Hz, 1H), 3.44˜3.38 (m, 3H), 1.57 (m, 4H), 1.39˜1.38 (m, 4H); LC-MS: m/z=517 (M+Na)⁺.

### Step 6

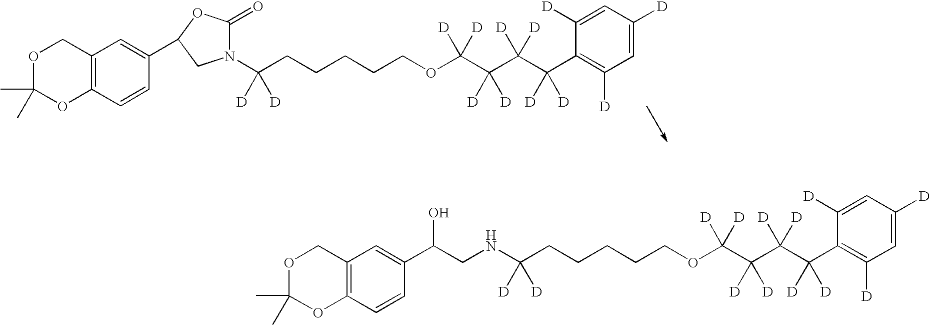

1-(2,2-dimethyl-4H-benzo[d][1,3]dioxin-6-yl)-2-(6-(4-d₃-phenyl-d₈-butoxy)-d₂-hexylamino)ethanol: The procedure of Example 2, Step 9 was followed but substituting 5-(2,2-dimethyl-4H-benzo[d][1,3]dioxin-6-yl)-3-(6-(4-d₃-phenyl-d₈-butoxy)-d₂-hexyl)oxazolidin-2-one for 5-(2,2-dimethyl-4H-benzo[d][1,3]dioxin-6-yl)-3-(6-(4-d₃-phenyl-d₈-butoxy)hexyl)oxazolidin-2-one. The title product was isolated as a yellow oil (300 mg; 75% yield). LC-MS: m/z=469 (M+H)⁺.

### Step 7

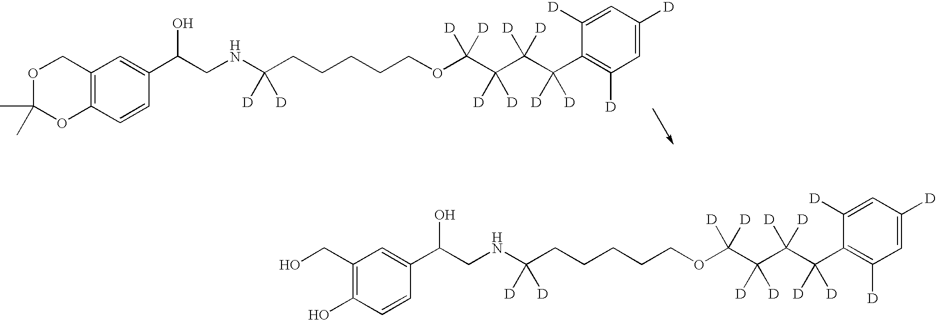

4-( 1-Hydroxy-2-(6-(4-d₃-phenyl-d₈-butoxy)-d₉-hexylamino)ethyl)-2-(hydroxymethyl)phenol: The procedure of Example 2, Step 10 was followed, but substituting 1-(2,2-dimethyl-4H-benzo[d][1,3]dioxin-6-yl)-2-(6-(4-d₃-phenyl-d₈-butoxy)-d₂-hexylamino)ethanol for 1-(2,2-dimethyl-4H-benzo[d][1,3]dioxin-6-yl)-2-(6-(4-d₃-phenyl-d₈-butoxy)hexylamino)ethanol. The title product was isolated as a white solid.

### Step 8

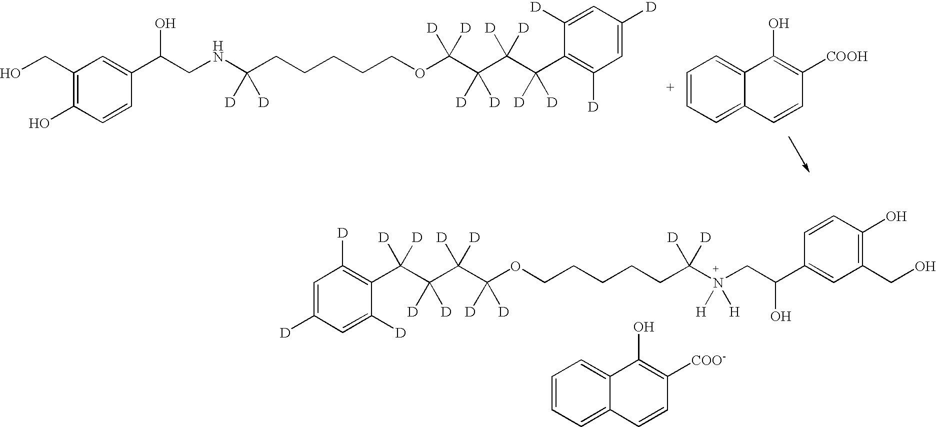

d₁₃-N-(2-Hydroxy-2-(4-hydroxy-3-(hydroxymethyl)phenyl)ethyl)-6-(4-d₃-phenyl-d₈-butoxy) hexan-1-d₂-aminium 1-hydroxy-2-naphthoate: The procedure of Example 2, Step 11 was followed, but substituting 4-(1-hydroxy-2-(6-(4-d₃-phenyl-d₈-butoxy)-d₂-hexylamino)ethyl)-2-(hydroxymethyl)phenol for 4-(1-hydroxy-2-(6-(4-d₃-phenyl-d₈-butoxy)hexylamino)ethyl)-2-(hydroxymethyl)phenol. The title product was isolated as a pale gray solid (85 mg; 48% yield). ¹H NMR (300 MHz, DMSO-d₆) δ 9.46 (s, 1H), 8.59 (s, 2H), 8.20 (d, J=8.1 Hz, 1H), 7.74˜7.69 (m, 2H), 7.48˜7.43 (m, 1H), 7.39˜7.34 (m, 2H), 7.18 (s, 2H), 7.06 (d, J=6.9 Hz, 1H), 6.99 (d, J=8.4 Hz, 1H), 6.77 (d, J=8.1 Hz, 1H), 6.04 (s, 1H), 5.05 (s, 1H), 4.81 (d, J=9.0 Hz, 1H), 4.50 (s, 2H), 3.32 (m, 2H), 3.07˜2.90 (m, 2H), 1.61 (m, 2H), 1.48˜1.46 (m, 2H), 1.30 (m, 4H); LC-MS: m/z=429 (M+H)⁺, 451 (M+Na)⁺.

### EXAMPLE 6

### d₃₇-2-(Hydroxymethyl)-4-[1-hydroxy-2-[6-(4-phenylbutoxy)hexylamino]ethyl]phenyl

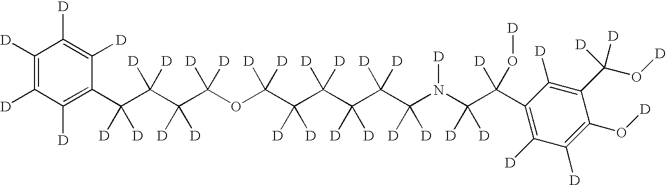

### Step 1

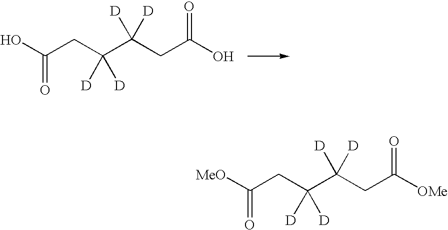

d₄-Hexanedioic acid dimethyl ester: 2.5N Hydrochloric acid is added to a solution of d₄-hexanedioic acid (available commercially from C/D/N Isotopes Inc., Pointe-Claire, Quebec, Canada H9R 1H1) in methanol. The solution is heated at reflux for about 16 hours. Standard extractive workup gives the title product.

### Step 2

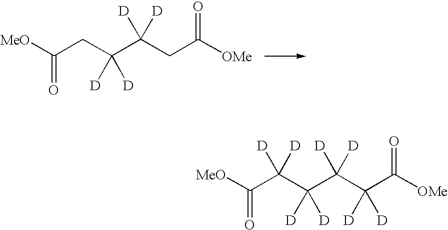

d₈-Hexanedioic acid dimethyl ester: d₃-Sodium methoxide is added to a solution of d₄-hexanedioic acid dimethyl ester in d₄-methanol (available commercially from Sigma-Aldrich Co., St. Louis, Mo. 63103). The solution is heated at reflux for about 16 hours. Standard extractive workup is performed to afford the title product.

### Step 3

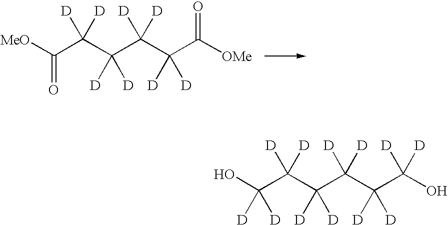

d₁₂-Hexane-1,6-diol: d₈-Hexanedioic acid dimethyl ester is added to a suspension of lithium aluminum deuteride in dry tetrahydrofuran at about 0° C. The mixture is heated at reflux until completion, as measured by thin layer chromatography. Standard extractive workup with ethyl acetate is performed to give the title product.

### Step 4

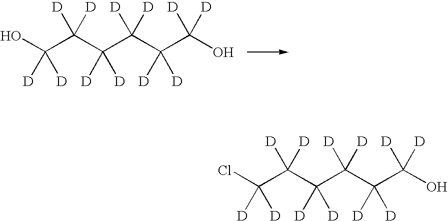

d₁₂-6-Chloro-hexan-1-ol: di-Hydrochloric acid is added to a stirred mixture of d₁₂-hexane-1,6-diol in deuterium oxide and toluene. The solution is heated at reflux for about 5 hours and then allowed to cool to ambient temperature. The reaction is diluted and the aqueous layer is removed. Standard extractive workup is performed to give the title product.

### Step 5

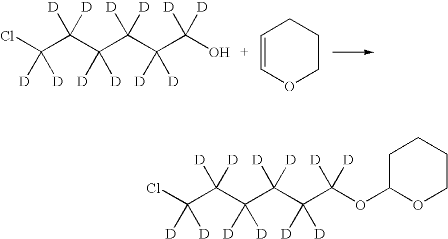

d₁₂-2-[(4-chlorohexyl)oxy]tetrahydropyran: The procedure of Step 5 is carried out using the method as described in U.S. Pat. No. 4,992,474. Dihydropyran (available from Aldrich, St. Louis Park, Mo.) is added to a mixture of d₁₂-cholorohexanol and hydrochloric acid at ambient temperature. The mixture is stirred for about 30 minutes and then washed with water, aqueous sodium bicarbonate and brine. Standard extractive workup with diethyl ether affords the title product.

### Step 6

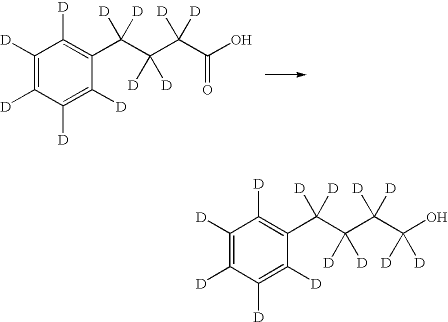

d₁₃-4-phenyl-l-butanol: At about 0° C., d₁₁-4-phenyl butyric acid (available commercially from C/D/N Isotopes Inc., Pointe-Claire, Quebec, Canada H9R 1H1) is added to a suspension of lithium aluminum deuteride in dry tetrahydrofuran. The mixture is heated at reflux until reaction completion, as measured by thin layer chromatography. The reaction is quenched under standard conditions, and filtered. The combined organic layers are dried over sodium sulfate, the solvent is removed under reduced pressure and the crude residue is purified by silica gel chromatography to give the title product.

### Step 7

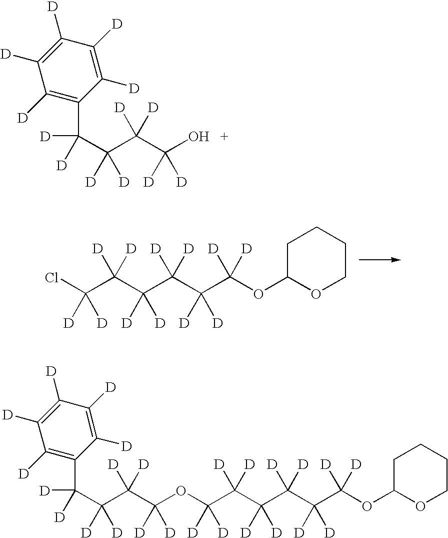

d₂₅-2-[[4-[(6-Phenylbutyl)oxy]hexyl]oxy]tetrahydropyran: The procedure of Step 7 is carried out using the method as described in U.S. Pat. No. 4,992,474. Sodium hydride is added to a mixture of d₁₃-4-phenyl-1-butanol, d₁₂-2-[(4-chlorohexyl)oxy]tetrahydropyran, potassium iodide and tetrahydrofuran. The mixture is then heated at reflux until reaction completion, as measured by thin layer chromatography, and then quenched with water. Standard extractive workup with diethyl ether affords the title product.

### Step 8

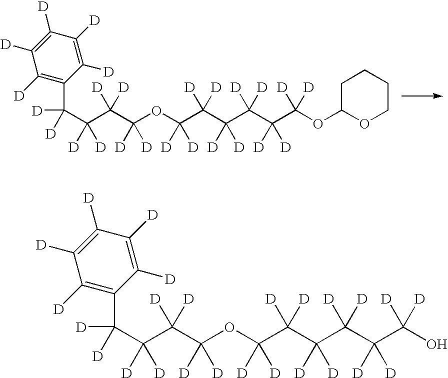

d₂₅-4-[(6-Phenylbutyl)oxy]hexan-1-ol: The procedure of Step 8 is carried out using the method as described in U.S. Pat. No. 4,992,474. A solution of d₂₅-2-[[4-[(6-phenylbutyl)oxy]hexyl]oxy]tetrahydropyran in methanol and 80% acetic acid is stirred at ambient temperature for about 24 hours. Aqueous sodium hydroxide is then added to the solution, and the mixture is heated at reflux for about 2 hours. Standard extractive workup with diethyl ether gives the title product.

### Step 9

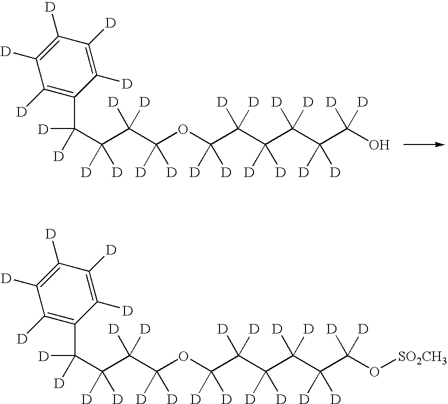

d₂₅-4-[(6-Phenylbutyl)oxy]hexan-1-ol methanesulphonate: The procedure of Step 9 is carried out using the method as described in U.S. Pat. No. 4,992,474. Methanesulphonyl chloride is added dropwise to a solution of d₂₅-4-[(6-phenylbutyl)oxy]hexan-1-ol and trietylamine in dichloromethane at about 0° C. The mixture is stirred at ambient temperature for about 25 minutes and then filtered. The filtrate is washed with saturated aqueous sodium bicarbonate and brine. Standard extractive workup with diethyl ether affords the title product.

### Step 10

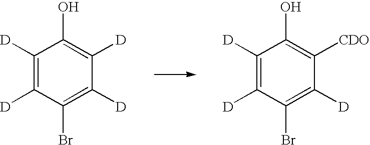

d₃-5-Bromo-2hydroxy-benzaldehyde: The procedure of step 10 is carried out following the protocol set forth in Zhang et al., Chinese Chemical Letters 2006, 17(2), 163-164. Sodium hydroxide and triethylamine is added to a solution of d₄-4-Bromo-phenol (available commercially from C/D/N Isotopes Inc., Pointe-Claire, Quebec, Canada H9R 1H1) in d₁-chloroform. The solution is heated at an elevated temperature until reaction completion, as measured by TLC. The reaction is quenched under standard conditions, and standard extractive workup with diethyl ether affords the title product.

### Step 11

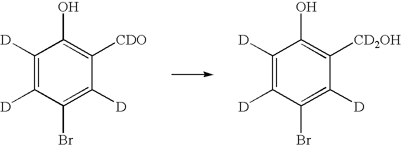

d₆-4-Bromo-2-hydroxymethyl-phenol: The procedure of step 11 is carried out following the protocol set forth in Gisch et al., Journal of Medicinal Chemistry 2007, 50(7), 1658-1667. At about 0° C., a suspension of lithium aluminum deuteride in dry tetrahydrofuran is added to a stirred solution of d₃-5-bromo-2-hydroxy-benzaldehyde. The mixture is heated at reflux for about 3 hours. Following standard extractive workup, the crude residue is purified by silica gel chromatography to give the title product.

### Step 12

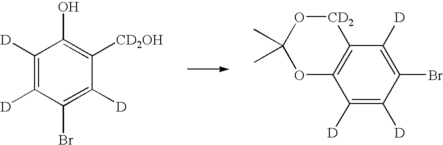

d₅-6-Bromo-2,2-dimethyl-4H-benzo[1,3]dioxine: The procedure of step 12 is carried out following the protocol set forth in Gisch et al., J Med Chem 2007, 50, 1658-1667. d₆-4-Bromo-2-hydroxymethyl-phenol is dissolved in acetone, 2,2-dimethoxypropane, p-toluenesulfonic acid monohydrate, and anhydrous sodium sulfate. The mixture is maintained at about 40° C. for about 3 days. The solvent is removed in vacuo and the resulting residue is dissolved in ethyl acetate and water. Following standard extractive workup with ethyl acetate, the crude product is purified by column chromatography on silica gel to give the title product.

### Step 13

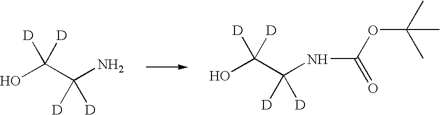

d₄-(2-Hydroxy-ethyl)-carbamic acid tert-butyl ester: The procedure of step 13 is carried out following the protocol set forth in Jia et al., Synlett Letter 2007, 5, 806-808. d₄-Ethanolamine (available commercially from Sigma-Aldrich, St. Louis, Mo., 63178) is added to di-tert-butyl dicarbonate at ambient temperature. The mixture is maintained at ambient temperature until reaction completion, as measured by thin layer chromotagraphy. The crude product is purified by silica gel column chromatography to yield the title product.

### Step 14

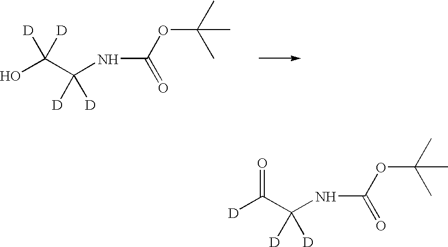

d₃-(2-Oxo-ethyl)-carbamic acid tert-butyl ester: The procedure of step 14 is carried out following the protocol set forth in de Napoli et al., Phytochemistry 1990, 29(3), 701-703. Pyridiniumchlorochromate is added to d₄-(2-hydroxy-ethyl)-carbamic acid tert-butyl ester in dry dichloromethane at ambient temperature. The mixture is maintained at ambient temperature until reaction completion, as measured by thin layer chromotagraphy. The reaction is stopped by adding dry ethanol. The crude product is purified by silica gel column chromatography to give the title product.

### Step 15

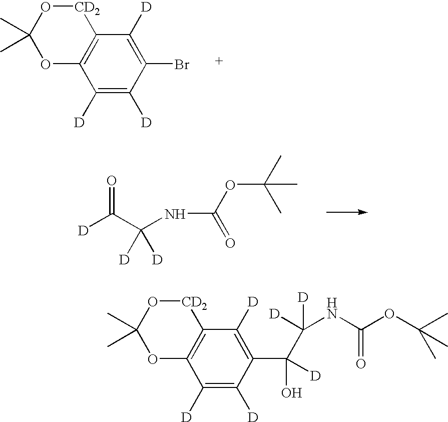

d₈-[2(2,2-Dimethyl-4H-benzo[1,3]dioxin-6-yl)-2-hydroxy-ethyl]-carbamic acid tert-butyl ester: At about −78° C., n-butyllithium is added to d₅-6-bromo-2,2-dimethyl-4H-benzo[1,3]dioxine in a tetrahydrofuran/ether/pentane mixture and maintained at about −78° C. for about 1 hour. d₃-(2-Oxo-ethyl)-carbamic acid tert-butyl ester in anhydrous tetrahydrofuran is added dropwise with a dry syringe. The mixture is maintained at ambient temperature until reaction completion, as measured by thin layer chromatography. The reaction is quenched with ammonium chloride. Standard extractive workup with ethyl acetate affords the title product.

### Step 16

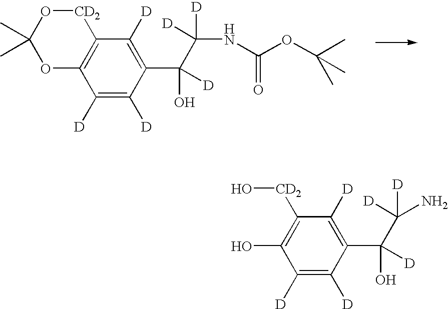

d₈-4-(2-Amino-1-hydroxy-ethyl)-2-hydroxymethyl-phenol: 6N HCL is added to d₈-[2(2,2-dimethyl-4H-benzo[1,3]dioxin-6-yl)-2-hydroxy-ethyl]-carbamic acid tert-butyl ester in methanol. The mixture is heated at reflux until reaction completion, as measured by thin layer chromatography. The solvent is removed in vacuo.

### Step 17

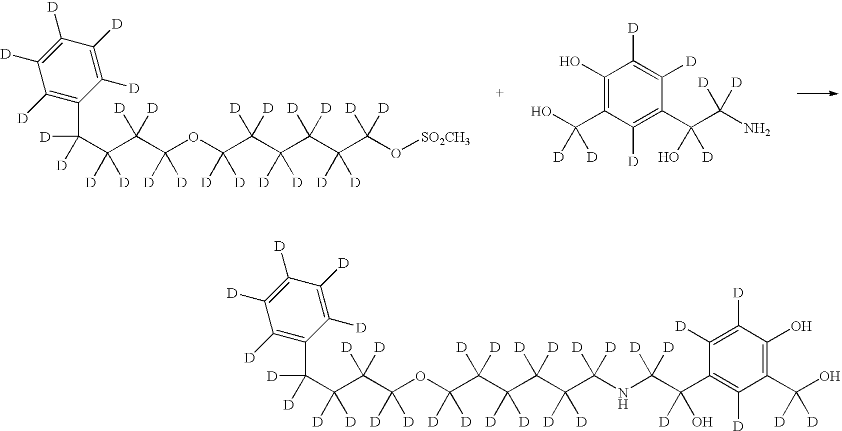

d₃₃-4-hydroxy-alpha-[[[4-[(6-phenylbutyl)oxy]hexyl]amino]-methyl]-1,3-benzenedimethanol: The procedure of Step 17 is carried out using the methods as described in U.S. Pat. No. 4,992,474. At about 70° C., d₂₅-4-[(6-phenylbutyl)oxy]hexan-1-ol methanesulphonate is added dropwise to a solution containing d₈-4-(2-amino-1-hydroxy-ethyl)-2-hydroxymethyl-phenol, potassium iodide and triethylamine in N,N-dimethylformamide. The solution is maintained at about 70° C. for about 1 hour, and then poured into water. Following standard extractive workup with ethyl acetate, the crude residue is purified by silica gel chromatography. Trituration with diethyl ether gives the title product.

### Step 18

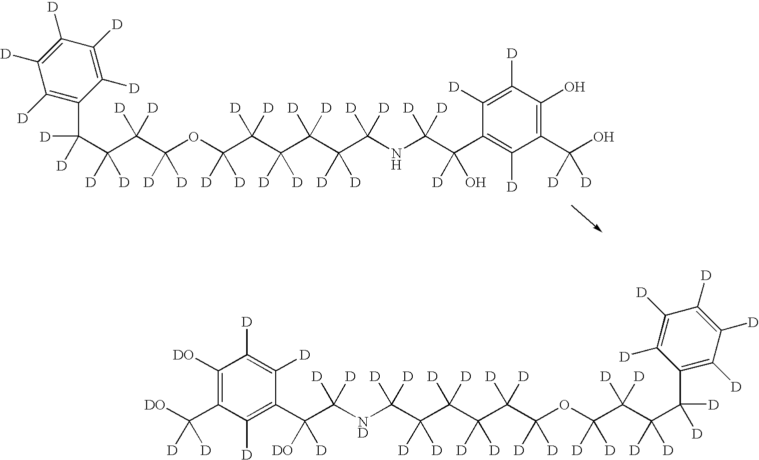

d₃₇-4-hydroxy-alpha-[[[4-[(6-phenylbutyl)oxy]hexyl]amino]-methyl]-1,3-benzenedimethanol: d₃₃-4-Hydroxy-alpha-[[[4-[(6-phenylbutyl)oxy]hexyl]amino]-methyl]-1,3-benzenedimethanol dissolved in d₄-methanol (0.5 ml) is added dropwise to a 0.1M solution of sodium carbonate in deuterium oxide (pH=11.4). The solution is maintained at ambient temperature for about 4 days. Standard extractive workup with dichloromethane affords the title compound.

The following compounds can generally be made using the methods described above. It is expected that these compounds when made will have activity similar to those described in the examples above.

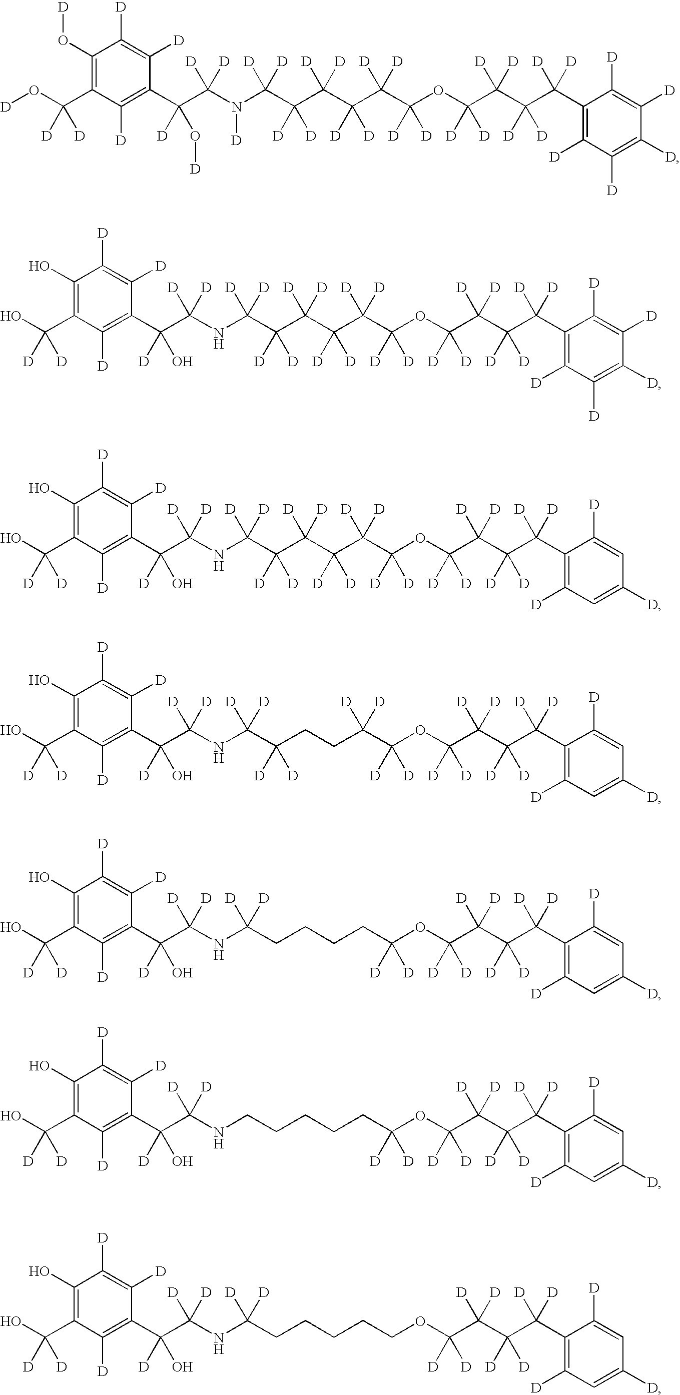

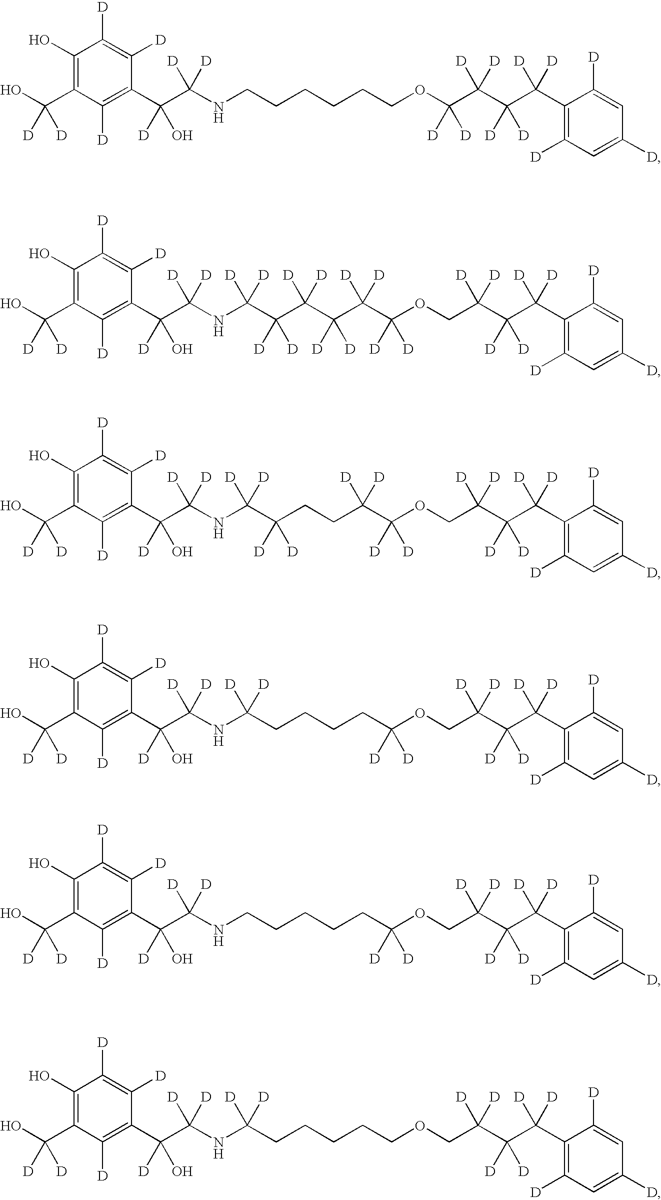

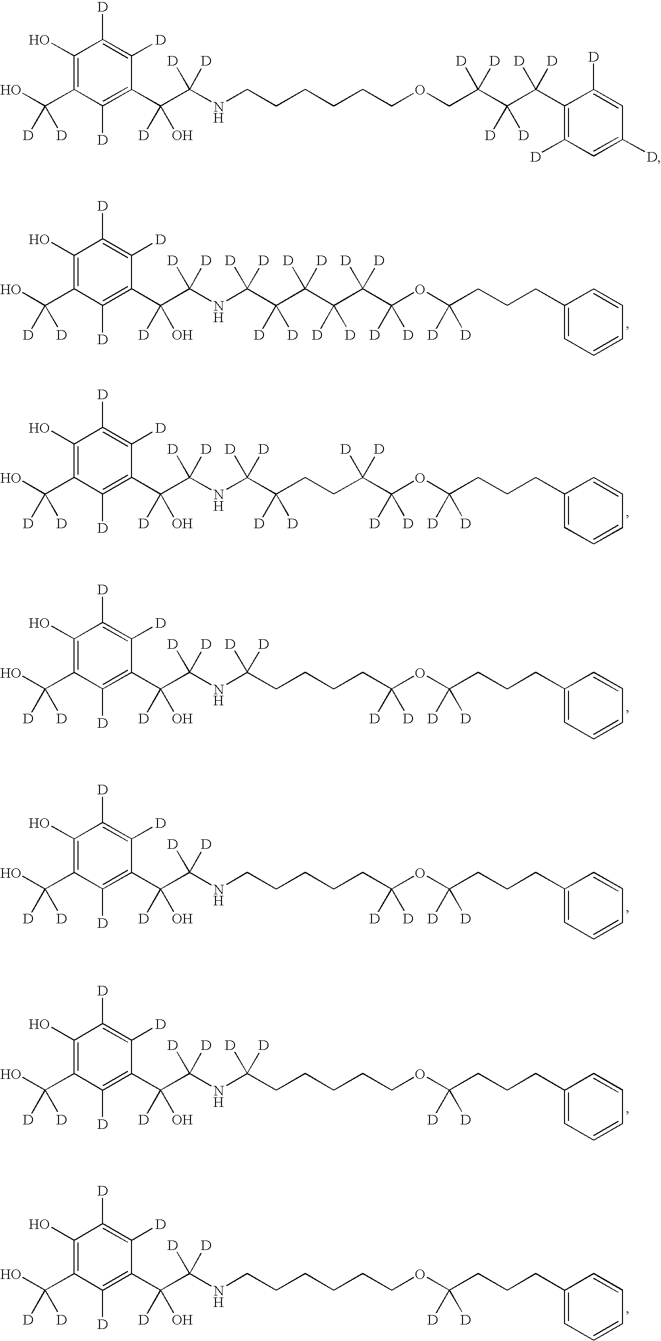

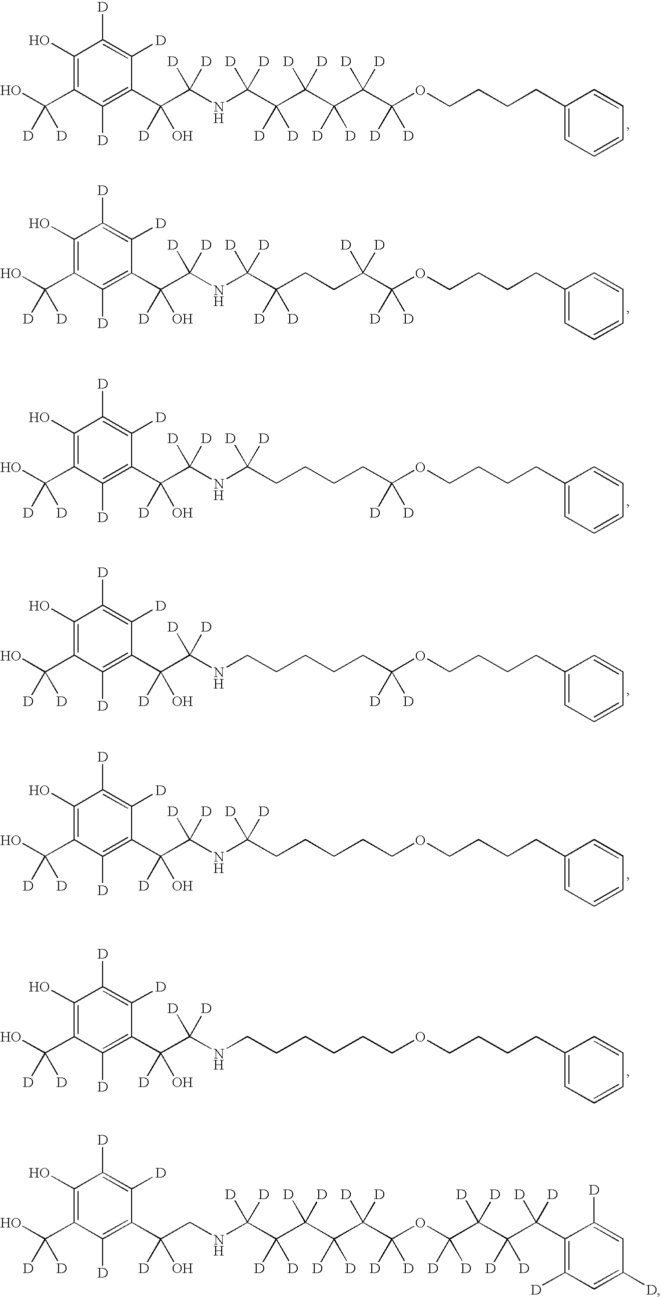

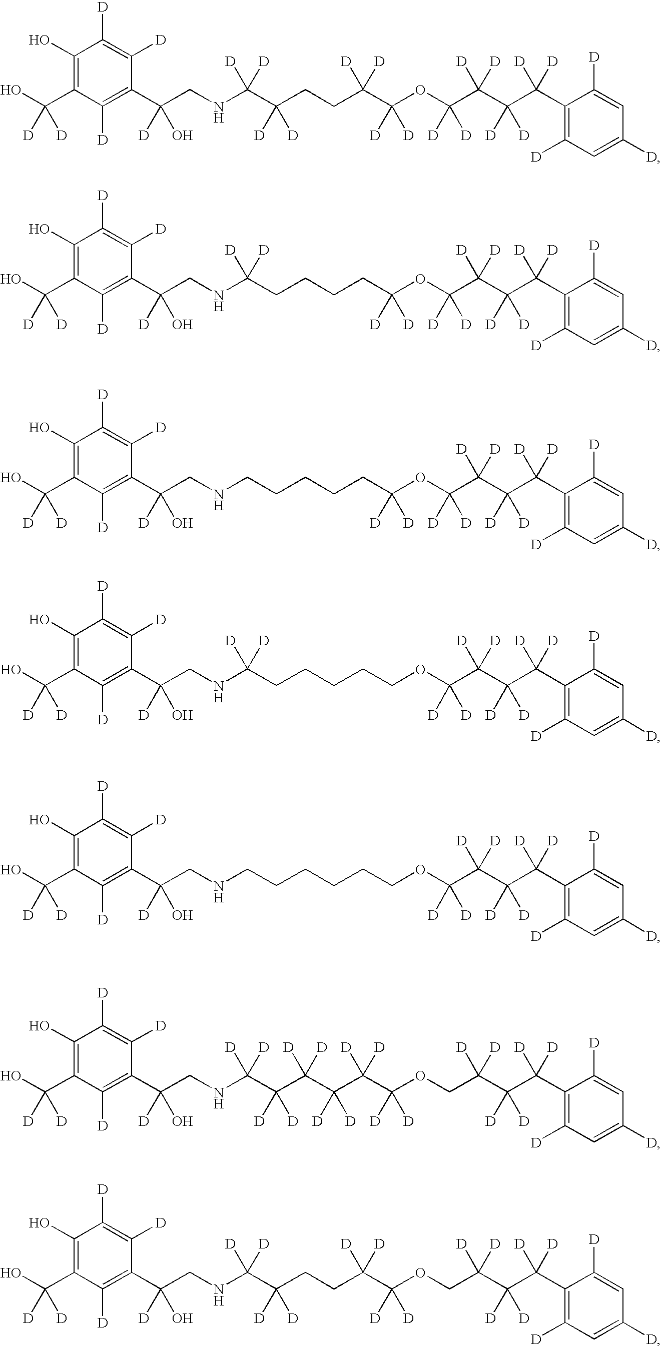

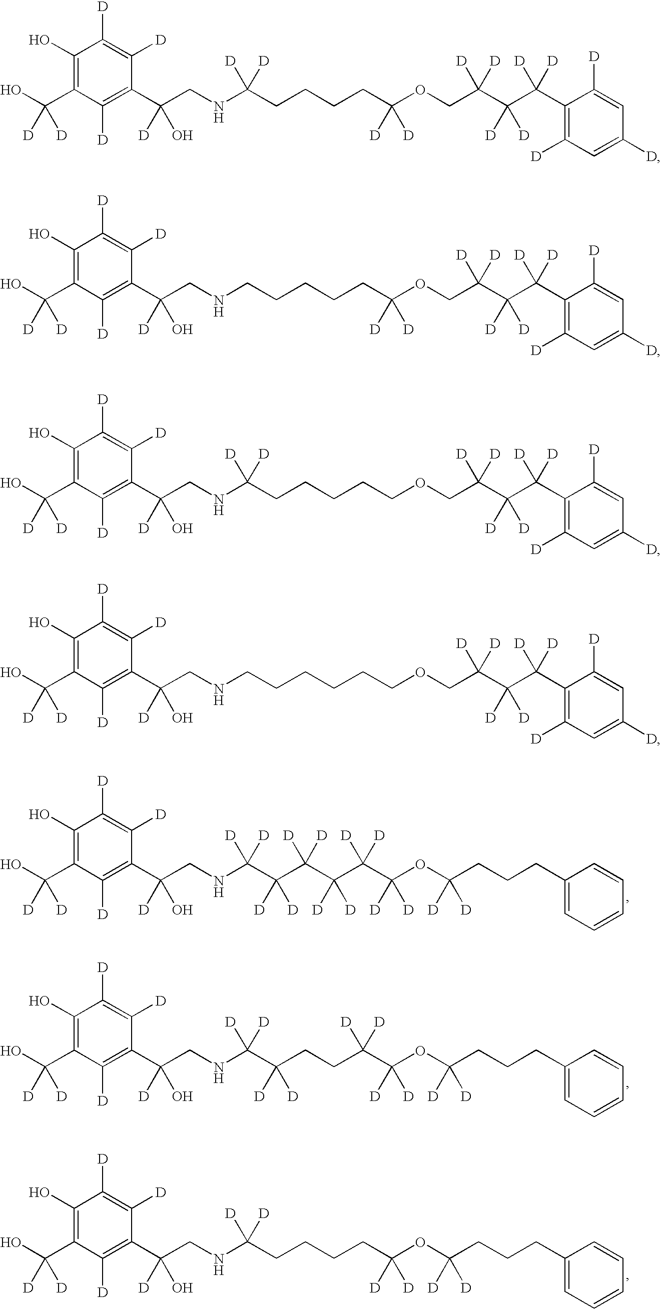

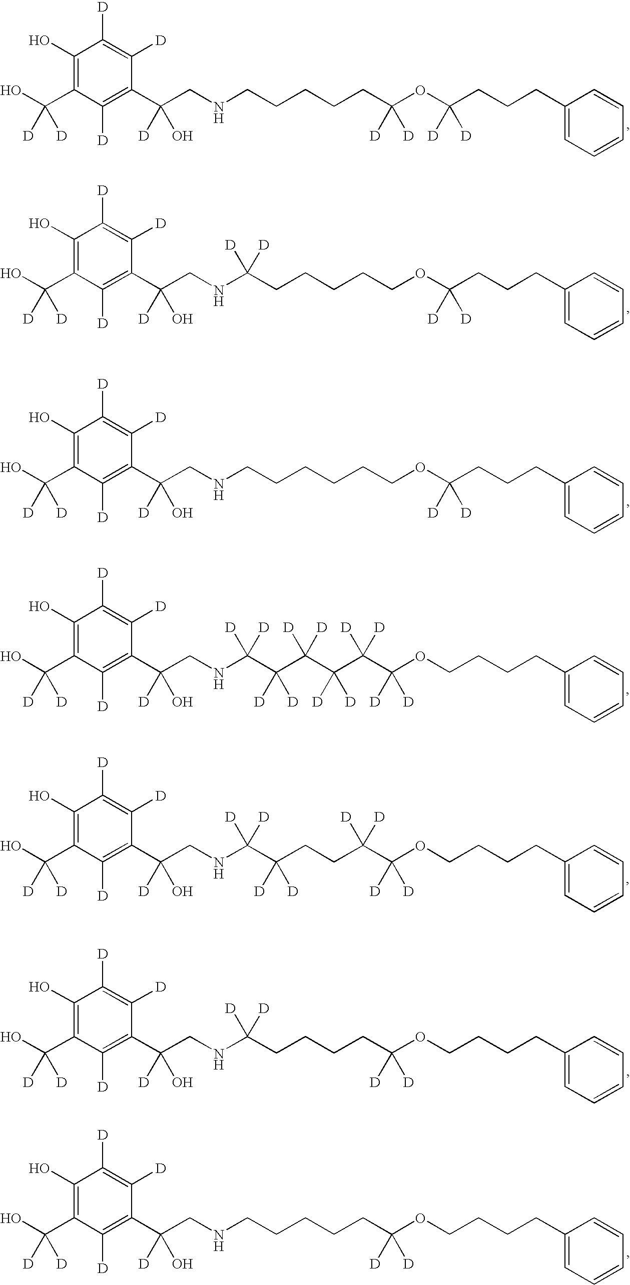

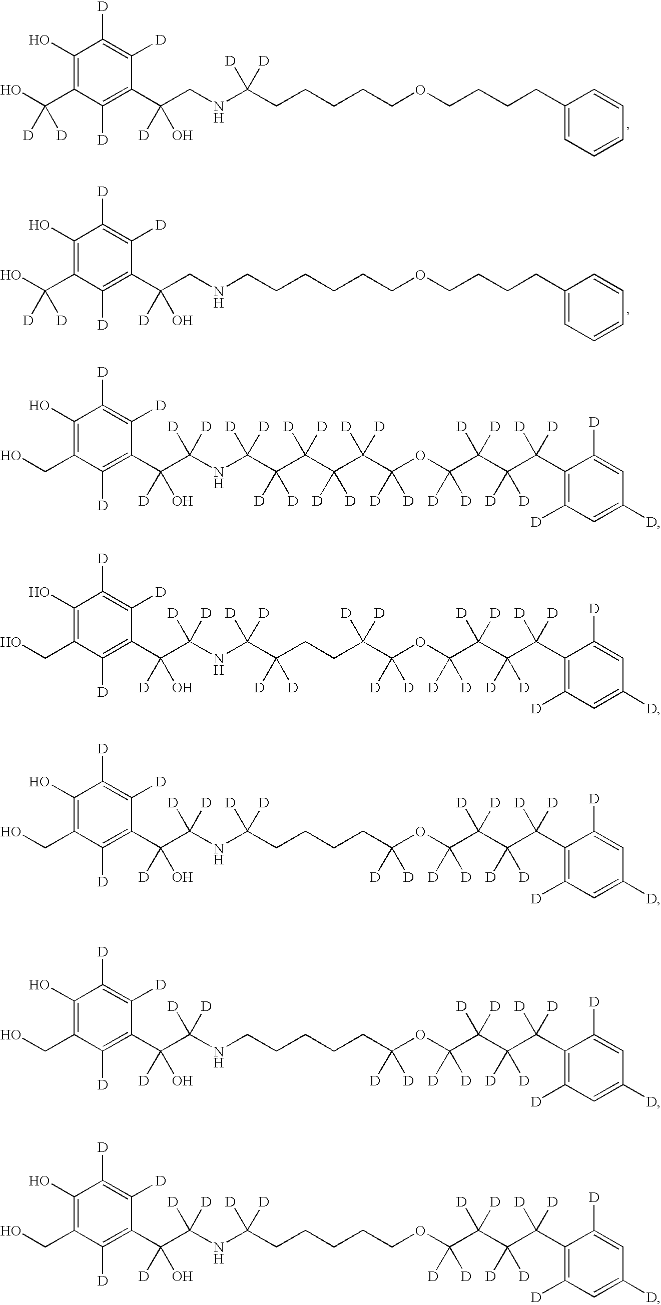

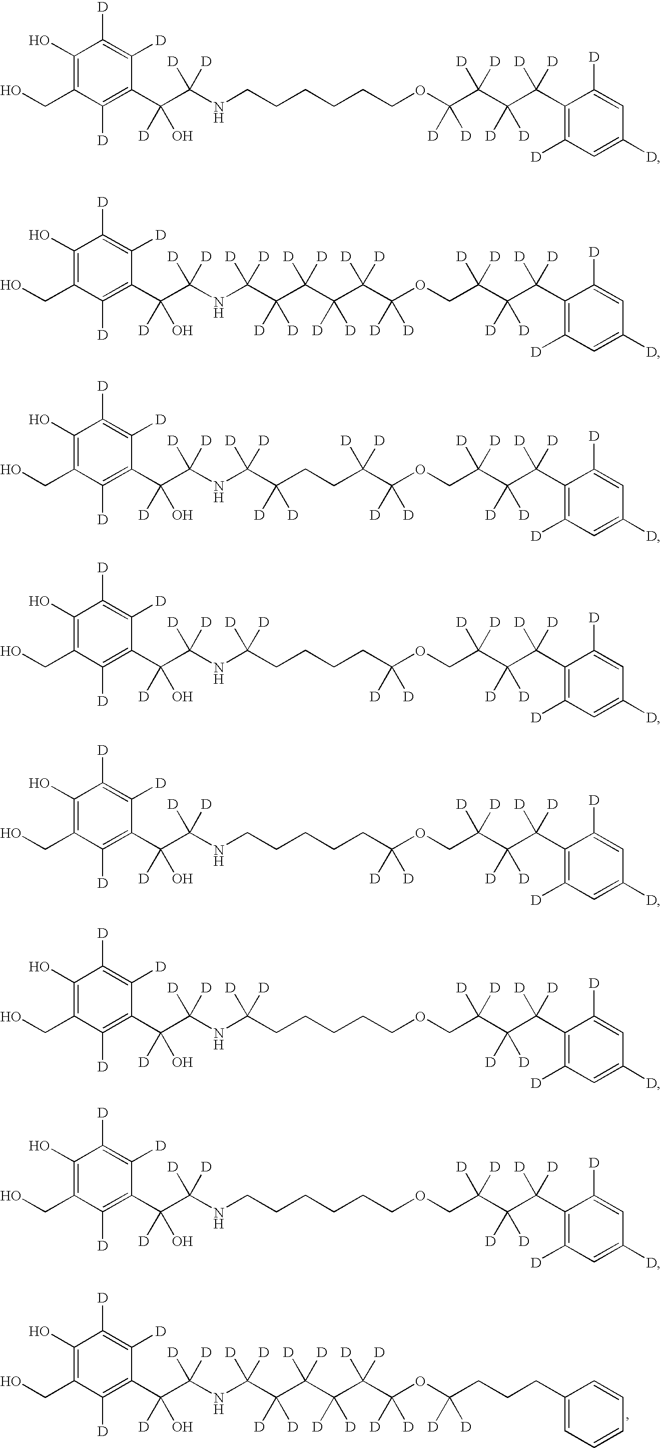

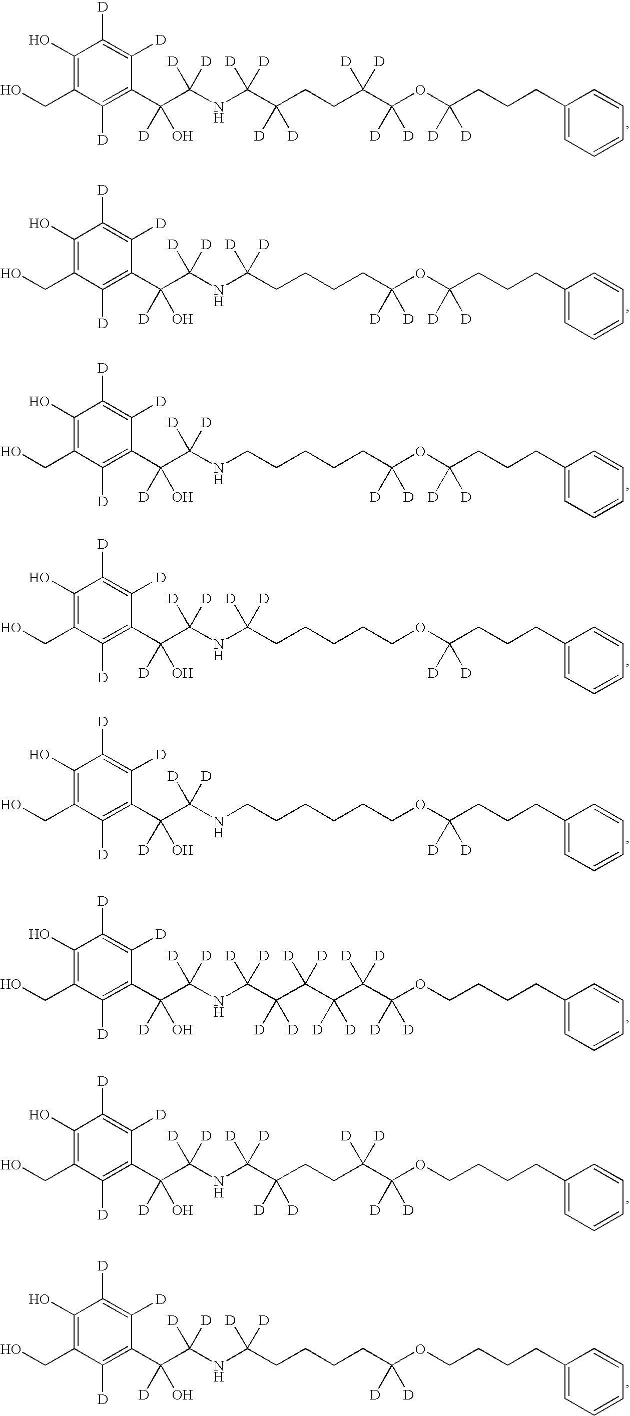

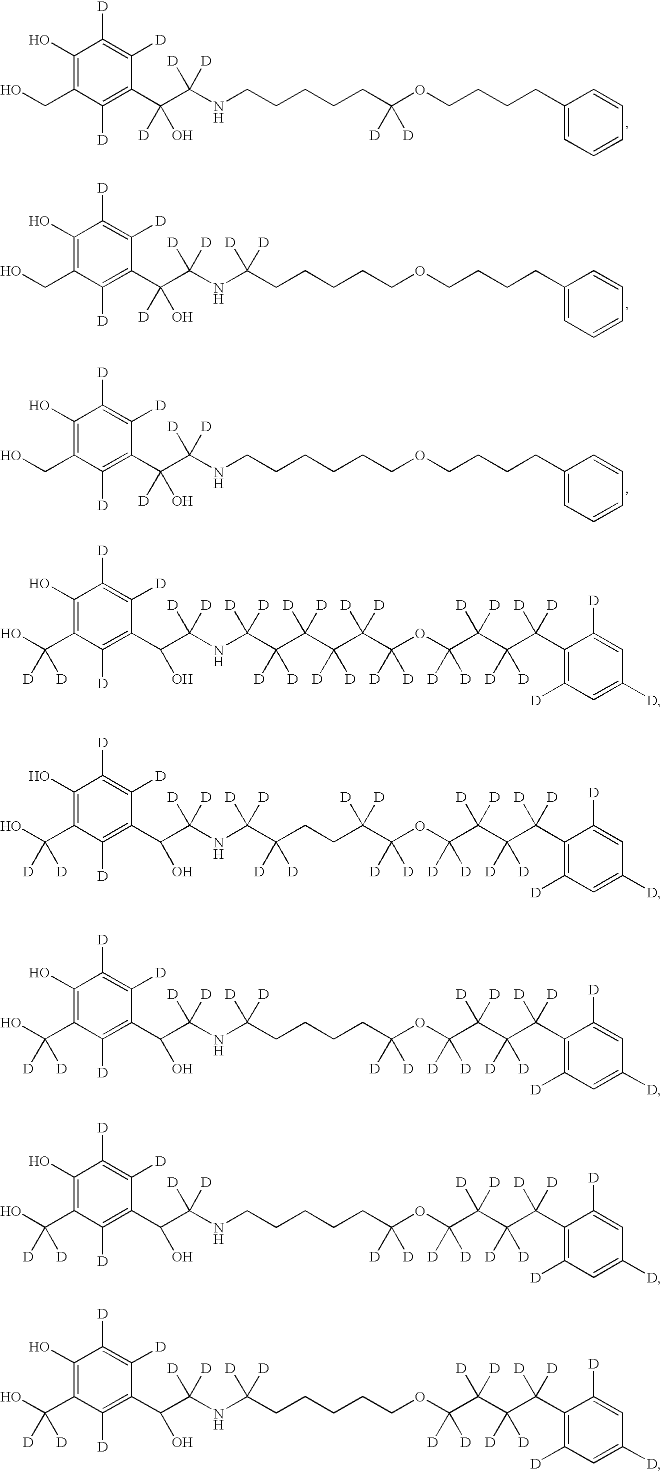

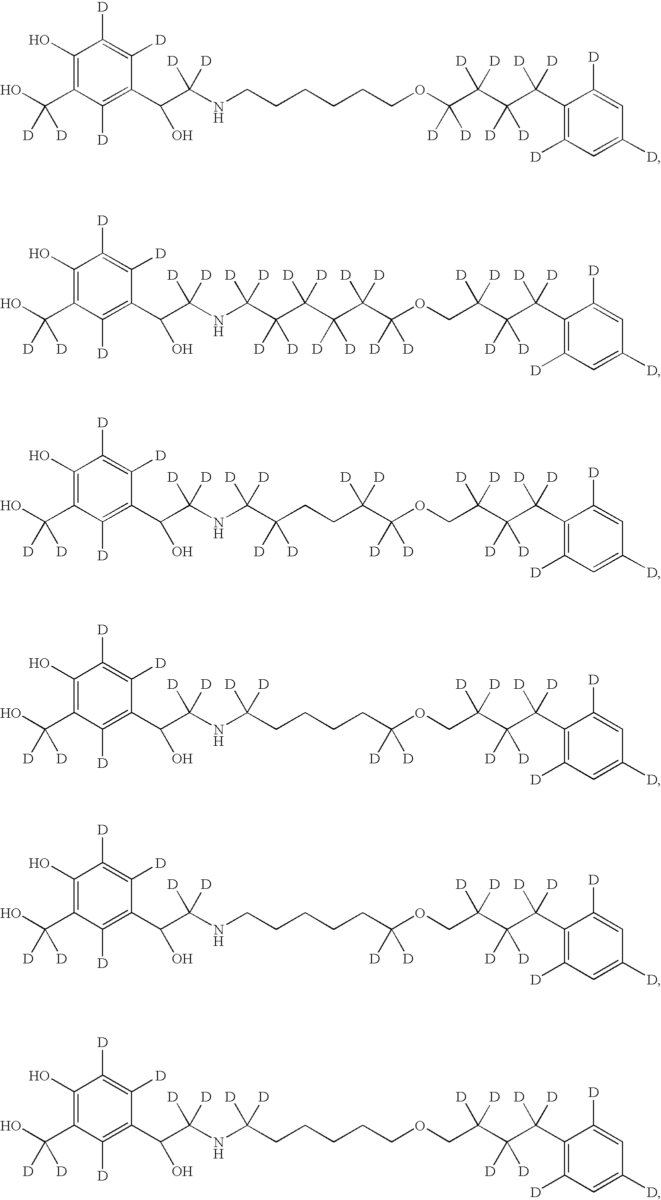

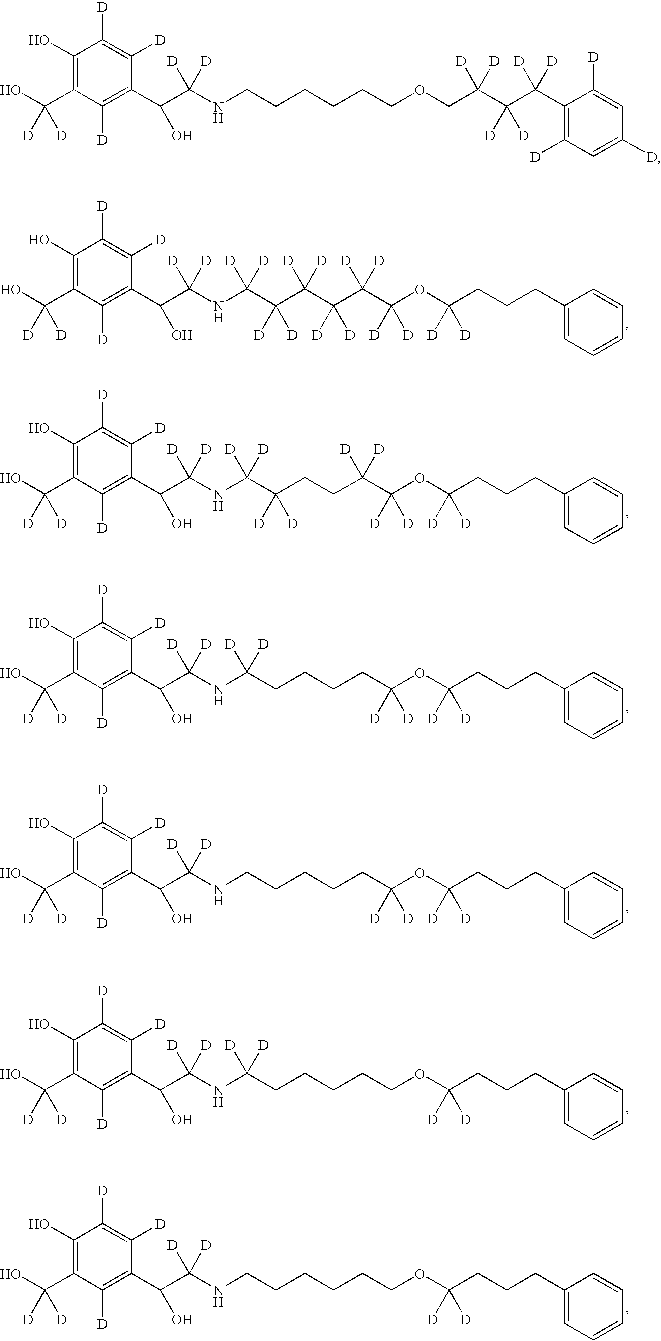

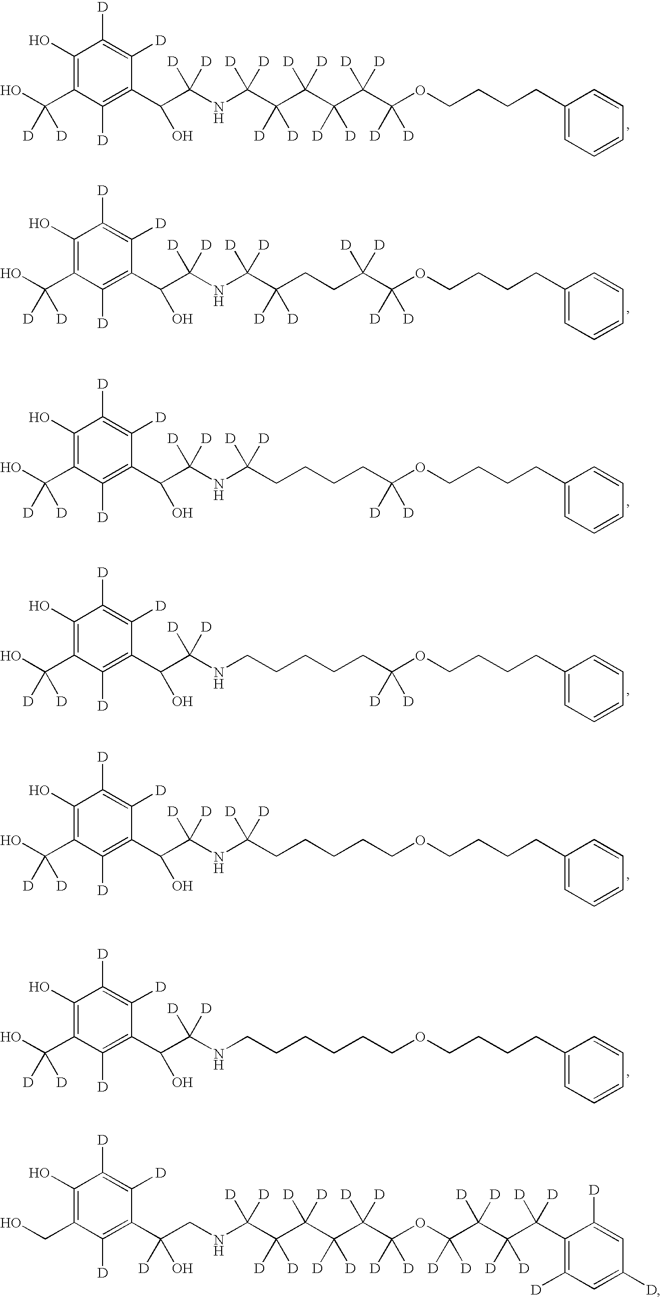

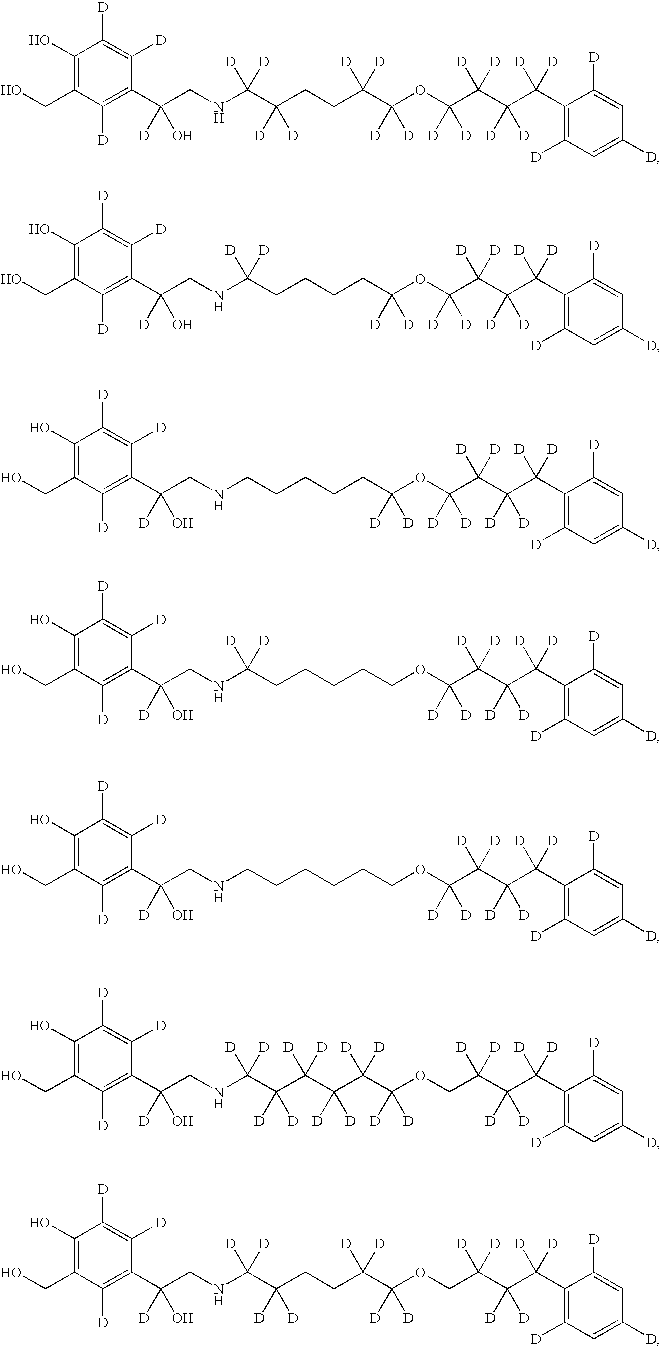

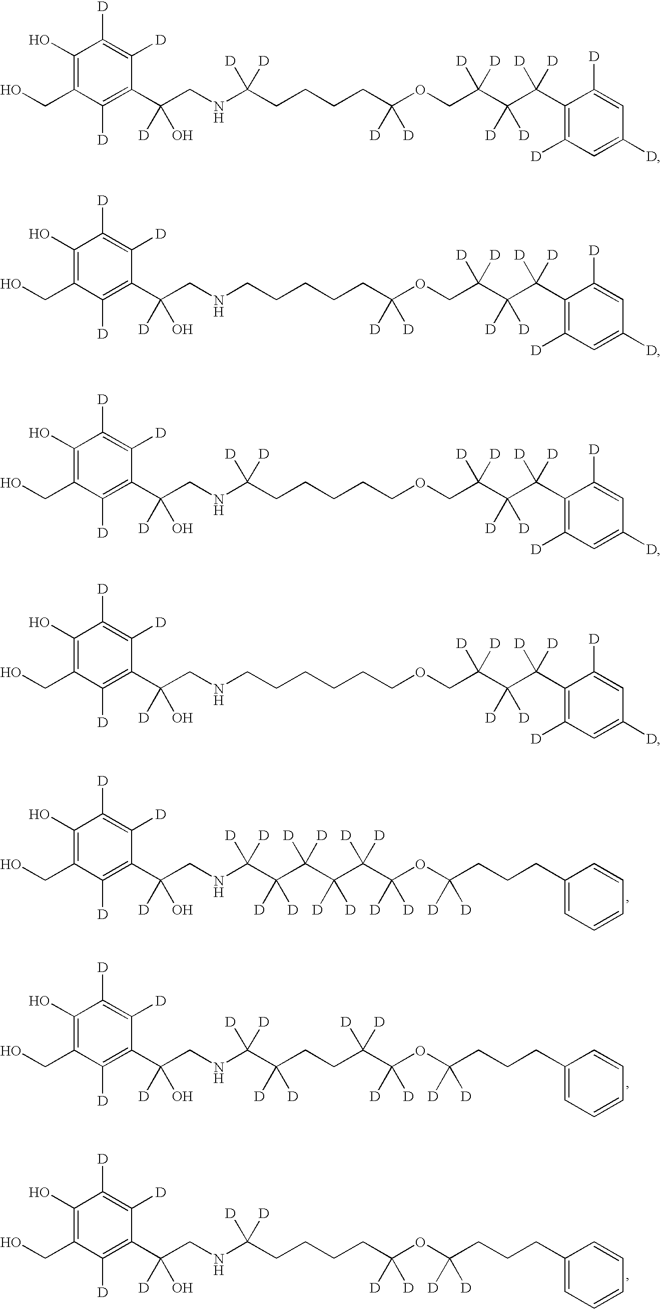

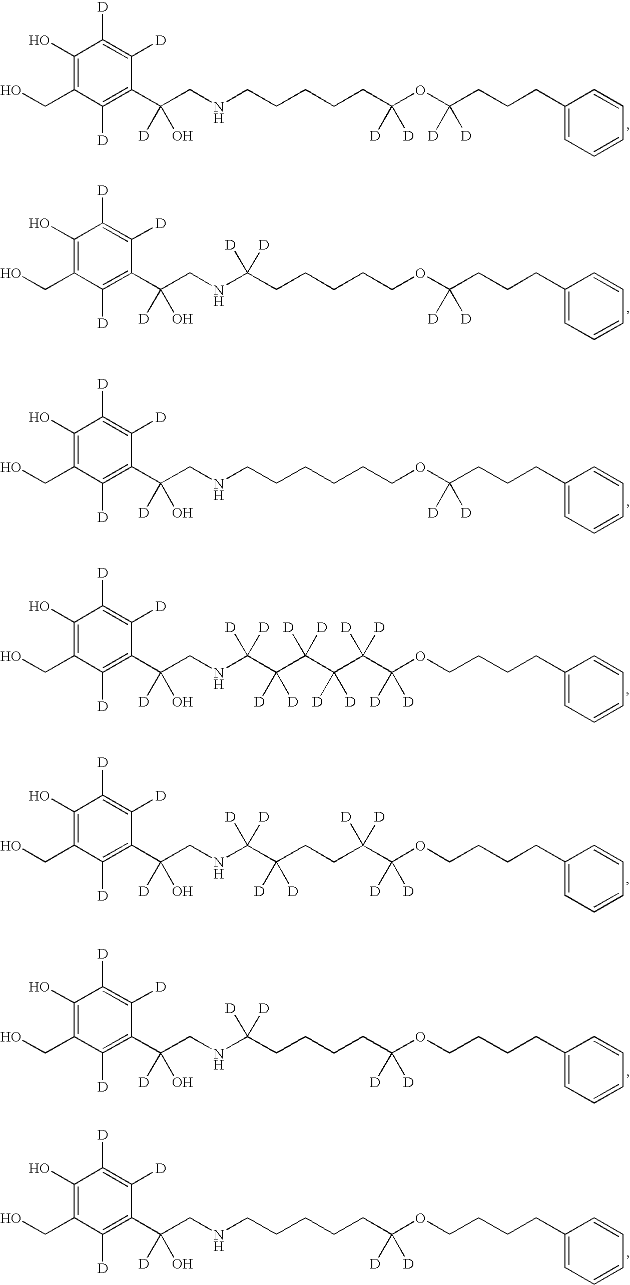

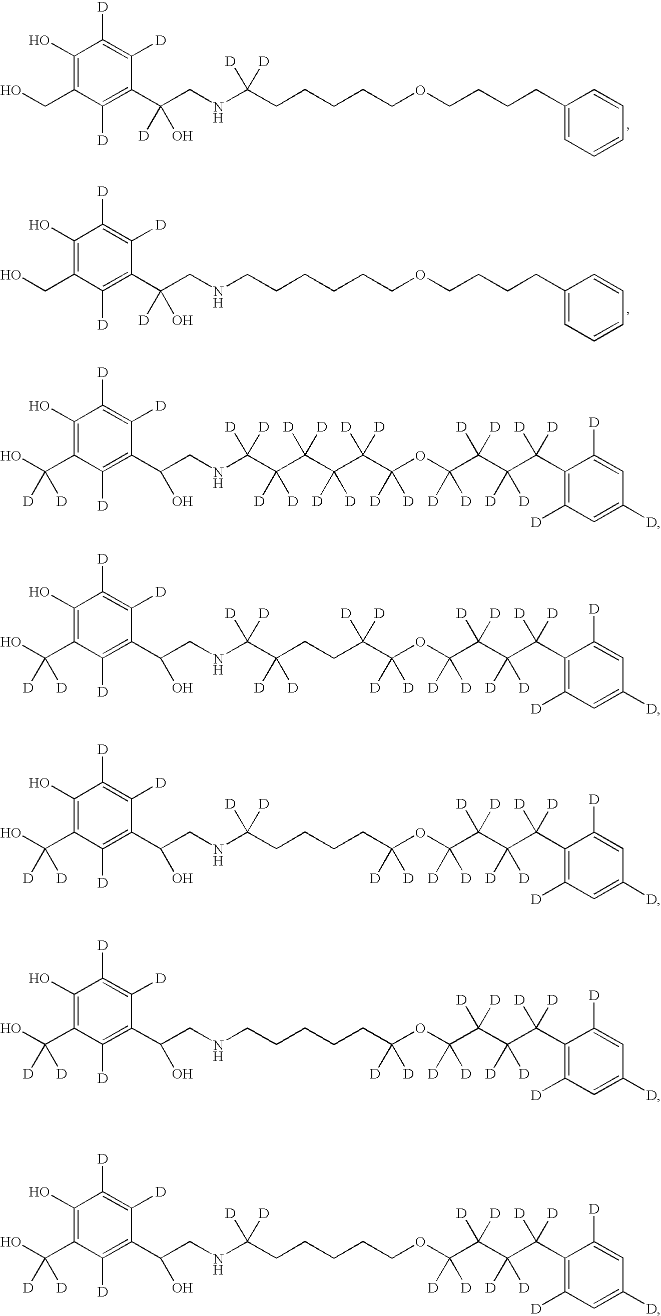

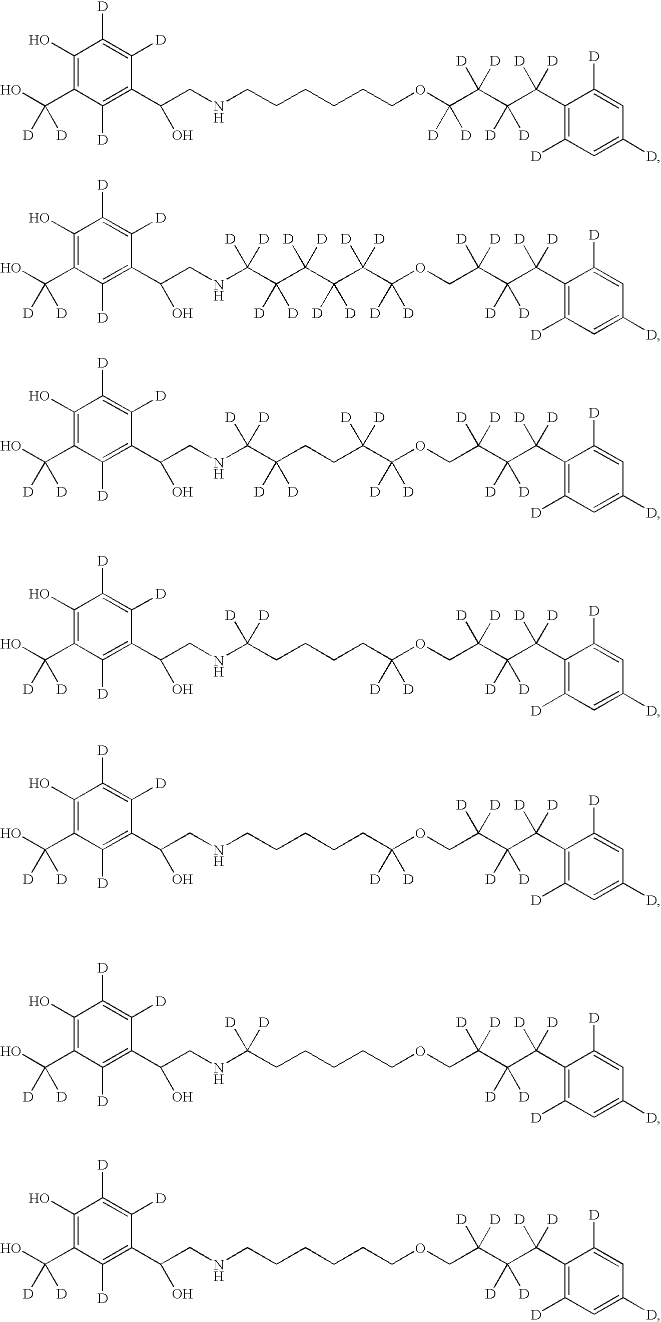

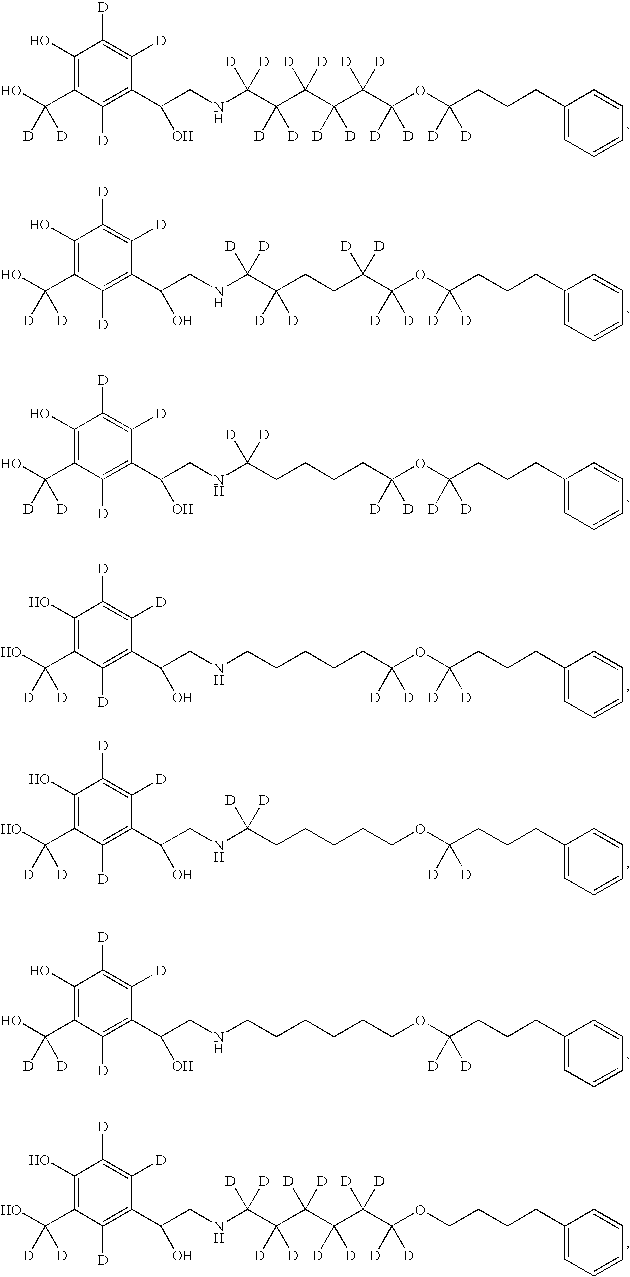

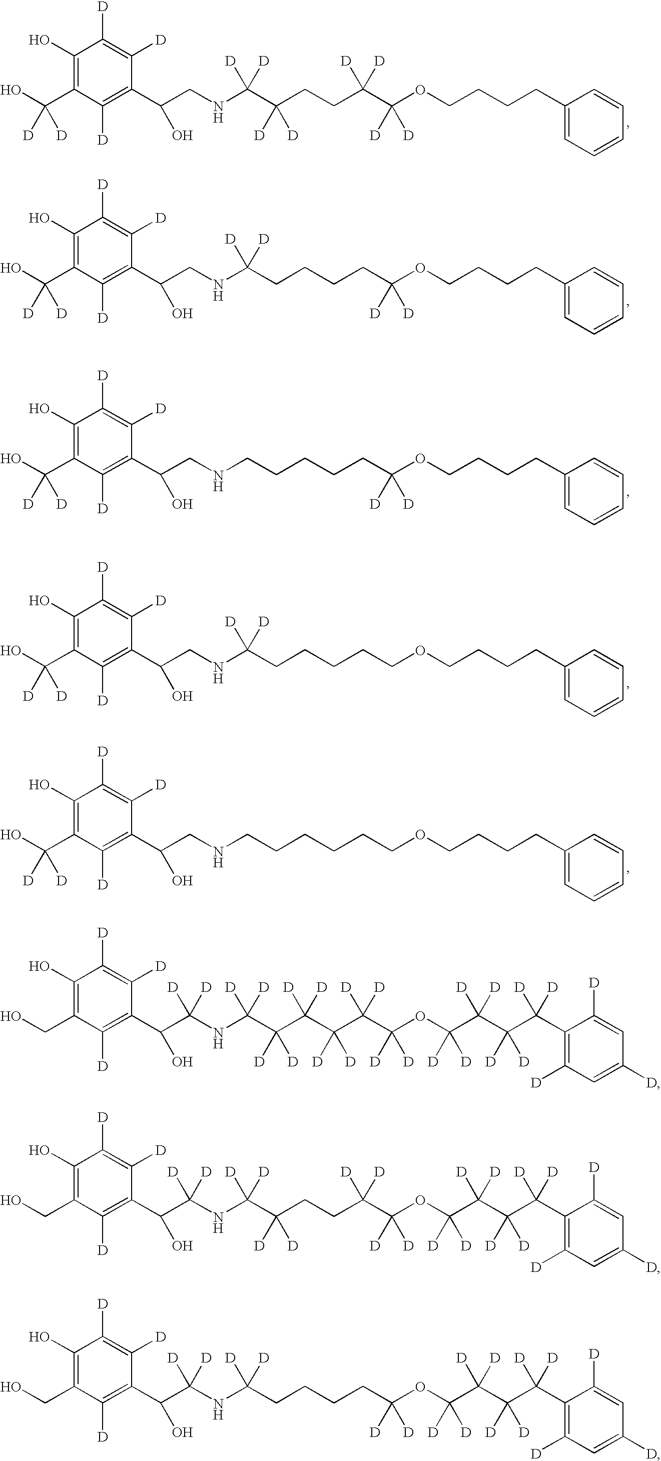

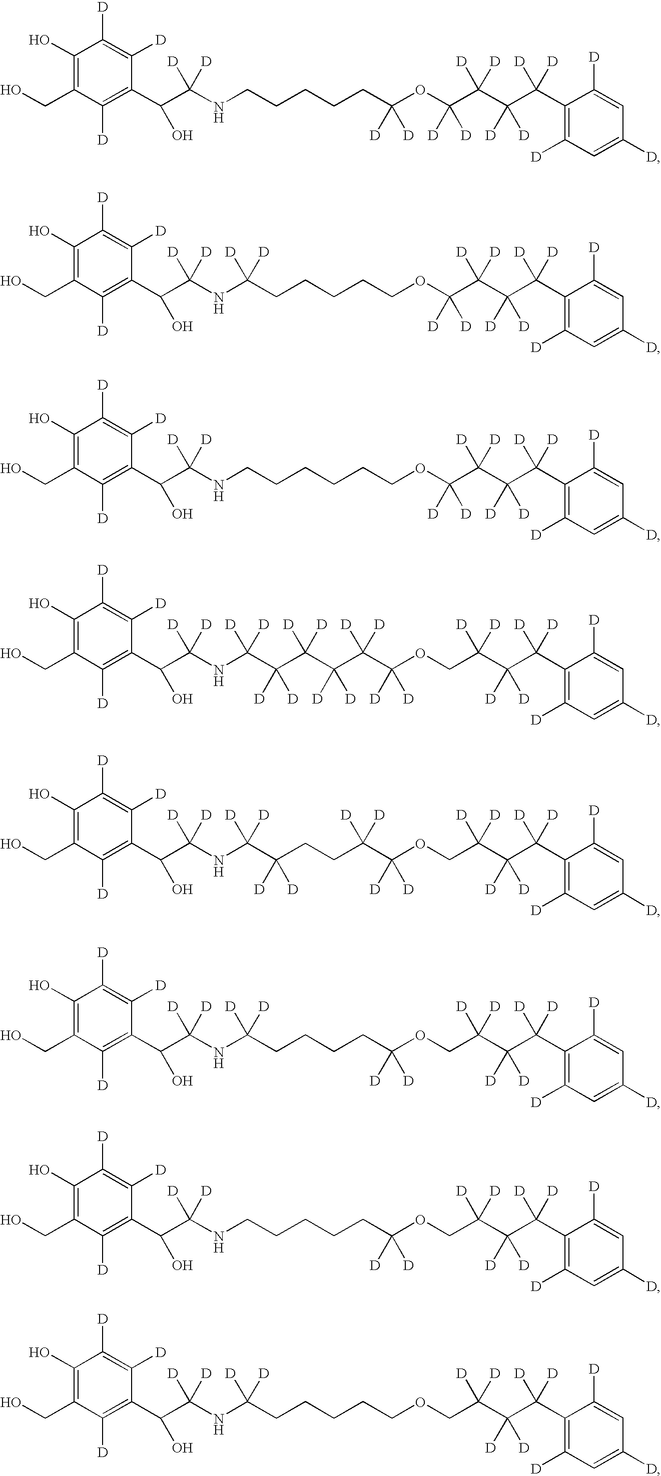

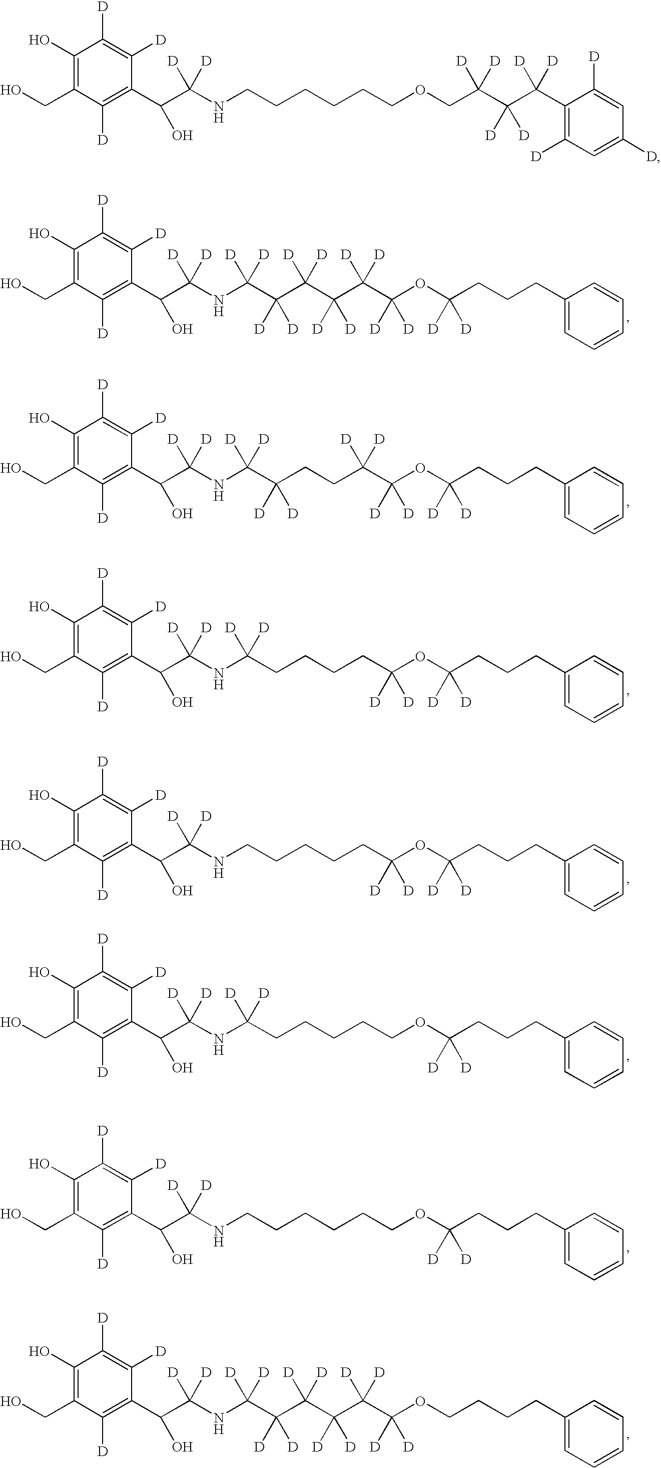

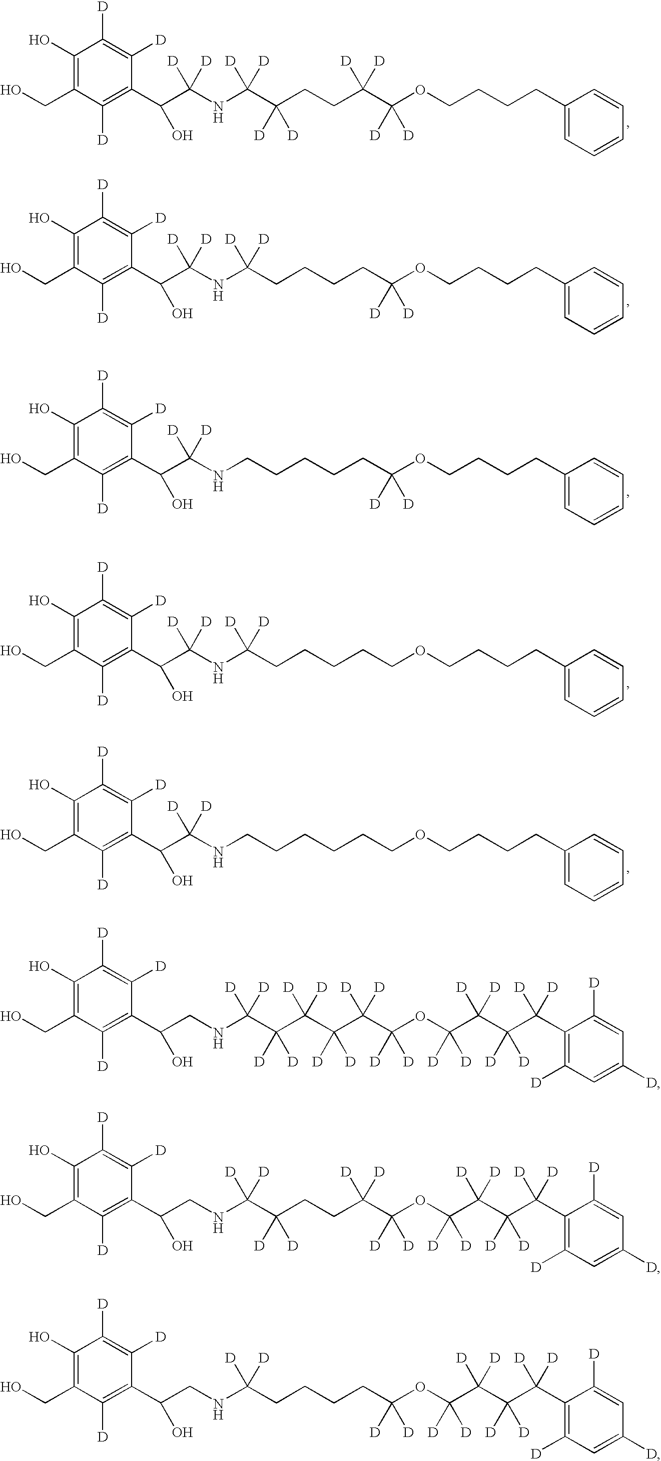

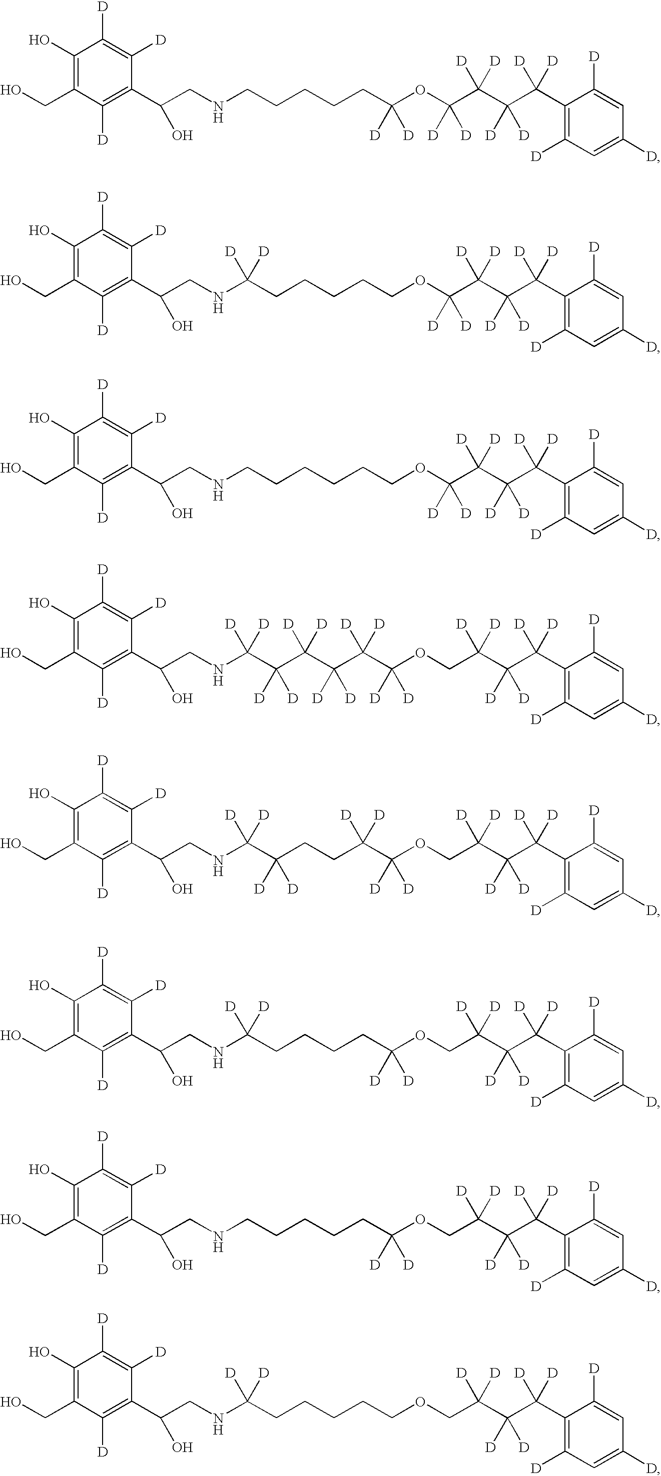

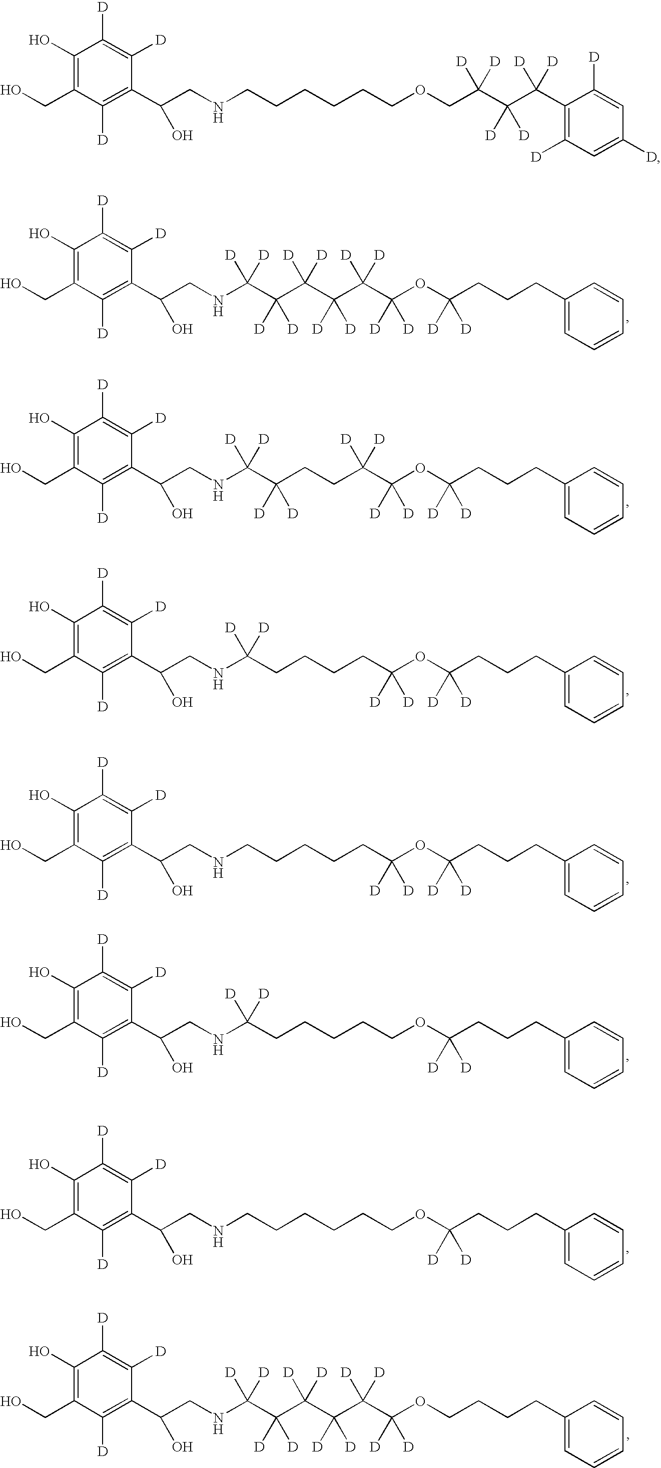

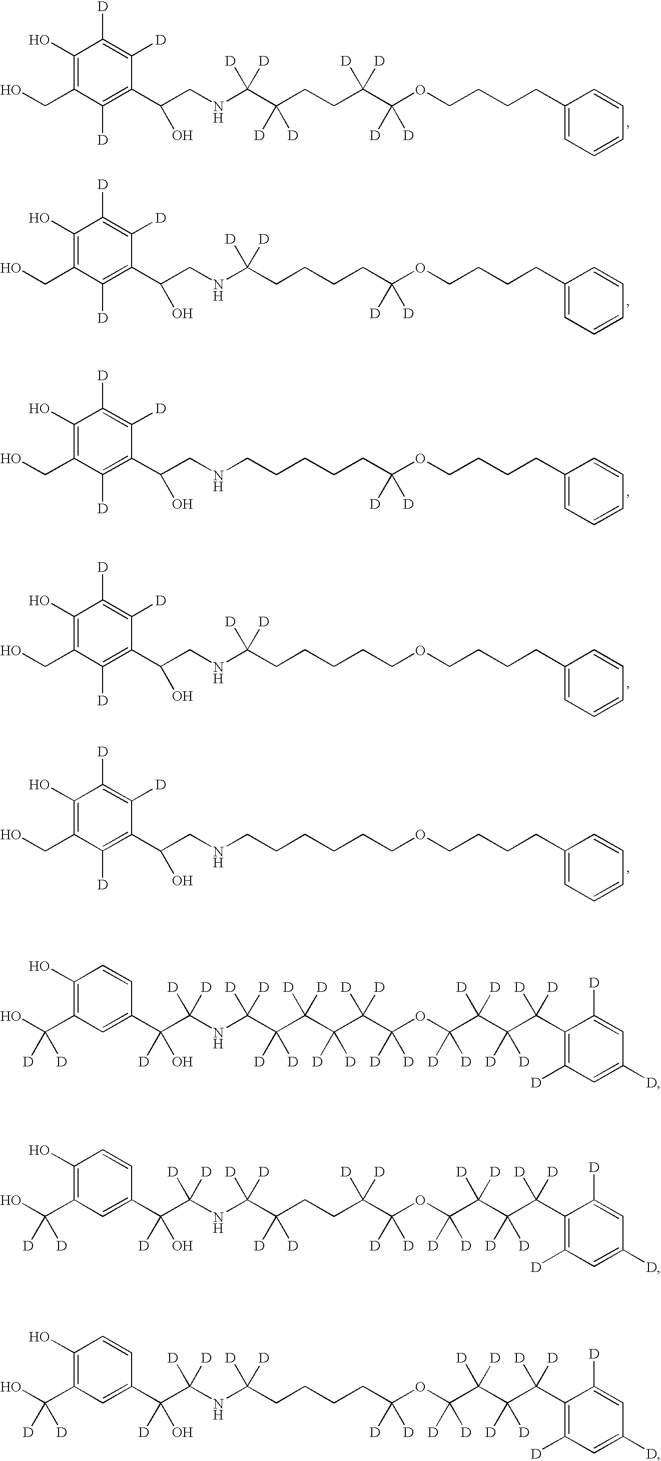

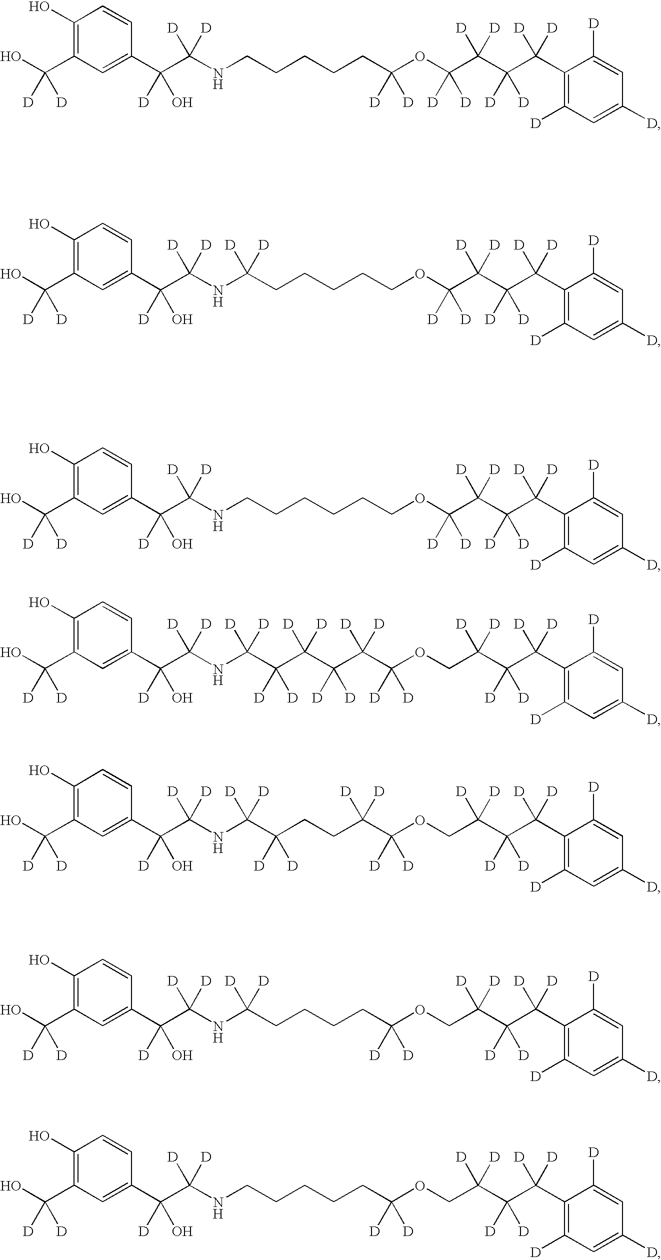

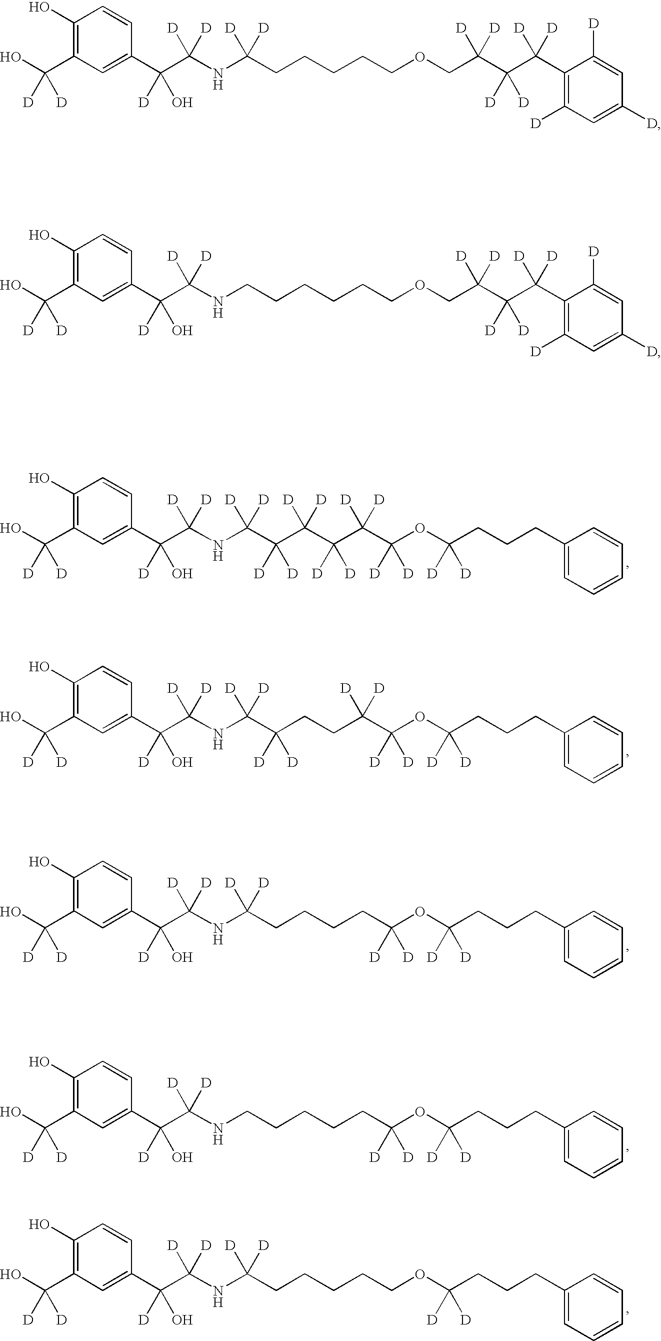

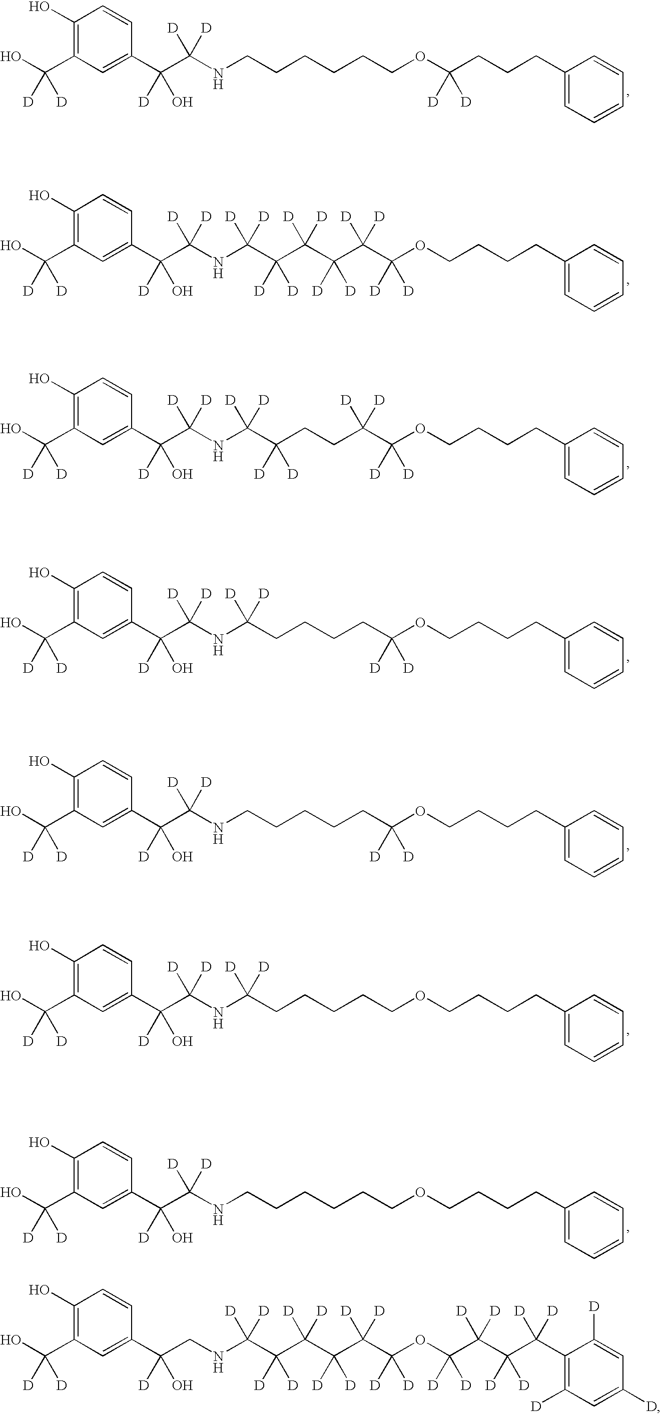

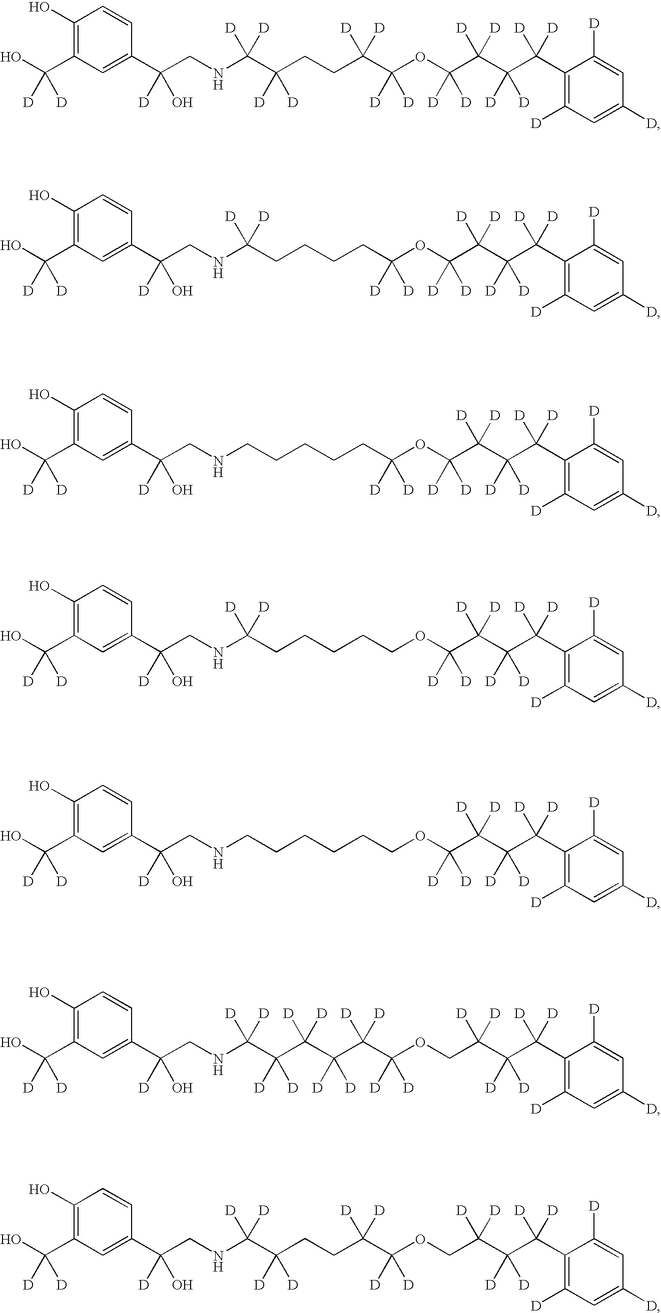

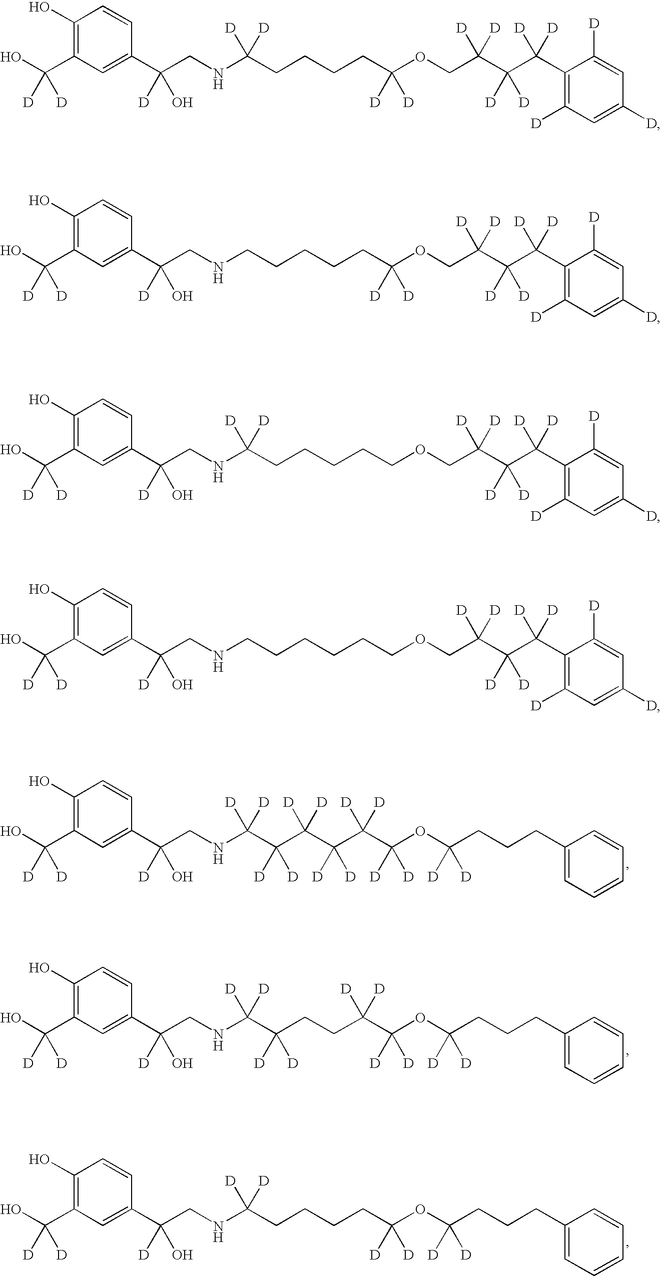

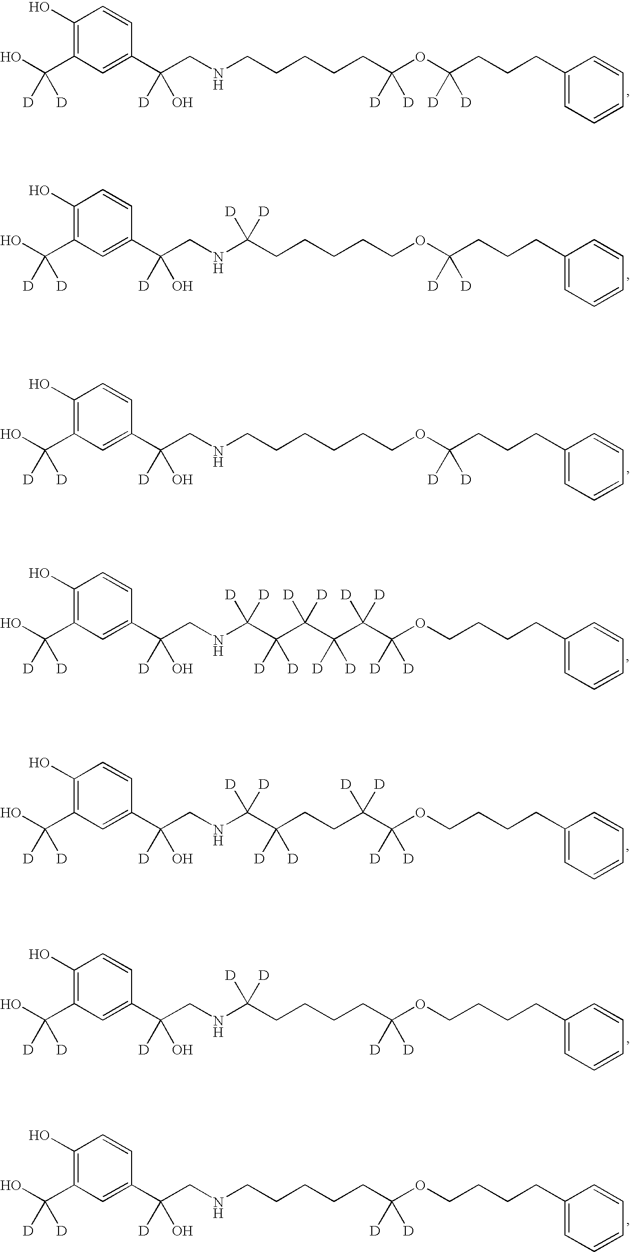

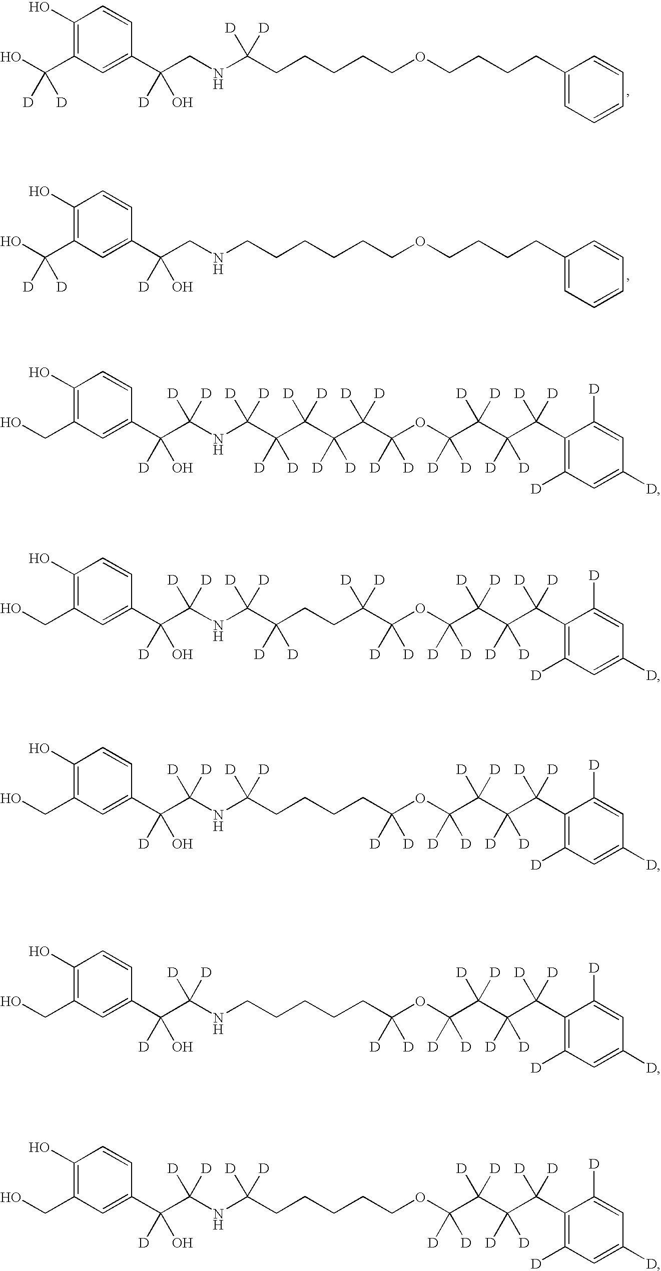

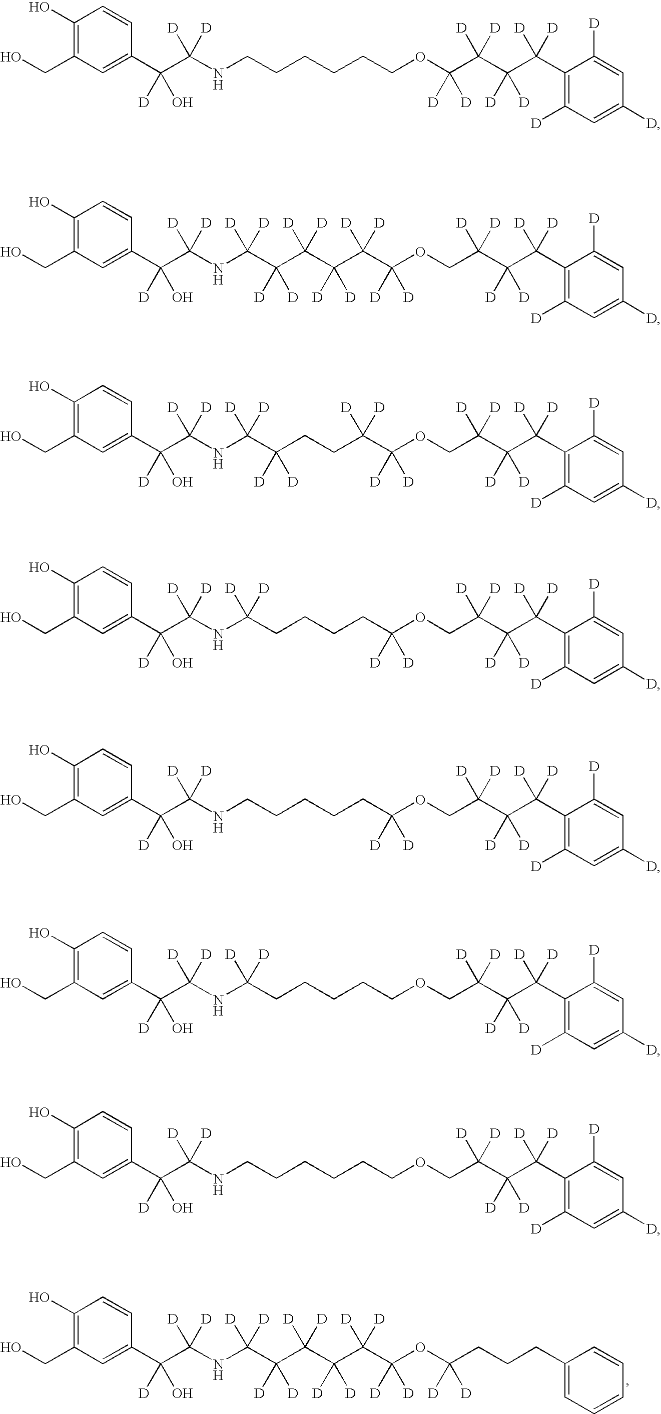

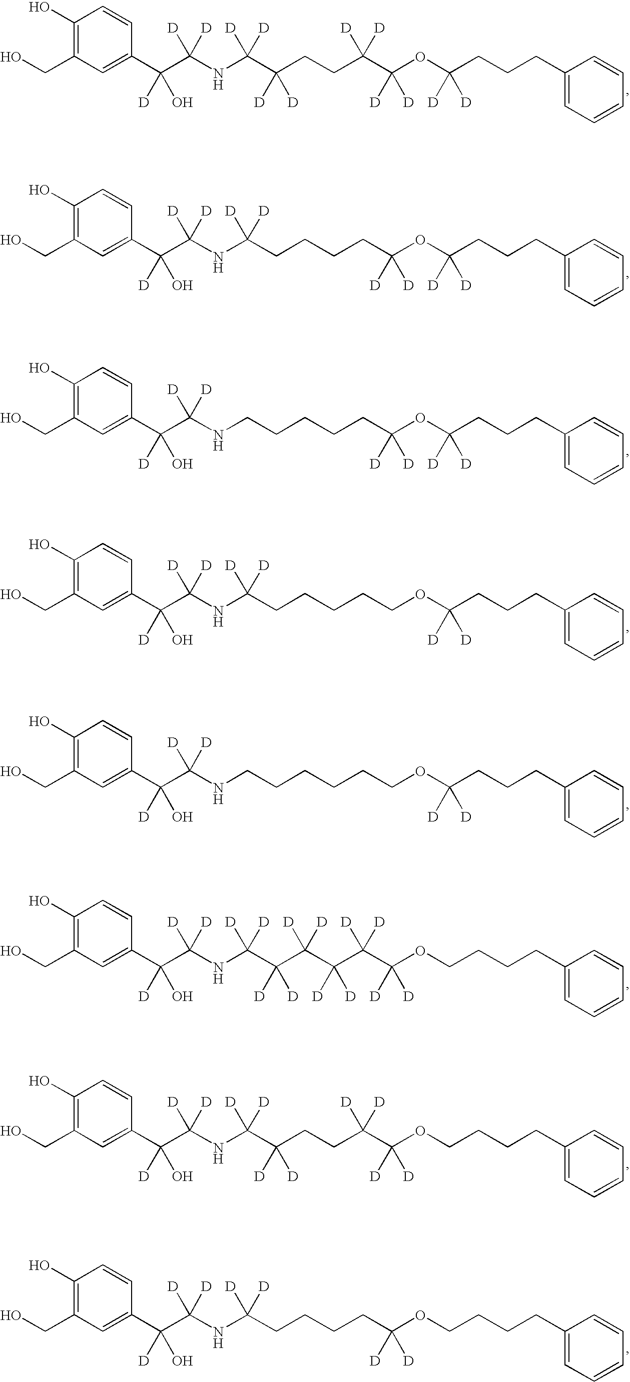

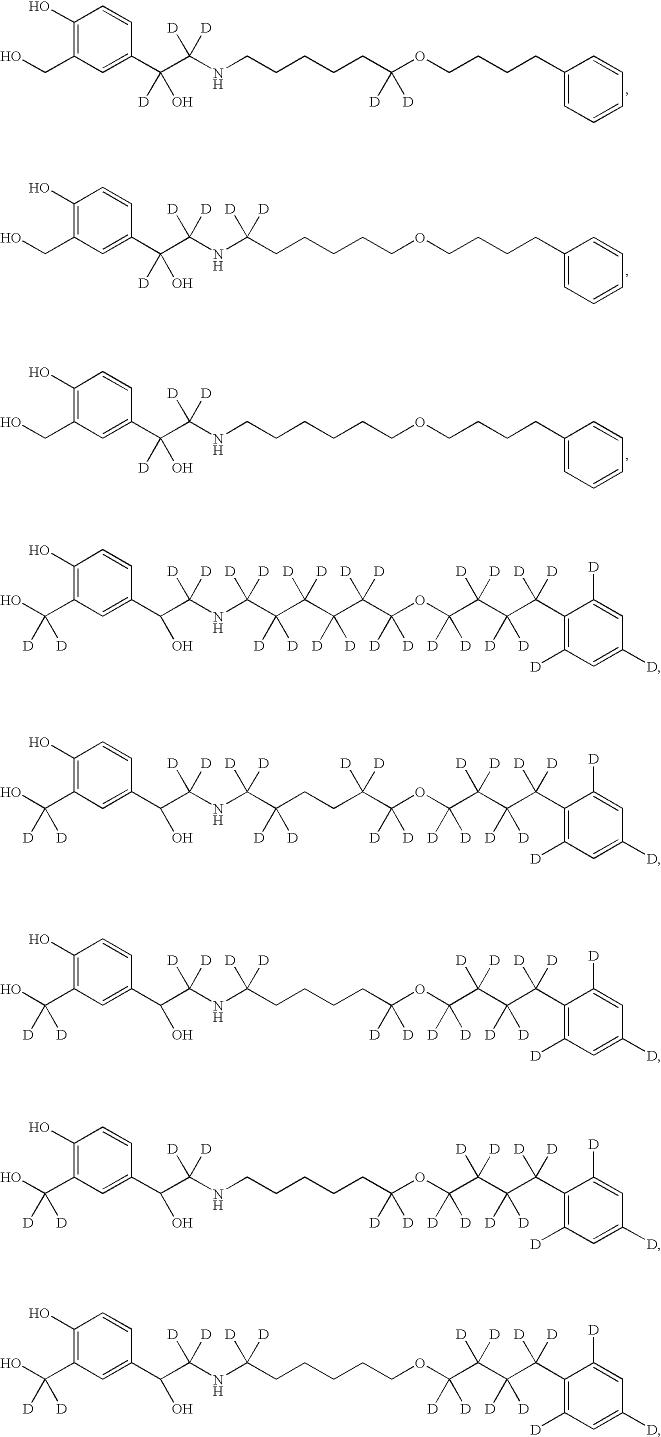

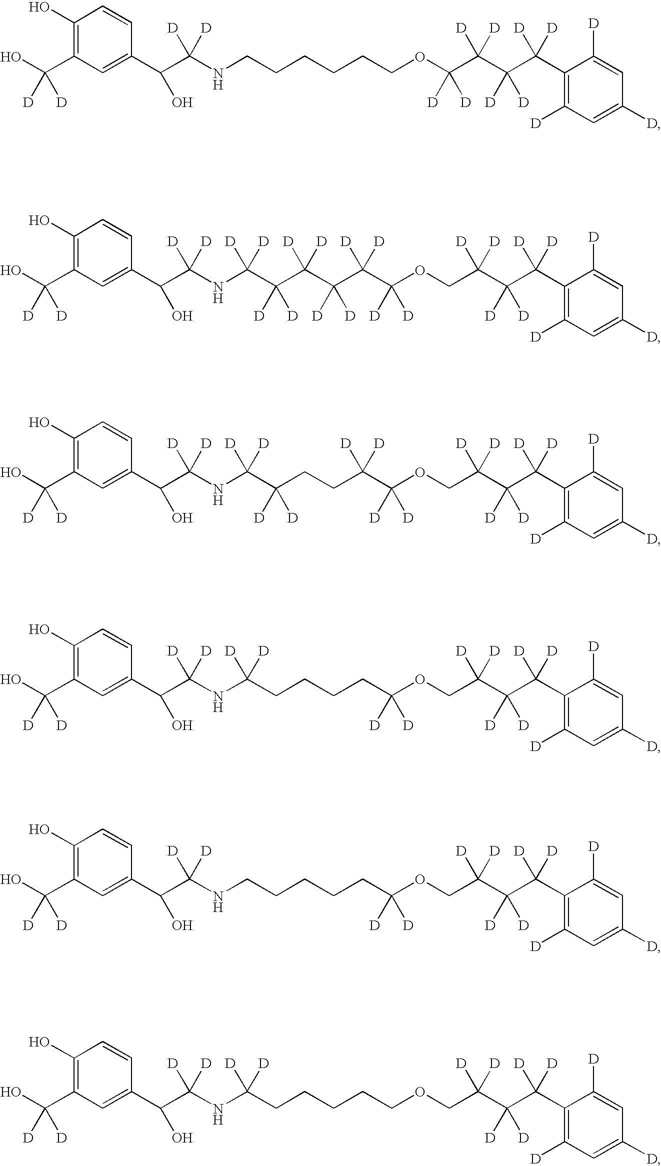

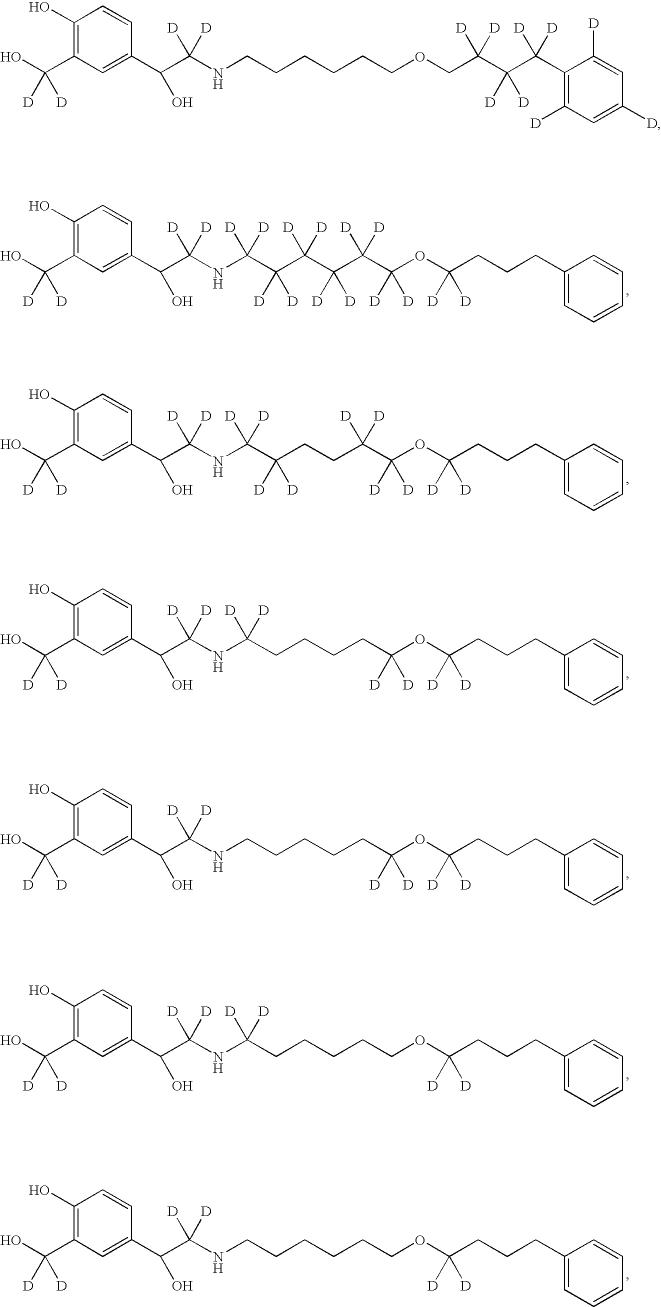

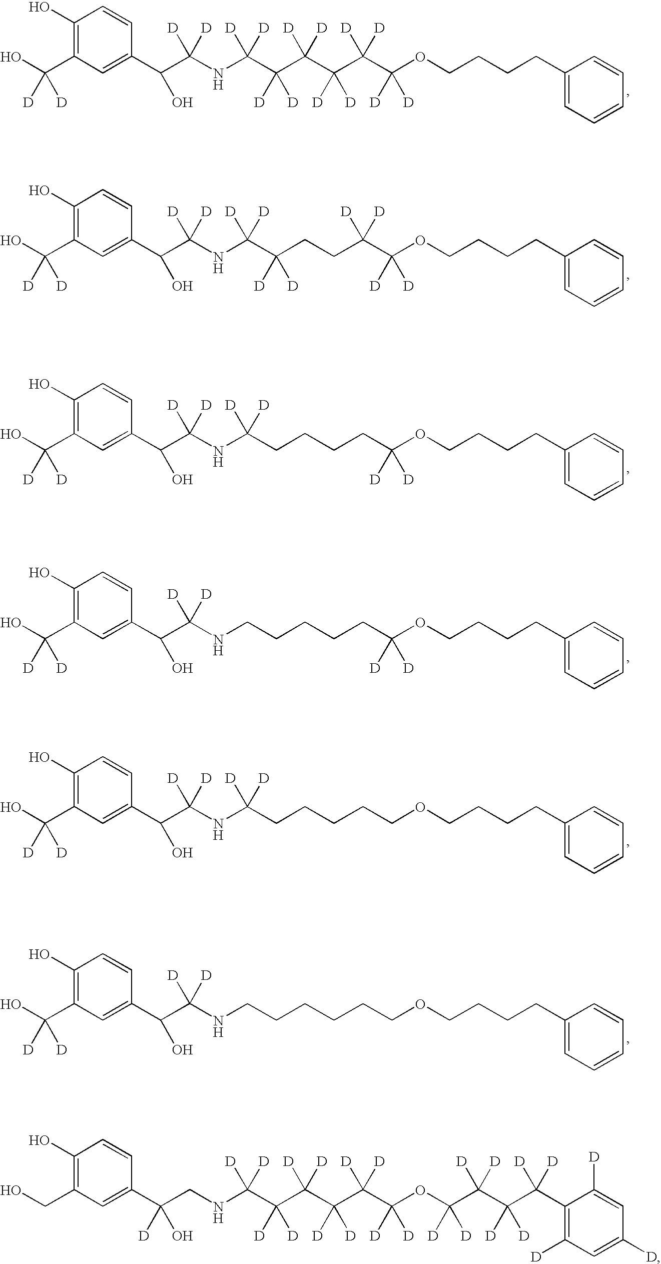

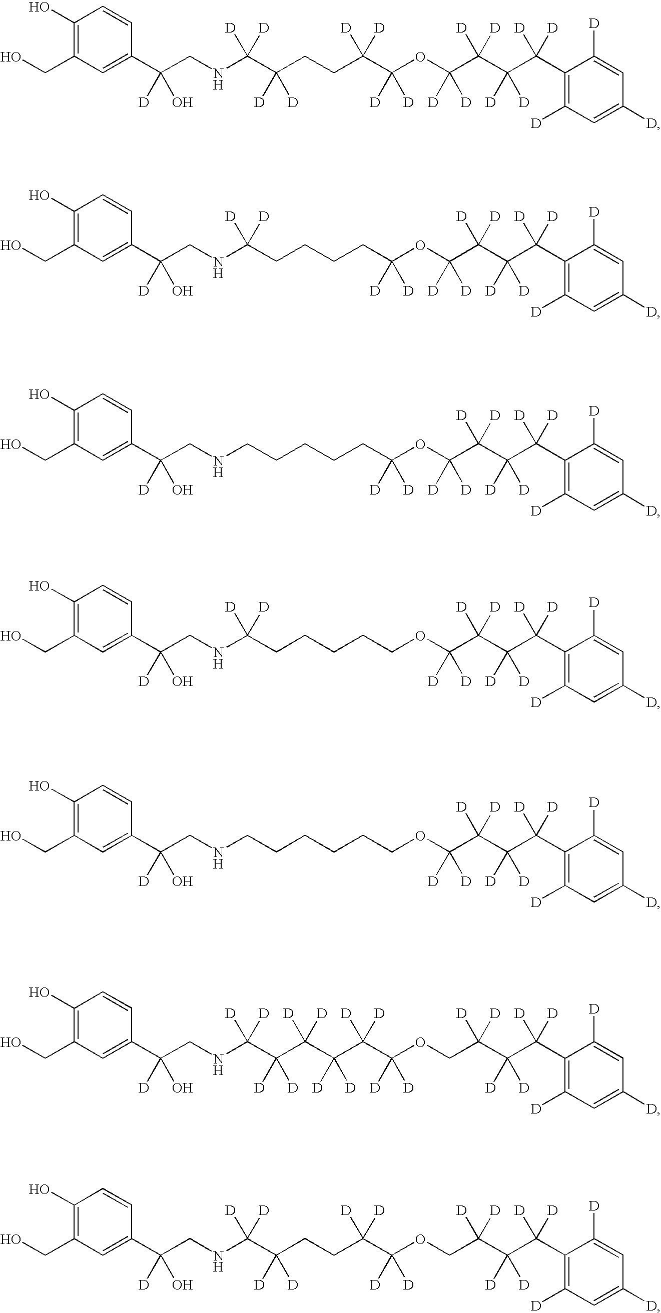

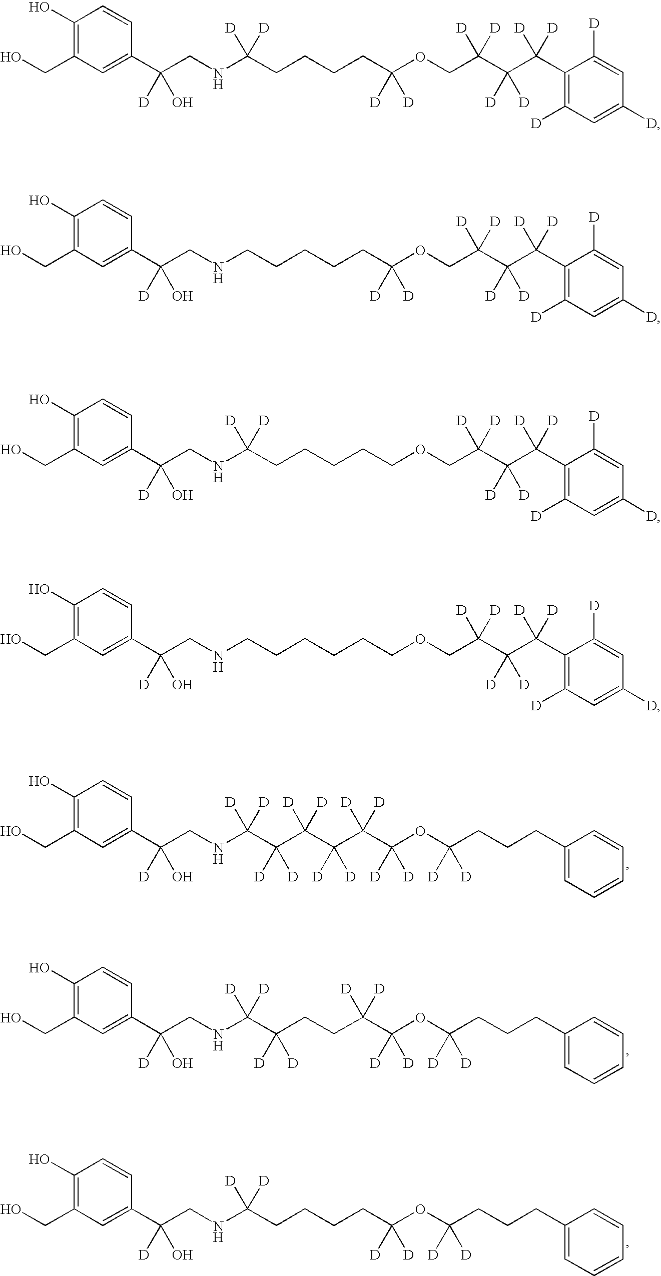

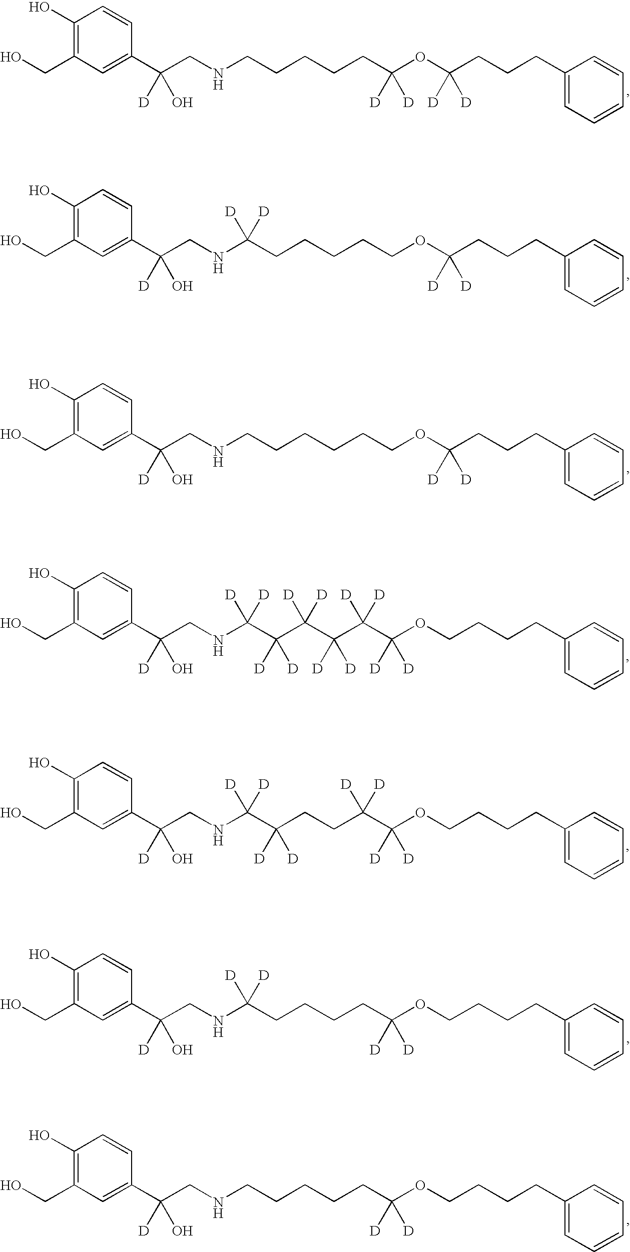

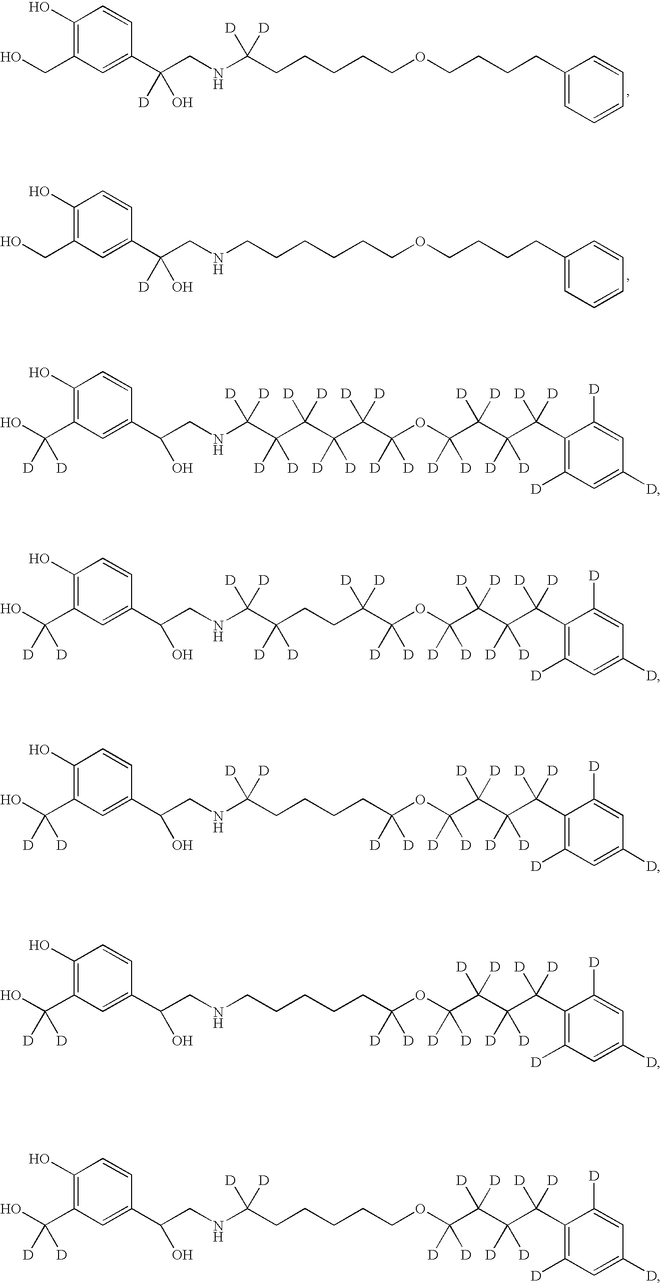

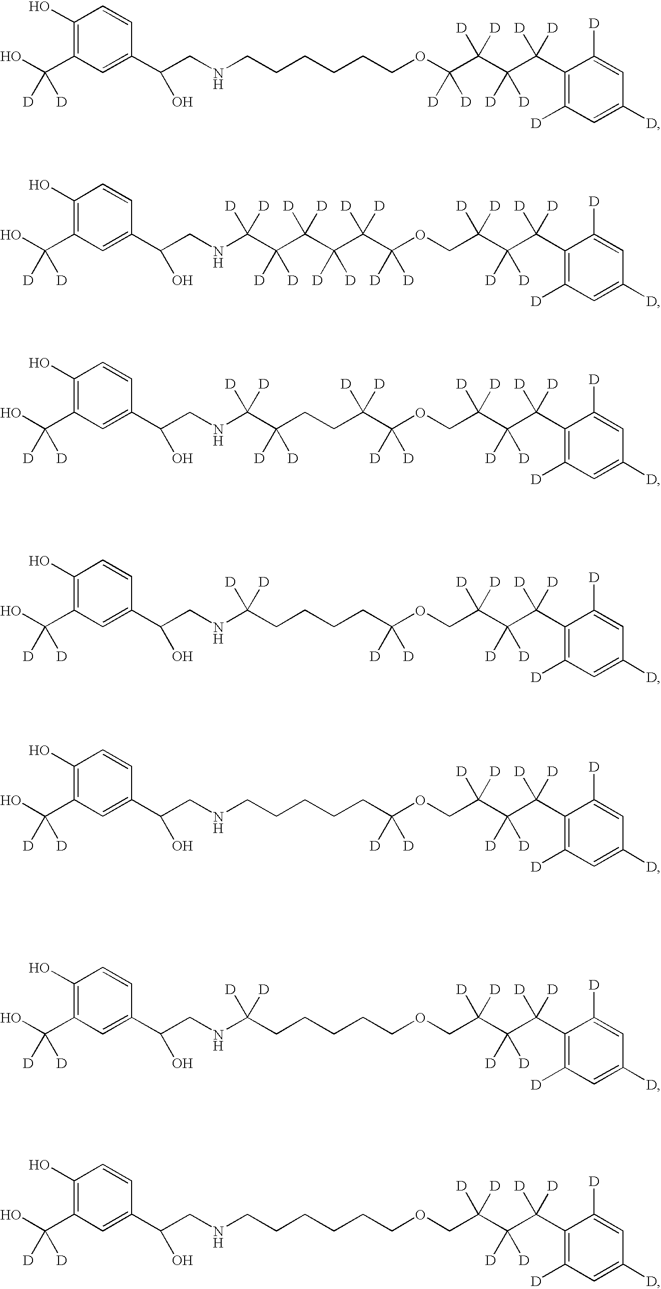

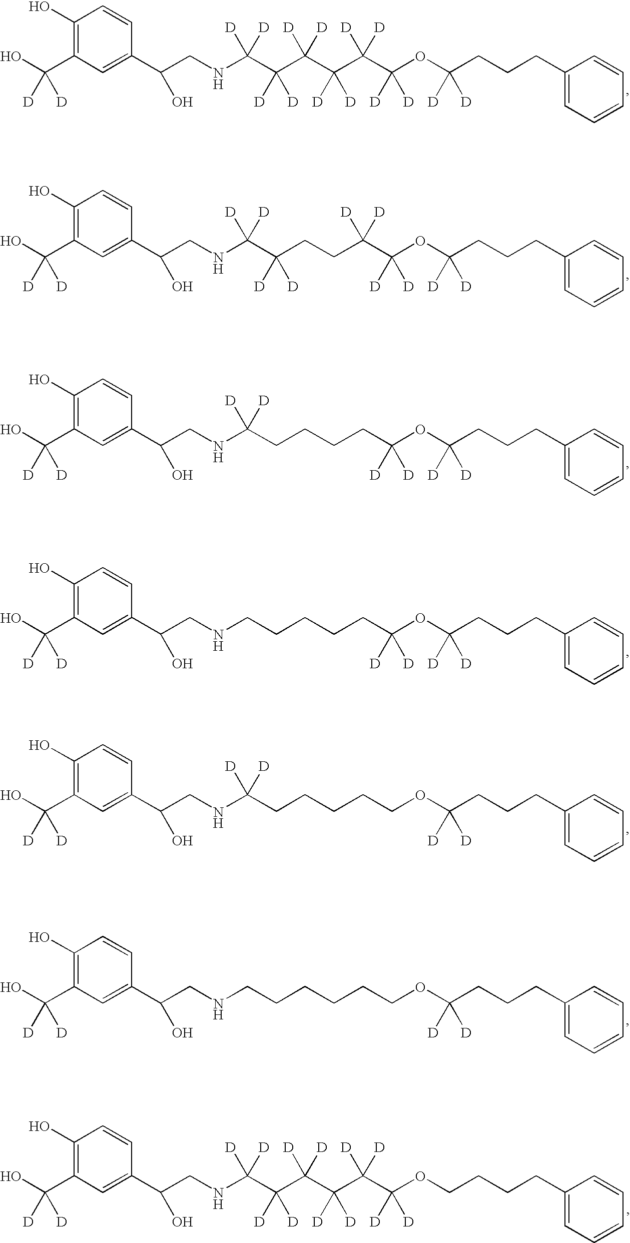

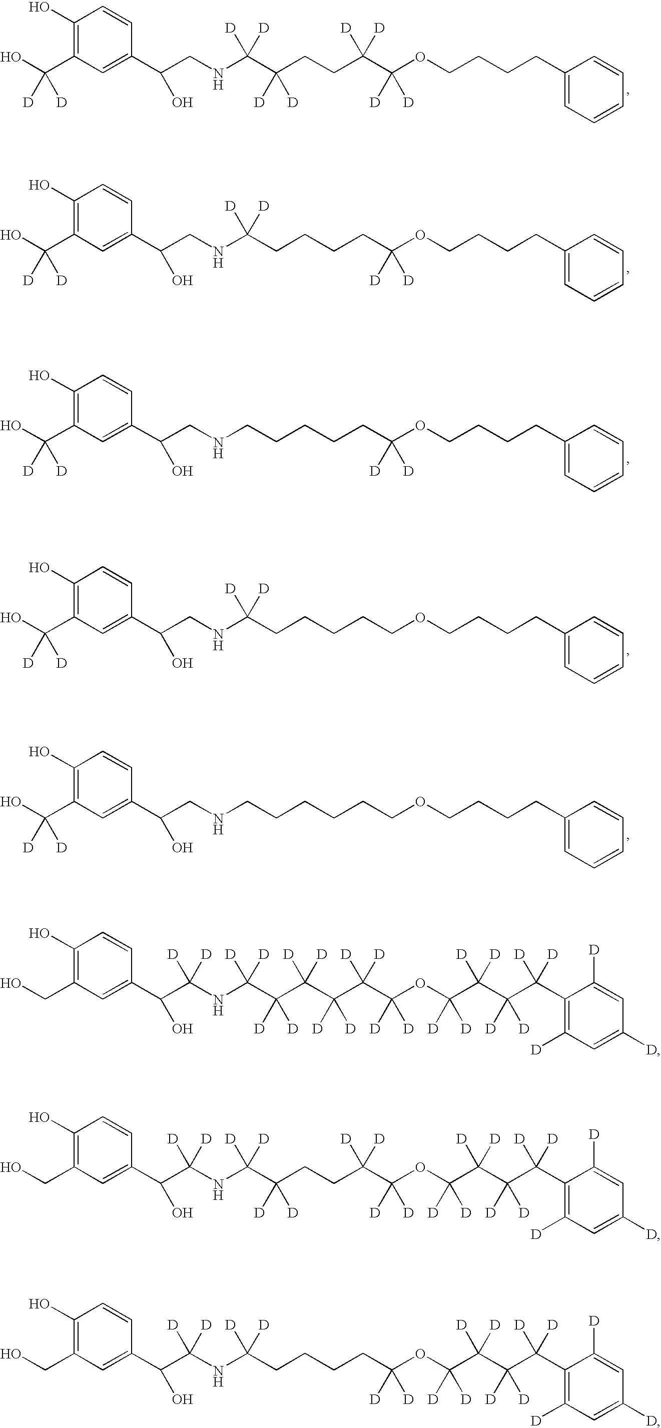

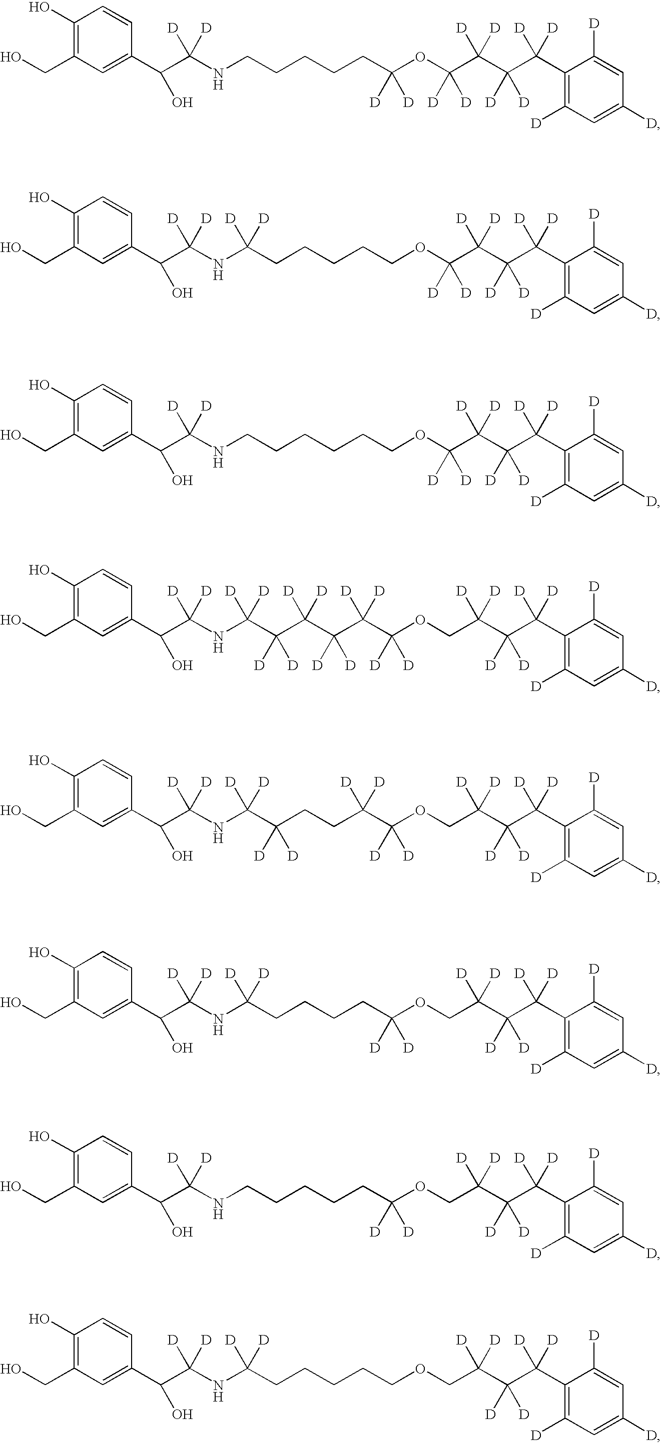

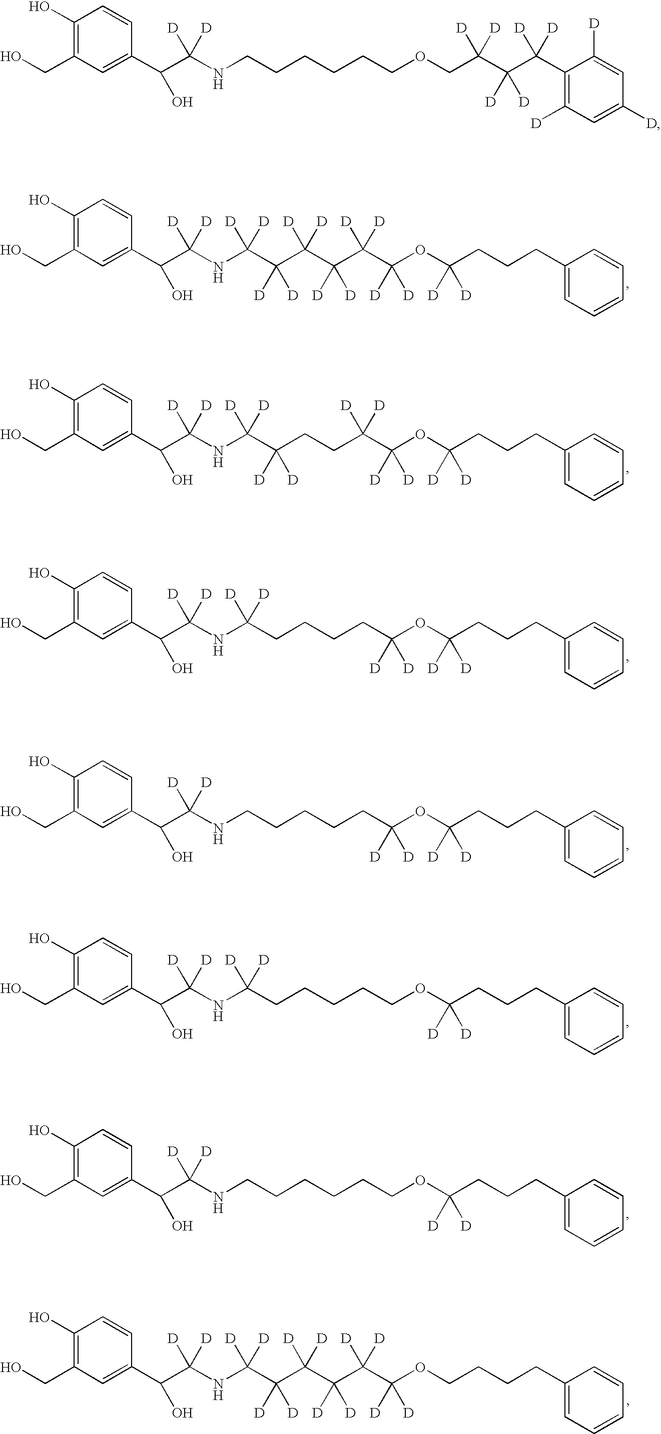

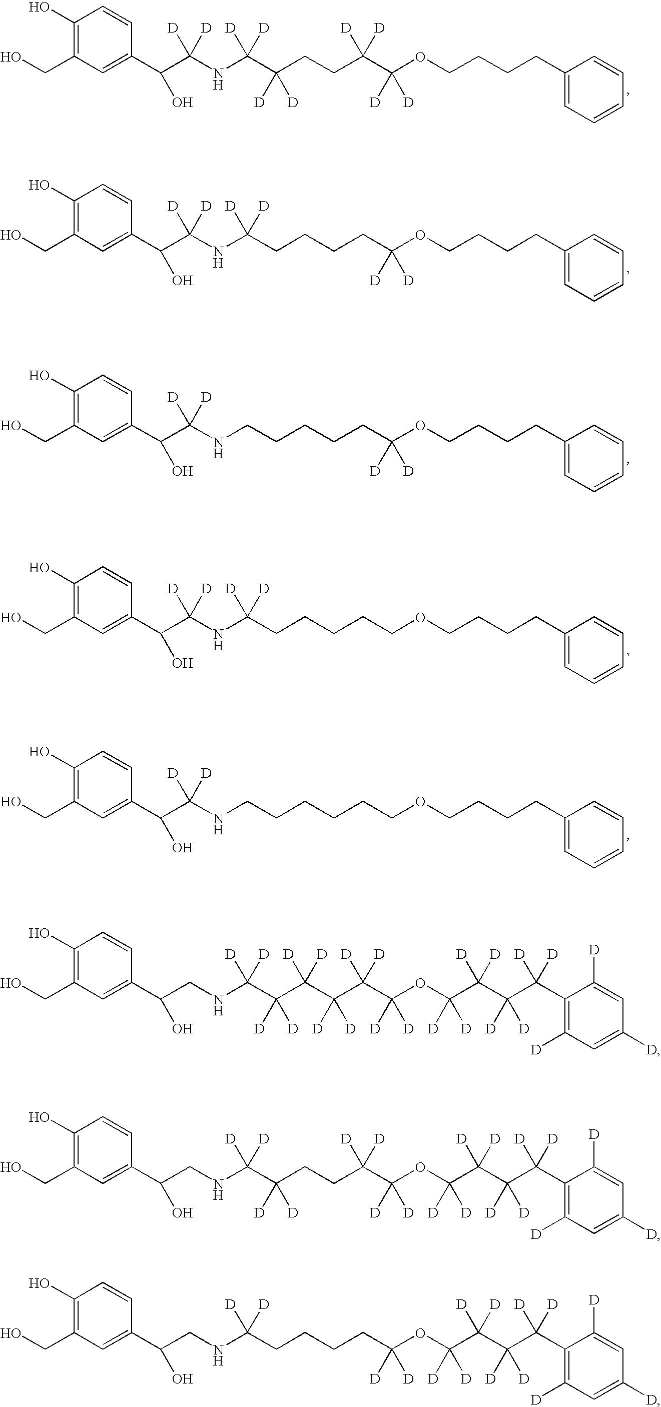

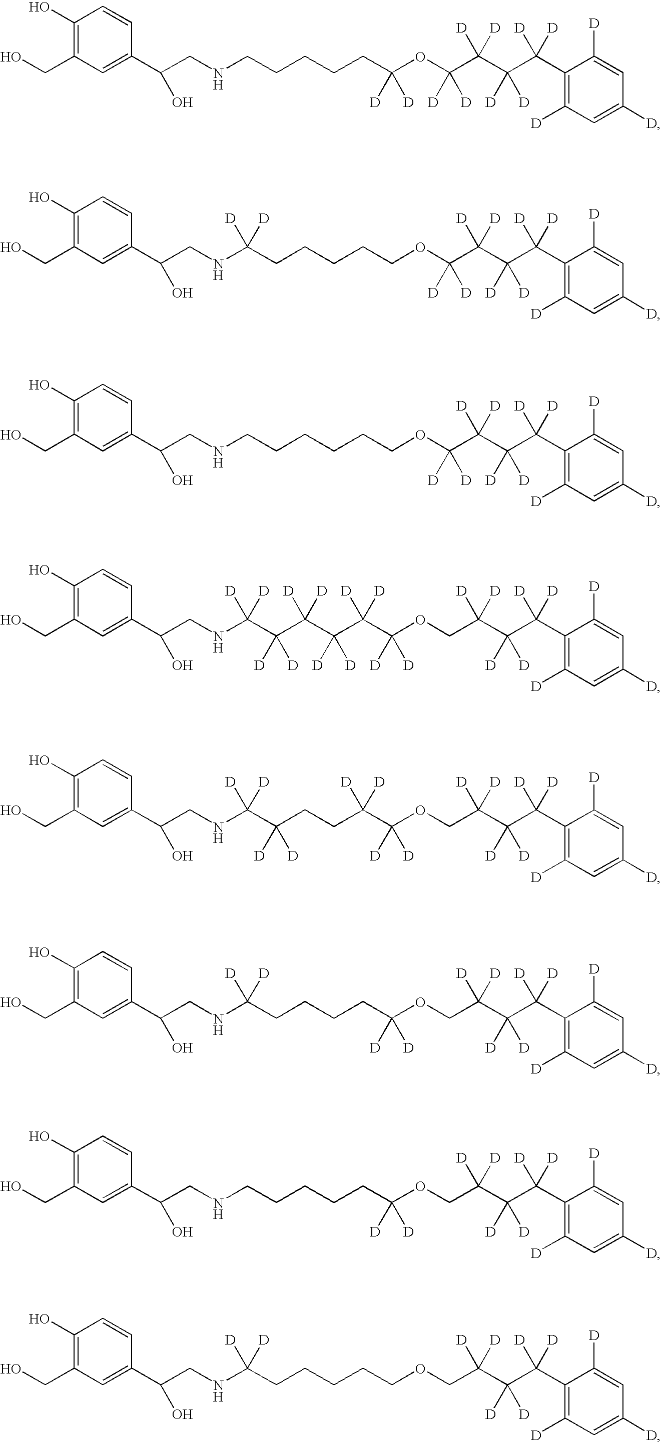

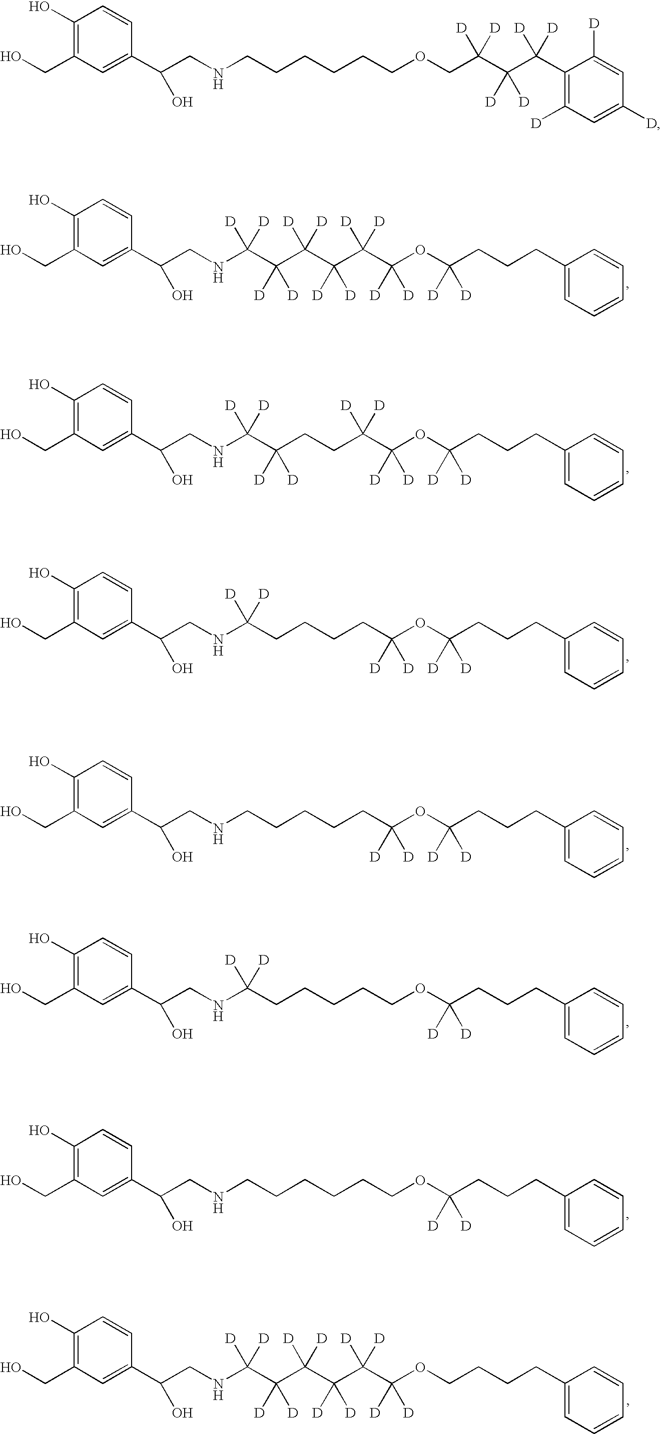

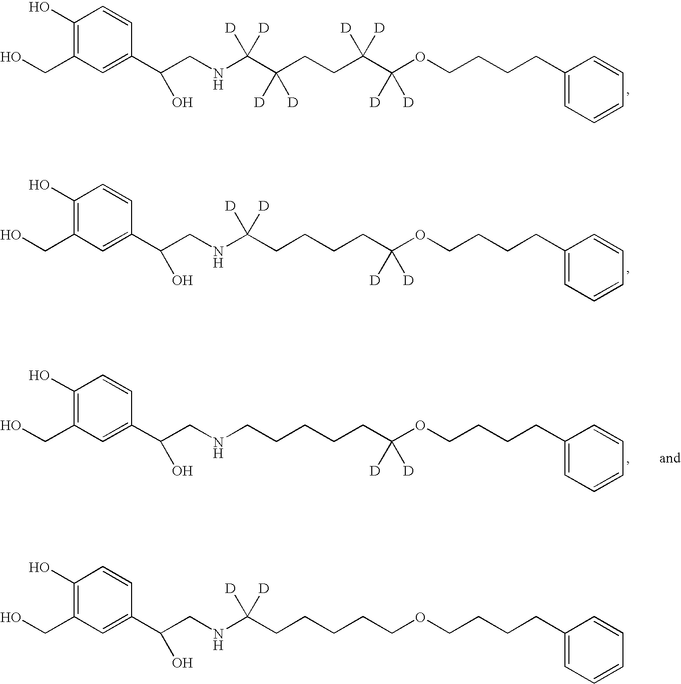

or a pharmaceutically acceptable salt, solvate, or prodrug thereof.

Changes in the metabolic properties of the compounds disclosed herein as compared to their non-isotopically enriched analogs can be shown using the following assays. Compounds listed above which have not yet been made and/or tested are predicted to have altered metabolic properties as shown by one or more of these assays as well.

### Biological Activity Assays

In vitro Liver Microsomal Stability Assay

Liver microsomal stability assays were conducted at 1 mg per mL liver microsome protein with an NADPH-generating system in 2% NaHCO₃ (2.2 mM NADPH, 25.6 mM glucose 6-phosphate, 6 units per mL glucose 6-phosphate dehydrogenase and 3.3 mM MgCl₂). Test compounds were prepared as solutions in 20% acetonitrile-water and added to the assay mixture (final assay concentration 1 μM) and incubated at 37° C. Final concentration of acetonitrile in the assay should be <1%. Aliquots (50L) were taken out at times 0, 0.25, 0.30, and 1 hours, and diluted with ice cold acetonitrile (200 μL) to stop the reactions. Samples were centrifuged at 12,000 RPM for 10 min to precipitate proteins. Supernatants were transferred to micro centrifuge tubes and stored for LC/MS/MS analysis of the degradation half-life of the test compounds. It has thus been found that compounds as disclosed in Examples 1-5 which have been tested in this assay showed improved degradation half-life, as compared to the non-isotopically enriched compound. Some of the compounds showed at least 13% increase in degradation half-life, as compared to the non-isotopically enriched drug. The degradation half-lives of Examples 1 through 5 (salmeterol and isotopically enriched drugs) are shown in table 1.

### Results of in vitro Human Liver Microsomal (HLM) Stability Assay

TABLE 1
% increase of HLM degradation half-life

| | | | |
|---|---|---|---|
| −25%-0% | 0%-50% | 50%-150% | >150% |

| | | |
|---|---|---|
| Example 1 | + | |
| Example 2 | | + |
| Example 3 | | + |
| Example 4 | | + |
| Example 5 | | + |

In vitro Metabolism using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula 1, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]—(S)-mephenytoin |
| CYP2C8 | Paclitaxel |
| CYP2C9 | Diclofenac |
| CYP2C19 | [¹³C]—(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### Monoamine Oxidase A Inhibition and Oxidative Turnover

The procedure is carried out using the methods described by Weyler, Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

### Monooamine Oxidase B Inhibition and Oxidative Turnover

The procedure is carried out as described in Uebelhack, Pharmacopsychiatry 1998, 31(5), 187-192, which is hereby incorporated by reference in its entirety.

Chiral assays for Detection of (R)-salmeterol and (R)-α-hydroxysalmeterol In Vitro

The procedure is carried out using the methods described by Zhang et al., Journal of Chromatography B 1999, 729, 225-230, which is hereby incorporated by reference in its entirety.

Radiochemical Detection of Salmeterol and α-hydroxysalmeterol In Vivo

The procedure is carried out using the methods described by Manchee et al., Drug Metab Dispos 1993, 21, 1022, which is hereby incorporated by reference in its entirety.

Detecting Salmeterol in Urine after Inhalation

The procedure is carried out using the methods described by De Boer et al., Recent Advances in Doping Analysis (4), Proceedings of Manfred Donike workshop, Cologne workshop on Dope Analysis, 14^{th} Cologne, Mar. 17-22, 1996 (1997), which is hereby incorporated by reference in its entirety.

### Detecting Salmeterol and its Metabolites, by HPLC, in Rat and Dog

The procedure is carried out using the methods described by Colthup et al., J of Pharmaceutical Sciences 1993, 82(3), 323-5, which is hereby incorporated by reference in its entirety.

From the foregoing description, one skilled in the art can ascertain the essential characteristics of this invention, and without departing from the spirit and scope thereof, can make various changes and modifications of the invention to adapt it to various usages and conditions.

What is claimed is:

1. A compound having structural Formula I 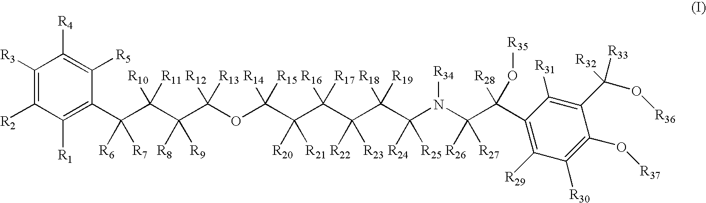
or a pharmaceutically acceptable salt thereof, wherein:
R₁-R₃₇ are independently selected from the group consisting of hydrogen and deuterium;
at least one of R₁-R₃₇ is deuterium; and
if R₃₂, R₃₃, and R₂₈ are deuterium then at least one of R₁-R₂₇, R₂₉-R₃₁, or R₃₄-R₃₇ is deuterium.

2. The compound as recited in claim 1 wherein at least one of R₁-R₃₇ independently has deuterium enrichment of no less than about 10%.

3. The compound as recited in claim 1 wherein at least one of R₁-R₃₇ independently has deuterium enrichment of no less than about 50%.

4. The compound as recited in claim 1 wherein at least one of R₁-R₃₇ independently has deuterium enrichment of no less than about 90%.

5. The compound as recited in claim 1 wherein at least one of R₁-R₃₇ independently has deuterium enrichment of no less than about 98%.

6. The compound as recited in claim 1 wherein said compound has a structural formula selected from the group consisting of: 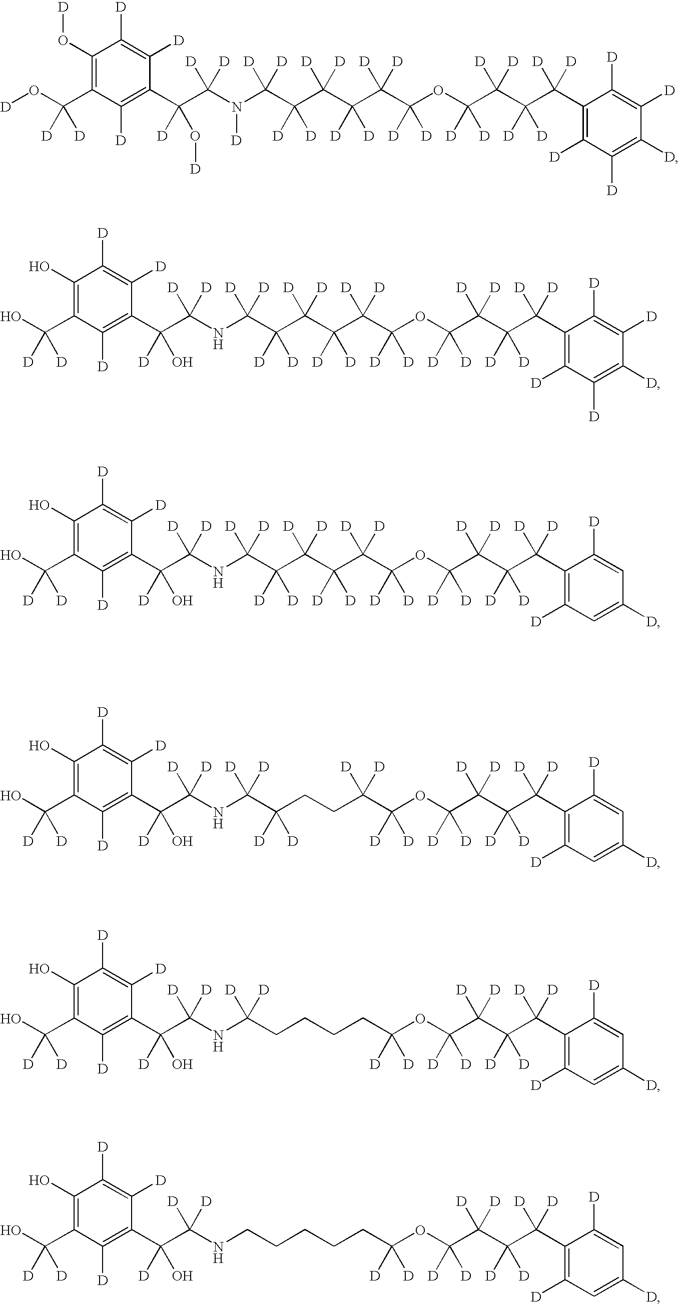 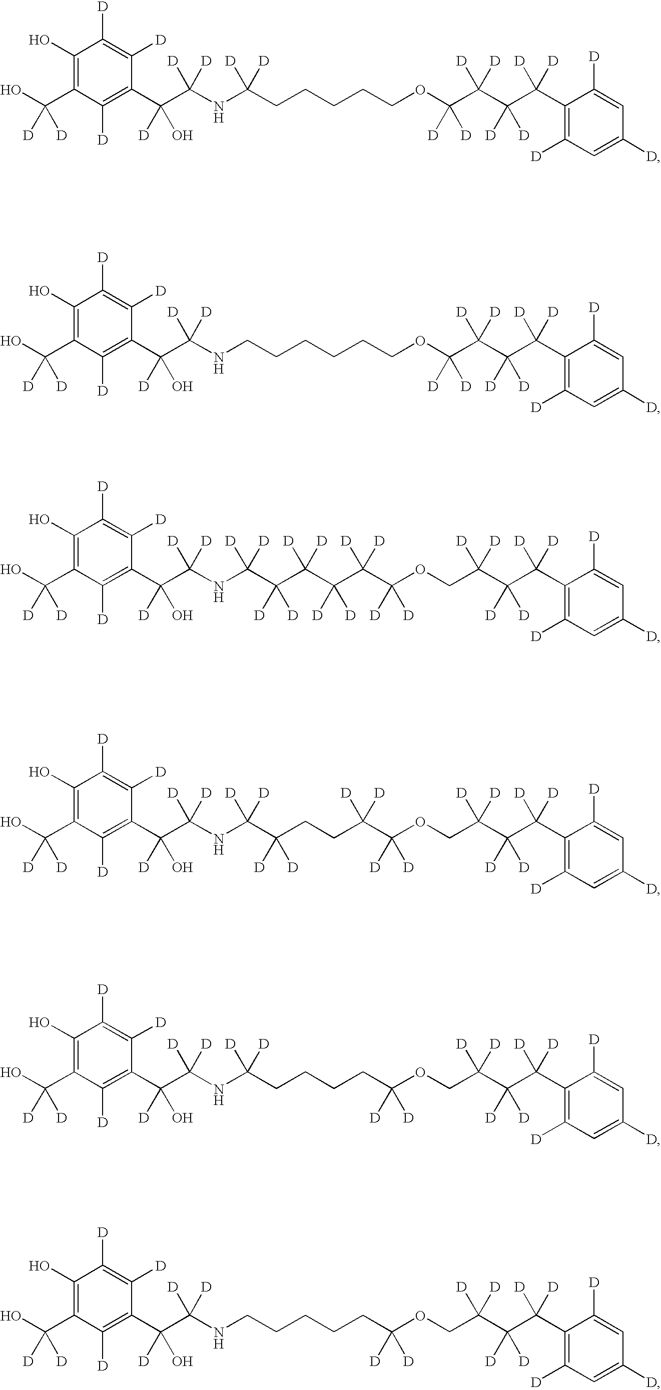 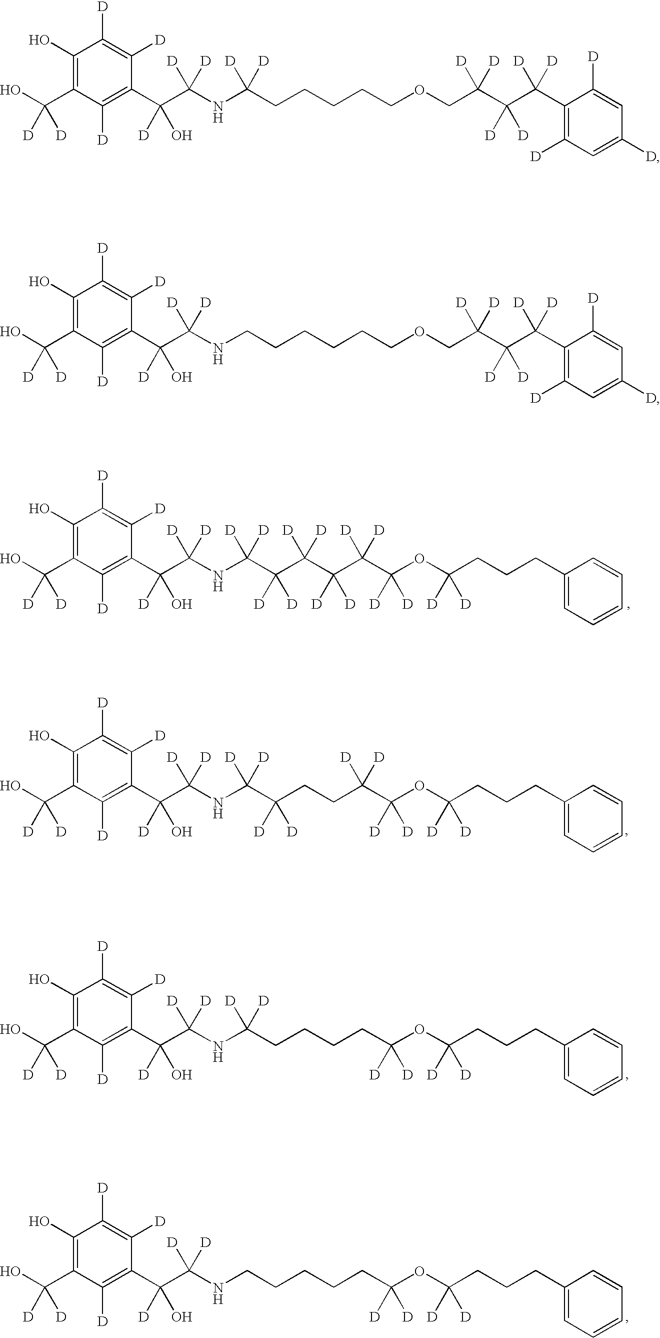 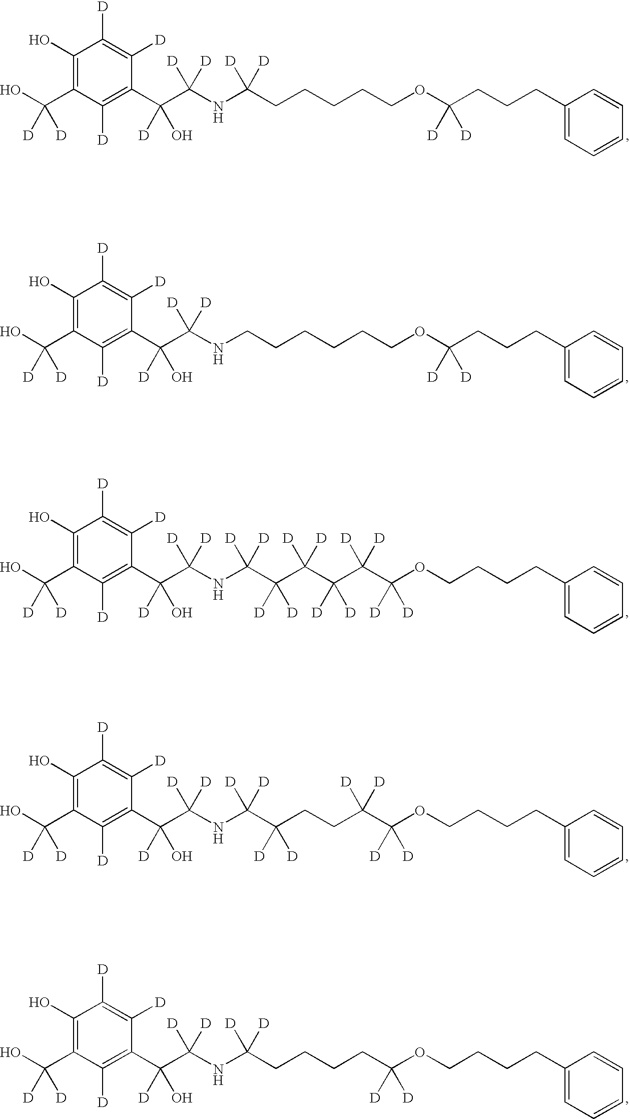 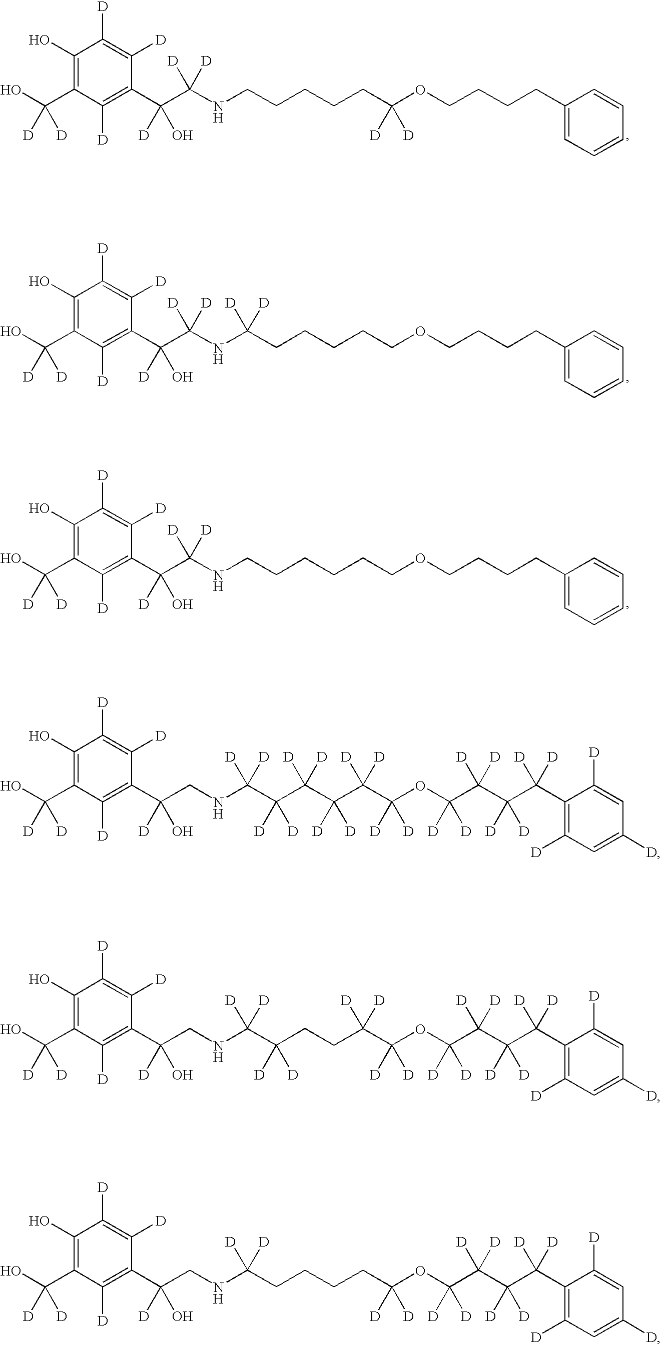 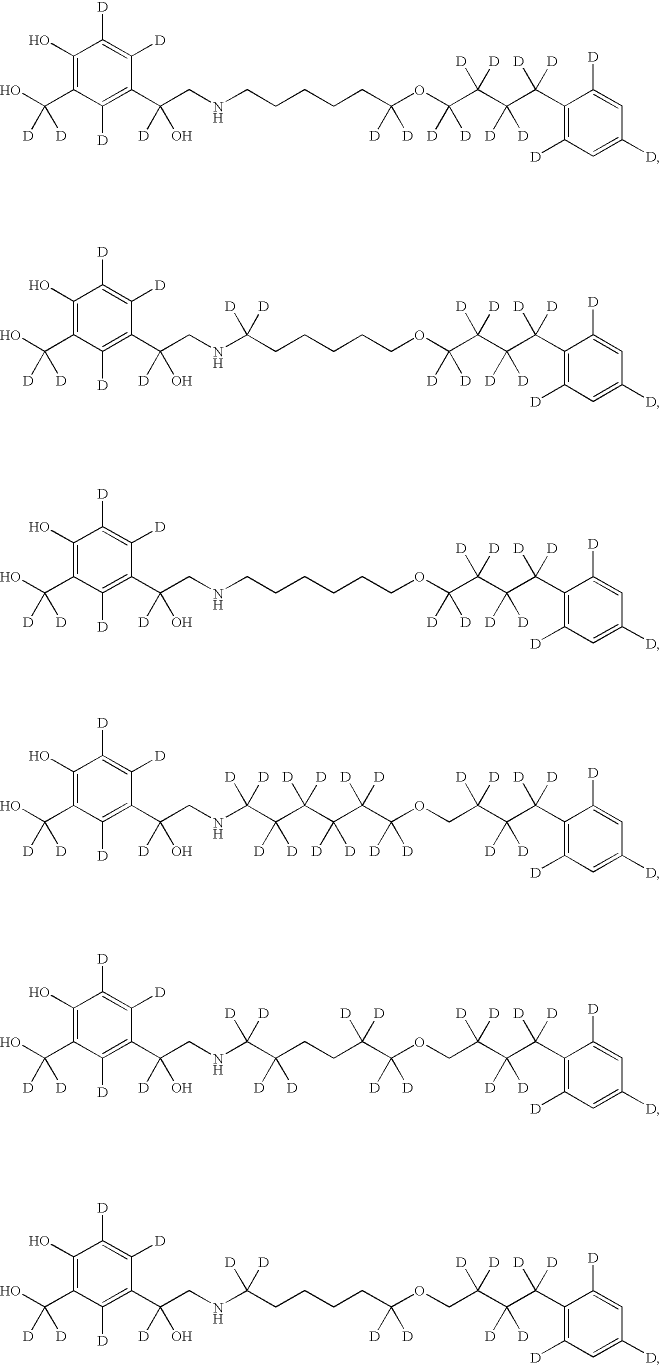 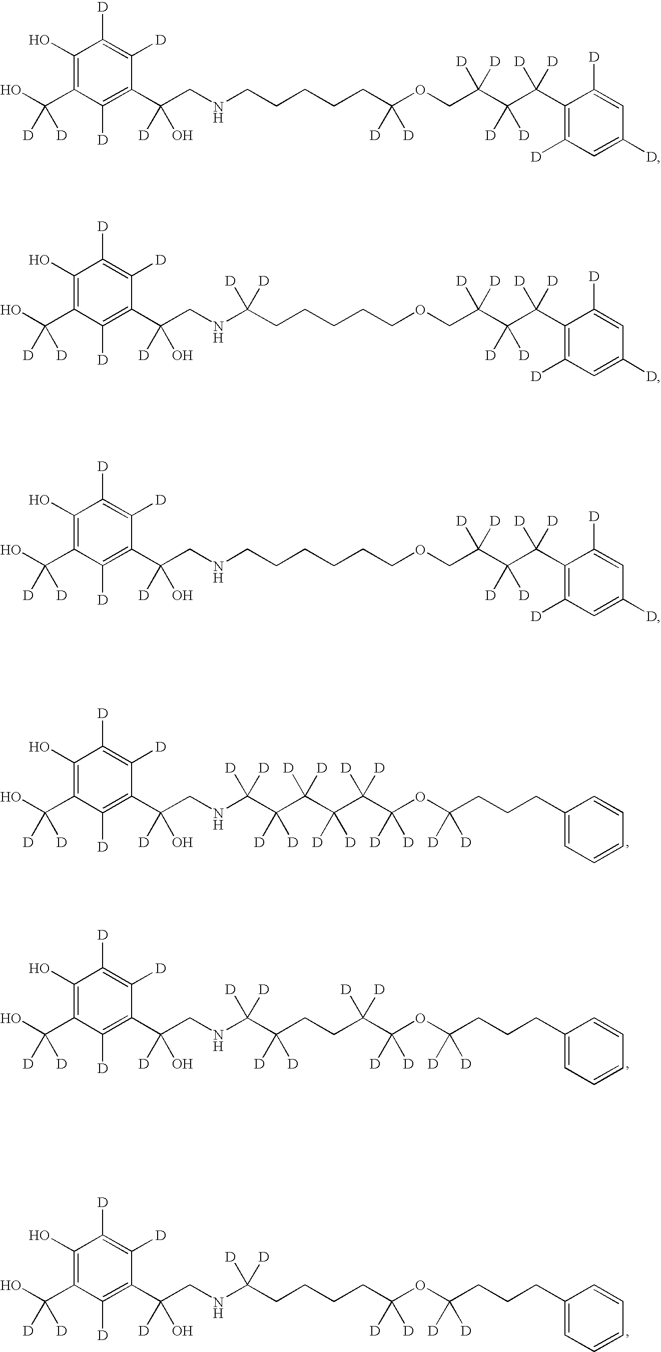 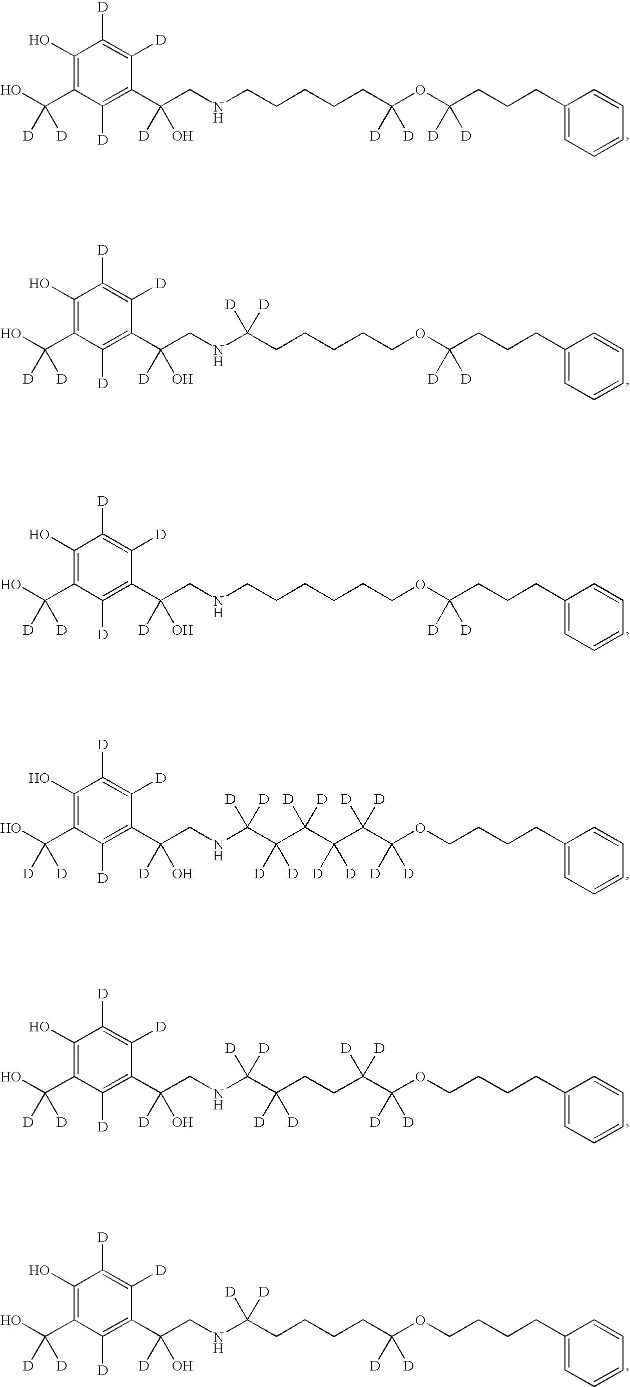 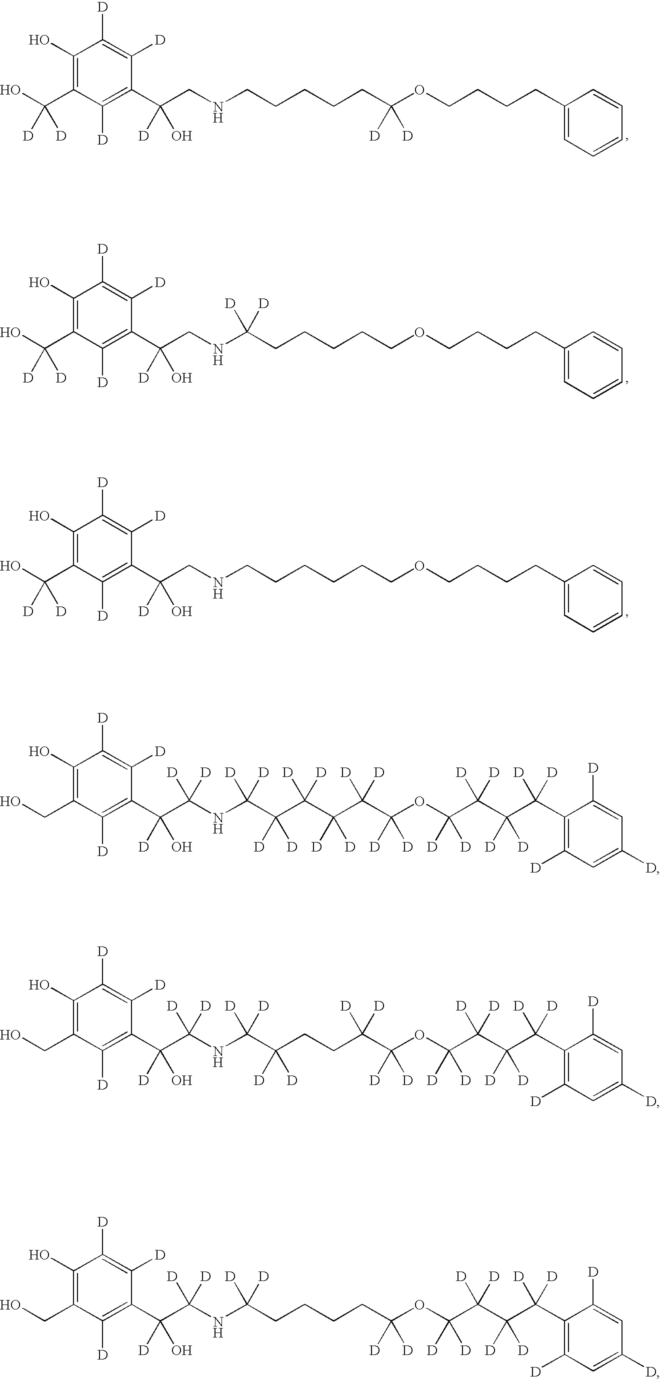 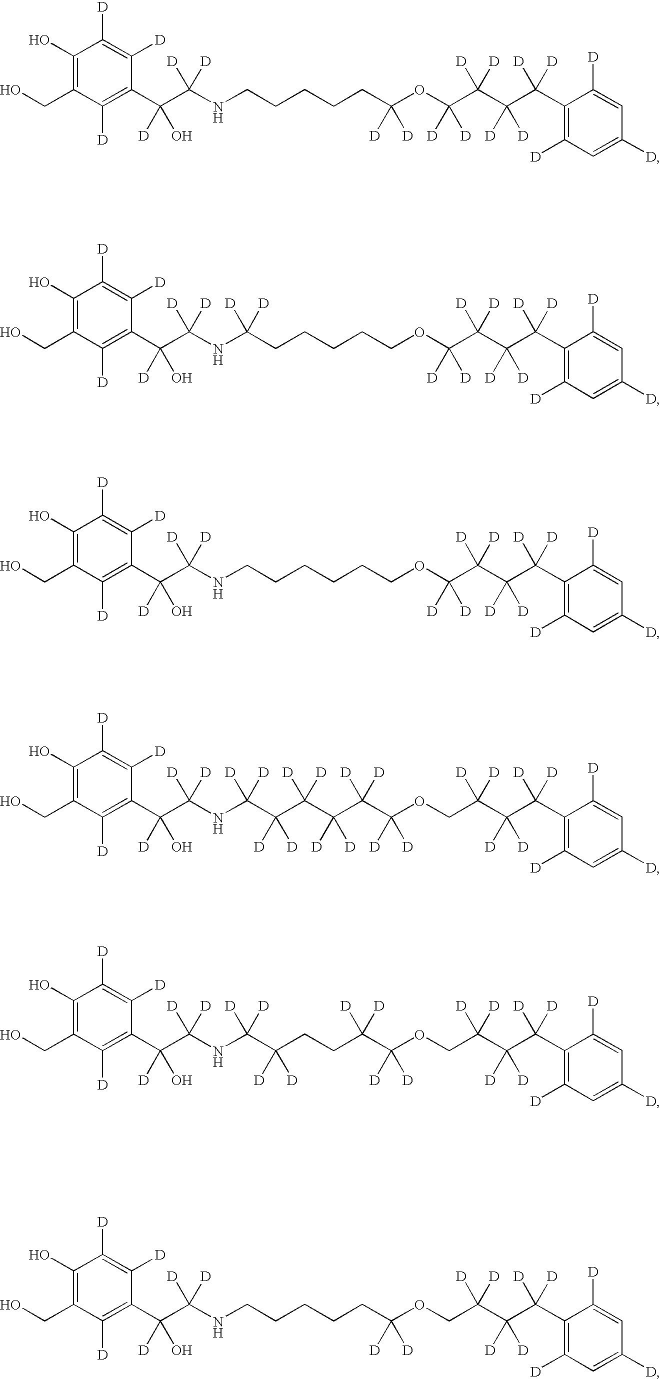 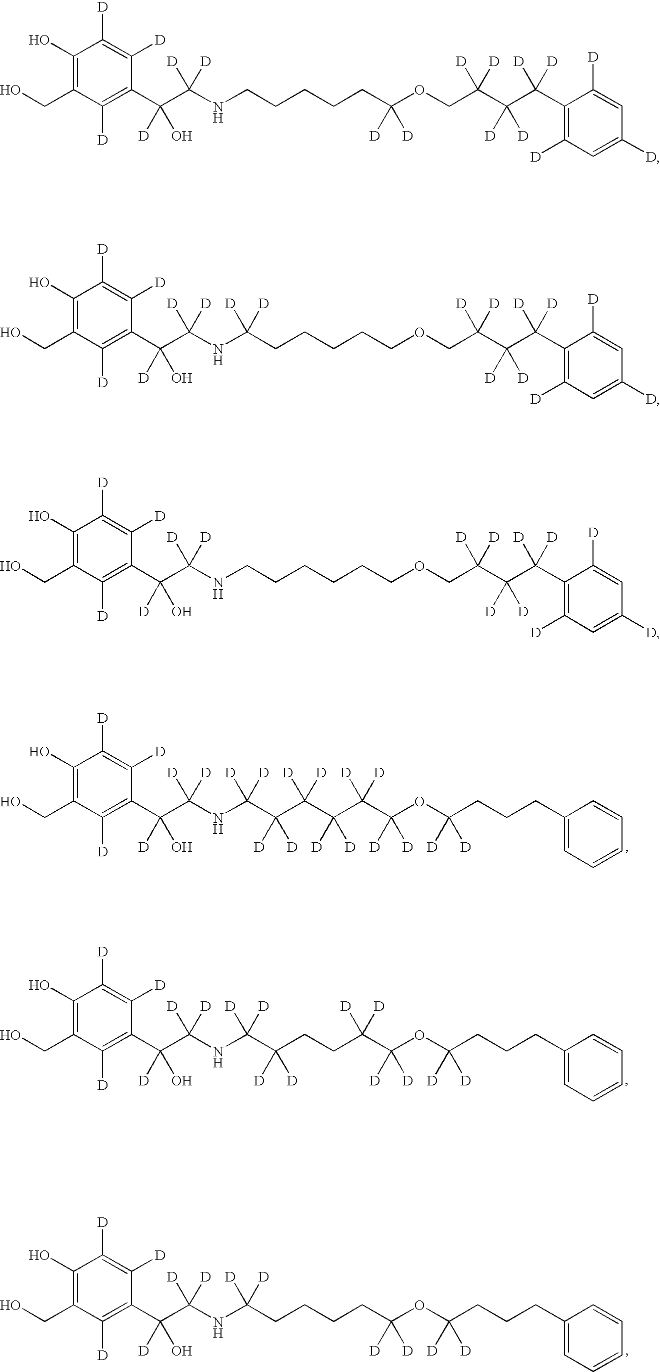 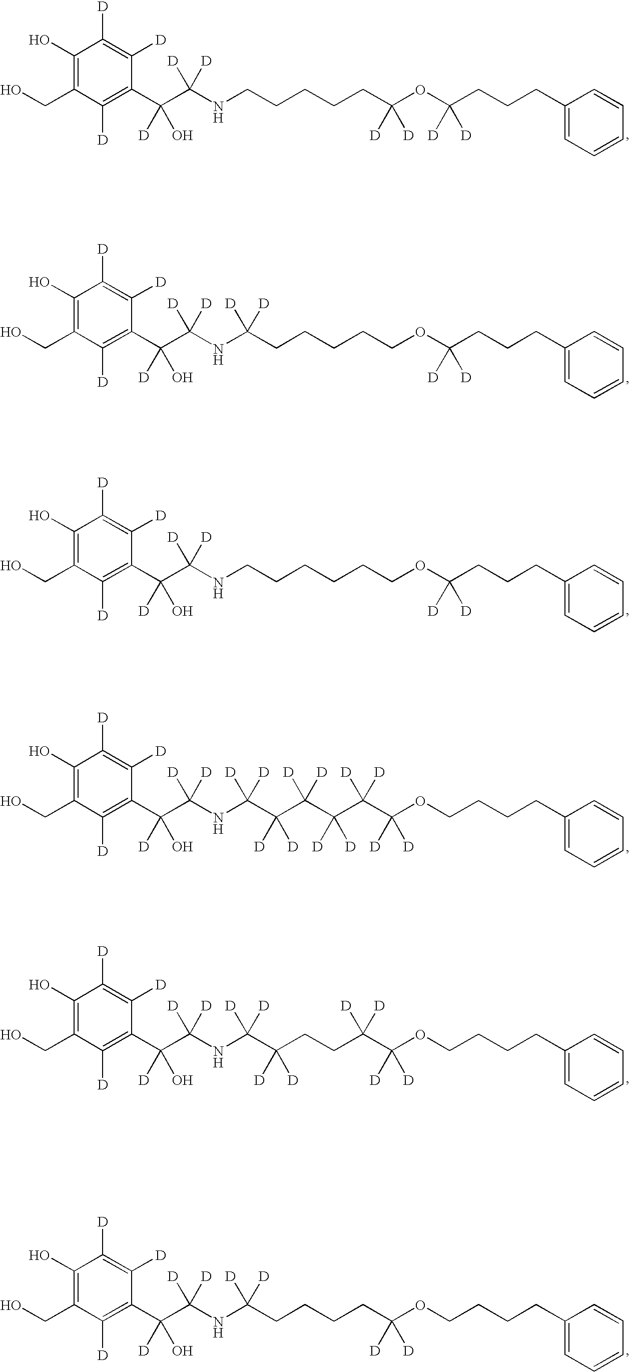 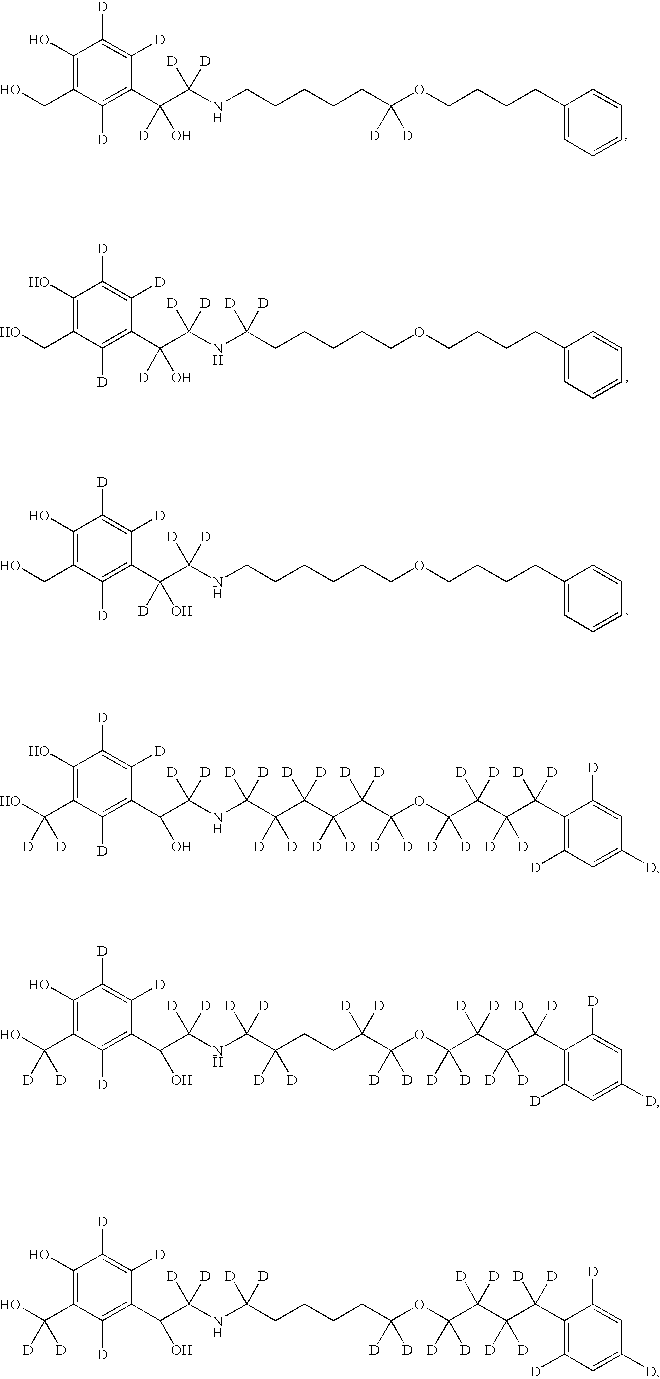 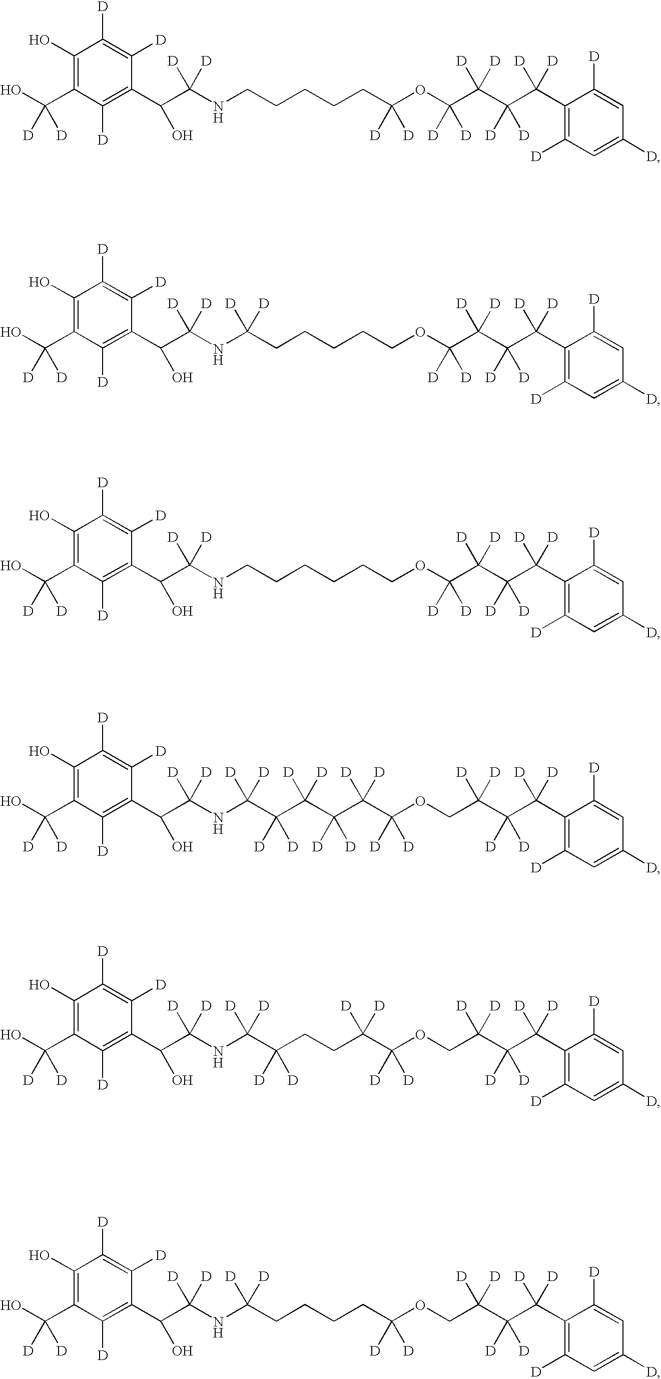 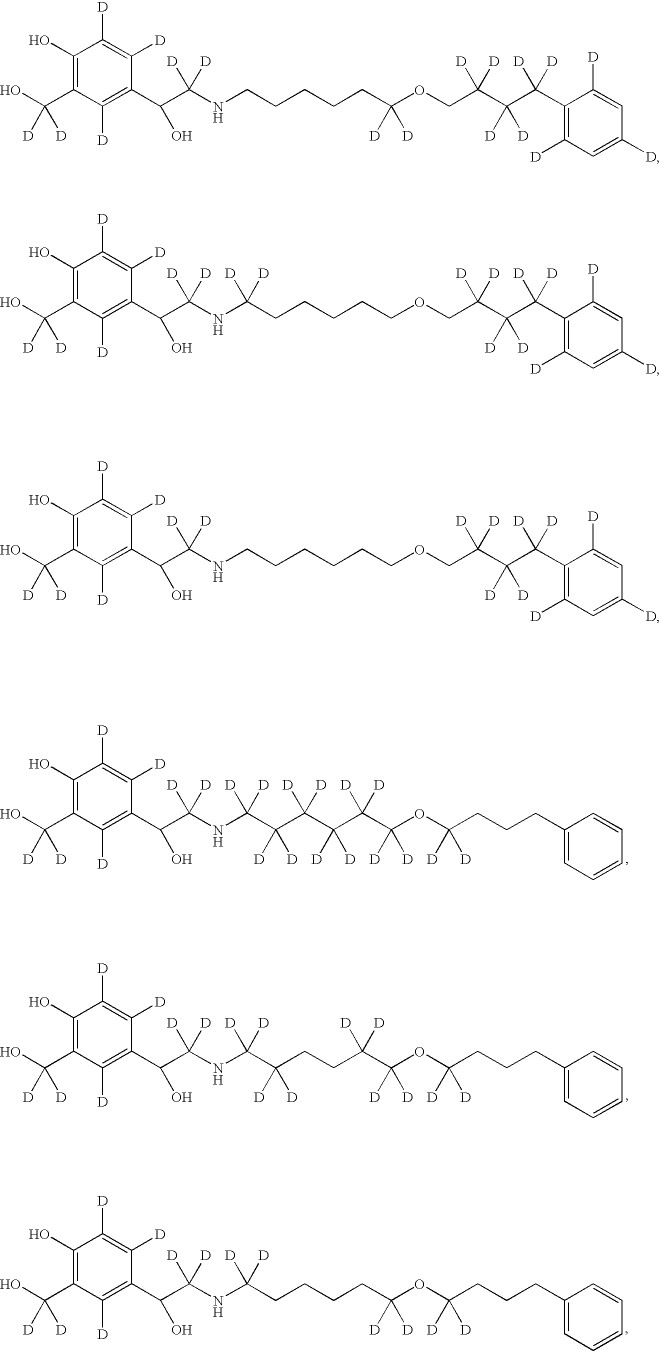 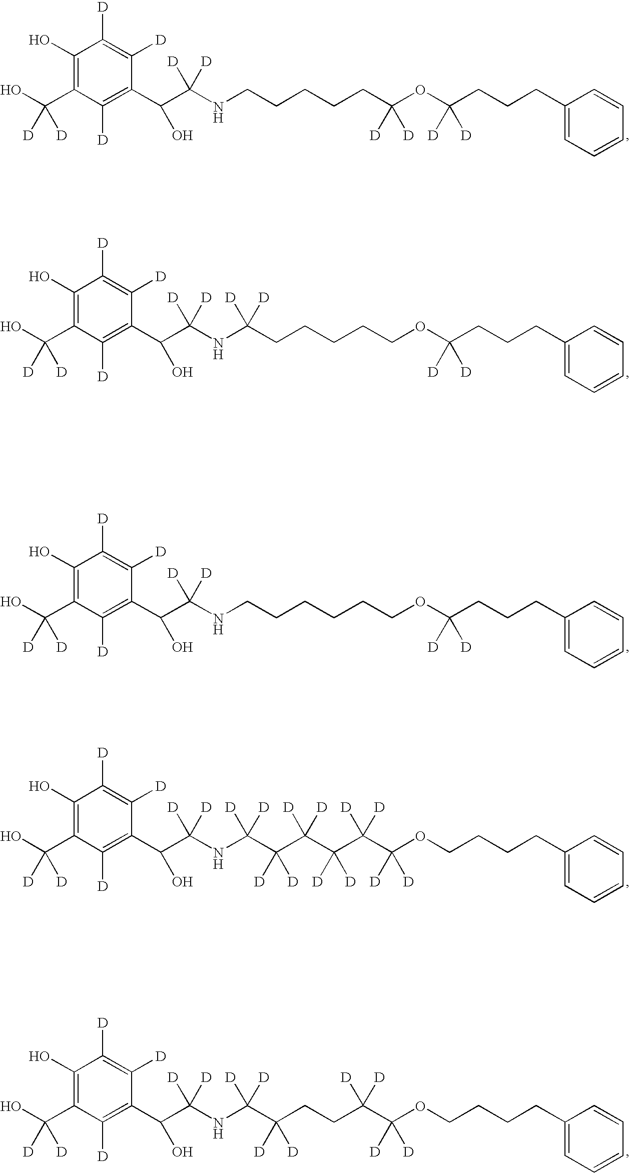 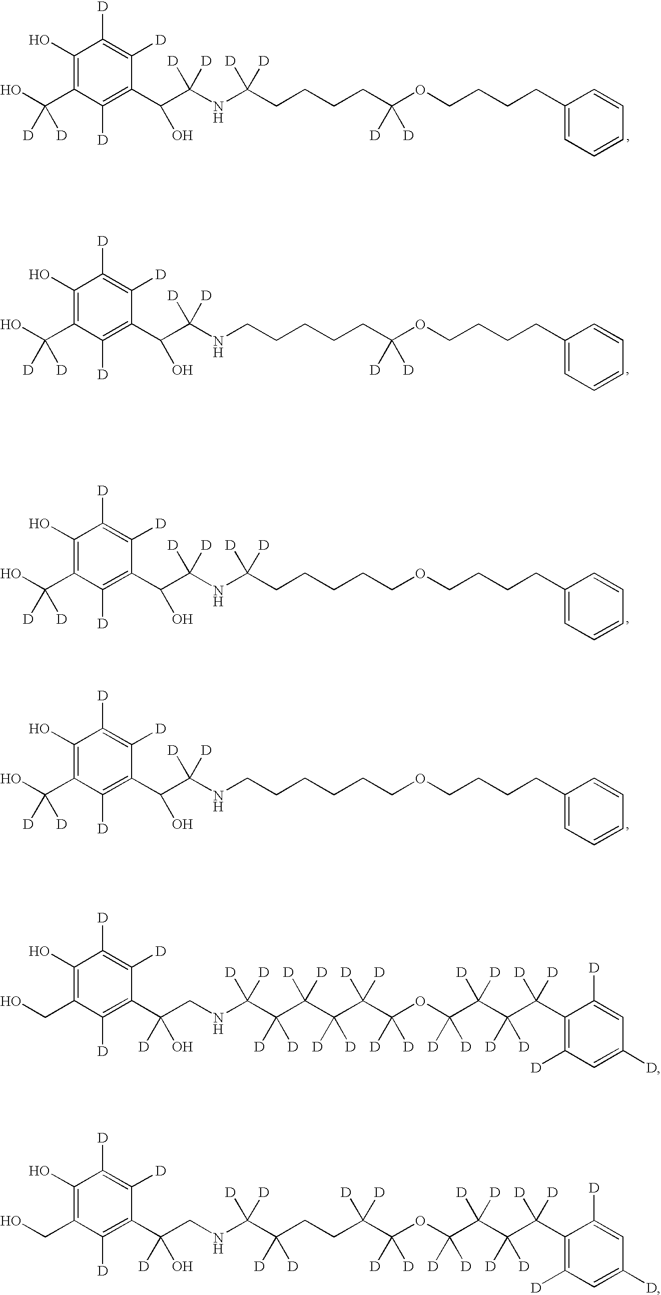 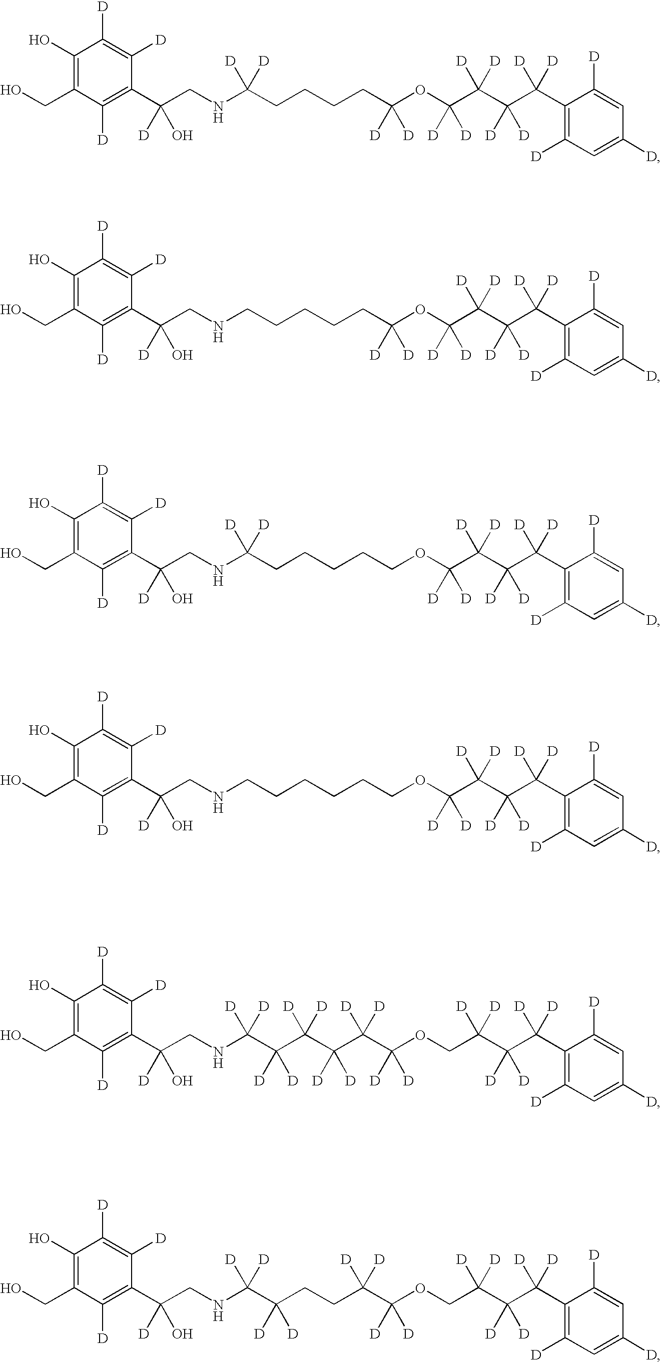 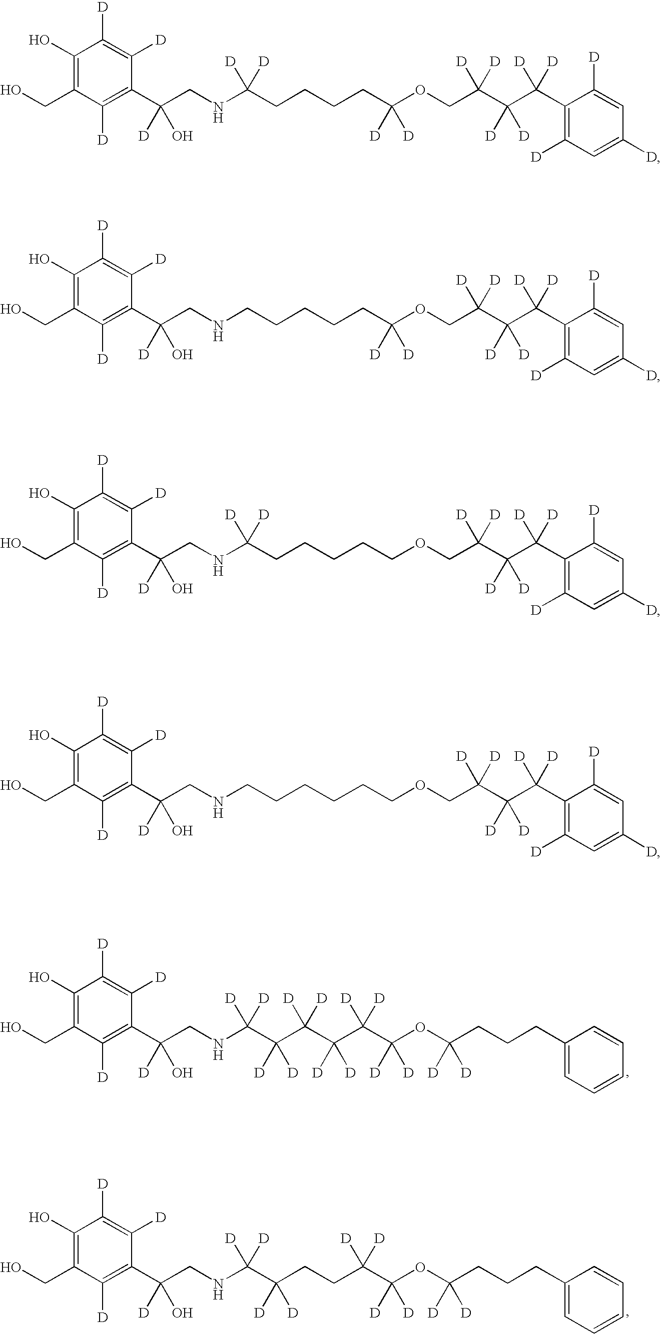 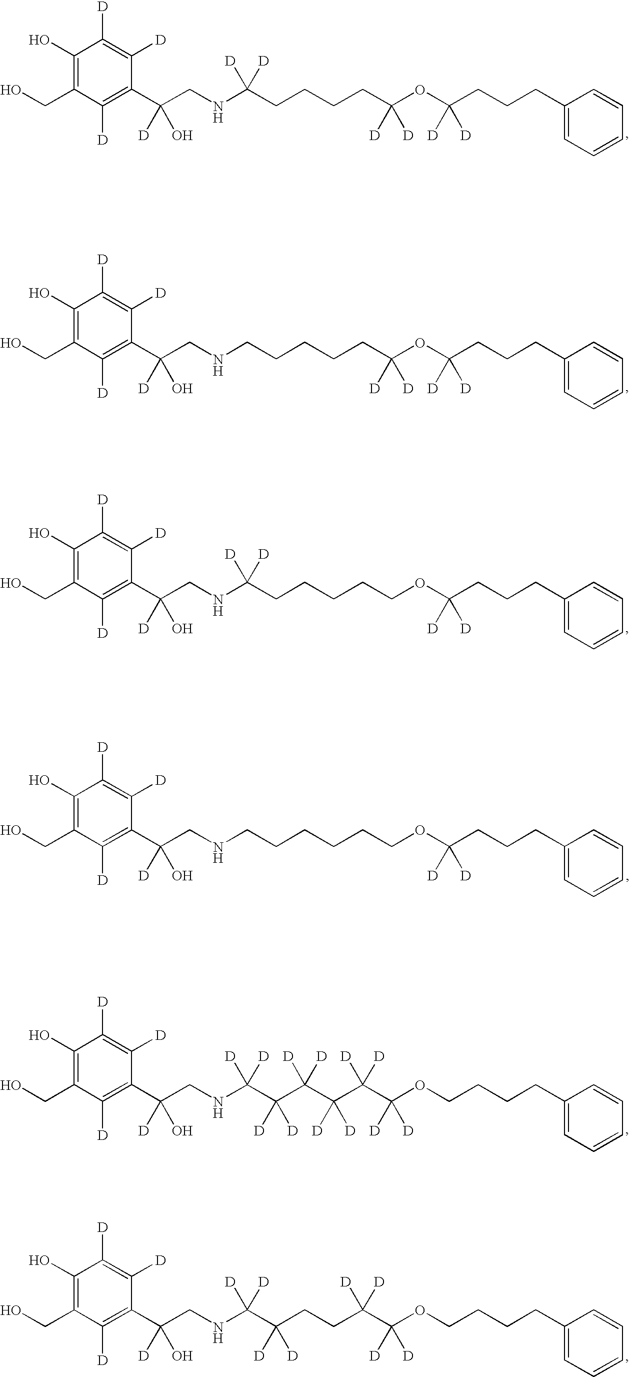 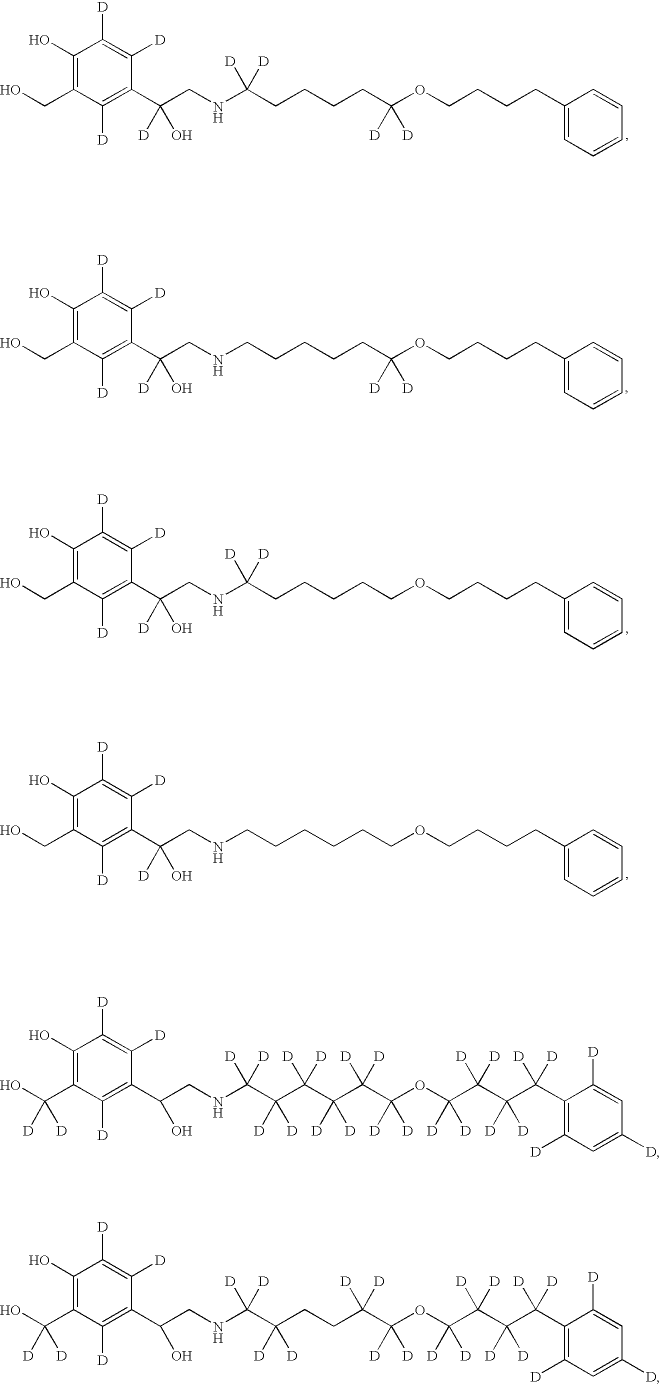 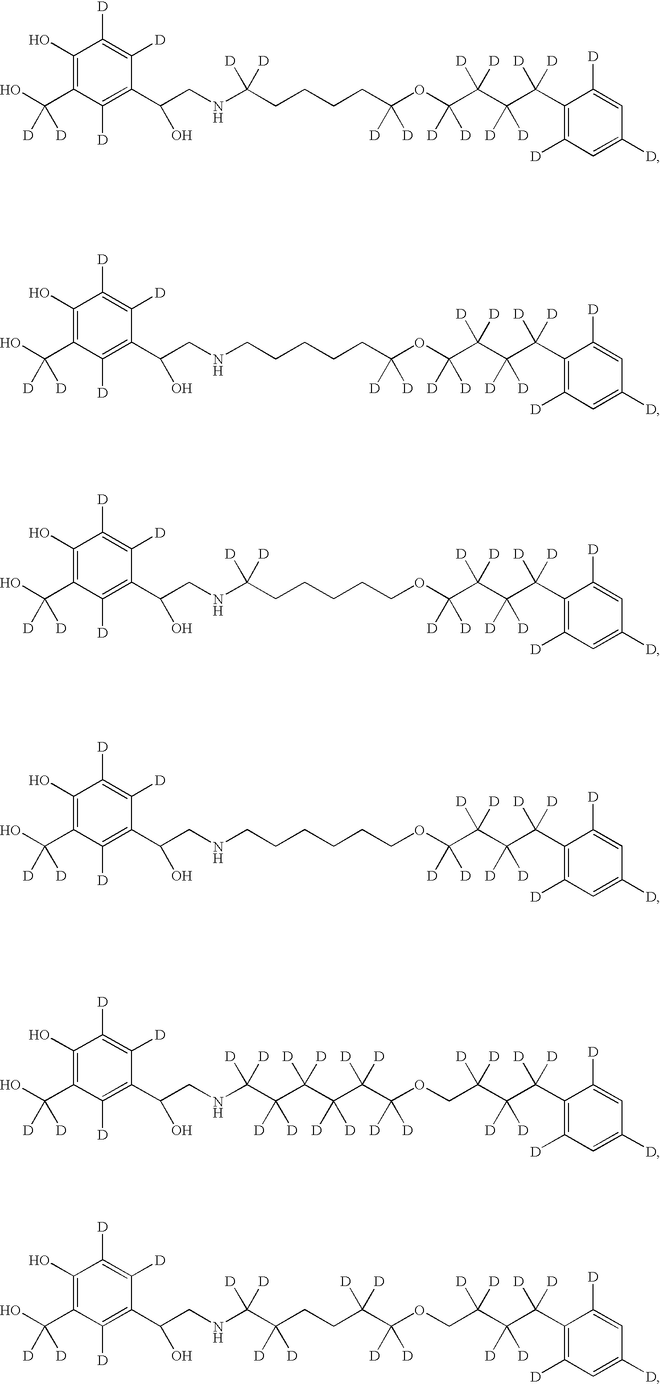 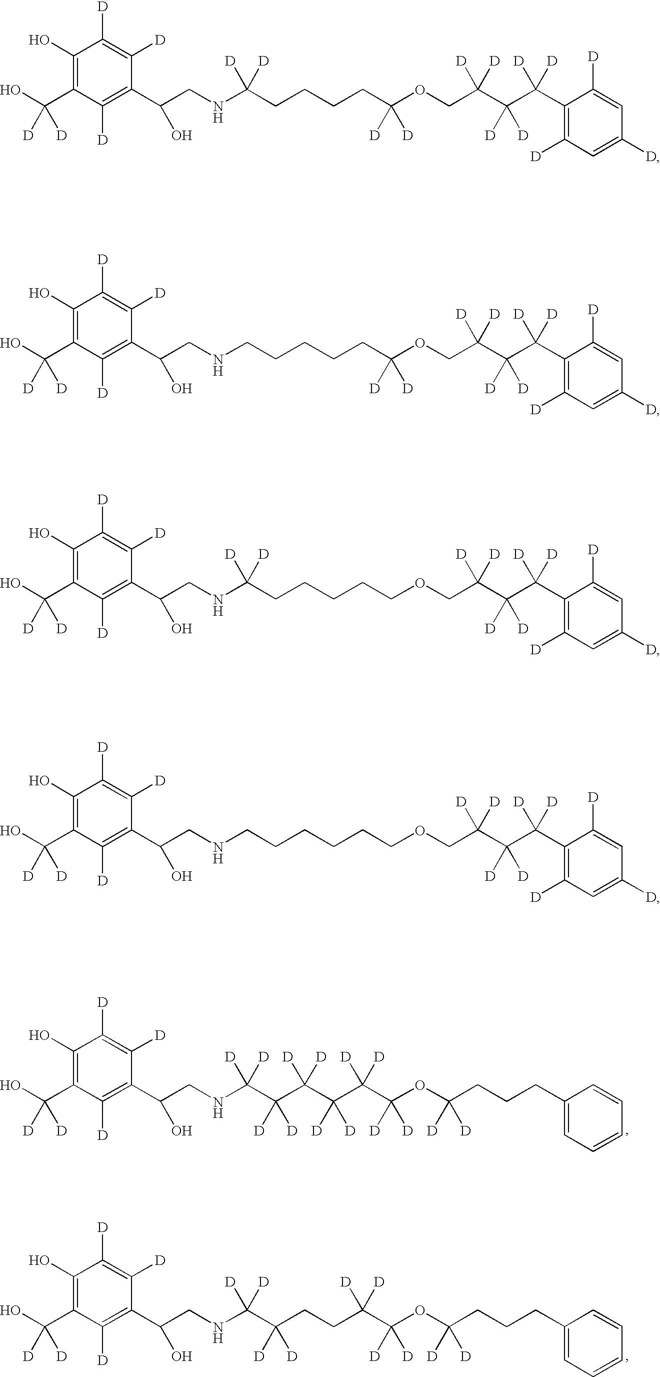 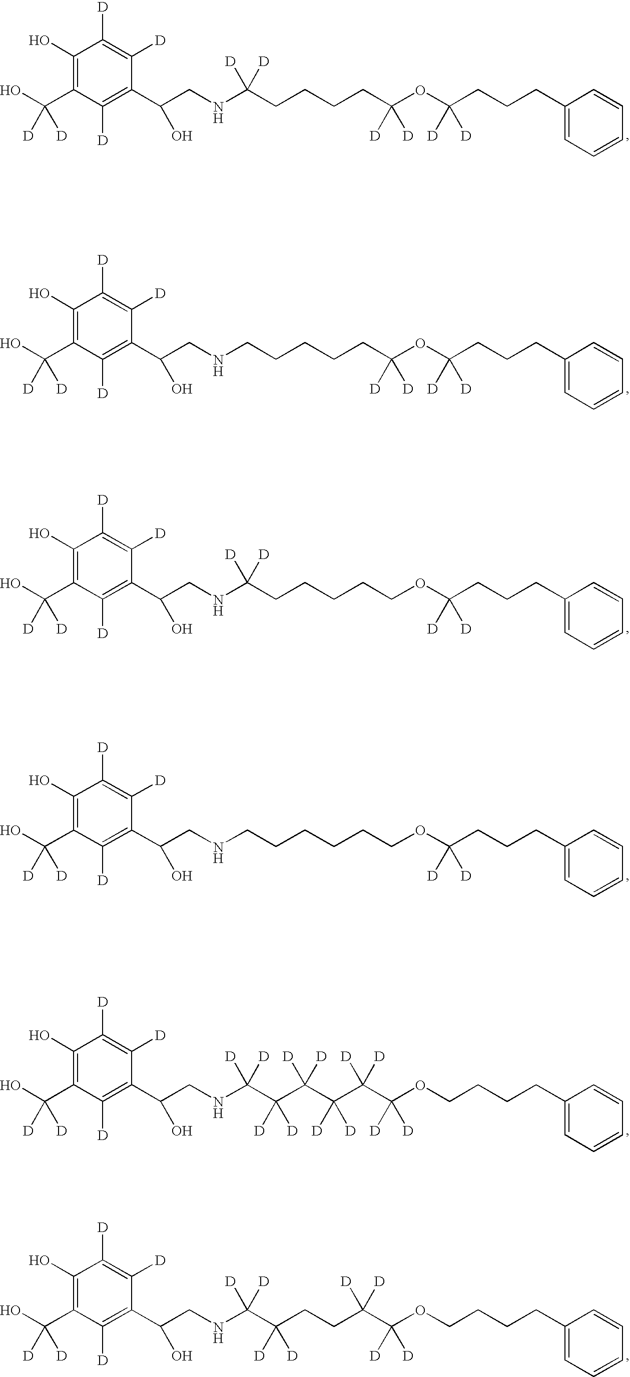 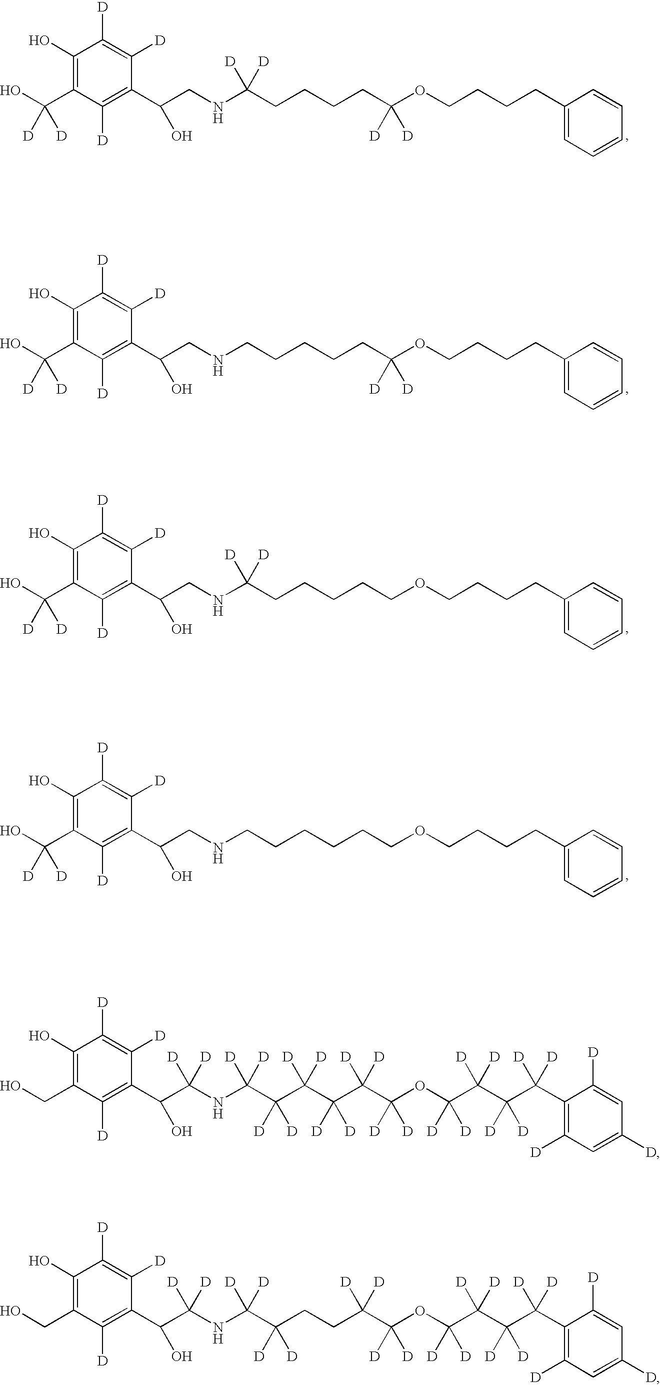 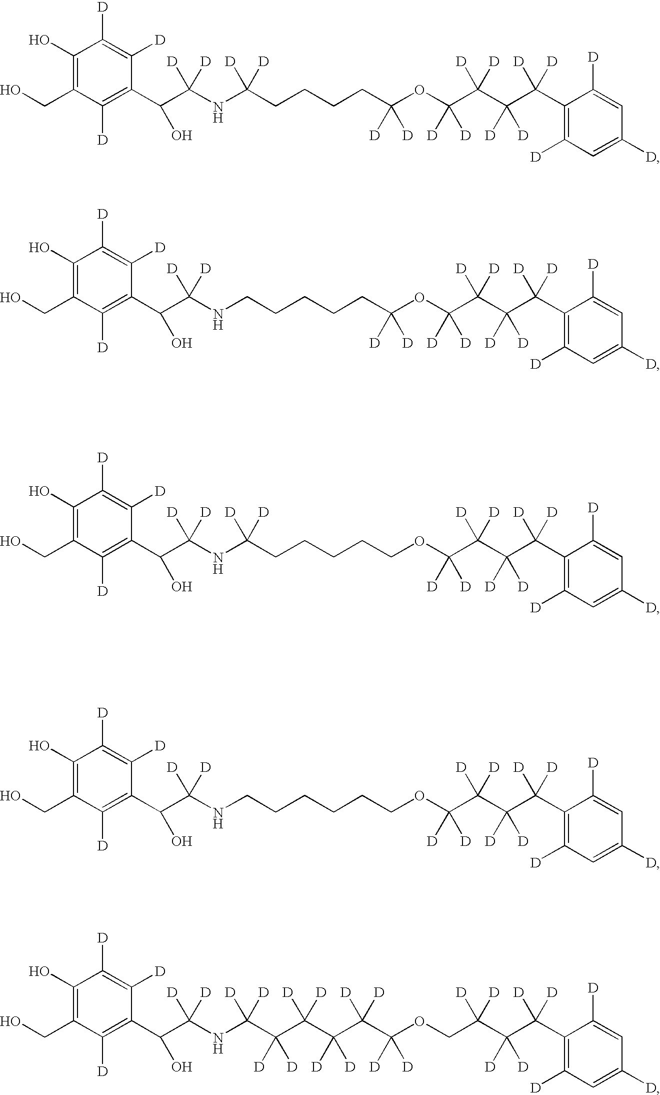 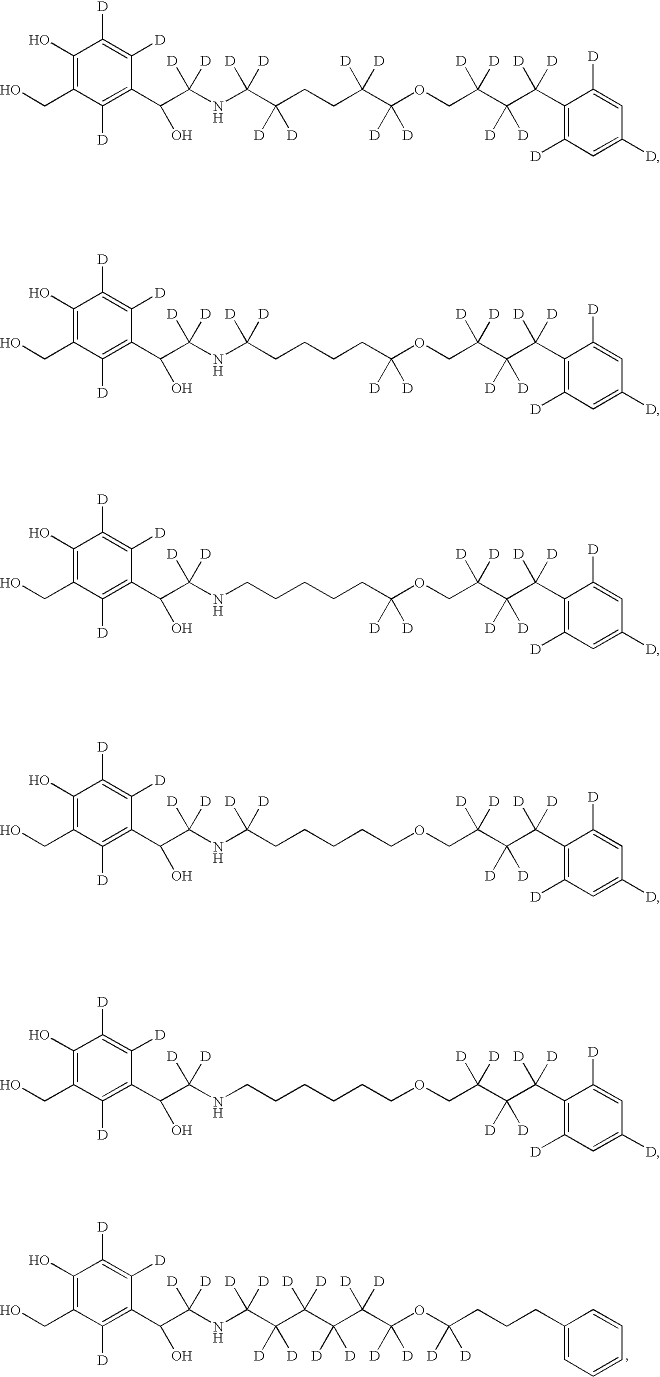 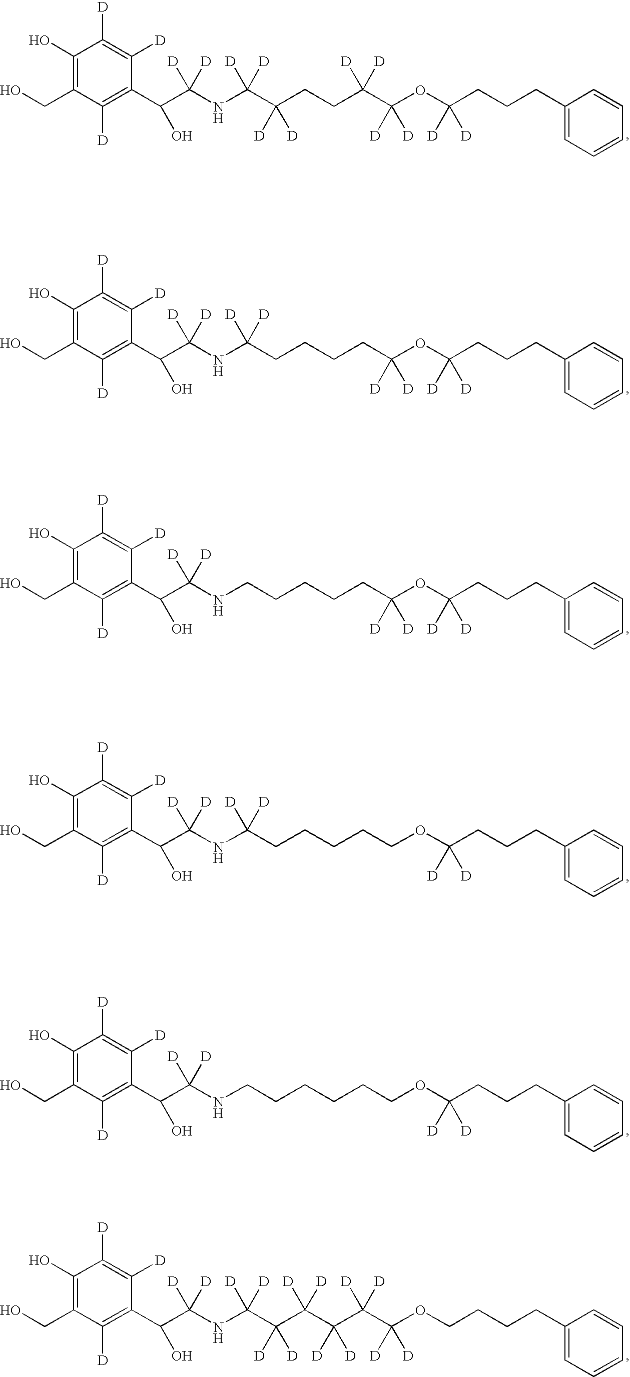 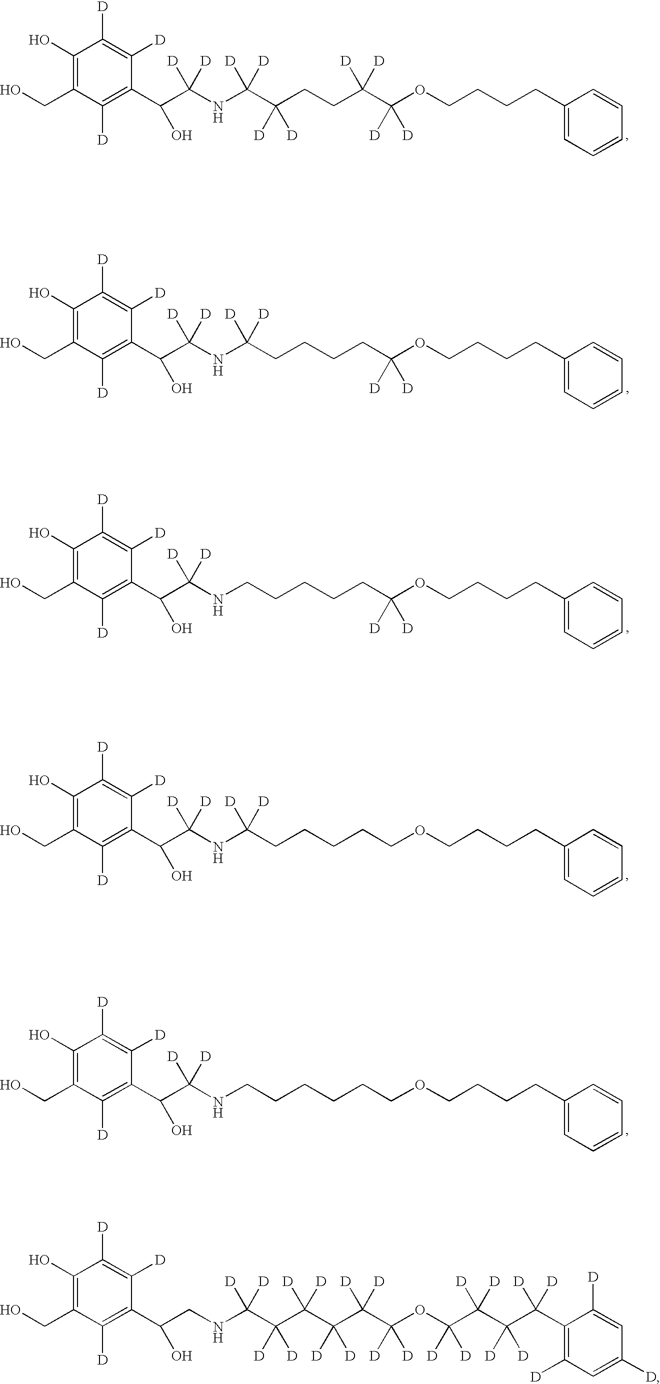 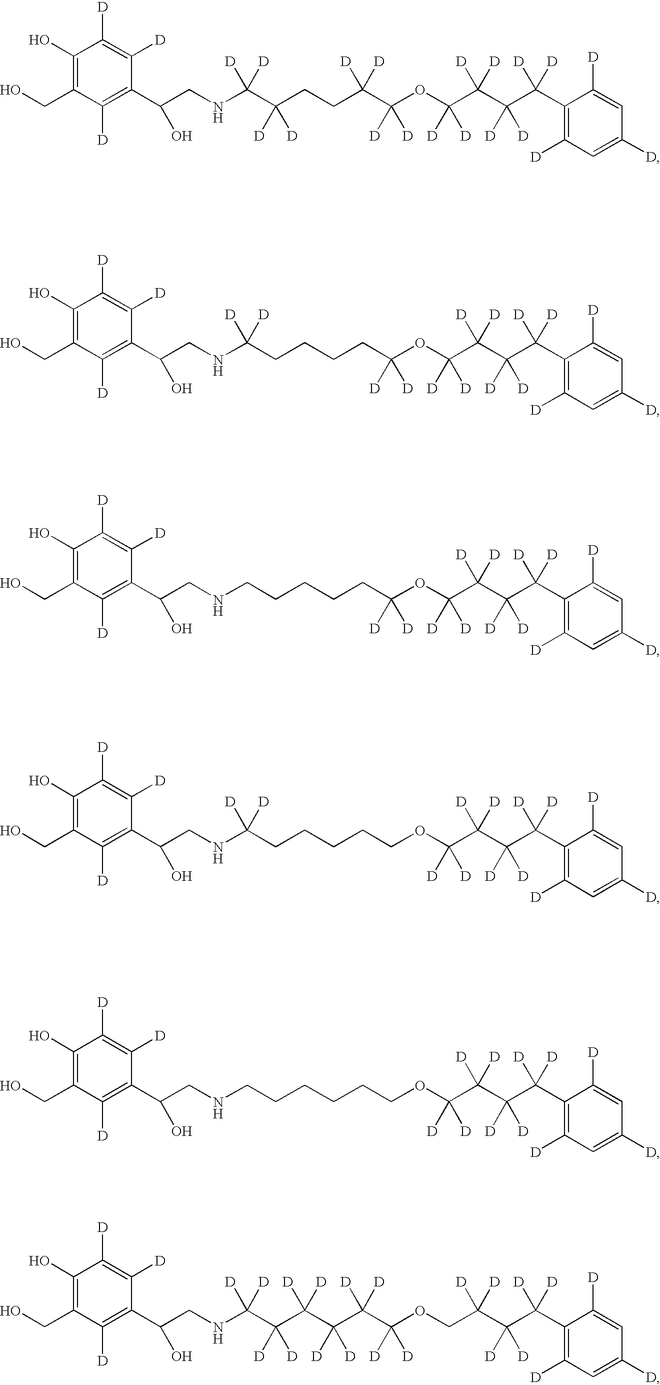 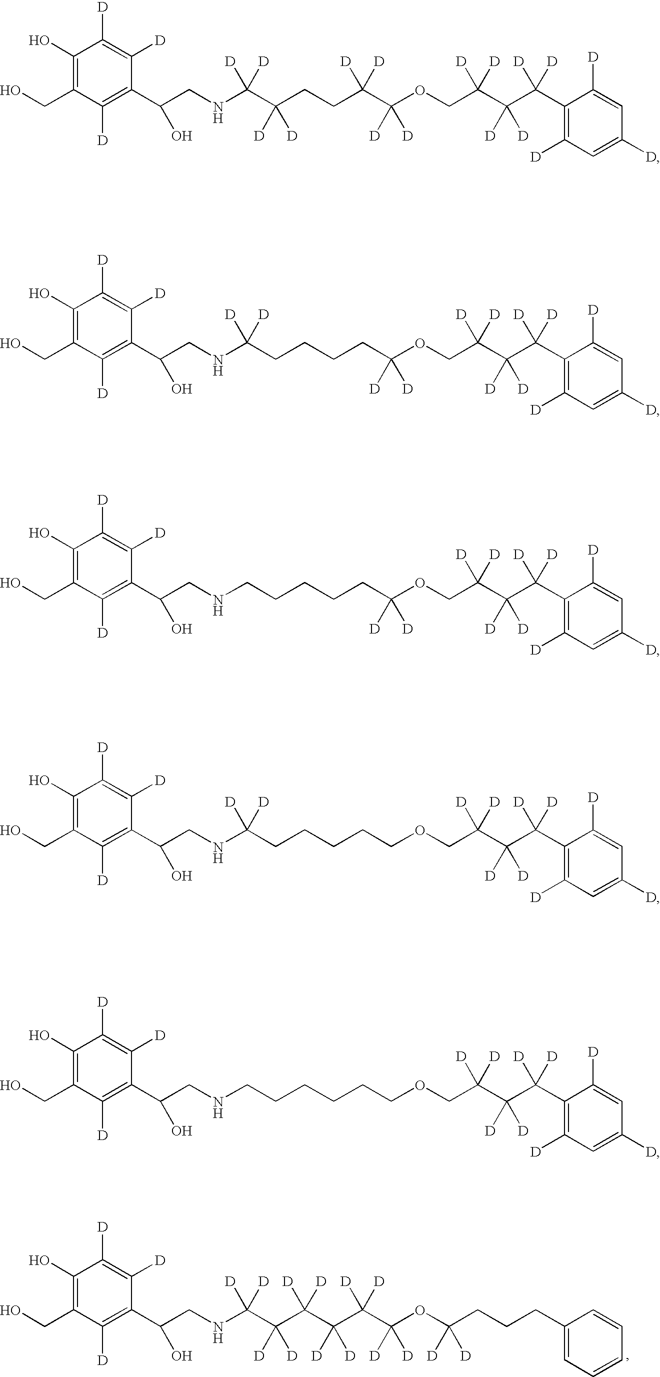 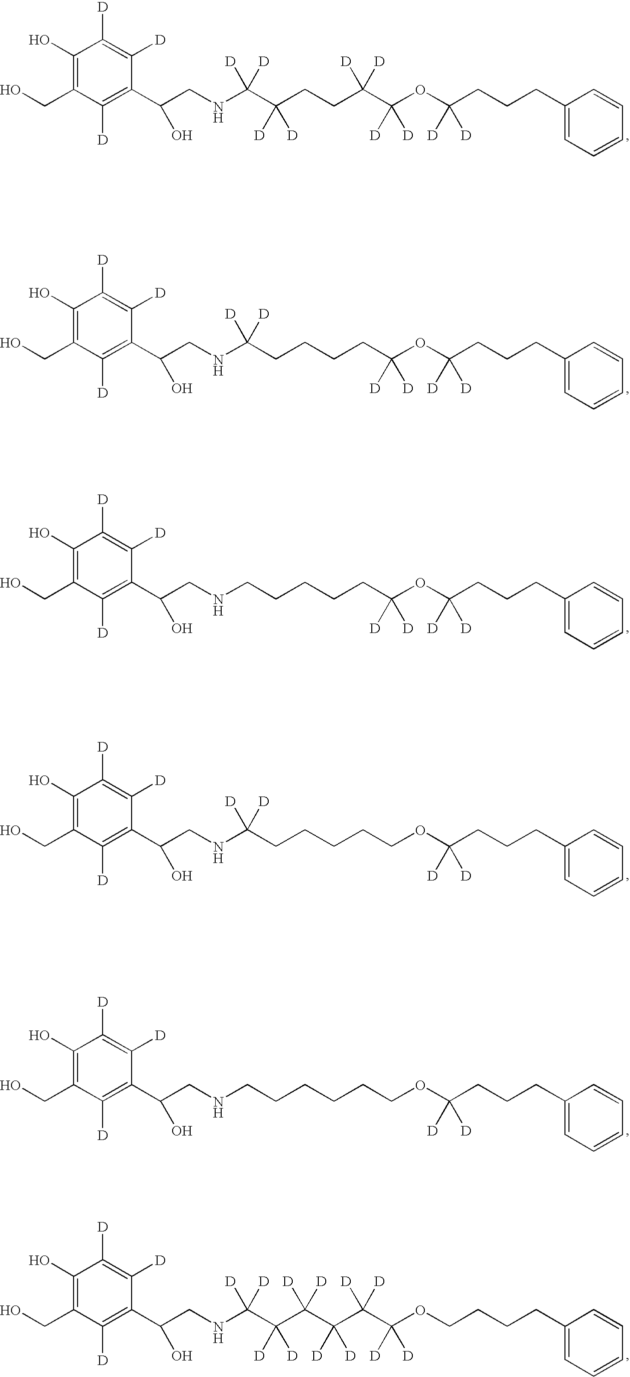 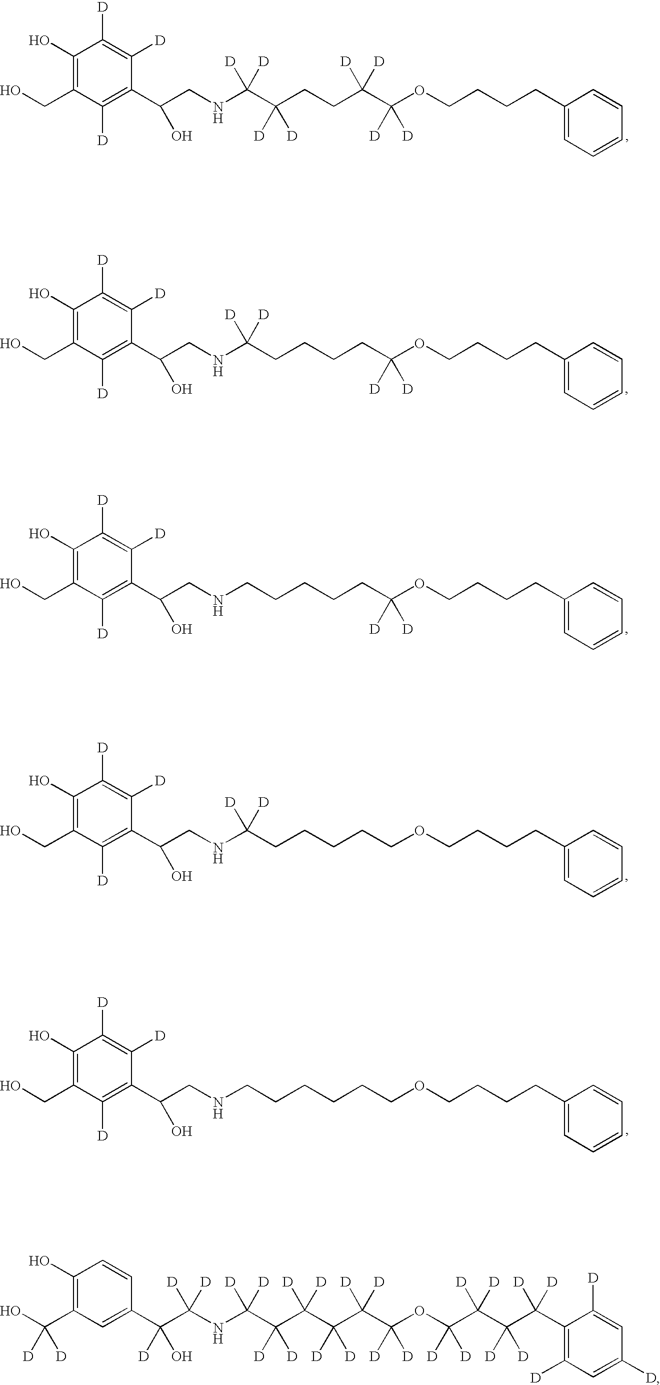 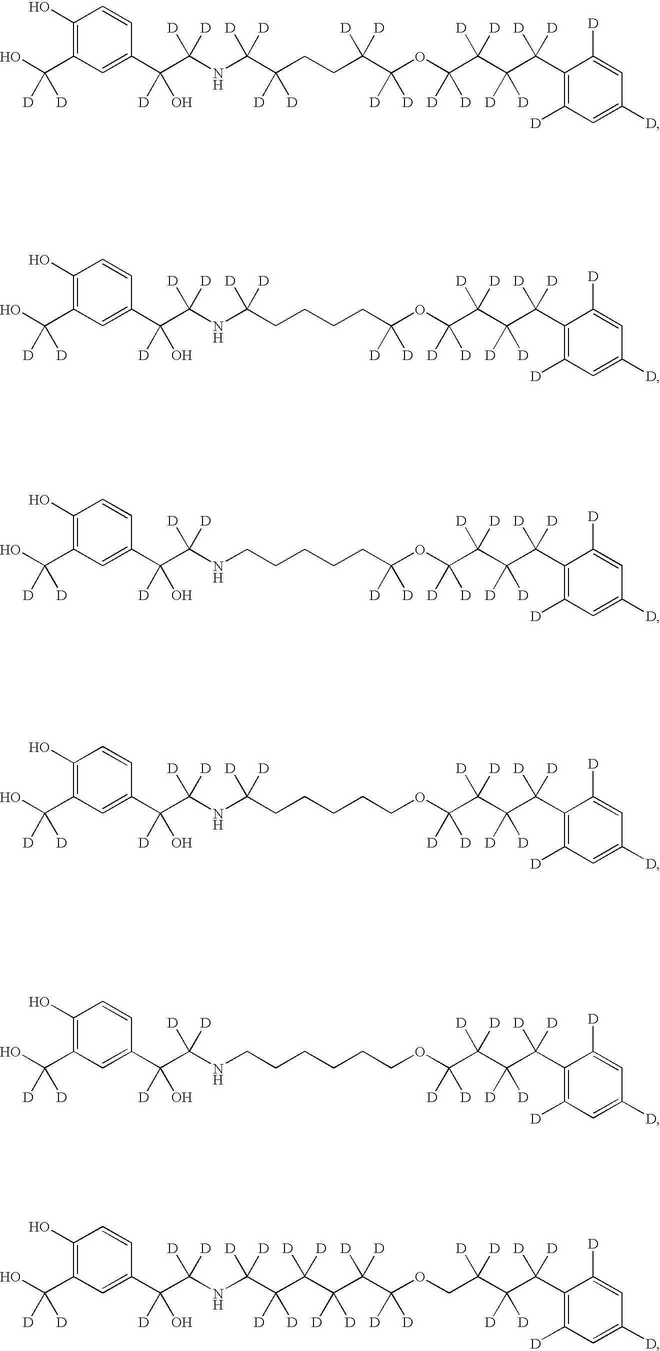 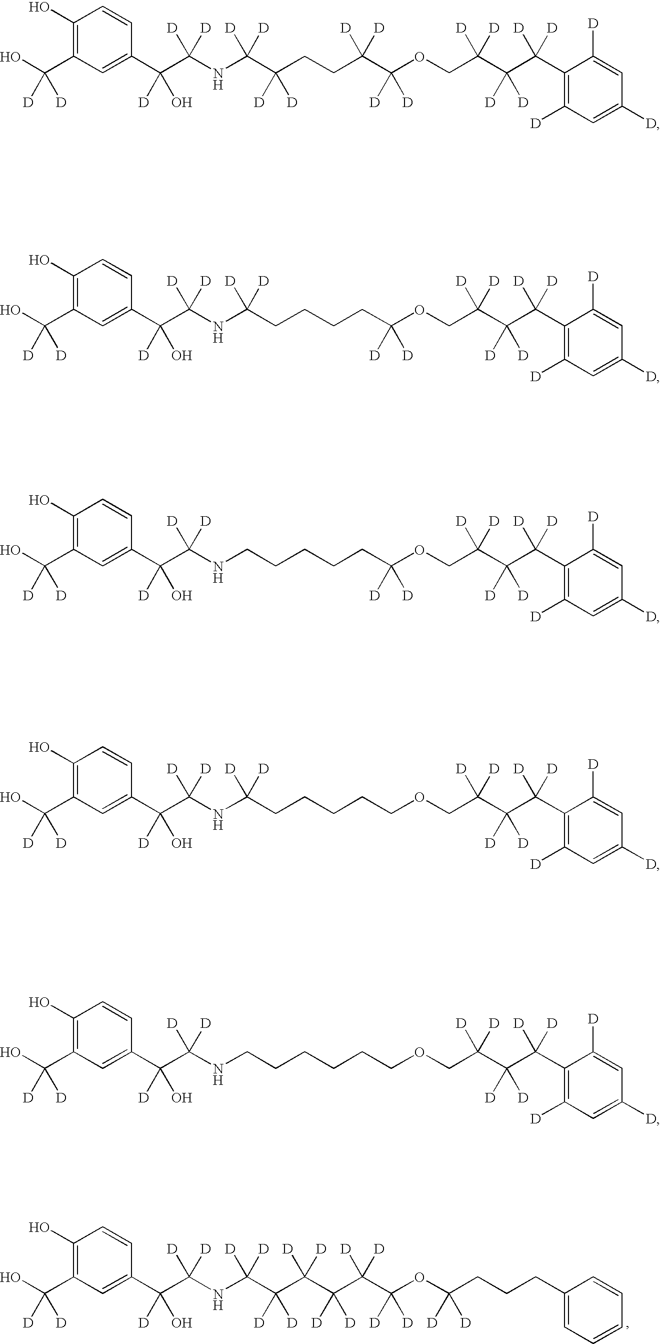 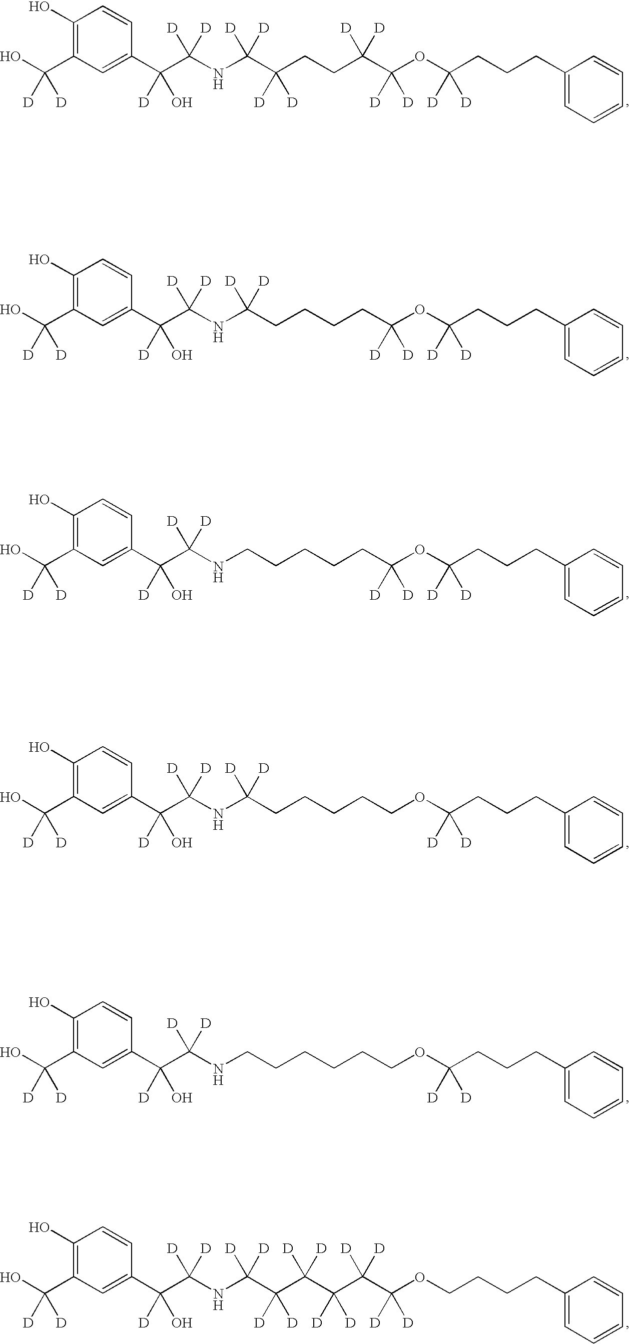 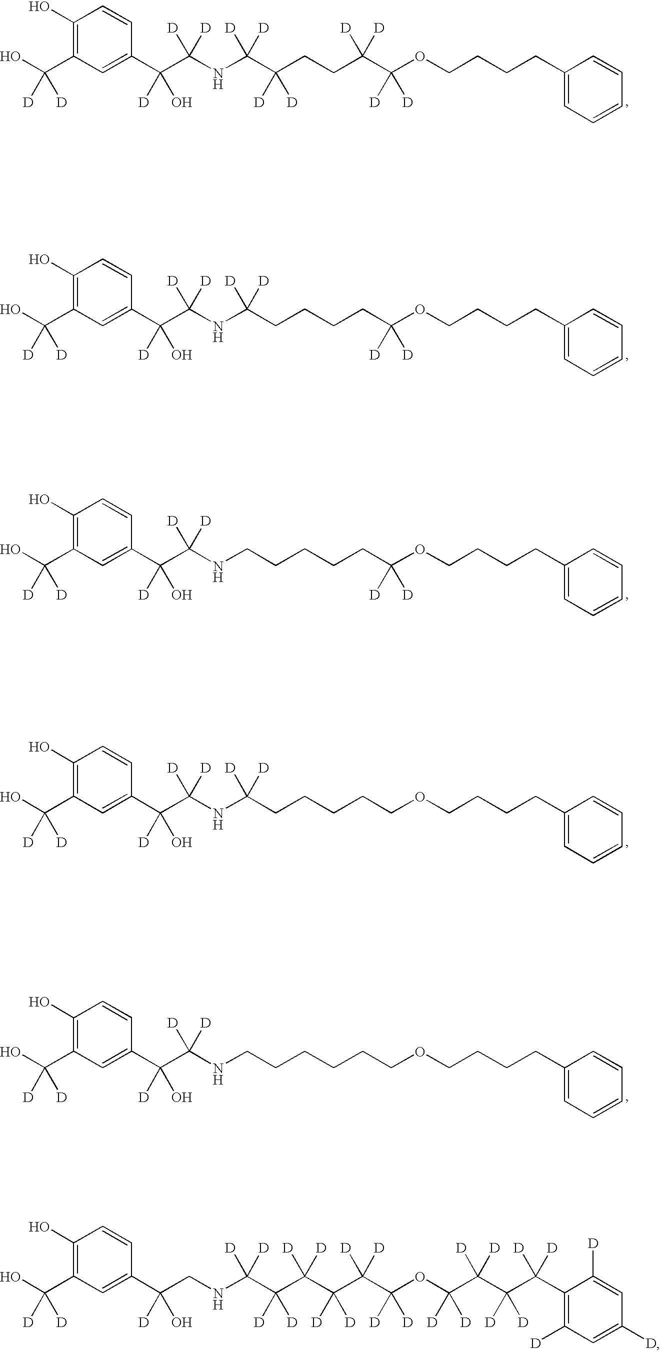 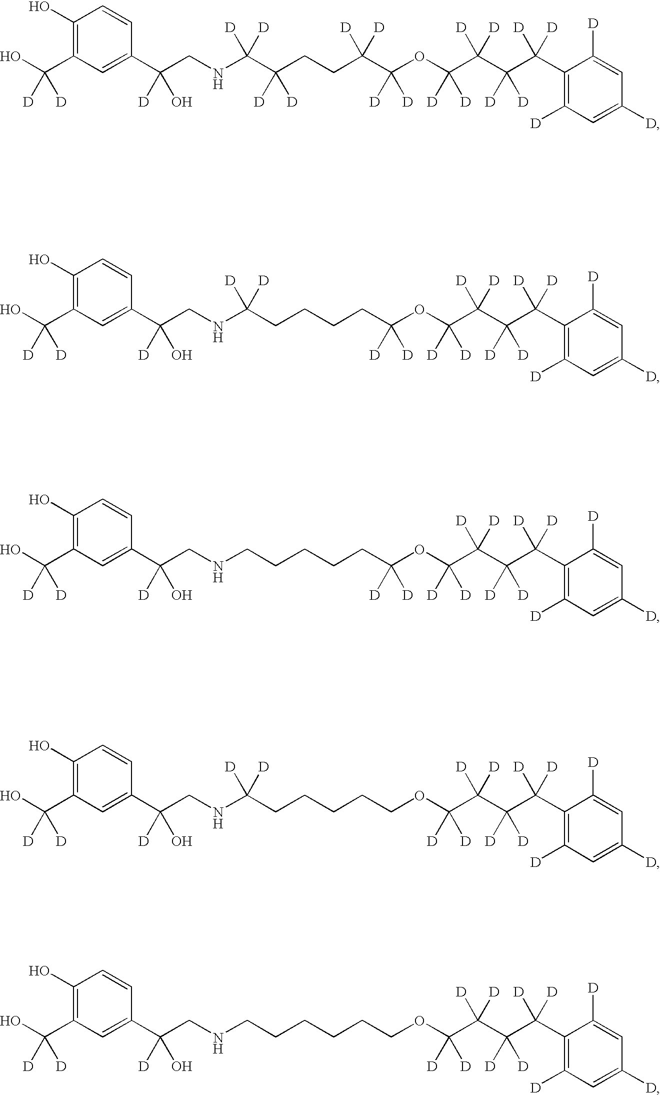 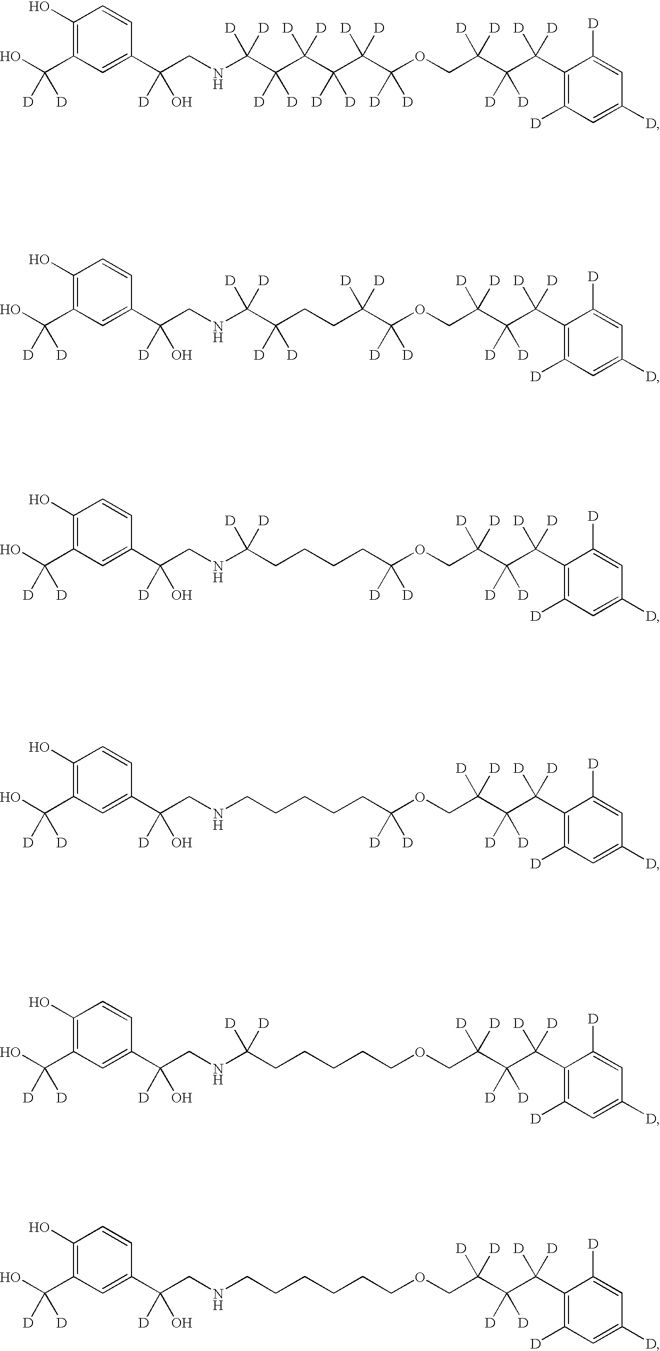 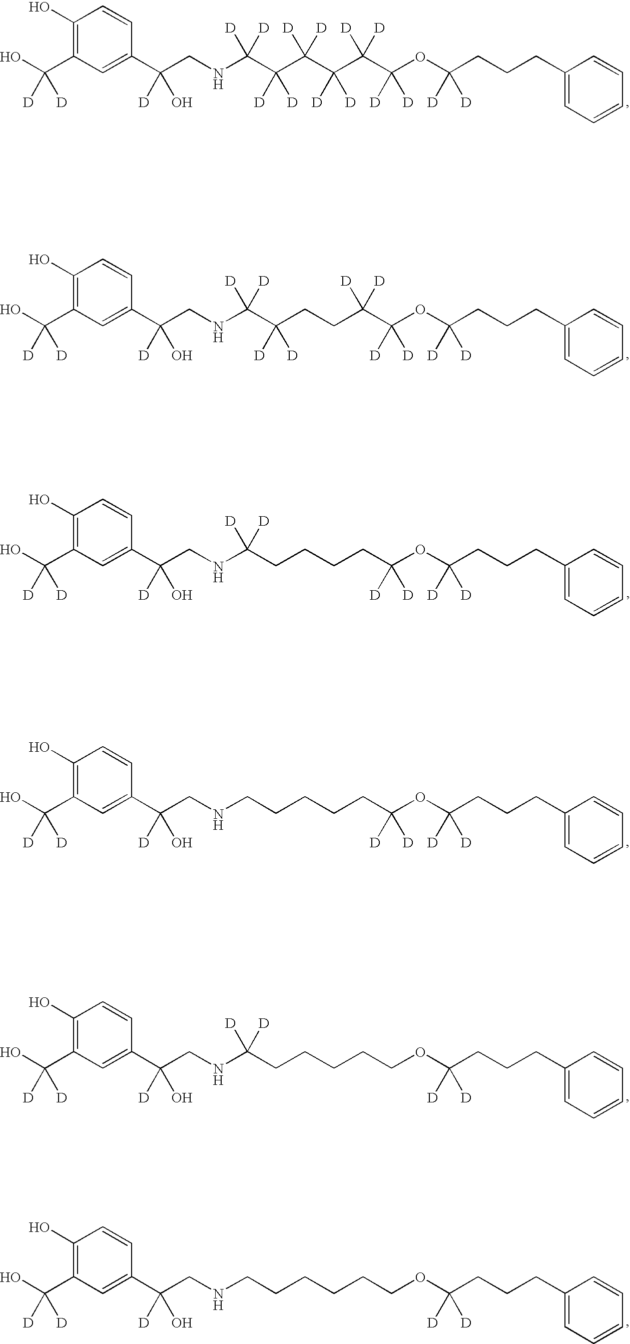 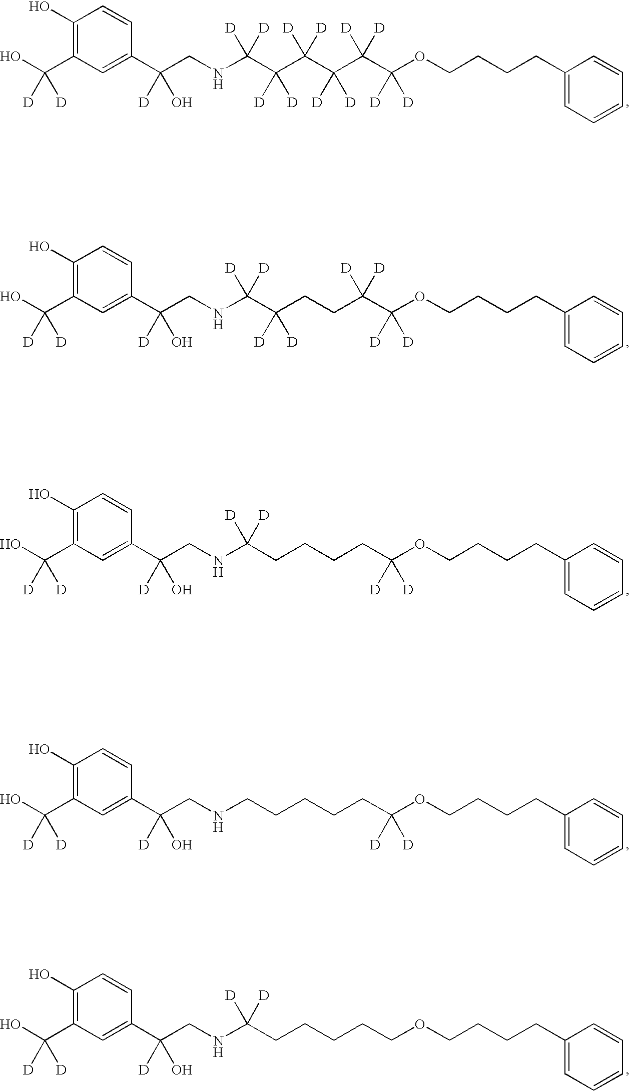 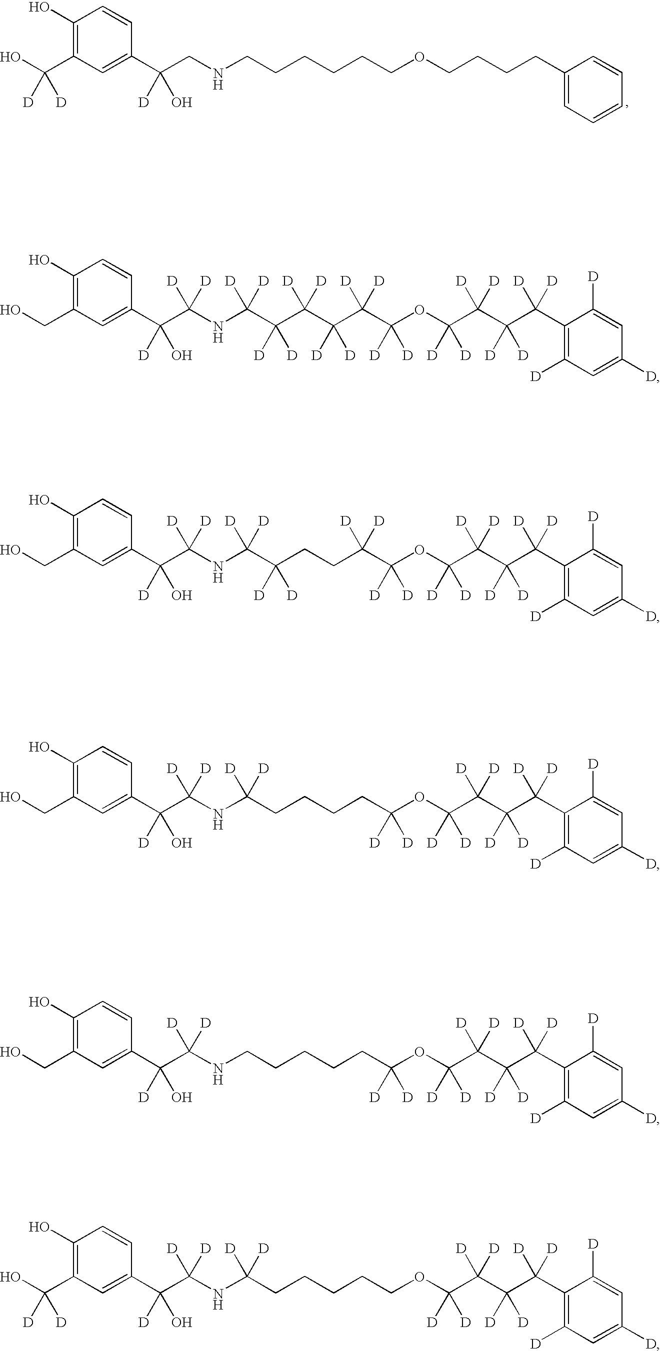 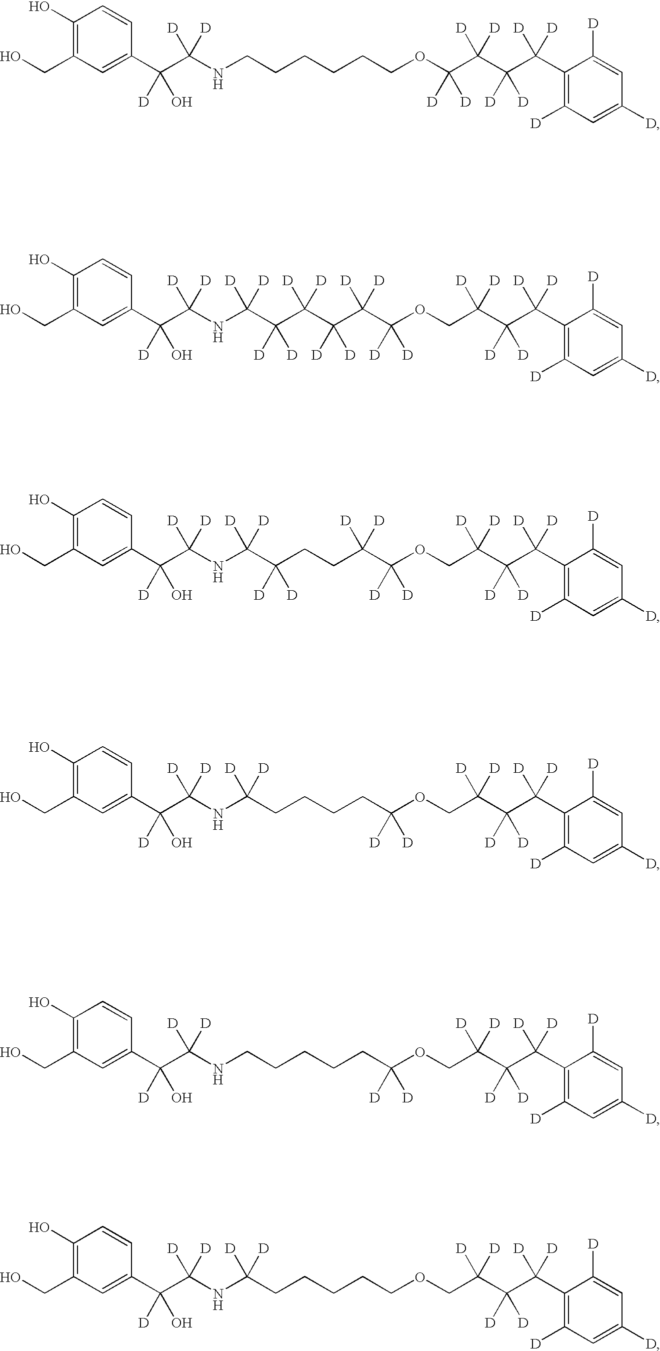 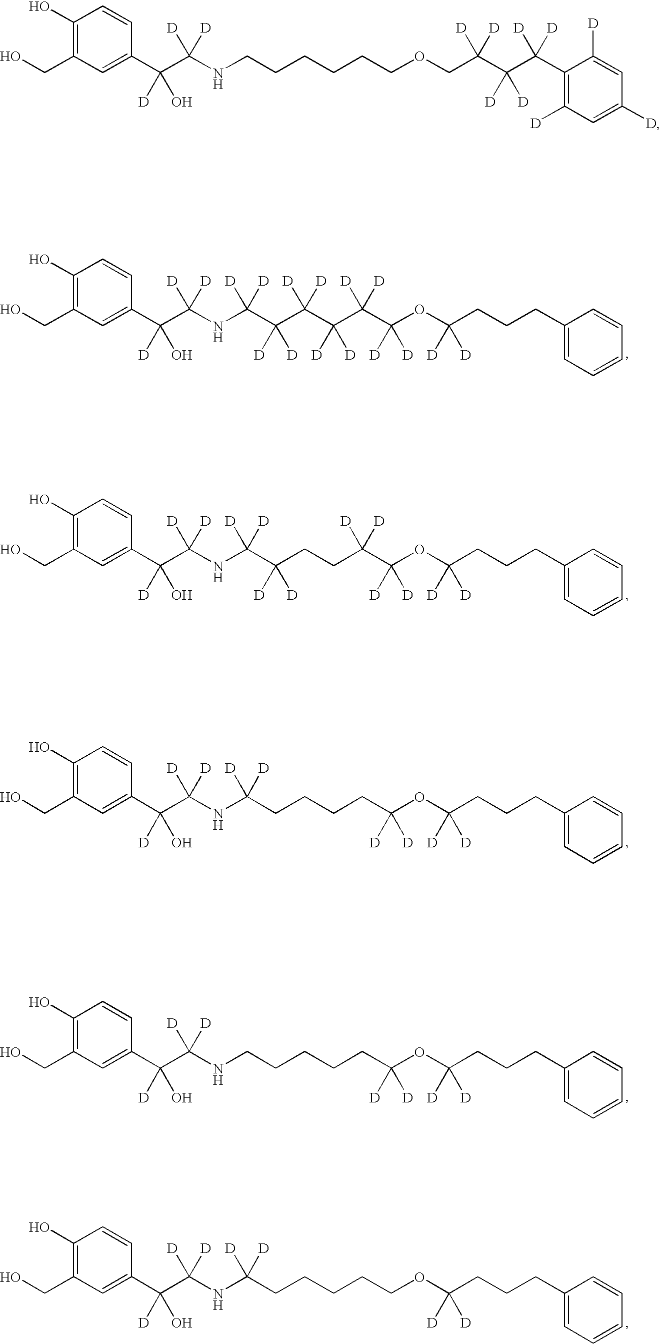 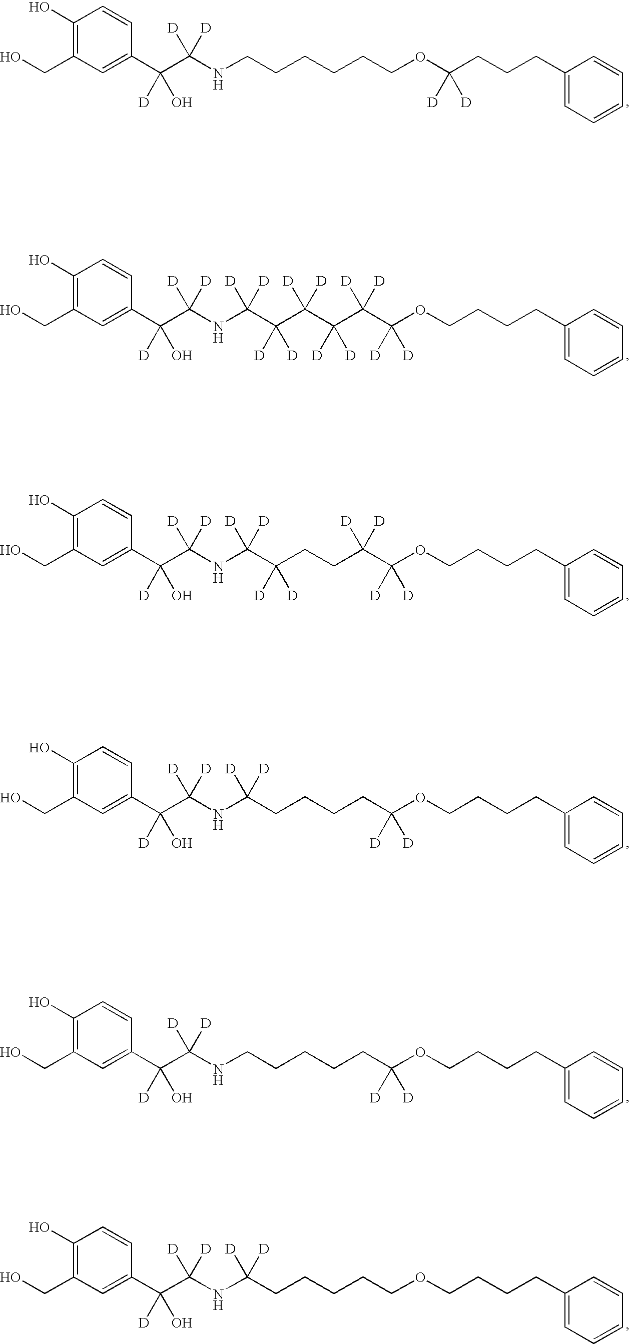 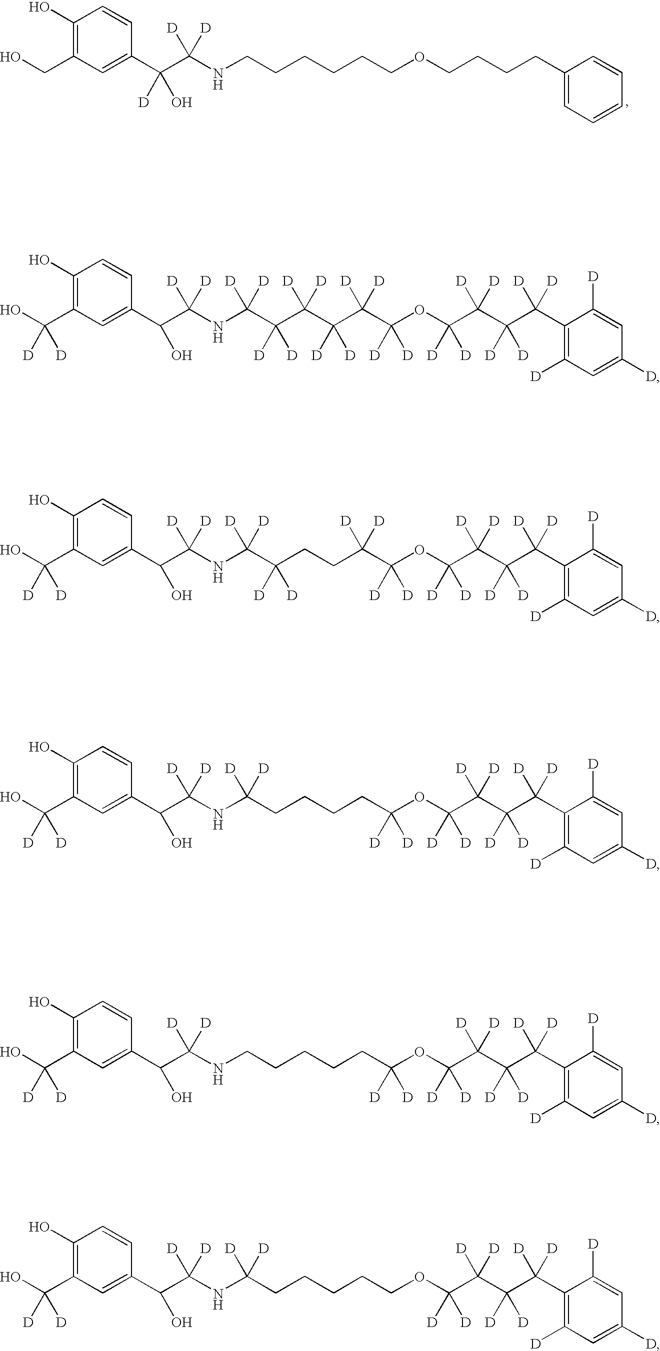 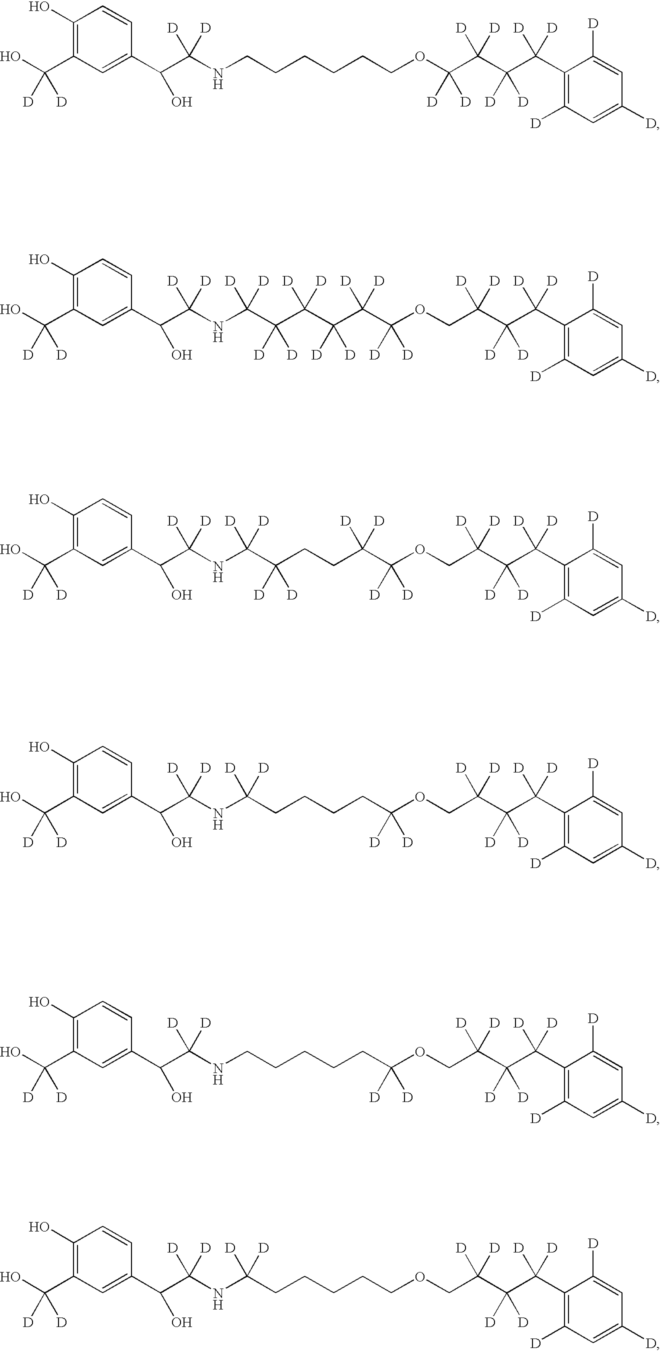 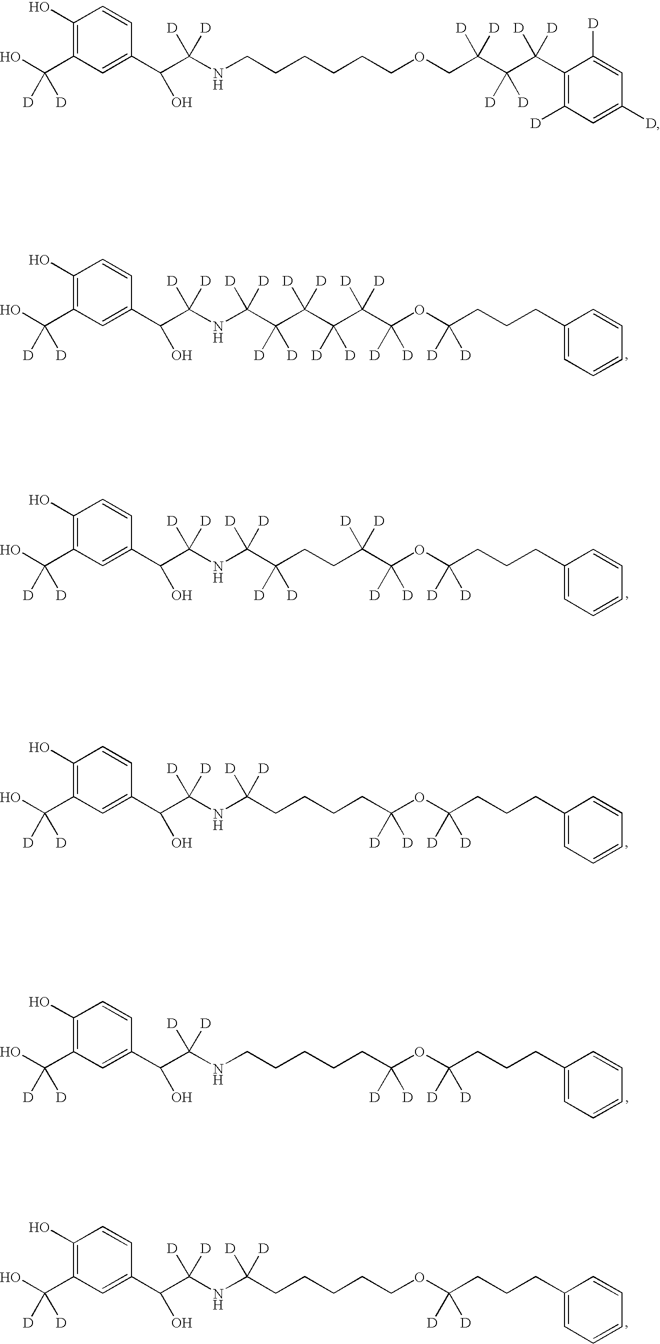 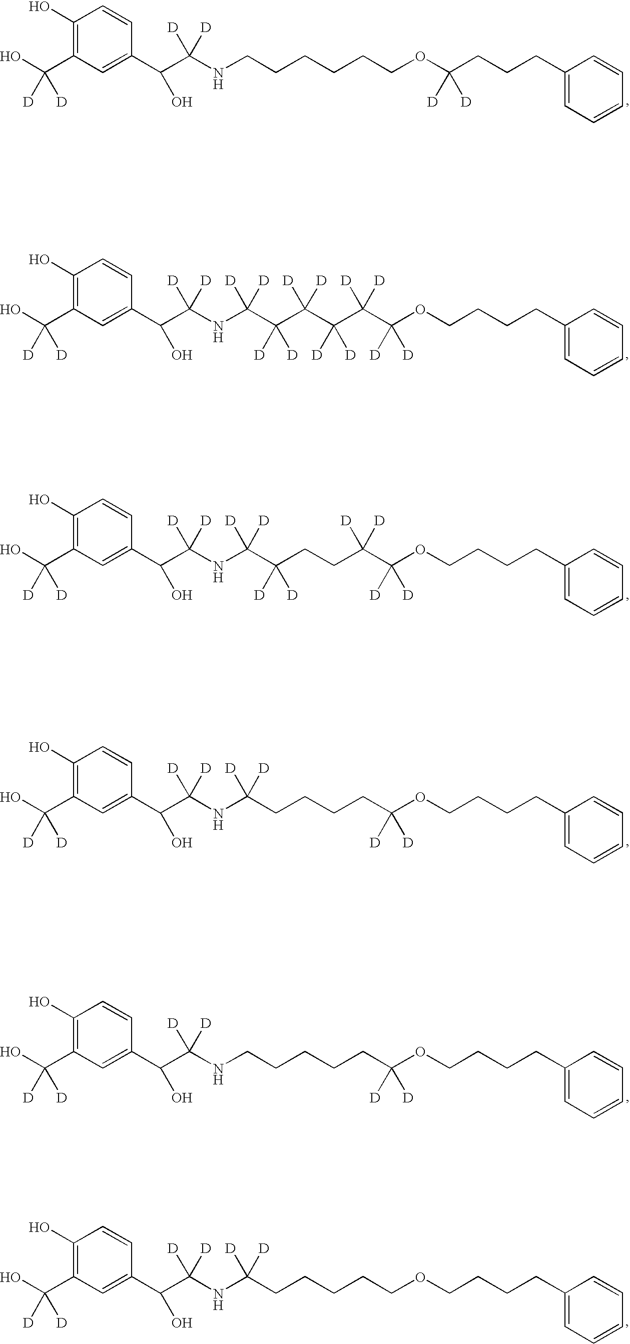 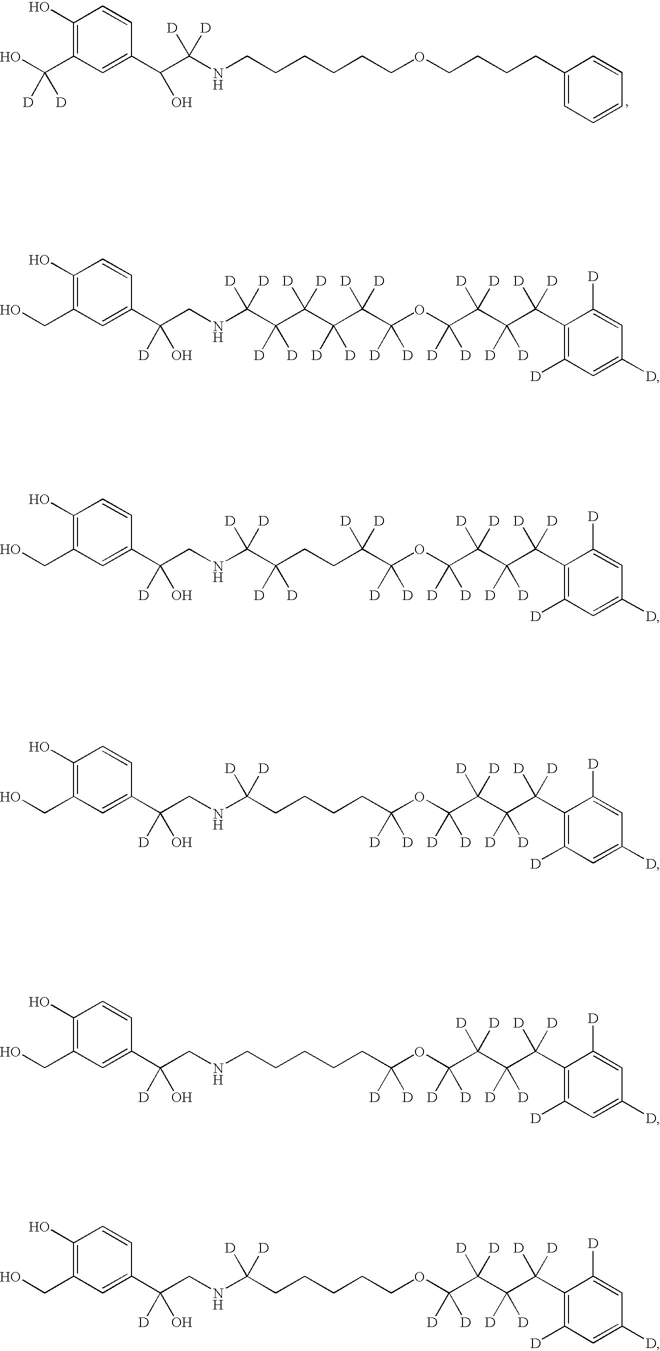 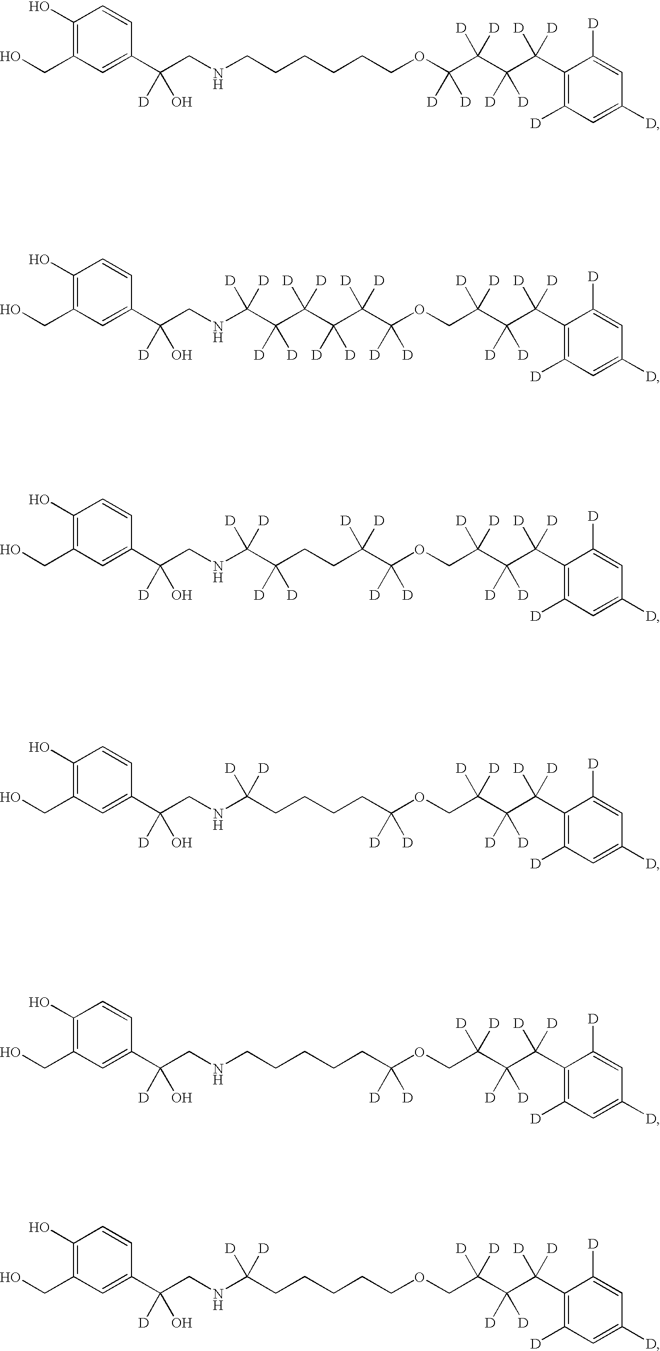 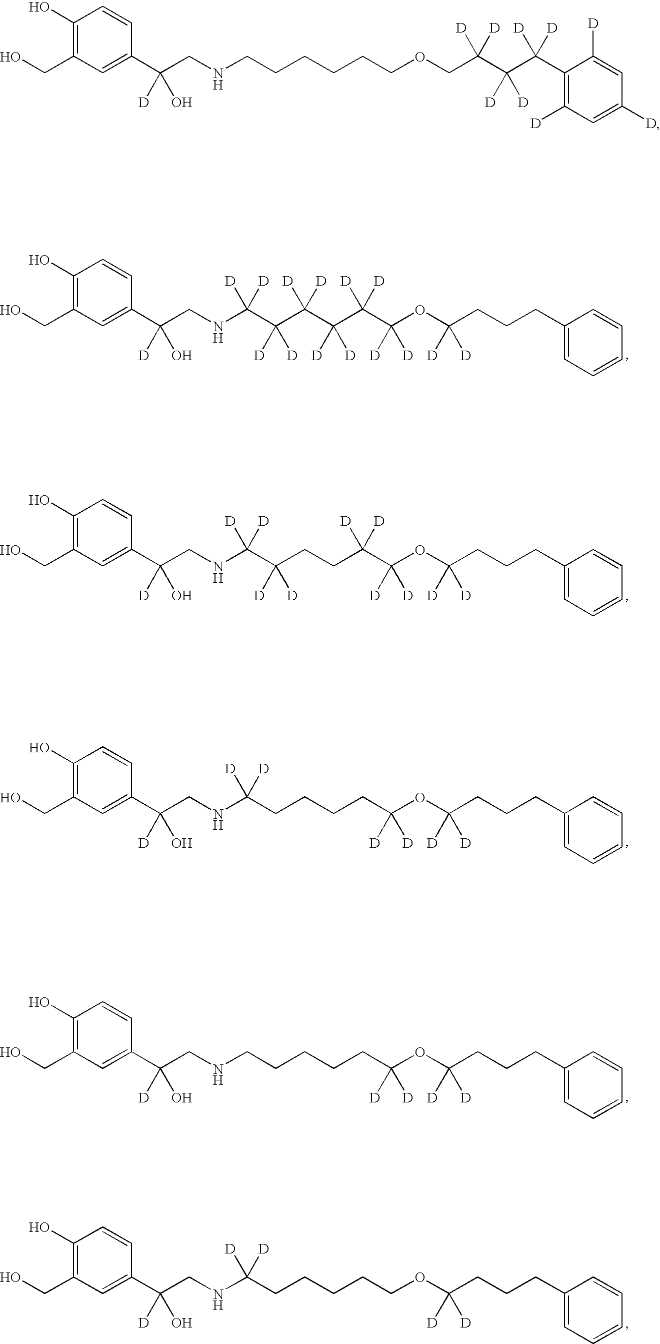 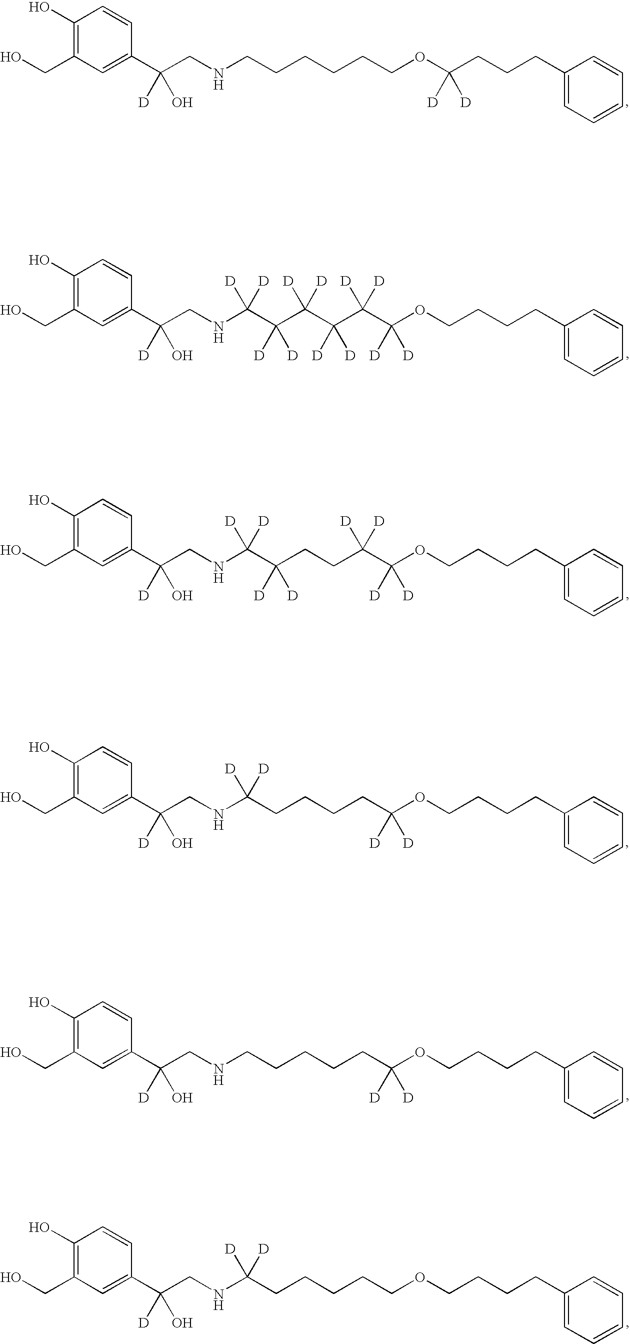 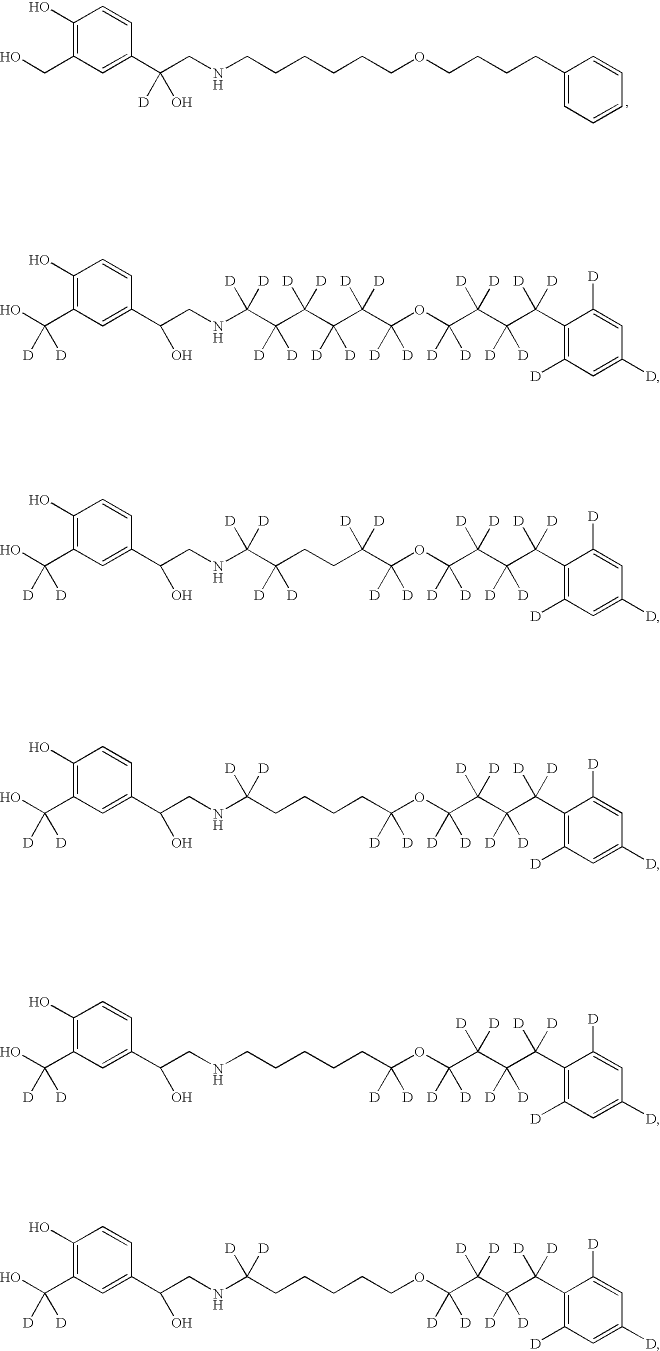 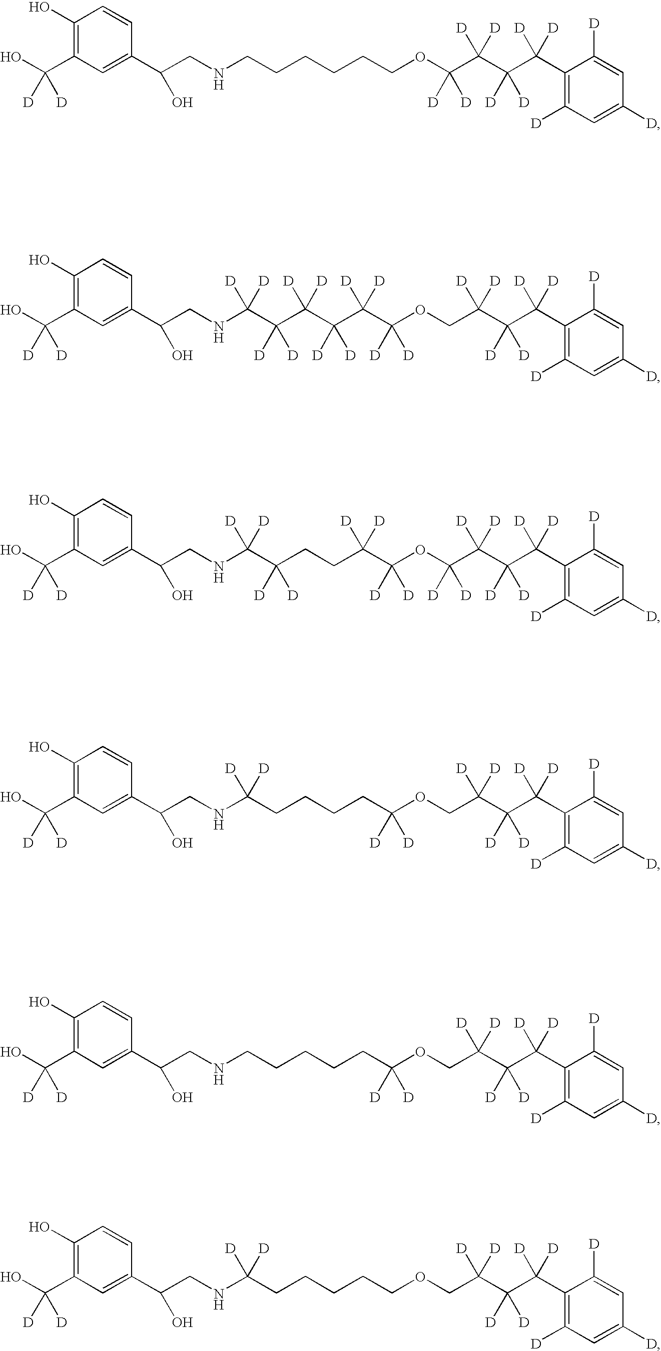 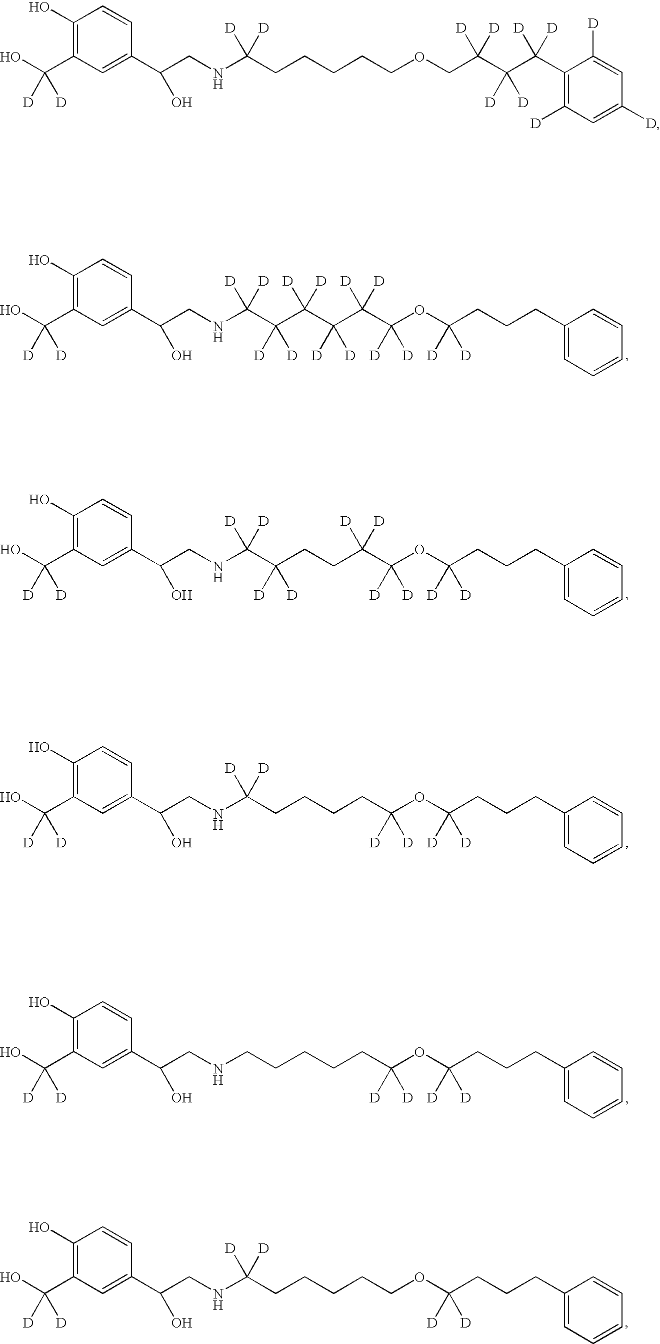 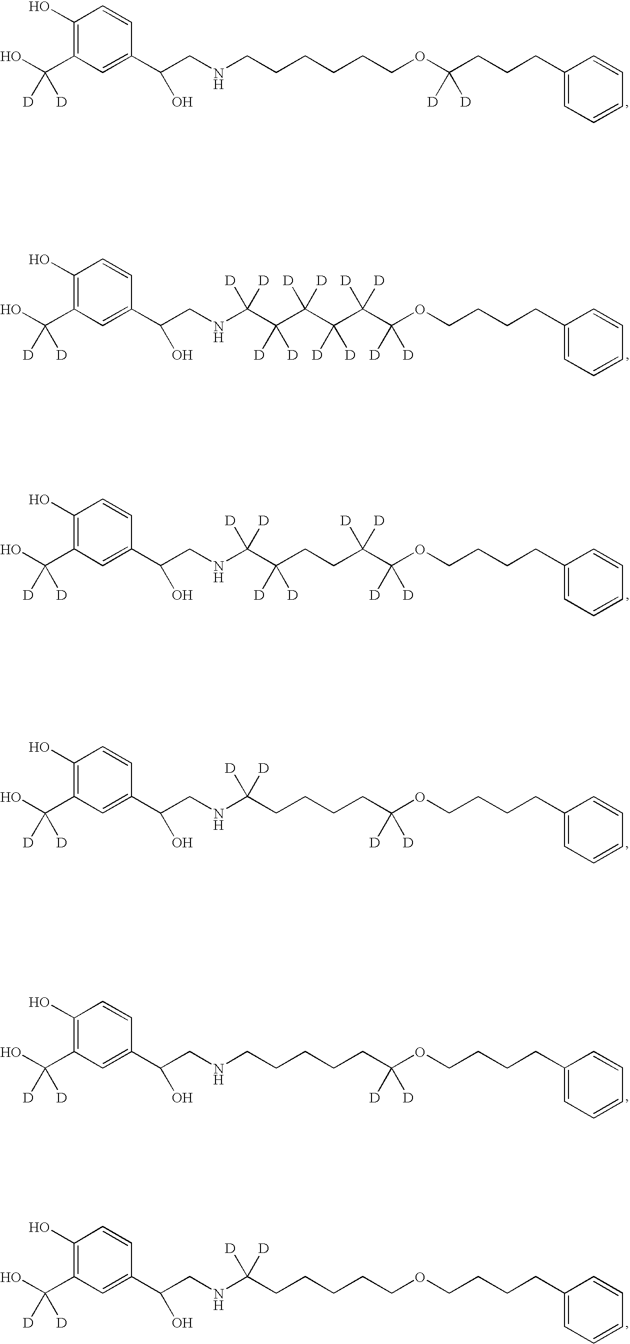 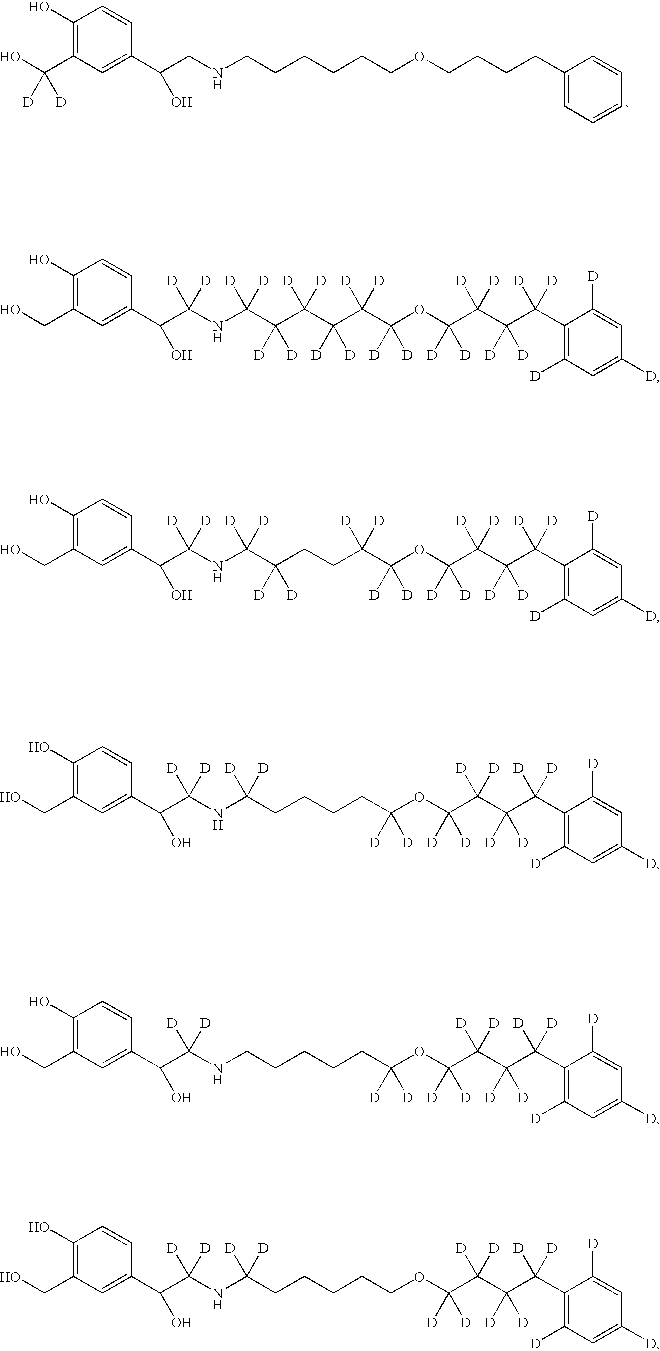 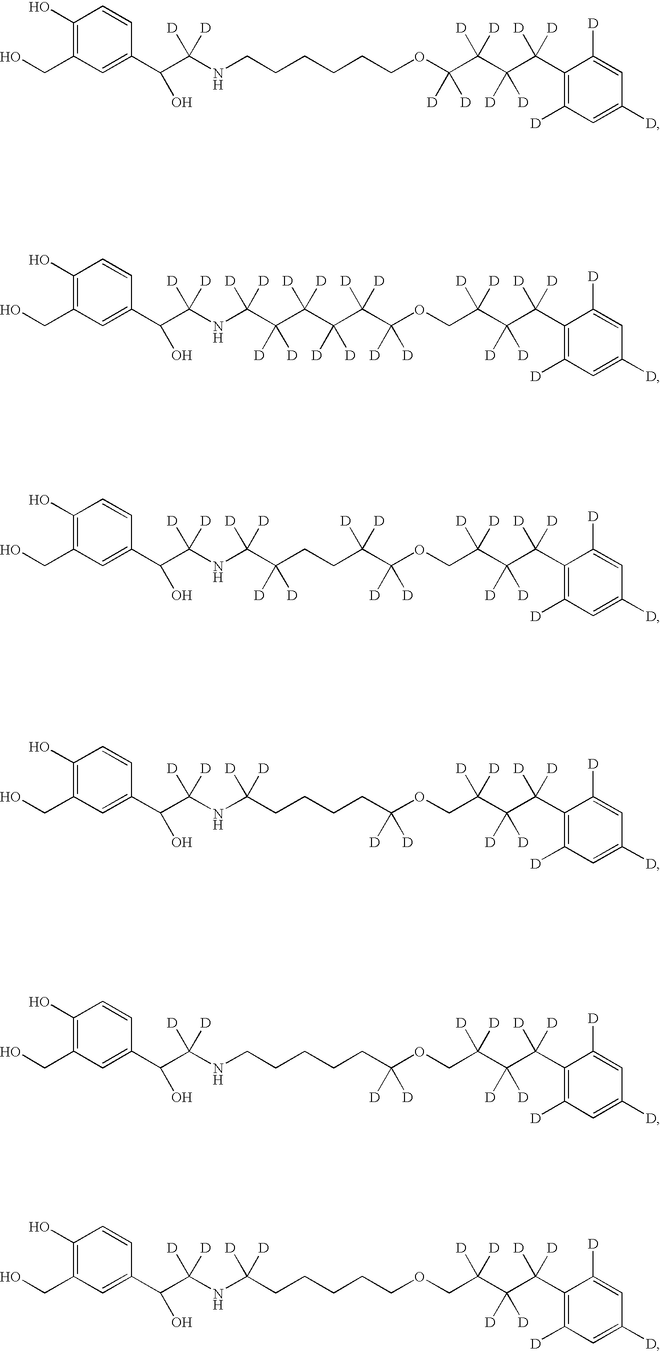 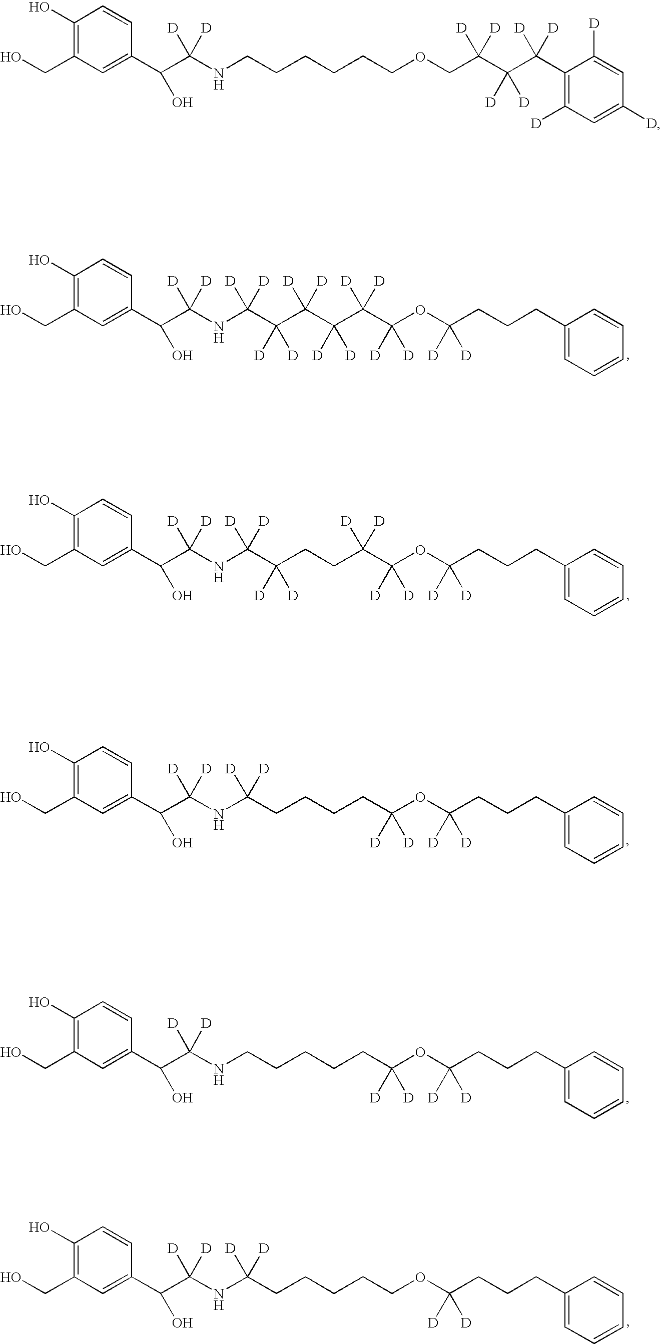 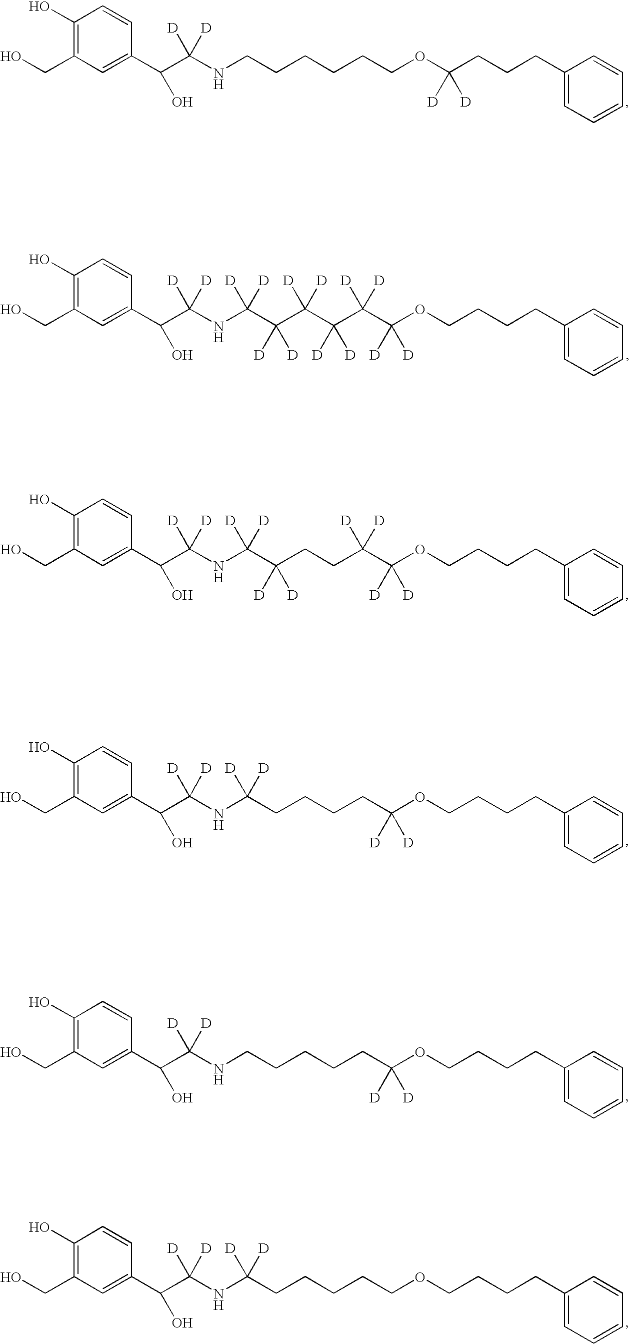 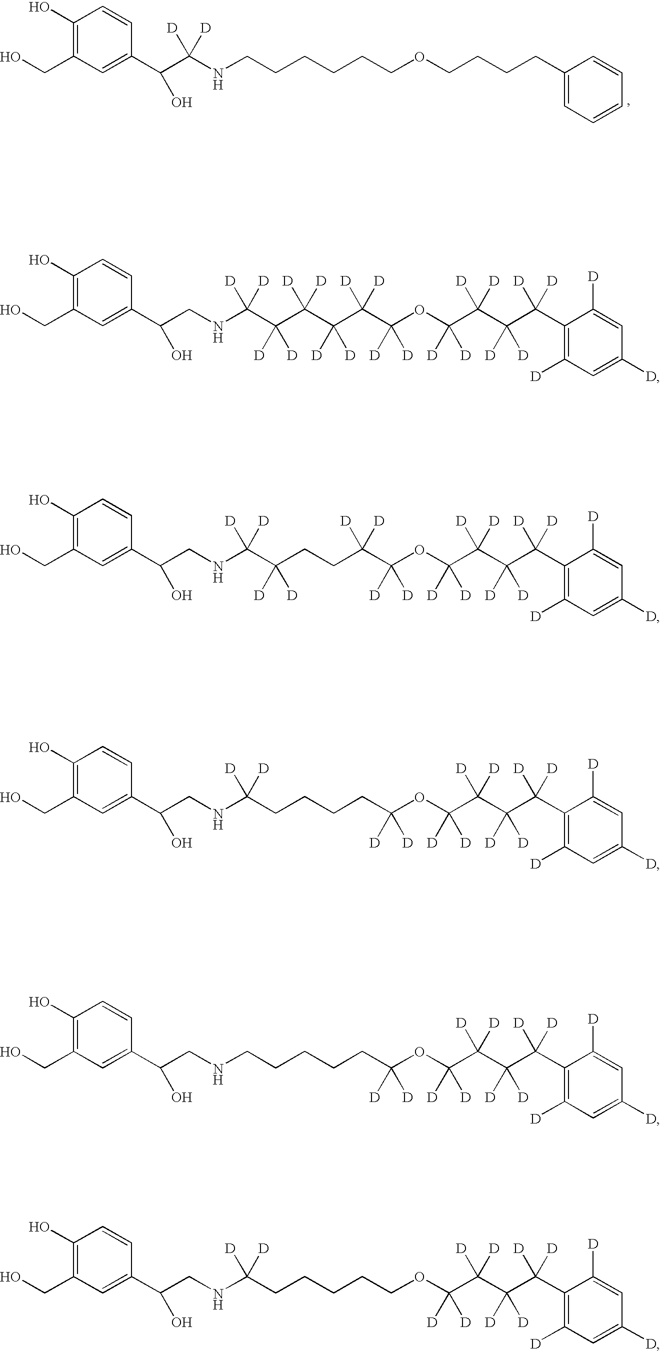 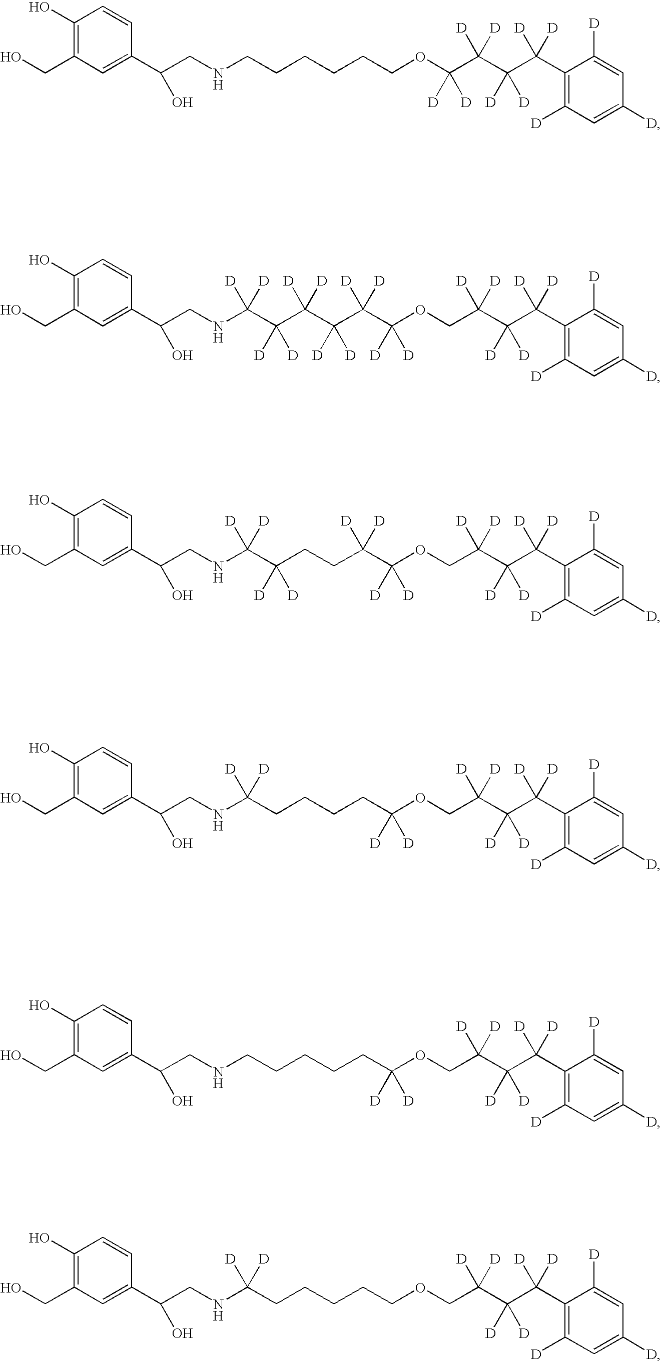 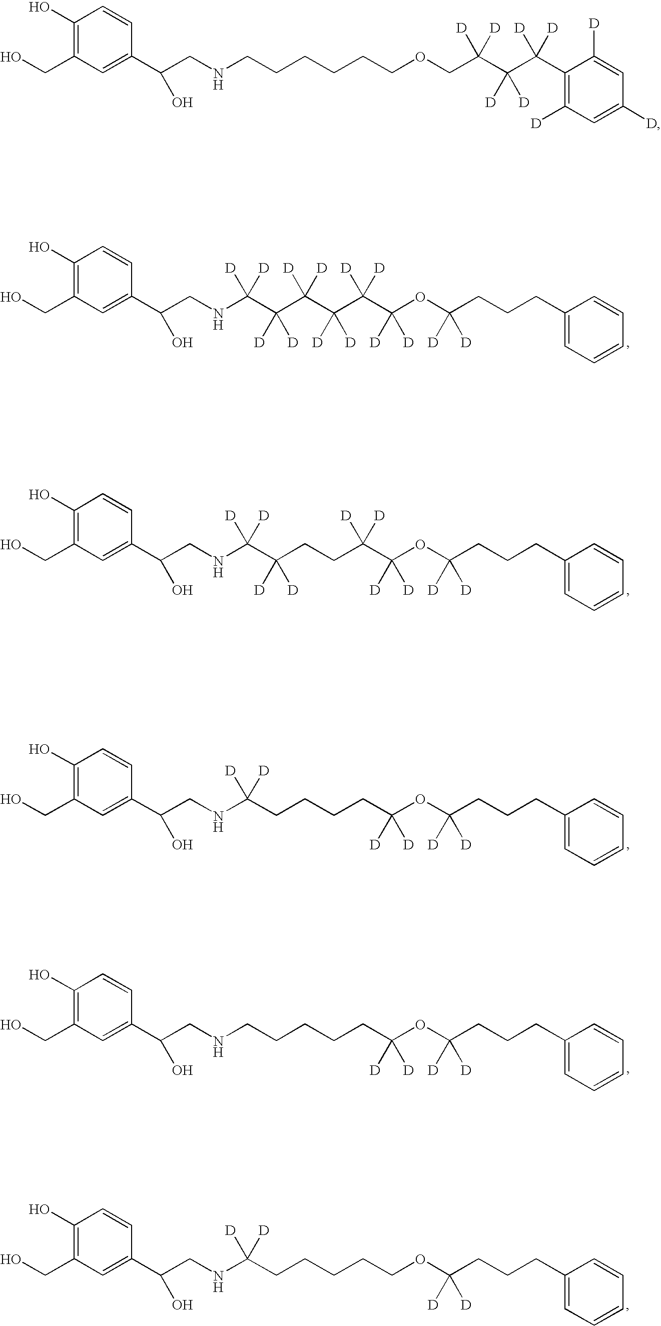 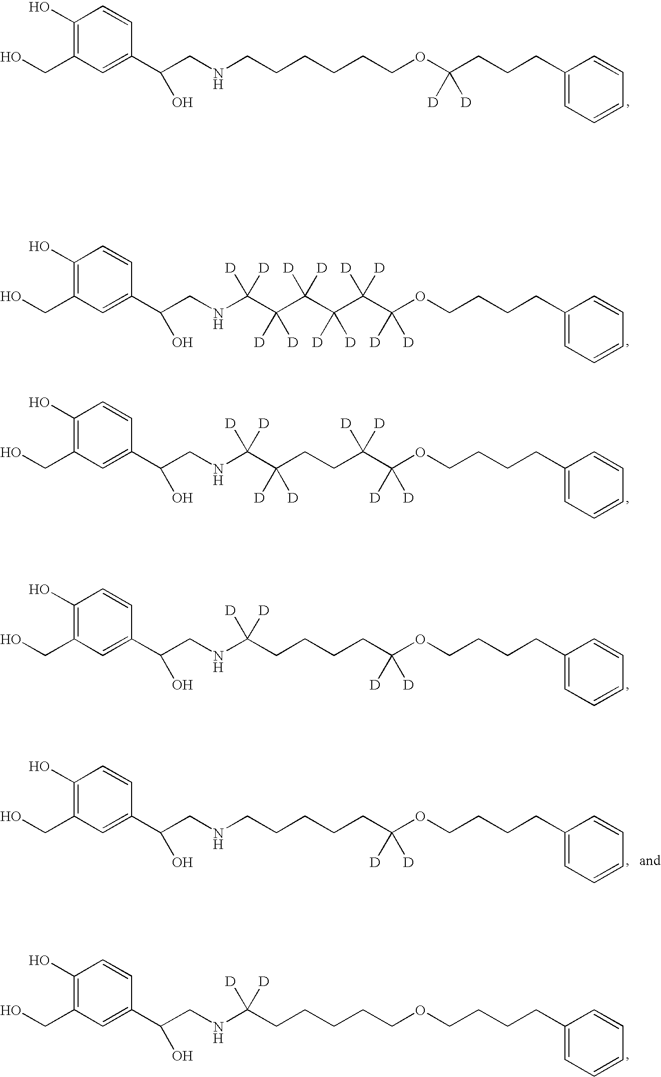
or a pharmaceutically acceptable salt thereof.

7. The compound as recited in claim 6 wherein each position represented as D has deuterium enrichment of no less than about 10%.

8. The compound as recited in claim 6 wherein each position represented as D has deuterium enrichment of no less than about 50%.

9. The compound as recited in claim 6 wherein each position represented as D has deuterium enrichment of no less than about 90%.

10. The compound as recited in claim 6 wherein each position represented as D has deuterium enrichment of no less than about 98%.

11. A pharmaceutical composition comprising a compound as recited in claim 1 together with a pharmaceutically acceptable carrier.

12. The compound as recited in claim 1 wherein said compound has a structural formula selected from the group consisting of:

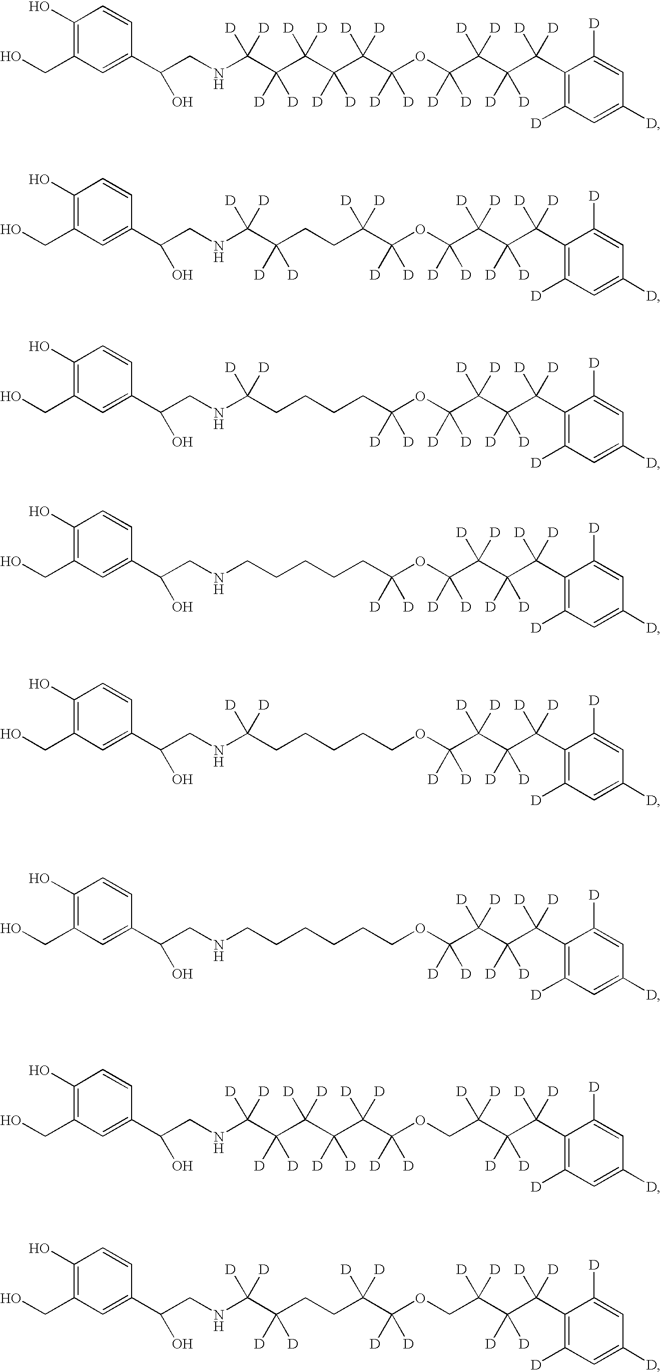

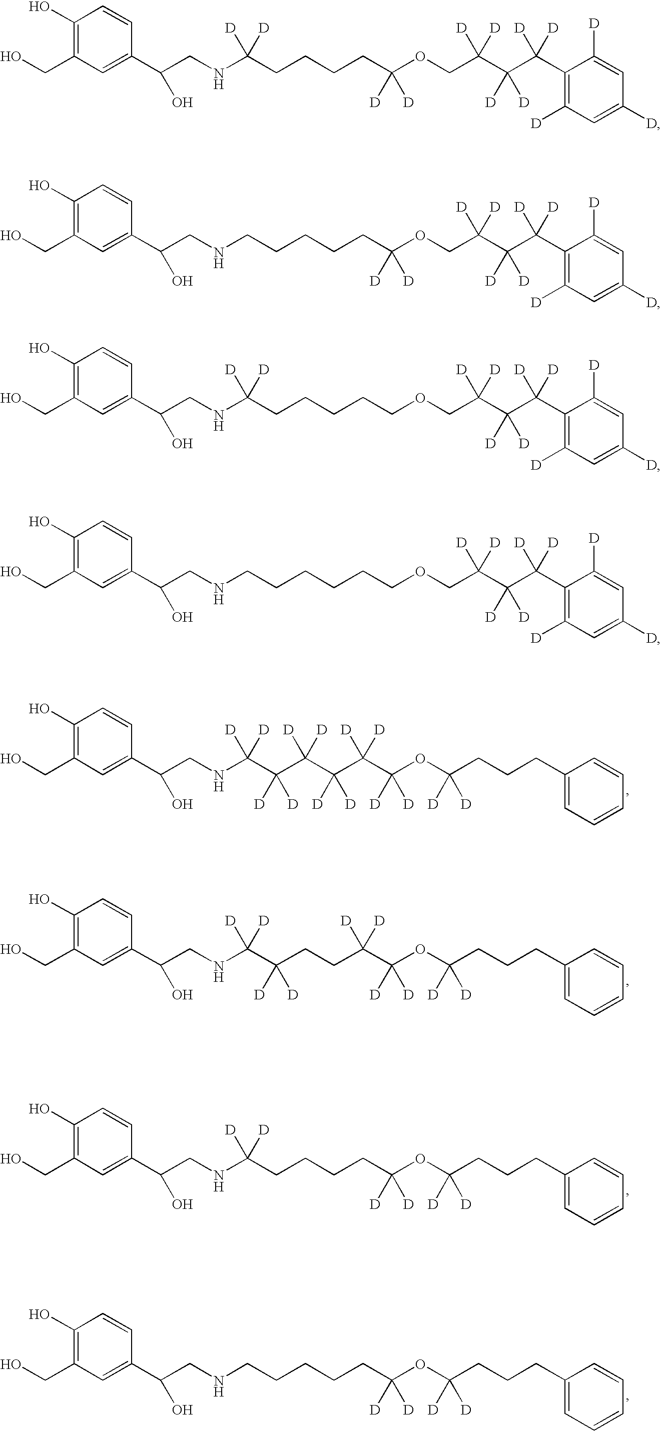

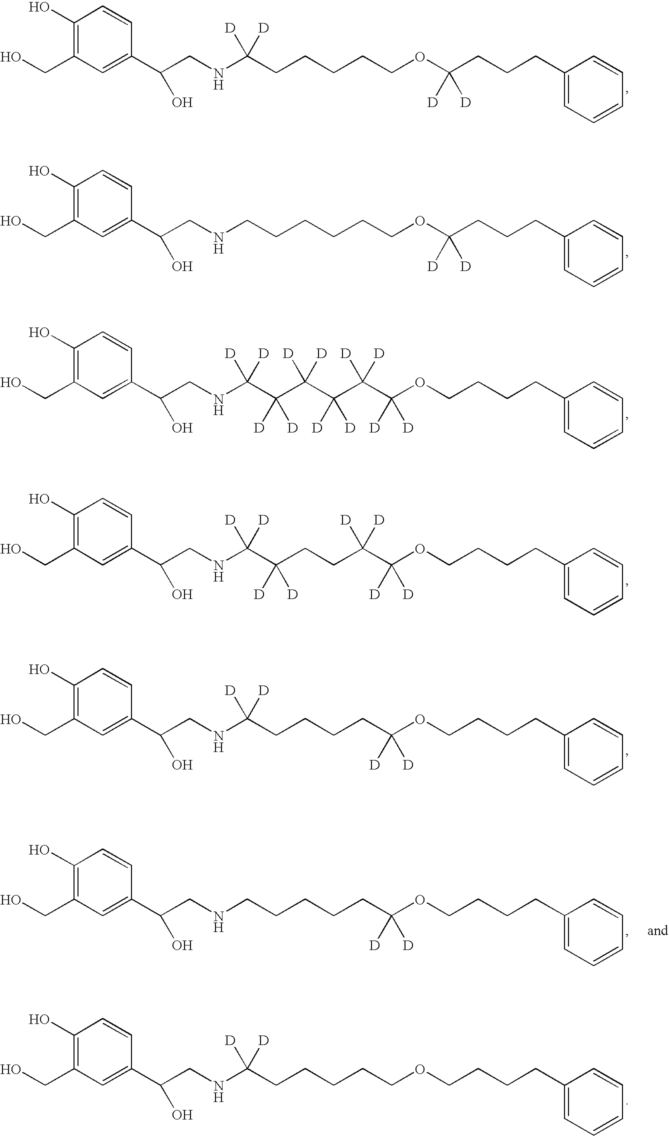

13. The compound as recited in claim 12 wherein each position represented as D has deuterium enrichment of no less than about 10%.

14. The compound as recited in claim 12 wherein each position represented as D has deuterium enrichment of no less than about 50%.

15. The compound as recited in claim 12 wherein each position represented as D has deuterium enrichment of no less than about 90%.

16. The compound as recited in claim 12 wherein each position represented as D has deuterium enrichment of no less than about 98%.

17. A method of treatment of an adrenergic receptor mediated disorder comprising the administration of a therapeutically effective amount of a compound having structural Formula I: 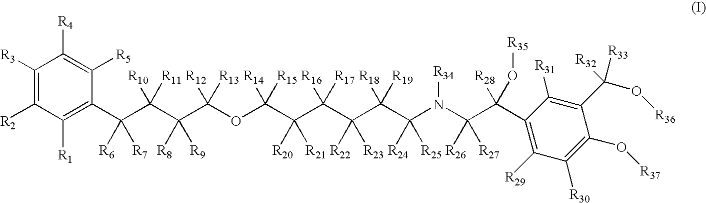
or a pharmaceutically acceptable salt thereof, wherein:
R₁-R₃₇ are independently selected from the group consisting of hydrogen and deuterium; and
at least one of R₁-R₃₇ is deuterium.

18. The method as recited in claim 17 wherein said adrenergic receptor mediated disorder is selected from the group consisting of asthma, chronic obstructive pulmonary disease, respiratory syncytial virus, pseudomonas aeruginosa, pneumoconiosis, exercise-induced bronchospasm, chronic bronchitis, and conditions associated with bronchoconstriction.

19. The method as recited in claim 17 further comprising the administration of an additional therapeutic agent.

20. The method as recited in claim 19 wherein said additional therapeutic agent is selected from the group consisting of adrenergics, anti-cholinergics, mast cell stabilizers, xanthines, leukotriene antagonists, glucocorticoids, decongestants, anti-tussives, mucolytics, anti-histamines, sepsis treatments, antibacterial agents, antifungal agents, anticoagulants, thrombolytics, non-steroidal anti-inflammatory agents, antiplatelet agents, NRIs, DARIs, SNRIs, sedatives, NDRIs, SNDRIs, monoamine oxidase inhibitors, hypothalamic phospholipids, ECE inhibitors, opioids, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIa Inhibitors and Factor Xa Inhibitors, renin inhibitors, NEP inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-muscarinic agents, beta-muscarinic agents, antiarrhythmic agents, diuretics, thrombolytic agents, anti-diabetic agents, mineralocorticoid receptor antagonists, growth hormone secretagogues, aP2 inhibitors, phosphodiesterase inhibitors, protein tyrosine kinase inhibitors, antiinflammatories, antiproliferatives, chemotherapeutic agents, immunosuppressants, anticancer agents and cytotoxic agents, antimetabolites, antibiotics, farnesyl-protein transferase inhibitors, hormonal agents, microtubule-disruptor agents, microtubule-stablizing agents, plant-derived products, epipodophyllotoxins, taxanes, topoisomerase inhibitors, prenyl-protein transferase inhibitors, cyclosporins, cytotoxic drugs, TNF-alpha inhibitors, anti-TNF antibodies and soluble TNF receptors, cyclooxygenase-2 (COX-2) inhibitors, and miscellaneous agents.

21. The method as recited in claim 20 wherein said additional therapeutic agent is is selected from the group consisting of adrenergics, anti-cholinergics, mast cell stabilizers, xanthines, leukotriene antagonists, glucocorticoids, decongestants, anti-tussives, mucolytics, and anti-histamines.

22. The method as recited in claim 21 wherein said glucocorticoid is fluticasone.

23. The method as recited in claim 17, further resulting in at least one effect selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

24. The method as recited in claim 17, further resulting in at least two effects selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

25. The method as recited in claim 17, wherein the method affects a decreased metabolism of the compound per dosage unit thereof by at least one polymorphically-expressed cytochrome P₄₅₀ isoform in the subject, as compared to the corresponding non-isotopically enriched compound.

26. The method as recited in claim 25, wherein the cytochrome P₄₅₀ isoform is selected from the group consisting of CYP2C8, CYP2C9, CYP2C19, and CYP2D6.

27. The method as recited claim 17, wherein said compound is characterized by decreased inhibition of at least one cytochrome P₄₅₀ or monoamine oxidase isoform in said subject per dosage unit thereof as compared to the non-isotopically enriched compound.

28. The method as recited in claim 27, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

29. The method as recited in claim 17, wherein the method reduces a deleterious change in a diagnostic hepatobiliary function endpoint, as compared to the corresponding non-isotopically enriched compound.

30. The method as recited in claim 29, wherein the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

31. A compound for use as a medicament, having structural Formula I: 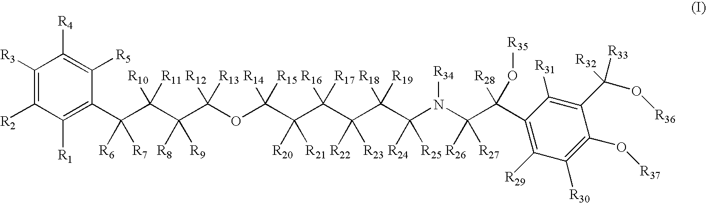
or a pharmaceutically acceptable salt thereof, wherein:
R₁-R₃₇ are independently selected from the group consisting of hydrogen and deuterium; and
at least one of R₁-R₃₇ is deuterium.

32. A compound for use in the manufacture of a medicament for the prevention or treatment of a disorder ameliorated by the modulation of adrenergic receptors, having structural Formula I: 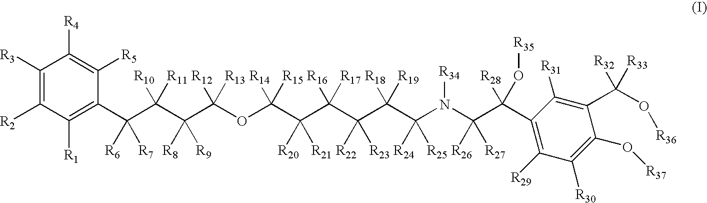
or a pharmaceutically acceptable salt thereof, wherein:
R₁-R₃₇ are independently selected from the group consisting of hydrogen or deuterium; and
at least one of R₁-R₃₇ is deuterium.

33. A deuterium-enriched compound of formula I or a pharmaceutically acceptable salt thereof: 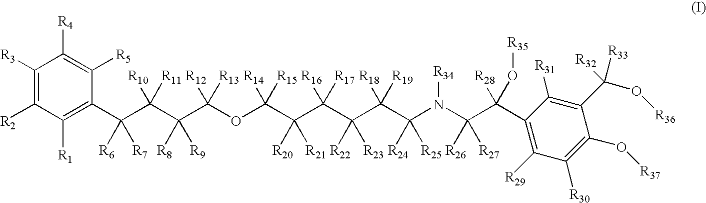
wherein R₁-R₃₇ are independently selected from H and D; and the abundance of deuterium in R₁-R₃₇ is at least 3%, provided that if R₅-R₆ and R₁₀ are D, then at least one other R is a D.

34. A deuterium-enriched compound of claim 33, wherein the abundance of deuterium in R₁-R₃₇ is selected from at least 3%, at least 5%, at least 11%, at least 16%, at least 22%, at least 27%, at least 32%, at least 38%, at least 43%, at least 49%, at least 54%, at least 59%, at least 65%, at least 70%, at least 76%, at least 81%, at least 86%, at least 92%, at least 97%, and 100%.

35. A deuterium-enriched compound of claim 33, wherein the abundance of deuterium in R₃₄-R₃₇ is selected from at least 25%, at least 50%, at least 75%, and 100%.

36. A deuterium-enriched compound of claim 33, wherein the abundance of deuterium in R₃₂-R₃₃, is selected from at least 50% and 100%.

37. A deuterium-enriched compound of claim 33, wherein the abundance of deuterium in R₂₉-R₃₁ is selected from at least 33%, at least 67%, and 100%.

38. A deuterium-enriched compound of claim 33, wherein the abundance of deuterium in R₂₆-R₂₈ is selected from at least 33%, at least 67%, and 100%.

39. A deuterium-enriched compound of claim 33, wherein the abundance of deuterium in R₁₄-R₂₅ is selected from at least 8%, at least 17%, at least 25%, at least 33%, at least 42%, at least 50%, at least 58%, at least 67%, at least 75%, at least 83%, at least 92%, and 100%.

40. A deuterium-enriched compound of claim 33, wherein the abundance of deuterium in R₆-R₁₃ is selected from at least 13%, at least 25%, at least 38%, at least 50%, at least 63%, at least 75%, at least 88%, and 100%.

41. A deuterium-enriched compound of claim 33, wherein the abundance of deuterium in R₁-R₅ is selected from at least 20%, at least 40%, at least 60%, at least 80%, and 100%.

42. A deuterium-enriched compound of claim 33, wherein the compound has a structural formula selected from the group consisting of:

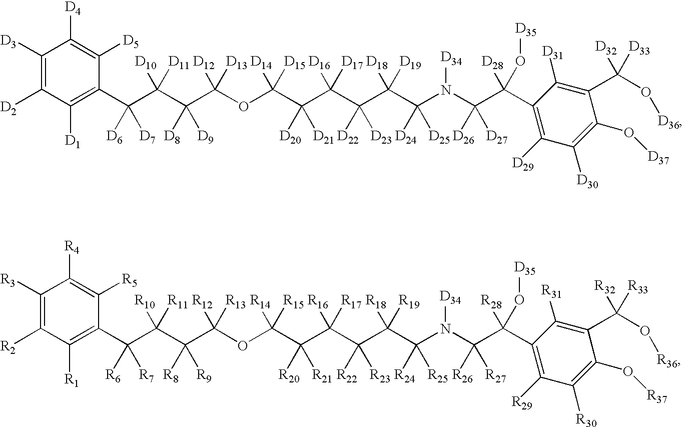

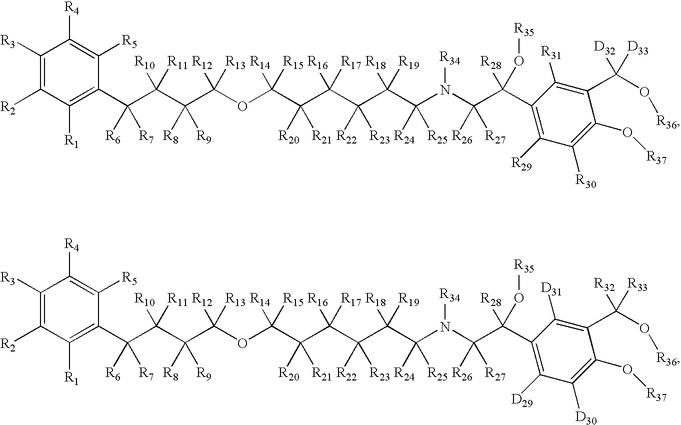

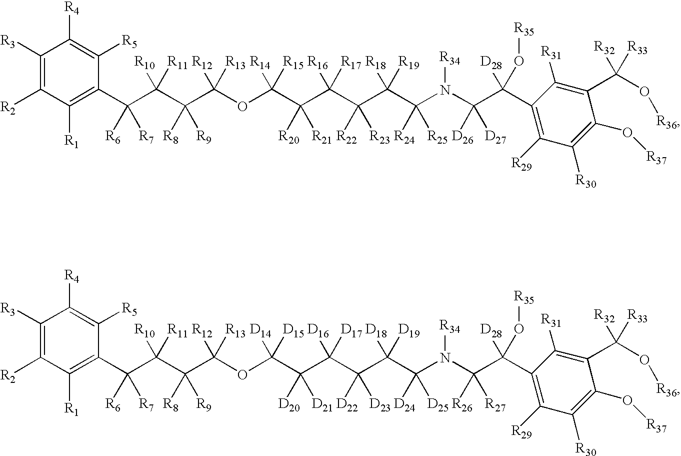

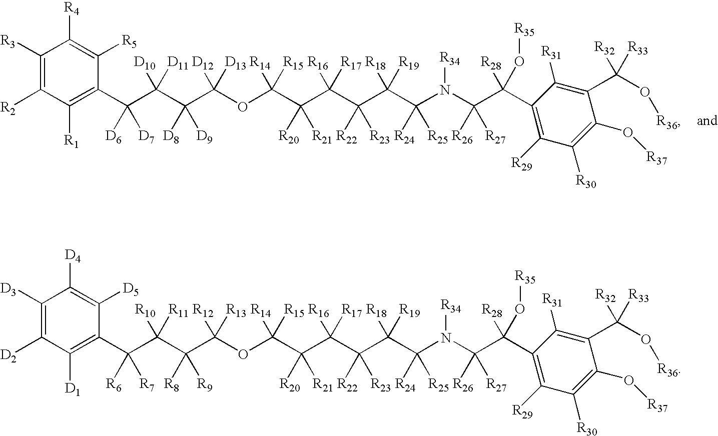

43. A deuterium-enriched compound of claim 33, wherein the compound has a structural formula selected from the group consisting of:

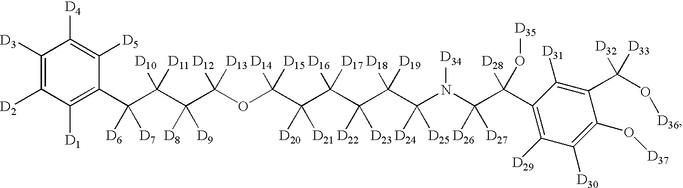

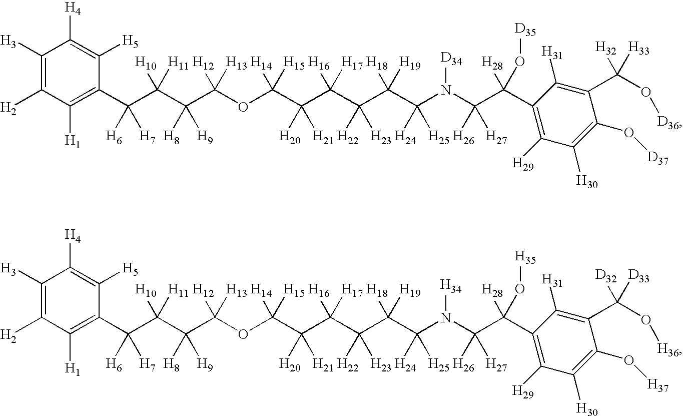

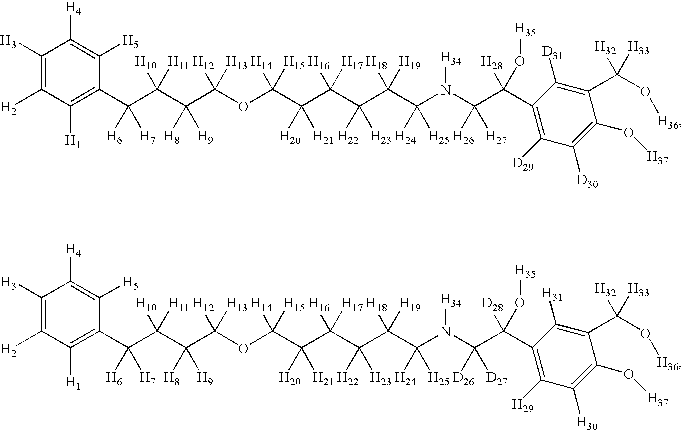

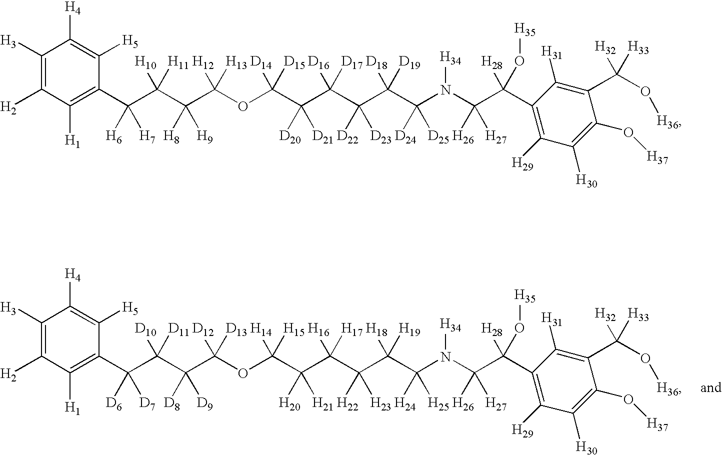

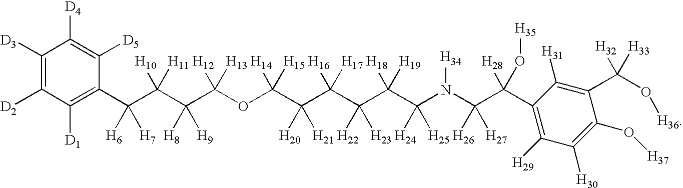

44. An isolated deuterium-enriched compound of formula lor a pharmaceutically acceptable salt thereof: 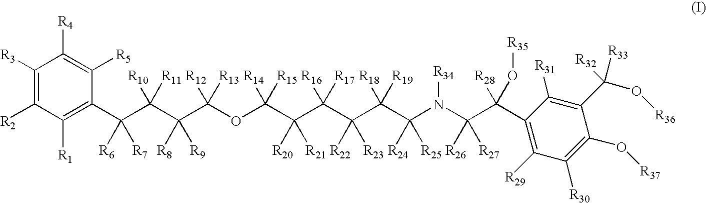
wherein R₁-R₃₇ are independently selected from H and D; and the abundance of deuterium in R₁-R₃₇ is at least 3%, provided that if R₅-R₆ and R₁₀ are D, then at least one other R is a D.

45. An isolated deuterium-enriched compound of claim 44, wherein the abundance of deuterium in R₁-R₃₇ is selected from at least 3%, at least 5%, at least 11%, at least 16%, at least 22%, at least 27%, at least 32%, at least 38%, at least 43%, at least 49%, at least 54%, at least 59%, at least 65%, at least 70%, at least 76%, at least 81%, at least 86%, at least 92%, at least 97%, and 100%.

46. An isolated deuterium-enriched compound of claim 44, wherein the abundance of deuterium in R₃₄-R₃₇ is selected from at least 25%, at least 50%, at least 75%, and 100%.

47. An isolated deuterium-enriched compound of claim 44, wherein the abundance of deuterium in R₃₂-R₃₃, is selected from at least 50% and 100%.

48. An isolated deuterium-enriched compound of claim 44, wherein the abundance of deuterium in R₂₉-R₃₁ is selected from at least 33%, at least 67%, and 100%.

49. A deuterium-enriched compound of claim 32, wherein the abundance of deuterium in R₂₆-R₂₈ is selected from at least 33%, at least 67%, and 100%.

50. An isolated deuterium-enriched compound of claim 44, wherein the abundance of deuterium in R₁₄-R₂₅ is selected from at least 8%, at least 17%, at least 25%, at least 33%, at least 42%, at least 50%, at least 58%, at least 67%, at least 75%, at least 83%, at least 92%, and 100%.

51. An isolated deuterium-enriched compound of claim 44, wherein the abundance of deuterium in R₆-R₁₃ is selected from at least 13%, at least 25%, at least 38%, at least 50%, at least 63%, at least 75%, at least 88%, and 100%.

52. An isolated deuterium-enriched compound of claim 44, wherein the abundance of deuterium in R₁-R₅ is selected from at least 20%, at least 40%, at least 60%, at least 80%, and 100%.

53. An isolated deuterium-enriched compound of claim 44, wherein the compound has a structural formula selected from the group consisting of:

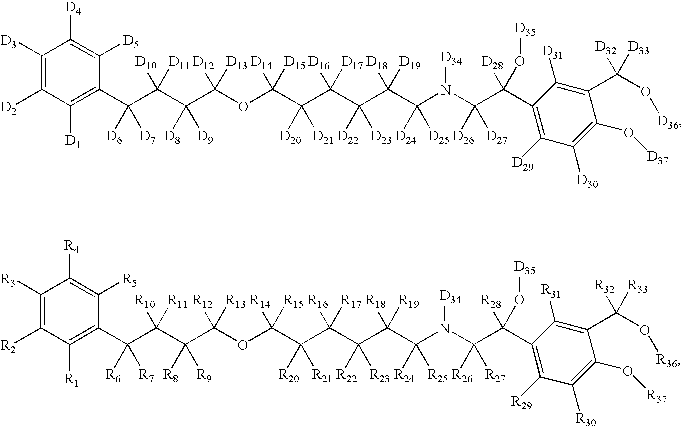

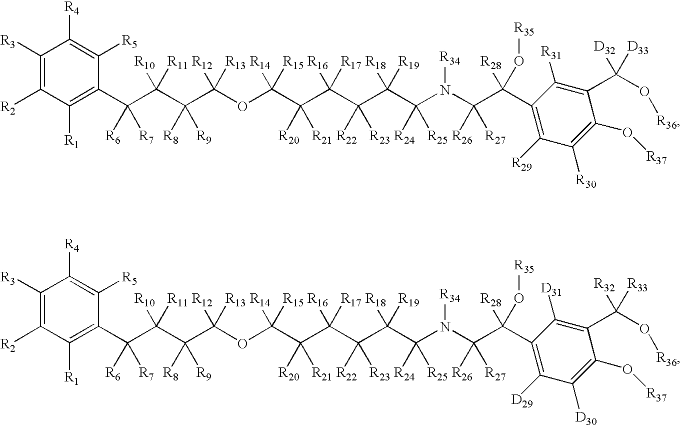

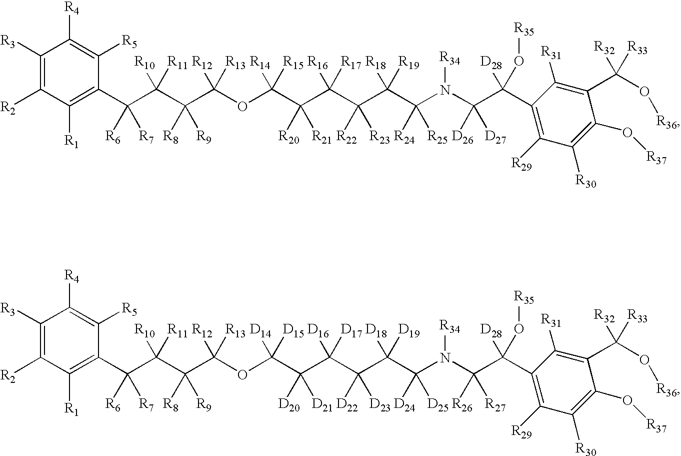

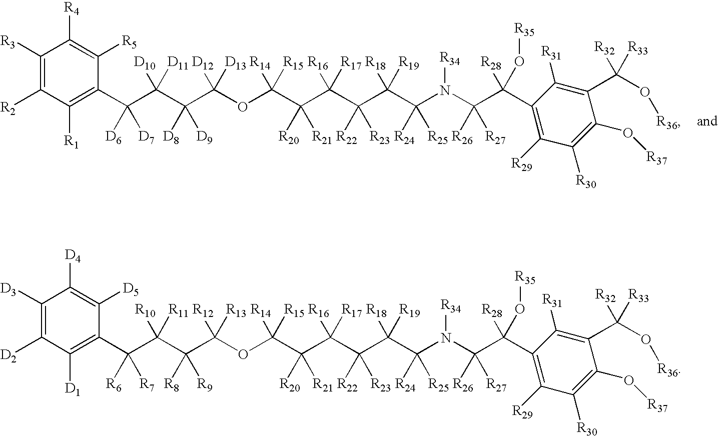

54. An isolated deuterium-enriched compound of claim 44, wherein the compound has a structural formula selected from the group consisting of:

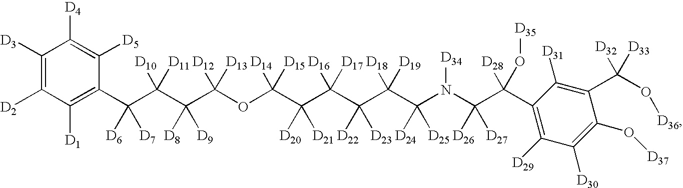

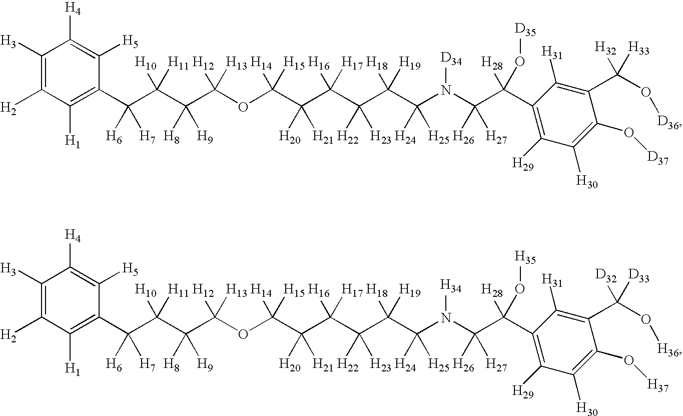

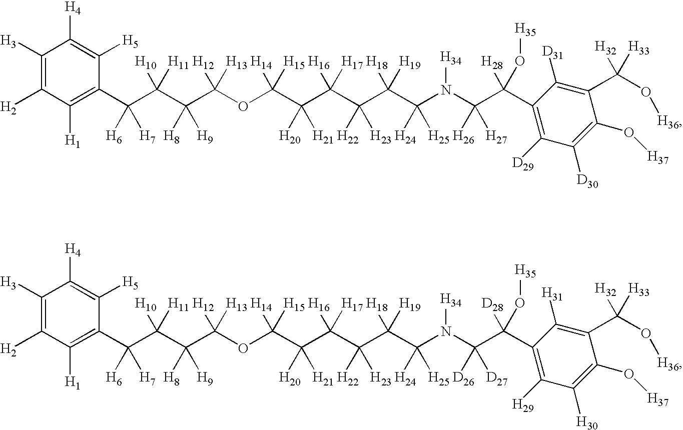

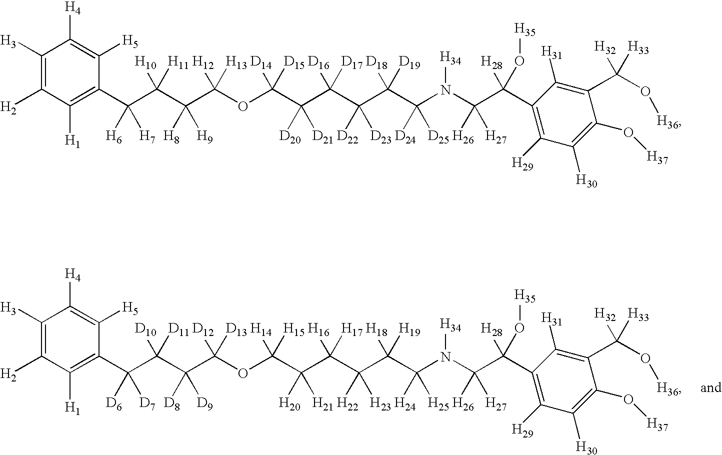

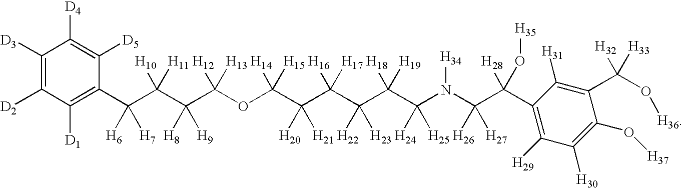

55. A mixture of deuterium-enriched compounds of formula lor a pharmaceutically acceptable salt thereof: 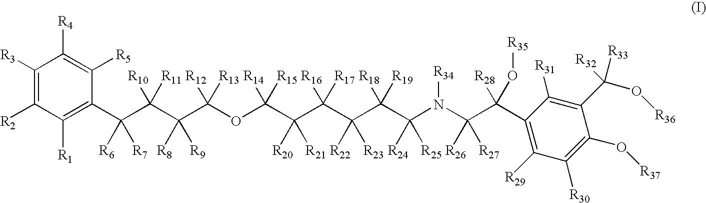
wherein R₁-R₃₇ are independently selected from H and D; and the abundance of deuterium in R₁-R₃₇ is at least 3%, provided that if R₅-R₆ and R₁₀ are D, then at least one other R is a D.

56. A mixture of deuterium-enriched compounds of claim 55, wherein the abundance of deuterium in R₁-R₃₇ is selected from at least 3%, at least 5%, at least 11%, at least 16%, at least 22%, at least 27%, at least 32%, at least 38%, at least 43%, at least 49%, at least 54%, at least 59%, at least 65%, at least 70%, at least 76%, at least 81%, at least 86%, at least 92%, at least 97%, and 100%.

57. A mixture of deuterium-enriched compounds of claim 55, wherein the abundance of deuterium in R₃₄-R₃₇ is selected from at least 25%, at least 50%, at least 75%, and 100%.

58. A mixture of deuterium-enriched compounds of claim 55, wherein the abundance of deuterium in R₃₂-R₃₃, is selected from at least 50% and 100%.

59. A mixture of deuterium-enriched compounds of claim 55, wherein the abundance of deuterium in R₂₉-R₃₁ is selected from at least 33%, at least 67%, and 100%.

60. A mixture of deuterium-enriched compounds of claim 55, wherein the abundance of deuterium in R₂₆-R₂₈ is selected from at least 33%, at least 67%, and 100%.

61. A mixture of deuterium-enriched compounds of claim 55, wherein the abundance of deuterium in R₁₄-R₂₅ is selected from at least 8%, at least 17%, at least 25%, at least 33%, at least 42%, at least 50%, at least 58%, at least 67%, at least 75%, at least 83%, at least 92%, and 100%.

62. A mixture of deuterium-enriched compounds of claim 55, wherein the abundance of deuterium in R₆-R₁₃ is selected from at least 13%, at least 25%, at least 38%, at least 50%, at least 63%, at least 75%, at least 88%, and 100%.

63. A mixture of deuterium-enriched compounds of claim 55, wherein the abundance of deuterium in R₁-R₅ is selected from at least 20%, at least 40%, at least 60%, at least 80%, and 100%.

64. A mixture of deuterium-enriched compounds of claim 55, wherein the compound has a structural formula selected from the group consisting of:

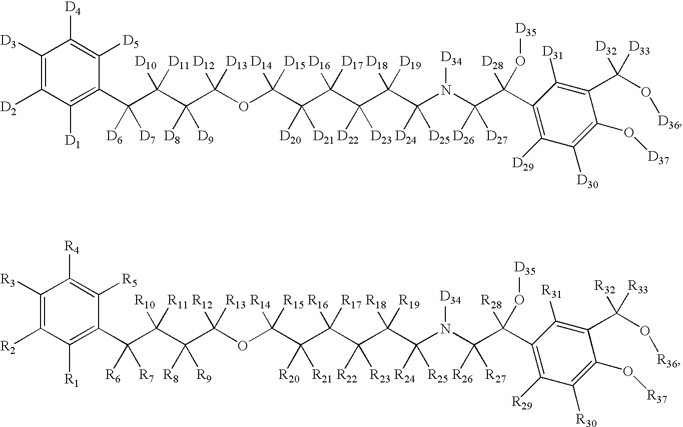

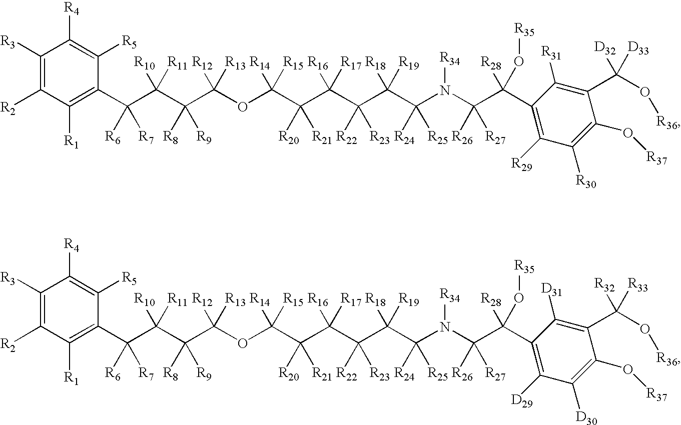

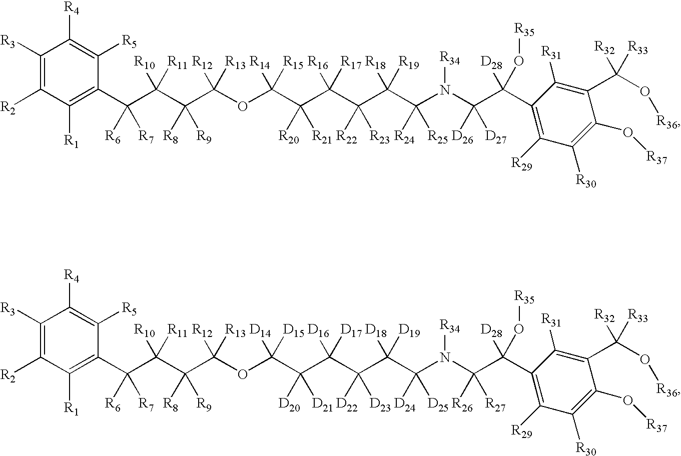

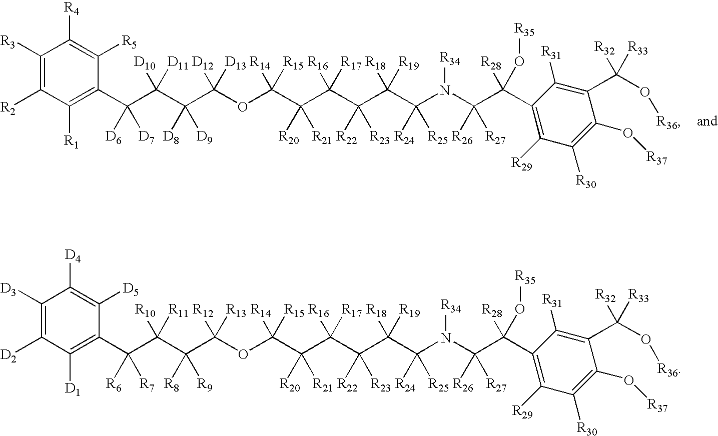

65. A mixture of deuterium-enriched compounds of claim 55, wherein the compound has a structural formula selected from the group consisting of:

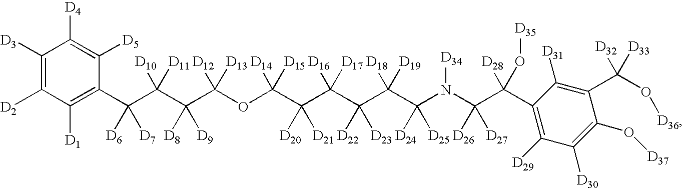

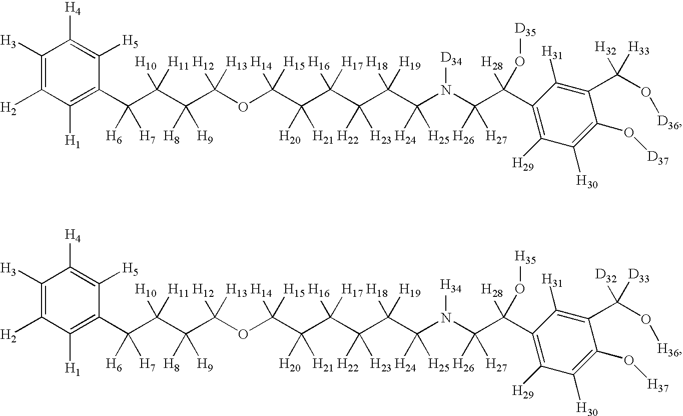

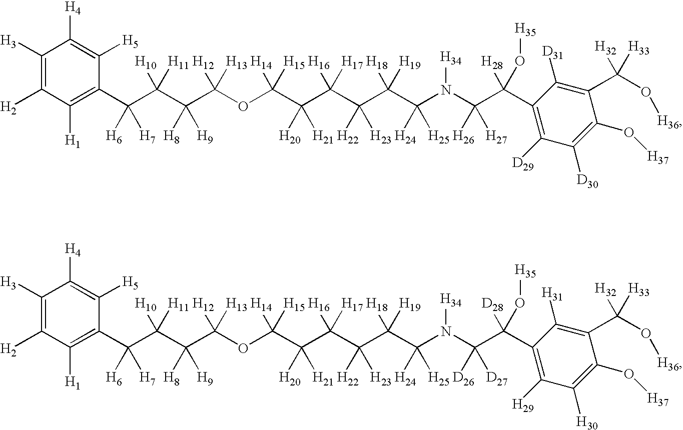

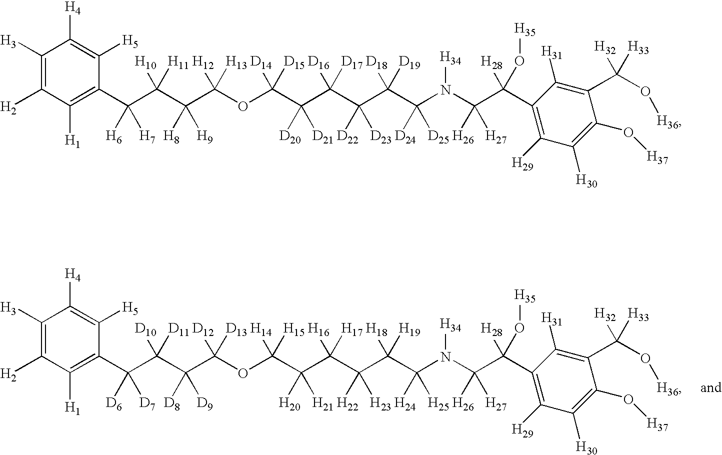

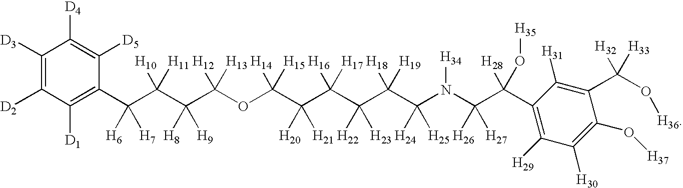

66. A pharmaceutical composition, comprising: a pharmaceutically acceptable carrier and a therapeutically effective amount of a compound of claim 33 or a pharmaceutically acceptable salt form thereof.

67. A method for treating asthma comprising: administering, to a patient in need thereof, a therapeutically effective amount of a compound of claim 33 or a pharmaceutically acceptable salt form thereof.
